SEQUENCE LISTING (79 sequences)

SEQ ID NO: 1 - protein, 5 aa, synthetic construct
  REGION 1..5: note = Synthetic Construct
INAMG

SEQ ID NO: 2 - protein, 19 aa, synthetic construct
  REGION 1..19: note = Synthetic Construct
GLSSNGDITRQNYAFYVKG

SEQ ID NO: 3 - protein, 6 aa, synthetic construct
  REGION 1..6: note = Synthetic Construct
MPPAST

SEQ ID NO: 4 - protein, 117 aa, synthetic construct
  REGION 1..117: note = Synthetic Construct
QVQLVESGGGLVQPGGSLRLSCSASGSTSNINAMGWYRQAPGKEREFVAGLSSNGDITRQNYAFYVKGRFTISRDNSKNTLYLQMNSLRAEDTAVYYCNEMPPASTWGQGTQVTVSS

SEQ ID NO: 5 - protein, 344 aa, synthetic construct
  REGION 1..344: note = Synthetic Construct
QVQLVESGGGLVQPGGSLRLSCSASGSTSNINAMGWYRQAPGKEREFVAGLSSNGDITRQNYAFYVKGRFTISRDNSKNTLYLQMNSLRAEDTAVYYCNEMPPASTWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 5 aa, synthetic construct
  REGION 1..5: note = Synthetic Construct
INAMA

SEQ ID NO: 7 - protein, 19 aa, synthetic construct
  REGION 1..19: note = Synthetic Construct
GVTSSGSIVRENYAFYVKG

SEQ ID NO: 8 - protein, 6 aa, synthetic construct
  REGION 1..6: note = Synthetic Construct
IPPYST

SEQ ID NO: 9 - protein, 117 aa, synthetic construct
  REGION 1..117: note = Synthetic Construct
EVQLVESGGGLVKPGGSLRLSCAASGSTSSINAMAWYRQAPGKQREFVAGVTSSGSIVRENYAFYVKGRFTISRDNAKNSLYLQMNSLRAEDTAVYYCNAIPPYSTWGQGTQVTVSS

SEQ ID NO: 10 - protein, 344 aa, synthetic construct
  REGION 1..344: note = Synthetic Construct
EVQLVESGGGLVKPGGSLRLSCAASGSTSSINAMAWYRQAPGKQREFVAGVTSSGSIVRENYAFYVKGRFTISRDNAKNSLYLQMNSLRAEDTAVYYCNAIPPYSTWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 10 aa, synthetic construct
  REGION 1..10: note = Synthetic Construct
RASSSVSYIH

SEQ ID NO: 12 - protein, 7 aa, synthetic construct
  REGION 1..7: note = Synthetic Construct
ATSNLAS

SEQ ID NO: 13 - protein, 9 aa, synthetic construct
  REGION 1..9: note = Synthetic Construct
QQWTSNPPT

SEQ ID NO: 14 - protein, 5 aa, synthetic construct
  REGION 1..5: note = Synthetic Construct
SYNMH

SEQ ID NO: 15 - protein, 17 aa, synthetic construct
  REGION 1..17: note = Synthetic Construct
AIYPGNGDTSYNQKFKG

SEQ ID NO: 16 - protein, 12 aa, synthetic construct
  REGION 1..12: note = Synthetic Construct
STYYGGDWYFNV

SEQ ID NO: 17 - protein, 106 aa, synthetic construct [sequence omitted]

SEQ ID NO: 18 - protein, 121 aa, synthetic construct [sequence omitted]

SEQ ID NO: 19 - protein, 213 aa, synthetic construct
  REGION 1..213: note = Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, synthetic construct
  REGION 1..451: note = Synthetic Construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 15 aa, synthetic construct
  REGION 1..15: note = Synthetic Construct
RASQSVSFAGTSLMH

SEQ ID NO: 22 - protein, 7 aa, synthetic construct
  REGION 1..7: note = Synthetic Construct
RASNLEA

SEQ ID NO: 23 - protein, 9 aa, synthetic construct
  REGION 1..9: note = Synthetic Construct
QQSREYPYT

SEQ ID NO: 24 - protein, 5 aa, synthetic construct
  REGION 1..5: note = Synthetic Construct
GYFMN

SEQ ID NO: 25 - protein, 17 aa, synthetic construct
  REGION 1..17: note = Synthetic Construct
RIHPYDGDTFYAQKFQG

SEQ ID NO: 26 - protein, 9 aa, synthetic construct
  REGION 1..9: note = Synthetic Construct
YDGSRAMDY

SEQ ID NO: 27 - protein, 111 aa, synthetic construct
  REGION 1..111: note = Synthetic Construct
DIVLTQSPASLAVSPGQRATITCRASQSVSFAGTSLMHWYQQKPGQPPKLLIYRASNLEAGVPARFSGSGSGTDFTLTINPVEANDAANYYCQQSREYPYTFGGGTKLEIK

SEQ ID NO: 28 - protein, 118 aa, synthetic construct
  REGION 1..118: note = Synthetic Construct
QVQLVQSGAEVKKPGASVKVSCKASGYTFTGYFMNWVRQAPGQGLEWIGRIHPYDGDTFYAQKFQGRVTMTRDTSTSTVYMELSSLRSEDTAVYYCTRYDGSRAMDYWGQGTTVTVSS

SEQ ID NO: 29 - protein, 218 aa, synthetic construct
  REGION 1..218: note = Synthetic Construct
DIVLTQSPASLAVSPGQRATITCRASQSVSFAGTSLMHWYQQKPGQPPKLLIYRASNLEAGVPARFSGSGSGTDFTLTINPVEANDAANYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 448 aa, synthetic construct
  REGION 1..448: note = Synthetic Construct
QVQLVQSGAEVKKPGASVKVSCKASGYTFTGYFMNWVRQAPGQGLEWIGRIHPYDGDTFYAQKFQGRVTMTRDTSTSTVYMELSSLRSEDTAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 11 aa, synthetic construct
  REGION 1..11: note = Synthetic Construct
RASQDVNTAVA

SEQ ID NO: 32 - protein, 7 aa, synthetic construct
  REGION 1..7: note = Synthetic Construct
SASFLYS

SEQ ID NO: 33 - protein, 9 aa, synthetic construct
  REGION 1..9: note = Synthetic Construct
QQHYTTPPT

SEQ ID NO: 34 - protein, 5 aa, synthetic construct
  REGION 1..5: note = Synthetic Construct
DTYIH

SEQ ID NO: 35 - protein, 17 aa, synthetic construct
  REGION 1..17: note = Synthetic Construct
RIYPTNGYTRYADSVKG

SEQ ID NO: 36 - protein, 11 aa, synthetic construct
  REGION 1..11: note = Synthetic Construct
WGGDGFYAMDY

SEQ ID NO: 37 - protein, 107 aa, synthetic construct [sequence omitted]

SEQ ID NO: 38 - protein, 120 aa, synthetic construct [sequence omitted]

SEQ ID NO: 39 - protein, 214 aa, synthetic construct
  REGION 1..214: note = Synthetic Construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 450 aa, synthetic construct
  REGION 1..450: note = Synthetic Construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 5 aa, synthetic construct
  REGION 1..5: note = Synthetic Construct
VNAYG

SEQ ID NO: 42 - protein, 16 aa, synthetic construct
  REGION 1..16: note = Synthetic Construct
IISAGGTTNYADSVKG

SEQ ID NO: 43 - protein, 10 aa, synthetic construct
  REGION 1..10: note = Synthetic Construct
QRRIGMLRDY

SEQ ID NO: 44 - protein, 118 aa, synthetic construct
  REGION 1..118: note = Synthetic Construct
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSS

SEQ ID NO: 45 - protein, 345 aa, synthetic construct
  REGION 1..345: note = Synthetic Construct
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 5 aa, synthetic construct
  REGION 1..5: note = Synthetic Construct
SYVMA

SEQ ID NO: 47 - protein, 17 aa, synthetic construct
  REGION 1..17: note = Synthetic Construct
SINWSSGRLIYADSVKG

SEQ ID NO: 48 - protein, 112 aa, synthetic construct
  REGION 1..112: note = Synthetic Construct
QVQLVESGGGLVQPGGSLRLSCAASGRTLESYVMAWFRQAPGKEREAVASINWSSGRLIYADSVKGRFTISRDNSKNTLYLQMNSLRAEDTAVYYCAAGRYWGQGTQVTVSS

SEQ ID NO: 49 - protein, 339 aa, synthetic construct
  REGION 1..339: note = Synthetic Construct
QVQLVESGGGLVQPGGSLRLSCAASGRTLESYVMAWFRQAPGKEREAVASINWSSGRLIYADSVKGRFTISRDNSKNTLYLQMNSLRAEDTAVYYCAAGRYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 11 aa, synthetic construct
  REGION 1..11: note = Synthetic Construct
KASQDVSIAVA

SEQ ID NO: 51 - protein, 7 aa, synthetic construct
  REGION 1..7: note = Synthetic Construct
SASYRYT

SEQ ID NO: 52 - protein, 9 aa, synthetic construct
  REGION 1..9: note = Synthetic Construct
QQHYITPLT

SEQ ID NO: 53 - protein, 5 aa, synthetic construct
  REGION 1..5: note = Synthetic Construct
NYGMN

SEQ ID NO: 54 - protein, 17 aa, synthetic construct
  REGION 1..17: note = Synthetic Construct
WINTYTGEPTYTDDFKG

SEQ ID NO: 55 - protein, 12 aa, synthetic construct
  REGION 1..12: note = Synthetic Construct
GGFGSSYWYFDV

SEQ ID NO: 56 - protein, 107 aa, synthetic construct
  REGION 1..107: note = Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 57 - protein, 121 aa, synthetic construct
  REGION 1..121: note = Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 58 - protein, 214 aa, synthetic construct
  REGION 1..214: note = Synthetic Construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 451 aa, synthetic construct
  REGION 1..451: note = Synthetic Construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 16 aa, synthetic construct
RSSQSLRDSDGKTYLN

SEQ ID NO: 61 - protein, 7 aa, synthetic construct
LVSKLDS

SEQ ID NO: 62 - protein, 9 aa, synthetic construct
WQGTHRPPT

SEQ ID NO: 63 - protein, 5 aa, synthetic construct
DYYMN

SEQ ID NO: 64 - protein, 17 aa, synthetic construct
DIDPGDGDTYYSQKFQG

SEQ ID NO: 65 - protein, 4 aa, synthetic construct
NLYI

SEQ ID NO: 66 - protein, 112 aa, synthetic construct
DVVMTQSPLSLPVTLGQPASISCRSSQSLRDSDGKTYLNWLQQRPGQSPRRLIYLVSKLDSGVPDRFSGSGSGTDFTLKISRVEAEDVGVYYCWQGTHRPPTFGQGTKLEIK

SEQ ID NO: 67 - protein, 113 aa, synthetic construct
QVQLVQSGAEVKKPGASVKLSCKASGYTFTDYYMNWVRQAPGQRLEWMGDIDPGDGDTYYSQKFQGRVTITRDTSASTAYMELSSLRSEDTAVYYCARNLYIYGQGTLVTVSS

SEQ ID NO: 68 - protein, 219 aa, synthetic construct
DVVMTQSPLSLPVTLGQPASISCRSSQSLRDSDGKTYLNWLQQRPGQSPRRLIYLVSKLDSGVPDRFSGSGSGTDFTLKISRVEAEDVGVYYCWQGTHRPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 443 aa, synthetic construct
QVQLVQSGAEVKKPGASVKLSCKASGYTFTDYYMNWVRQAPGQRLEWMGDIDPGDGDTYYSQKFQGRVTITRDTSASTAYMELSSLRSEDTAVYYCARNLYIYGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 15 aa, synthetic construct
KASQSVDYEGDSFLN

SEQ ID NO: 71 - protein, 7 aa, synthetic construct
AASNLES

SEQ ID NO: 72 - protein, 9 aa, synthetic construct
QQSNEDPLT

SEQ ID NO: 73 - protein, 5 aa, synthetic construct
SYWIE

SEQ ID NO: 74 - protein, 17 aa, synthetic construct
EILPGGGDTNYNEIFKG

SEQ ID NO: 75 - protein, 8 aa, synthetic construct
RVPIRLDY

SEQ ID NO: 76 - protein, 111 aa, synthetic construct [sequence omitted]

SEQ ID NO: 77 - protein, 117 aa, synthetic construct [sequence omitted]

SEQ ID NO: 78 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 447 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0041782 A1
(43) Pub. Date: Feb. 12, 2026

(54) ANTIBODY-DRUG CONJUGATES, PHARMACEUTICAL COMPOSITIONS, AND THERAPEUTIC APPLICATIONS

(71) Applicant: Shanghai Ruotuo Biosciences, Shanghai (CN)

(72) Inventors: Hong Fu, Belmont, CA (US); Ziyang Zhong, Belmont, CA (US)

(21) Appl. No.: 19/099,553

(22) PCT Filed: Aug. 14, 2023
(86) PCT No.: PCT/CN2023/112924

§ 371 Date: Jan. 29, 2025

Related U.S. Application Data
(60) Provisional application No. 63/371,400, filed on Aug. 15, 2022.
(60) Provisional application No. 63/483,739, filed on Feb. 7, 2023.

(51) Int. Cl. A61K 47/68; A61K 9/00; A61P 35/00; C07K 16/18; C07K 16/28; C07K 16/30; C07K 16/32

(52) U.S. Cl. CPC A61K 47/68037; A61K 9/0019; A61K 47/6849; A61K 47/6855; A61K 47/6889; A61P 35/00; C07K 16/18; C07K 16/28; C07K 16/2803; C07K 16/2827; C07K 16/2887; C07K 16/30; C07K 16/32

(57) ABSTRACT

Provided herein are antibody-drug conjugates, for example, a compound of Formula (I), and pharmaceutical compositions thereof. Also provided herein are methods of their use for treating, preventing, or ameliorating one or more symptoms of a proliferative disease. Additionally, provided herein are functionalized compounds, for example, a compound of Formula (IA), for conjugation with an antibody.

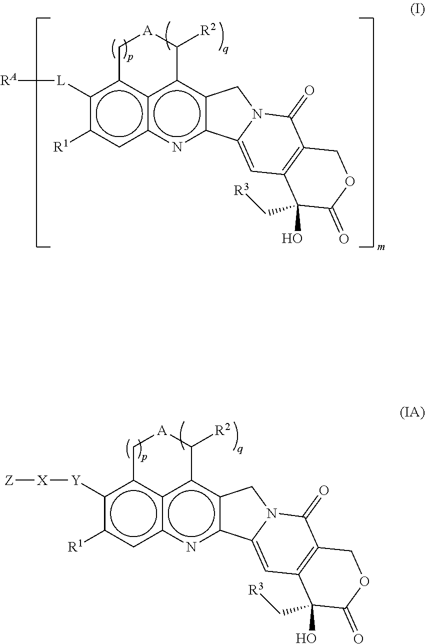

### CROSS REFERENCE TO RELATED APPLICATION

This application claims the benefit of the priority of U.S. Provisional Application Nos. 63/371,400, filed Aug. 15, 2022; and 63/483,739, filed Feb. 7, 2023; the disclosure of each of which is incorporated herein by reference in its entirety.

### FIELD

Provided herein are antibody-drug conjugates and pharmaceutical compositions thereof. Also provided herein are methods of their use for treating, preventing, or ameliorating one or more symptoms of a proliferative disease. Additionally, provided herein are functionalized compounds for conjugation with an antibody.

### REFERENCE TO A SEQUENCE LISTING

The present specification is being filed with a Sequence Listing entitled 216A018WO01_SEQLIST_ST26. XML of 98,618 bytes in size and created Aug. 13, 2023; the content of which is incorporated herein by reference in its entirety.

### BACKGROUND

An antibody-drug conjugate (ADC) is a monoclonal antibody tethered to a cytotoxic drug (also known as a payload or warhead) via a chemical linker. Polakis, Pharmacol. Rev. 2016, 68, 3-19; Beck et al., Nat. Rev. Drug. Discov. 2017, 16, 315-37; Birrer et al., J. Natl. Cancer Inst. 2019, 111, 538-49; Chau et al., Lancet 2019, 394, 793-804. Thus, an ADC is a targeted biopharmaceutical drug that, via the specificity of its monoclonal antibody to a cancer cell surface antigen, delivers and releases its cytotoxic payload directly to targeted cancer cells, resulting in a greater potency and a broader therapeutic window as compared with the cytotoxic drug by itself. Polakis, Pharmacol. Rev. 2016, 68, 3-19; Chau et al., Lancet 2019, 394, 793-804. Mechanistically, an ADC exerts its biological functions by binding to a targeted cancer cell selectively, followed by the internalization of an ADC-antigen complex formed on the cell surface via clathrin-mediated endocytosis and the release of the cell-killing payload inside the targeted cancer cell. Chau et al., Lancet 2019, 394, 793-804.

Despite the advances in cancer treatment, cancer remains a major worldwide public health problem. It was estimated that there will be 1,918,030 new cancer cases diagnosed and 609,360 cancer deaths in the US alone in 2022. Cancer Facts & Figures 2022. Cancer is the second-leading cause of death worldwide. Therefore, there is a need for effective therapeutics for cancer treatment.

### SUMMARY OF THE DISCLOSURE

Provided herein is a compound of Formula (I):

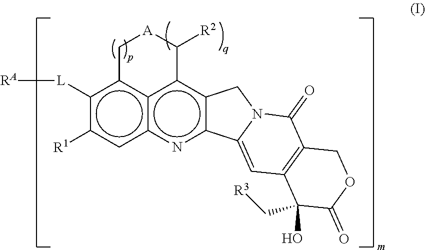

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each A is independently O, S, S(O), S(O₂), or N(R⁴);
each L is independently a linker;
each R¹, R², and R³ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O) NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O) NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁴ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR¹a)NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
R^{A} is an antibody or an antigen-binding fragment thereof;
m is an integer of 1, 2, 3, 4, 5, 6, 7, 8, 9, 10, 11, 12, 13, 14, 15, or 16; and
each p and q is independently an integer of 0, 1, or 2; with the proviso that the total of p and q is greater than 0;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O) OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{e}S(O)₂R^{d}, —NR^{e}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S) NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S) NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally provided herein is a method of treating, preventing, or ameliorating one or more symptoms of a proliferative disease in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of inhibiting the growth of a cell, comprising contacting the cell with a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

Provided herein is a compound of Formula (IA):

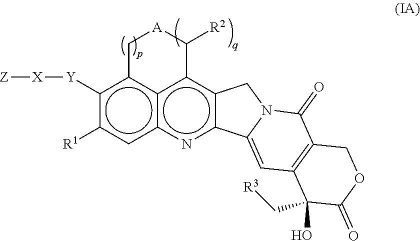

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein:
A is O, S, S(O), S(O₂), or N(R⁴);
X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
Y is a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene;
Z is

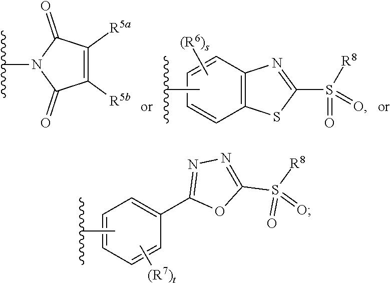

each R¹, R², R³, R^{5a}, and R^{5b} is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O) NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O) NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O) NR^{1b}R^{1c}, —NR^{1a}C(O)SR^{1d}, —NR^{1a}C(NR^{1d})NR^{1b}R^{1c}, —NR^{1d}C(S)R^{1d}, —NR^{1a}C(S)OR^{1d}, —NR^{1d}C(S)NR^{1b}R^{1c}, —NR^{1d}S(O)R^{1d}, —NR^{1a}S(O)₂R^{1d}, —NR^{1d}S(O)NR^{1b}R^{1c}, —NR^{1a}S(O)₂NR^{1b}R^{1c}, —SR^{1a}, —S(O)R^{1d}, —S(O)₂R^{1a}, —S(O)NR^{1b}R^{1c}, or —S(O)₂NR^{1b}R^{1c};
R⁴ is (i) hydrogen or deuterium; (ii) C₁₋₆ alkyl, C₁₋₆ heteroalkyl, C₂₋₆ alkenyl, C₂₋₆alkynyl, C₃₋₁₀ cycloalkyl, C₆₋₁₄ aryl, C₇₋₁₅ aralkyl, heteroaryl, or heterocyclyl; or (iii) —C(O)R¹, —C(O)OR^{1a}, —C(O)NR^{1b}R^{1c}, —C(O)SR^{1a}, —C(NR^{1a})NR^{1b}R^{1c}, —C(S)R^{1d}, —C(S)OR^{1a}, —C(S)NR^{1b}R^{1c}, —S(O)R^{1d}, —S(O)₂R^{1a}, —S(O)NR^{1b}R^{1c}, or —S(O)₂NR^{1b}R^{1c};
each R⁶ and R⁷ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1d}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1d}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1d}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1d}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁸ is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
p and q are each independently an integer of 0, 1, or 2; with the proviso that the total of p and q is greater than 0;
s is an integer of 0, 1, 2, or 3; and
t is an integer of 0, 1, 2, 3, or 4;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O) OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};

wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S) NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{e}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S) NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g} and —S(O)₂NR^{f}R^{g}: wherein each R^{e}, R^{f}, R^{g} and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

### BRIEF DESCRIPTION OF THE DRAWINGS

FIG. 1 shows the anticancer effects of ADC compounds ADC-C2, ADC-C8, ADC-C29, ADC-C52, and trastuzumab deruxtecan (DS-8201) in a xenograft mouse model for human breast cancer (JIMT-1).

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, immunology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause (s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, and commensurate with a reasonable benefit/risk ratio. See, e.g., Remington: The Science and Practice of Pharmacy, 22nd ed.; Allen Ed.; Pharmaceutical Press: London, 2012; Handbook of Pharmaceutical Excipients, 8th ed.; Sheskey et al., Eds.; Pharmaceutical Press: London, 2017; Handbook of Pharmaceutical Additives, 3rd ed.; Ash and Ash Eds.; Synapse Information Resources: 2007; Pharmaceutical Preformulation and Formulation, 2nd ed.; Gibson Ed.; Drugs and the Pharmaceutical Sciences 199; Informa Healthcare: New York, NY, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The term “heteroalkyl” refers to a linear or branched saturated monovalent hydrocarbon radical that contains one or more heteroatoms on its main chain, each independently selected from O, S, and N. The heteroalkyl is optionally substituted with one or more substituents Q as described herein. For example, C₁₋₆ heteroalkyl refers to a linear saturated monovalent hydrocarbon radical of 1 to 6 carbon atoms or a branched saturated monovalent hydrocarbon radical of 3 to 6 carbon atoms. In certain embodiments, the heteroalkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkyl groups are also referred as “lower heteroalkyl.” Examples of heteroalkyl groups include, but are not limited to, —OCH₃, —OCH₂CH₃, —CH₂OCH₃, —NHCH₃, —ONHCH₃, —NHOCH₃, —SCH₃, —CH₂NHCH₂CH₃, and —NHCH₂CH₂CH₃. Examples of substituted heteroalkyl groups include, but are not limited to, —CH₂NHC(O)CH₃ and —NHC(O)CH₂CH₃.

The terms “alkylene” and “alkanediyl” are used interchangeably herein in reference to a linear or branched saturated divalent hydrocarbon radical, wherein the alkanediyl is optionally substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1, 1-diyl and ethane-1, 2-diyl), propanediyl (including all isomeric forms, e.g., propane-1, 1-diyl, propane-1, 2-diyl, and propane-1, 3-diyl), butanediyl (including all isomeric forms, e.g., butane-1, 1-diyl, butane-1, 2-diyl, butane-1, 3-diyl, and butane-1, 4-diyl), pentanediyl (including all isomeric forms, e.g., pentane-1, 1-diyl, pentane-1, 2-diyl, pentane-1, 3-diyl, and pentane-1, 5-diyl), and hexanediyl (including all isomeric forms, e.g., hexane-1, 1-diyl, hexane-1, 2-diyl, hexane-1, 3-diyl, and hexane-1, 6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O) (CH₂)₆—, —C(O)(CH₂)₇—, —C(O)(CH₂)₈—, —C(O)(CH₂)₉—, —C(O)(CH₂)₁₀—, —C(O) CH₂C(O)—, —C(O)(CH₂)₂C(O)—, —C(O)(CH₂)₃C(O)—, —C(O)(CH₂)₄C(O)—, or —C(O)(CH₂)₅C(O)—.

The terms “heteroalkylene” and “heteroalkanediyl” are used interchangeably herein in reference to a linear or branched saturated divalent hydrocarbon radical that contains one or more heteroatoms in its main chain, each independently selected from O, S, and N. The heteroalkylene is optionally substituted with one or more substituents Q as described herein. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —(CH₂)₂O—, —(CH₂)₃O—, —(CH₂)₄O—, —(CH₂)₅O—, —(CH₂)₆O—, —(CH₂)₇O—, —(CH₂)₈O—, —(CH₂)₉O—, —(CH₂)₁₀O—, —CH₂OCH₂—, —CH₂CH₂O—, —(CH₂CH₂O)₂—, —(CH₂CH₂O)₃—, —(CH₂CH₂O)₄—, —(CH₂CH₂O)₅—, —CH₂NH—, —CH₂NHCH₂—, —CH₂CH₂NH—, —CH₂S—, —CH₂SCH₂—, and —CH₂CH₂S—. Examples of substituted heteroalkylene groups include, but are not limited to, —C(O)CH₂O—, —C(O)(CH₂)₂O—, —C(O)(CH₂)₃O—, —C(O)(CH₂)₄O—, —C(O)(CH₂)₅O—, —C(O)(CH₂)₆O—, —C(O)(CH₂)₇O—, —C(O)(CH₂)₉O—, —C(O)(CH₂)₉O—, —C(O)(CH₂)₁₀O—, —C(O)CH₂OCH₂CH₂O—, —C(O)CH₂O(CH₂CH₂O), —C(O)CH₂O (CH₂CH₂)₃—, —C(O)CH₂O(CH₂CH₂O)₄, —C(O)CH₂O(CH₂CH₂O)₅—, —CH₂NHC(O)CH₂—, or —CH₂CH₂C(O)NH—.

The term “alkenyl” refers to a linear or branched monovalent hydrocarbon radical, which contains one or more, in one embodiment, one, two, three, or four, in another embodiment, one, carbon-carbon double bond (s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond (s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1, 1-diyl and ethene-1, 2-diyl), propenediyl (including all isomeric forms, e.g., 1-propene-1, 1-diyl, 1-propene-1, 2-diyl, and 1-propene-1, 3-diyl), butenediyl (including all isomeric forms, e.g., 1-butene-1, 1-diyl, 1-butene-1, 2-diyl, and 1-butene-1, 4-diyl), pentenediyl (including all isomeric forms, e.g., 1-pentene-1, 1-diyl, 1-pentene-1, 2-diyl, and 1-pentene-1, 5-diyl), and hexenediyl (including all isomeric forms, e.g., 1-hexene-1, 1-diyl, 1-hexene-1, 2-diyl, 1-hexene-1, 3-diyl, 1-hexene-1, 4-diyl, 1-hexene-1, 5-diyl, and 1-hexene-1, 6-diyl).

The terms “heteroalkenylene” and “heteroalkenediyl” are used interchangeably herein in reference to a linear or branched divalent hydrocarbon radical, which contains one or more, in one embodiment, one, two, three, or four, in another embodiment, one, carbon-carbon double bond (s), and which contains one or more heteroatoms each independently selected from O, S, and N in the hydrocarbon chain. The heteroalkenylene is optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, or four, in another embodiment, one, carbon-carbon triple bond (s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond (s). The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1, 3-diyl and 1-propyne-3, 3-diyl), butynediyl (including all isomeric forms, e.g., 1-butyne-1, 3-diyl, 1-butyne-1, 4-diyl, and 2-butyne-1, 1-diyl), pentynediyl (including all isomeric forms, e.g., 1-pentyne-1, 3-diyl, 1-pentyne-1, 4-diyl, and 2-pentyne-1, 1-diyl), and hexynediyl (including all isomeric forms, e.g., 1-hexyne-1, 3-diyl, 1-hexyne-1, 4-diyl, and 2-hexyne-1, 1-diyl).

The terms “heteroalkynylene” and “heteroalkynediyl” are used interchangeably herein in reference to a linear or branched divalent hydrocarbon radical, which contains one or more, in one embodiment, one, two, three, or four, in another embodiment, one, carbon-carbon triple bond (s), and which contains one or more heteroatoms in its main chain, each independently selected from O, S, and N. The heteroalkynylene is optionally substituted with one or more substituents Q as described herein. For example, C₂₋₆ heteroalkynylene refers to a linear unsaturated divalent hydrocarbon radical of 2 to 6 carbon atoms or a branched unsaturated divalent hydrocarbon radical of 4 to 6 carbon atoms. In certain embodiments, the heteroalkynylene is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of heteroalkynylene groups include, but are not limited to, —C≡CCH₂O—, —C≡CCH₂S—, or —C≡CCH₂NH—.

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo [1.1.1]pentyl, bicyclo [2.1.1]hexyl, bicyclo [2.2.1]heptyl, bicyclo [2.2.2]-octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃_7) carbon atoms. Examples of cycloalkanediyl groups include, but are not limited to, cyclopropanediyl (including all isomeric forms, e.g., cyclopropane-1, 1-diyl and cyclopropane-1, 2-diyl), cyclobutanediyl (including all isomeric forms, e.g., cyclobutane-1, 1-diyl, cyclobutane-1, 2-diyl, and cyclobutane-1, 3-diyl), cyclopentanediyl (including all isomeric forms, e.g., cyclopentane-1, 1-diyl, cyclopentane-1, 2-diyl, and cyclopentane-1, 3-diyl), cyclohexanediyl (including all isomeric forms, e.g., cyclohexane-1, 1-diyl, cyclohexane-1, 2-diyl, cyclohexane-1, 3-diyl, and cyclohex-1, 4-diyl), cycloheptanediyl (including all isomeric forms, e.g., cycloheptane-1, 1-diyl, cycloheptane-1, 2-diyl, cycloheptane-1, 3-diyl, and cycloheptane-1, 4-diyl), decalinediyl (including all isomeric forms, e.g., decaline-1, 1-diyl, decaline-1, 2-diyl, and decaline-1, 8-diyl), and adamantdiyl (including all isomeric forms, e.g., adamant-1, 2-diyl, adamant-1, 3-diyl, and adamant-1, 8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1, 2-diyl, phen-1, 3-diyl, and phen-1, 4-diyl), naphthylene (including all isomeric forms, e.g., naphth-1, 2-diyl, naphth-1, 3-diyl, and naphth-1, 8-diyl), fluorenylene (including all isomeric forms, e.g., fluoren-1, 2-diyl, fluoren-1, 3-diyl, and fluoren-1, 8-diyl), azulenylene (including all isomeric forms, e.g., azulen-1, 2-diyl, azulen-1, 3-diyl, and azulen-1, 8-diyl), anthrylene (including all isomeric forms, e.g., anthr-1, 2-diyl, anthr-1, 3-diyl, and anthr-1, 8-diyl), phenanthrylene (including all isomeric forms, e.g., phenanthr-1, 2-diyl, phenanthr-1, 3-diyl, and phenanthr-1, 8-diyl), pyrenylene (including all isomeric forms, e.g., pyren-1, 2-diyl, pyren-1, 3-diyl, and pyren-1, 8-diyl), biphenylene (including all isomeric forms, e.g., biphen-2, 3-diyl, biphen-3, 4′-diyl, and biphen-4, 4′-diyl), and terphenylene (including all isomeric forms, e.g., terphen-2, 3-diyl, terphen-3, 4′-diyl, and terphen-4, 4′-diyl). Arylene also refers to bicyclic or tricyclic carbon rings, where one of the rings is aromatic and the others of which may be saturated, partially unsaturated, or aromatic, for example, dihydronaphthylene (including all isomeric forms, e.g., dihydronaphth-1, 2-diyl and dihydronaphth-1, 8-diyl), indenylene (including all isomeric forms, e.g., inden-1, 2-diyl, inden-1, 5-diyl, and inden-1, 7-diyl), indanylene (including all isomeric forms, e.g., indan-1, 2-diyl, indan-1, 5-diyl, and indan-1, 7-diyl), or tetrahydronaphthylene (tetralinylene) (including all isomeric forms, e.g., tetrahydronaphth-1, 2-diyl, tetrahydronaphth-1, 5-diyl, and tetrahydronaphth-1, 8-diyl). In certain embodiments, arylene is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “aralkylene” or “arylalkylene” refers to a divalent alkyl group substituted with one or more aryl groups. In certain embodiments, the aralkylene has from 7 to 30 (C₇₋₃₀), from 7 to 20 (C₇₋₂₀), or from 7 to 16 (C₇₋₁₆) carbon atoms. Examples of aralkylene groups include, but are not limited to, benzylene (including all isomeric forms, e.g., phenylmethdiyl), phenylethylene (including all isomeric forms, e.g., 2-phenyl-ethan-1, 1-diyl and 2-phenyl-ethan-1, 2-diyl), and phenylpropylene (including all isomeric forms, e.g., 3-phenyl-propan-1, 1-diyl, 3-phenyl-propan-1, 2-diyl, and 3-phenyl-propan-1, 3-diyl). In certain embodiments, the aralkylene is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo [2, 3-b]pyridinyl, furo [2, 3-c]pyridinyl, furo [3, 2-b]pyridinyl, furo [3, 2-c]pyridinyl, furo [3, 4-b]pyridinyl, and furo [3, 4-c]pyridinyl), imidazopyridinyl (including all isomeric forms, e.g., imidazo [1, 2-a]pyridinyl, imidazo [4, 5-b]pyridinyl, and imidazo [4, 5-c]pyridinyl), imidazothiazolyl (including all isomeric forms, e.g., imidazo [2, 1-b]thiazolyl and imidazo [4, 5-d]thiazolyl), indazolyl, indolizinyl, indolyl, isobenzofuranyl, isobenzothienyl (i.e., benzo [c]thienyl), isoindolyl, isoquinolinyl, naphthyridinyl (including all isomeric forms, e.g., 1, 5-naphthyridinyl, 1, 6-naphthyridinyl, 1, 7-naphthyridinyl, and 1, 8-naphthyridinyl), oxazolopyridinyl (including all isomeric forms, e.g., oxazolo [4, 5-b]pyridinyl, oxazolo [4, 5-c]pyridinyl, oxazolo [5, 4-b]pyridinyl, and oxazolo [5, 4-c]pyridinyl), phthalazinyl, pteridinyl, purinyl, pyrrolopyridyl (including all isomeric forms, e.g., pyrrolo [2, 3-b]pyridinyl, pyrrolo [2, 3-c]pyridinyl, pyrrolo [3, 2-b]pyridinyl, and pyrrolo [3,2-c]pyridinyl), quinolinyl, quinoxalinyl, quinazolinyl, thiadiazolopyrimidyl (including all isomeric forms, e.g., [1, 2, 5]thiadiazolo [3,4-d]pyrimidinyl and [1, 2, 3]thiadiazolo [4, 5-d]pyrimidinyl), and thienopyridyl (including all isomeric forms, e.g., thieno [2, 3-b]pyridinyl, thieno [2, 3-c]pyridinyl, thieno [3, 2-b]pyridinyl, and thieno [3, 2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1, 5-phenanthrolinyl, 1, 6-phenanthrolinyl, 1, 7-phenanthrolinyl, 1, 9-phenanthrolinyl, and 2, 10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. A heteroarylene group has at least one linkage to the rest of a molecule via its aromatic ring (s). Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms, e.g., furo [2, 3-b]pyridindiyl, furo [2, 3-c]pyridindiyl, furo [3, 2-b]pyridindiyl, furo [3, 2-c]pyridindiyl, furo [3, 4-b]pyridindiyl, and furo [3, 4-c]pyridindiyl), imidazopyridindiyl (including all isomeric forms, e.g., imidazo [1, 2-a]pyridindiyl, imidazo [4, 5-b]pyridindiyl, and imidazo [4, 5-c]pyridindiyl), imidazothiazoldiyl (including all isomeric forms, e.g., imidazo [2, 1-b]thiazoldiyl and imidazo [4, 5-d]thiazoldiyl), indazoldiyl, indolizindiyl, indoldiyl, isobenzofurandiyl, isobenzothiendiyl (i.e., benzo [c]thiendiyl), isoindoldiyl, isoquinolindiyl, naphthyridindiyl (including all isomeric forms, e.g., 1, 5-naphthyridindiyl, 1, 6-naphthyridindiyl, 1, 7-naphthyridindiyl, and 1, 8-naphthyridindiyl), oxazolopyridindiyl (including all isomeric forms, e.g., oxazolo [4, 5-b]pyridindiyl, oxazolo [4, 5-c]pyridindiyl, oxazolo [5, 4-b]pyridindiyl, and oxazolo [5, 4-c]pyridindiyl), phthalazindiyl, pteridindiyl, purindiyl, pyrrolopyridindiyl (including all isomeric forms, e.g., pyrrolo [2, 3-b]pyridindiyl, pyrrolo [2, 3-c]pyridindiyl, pyrrolo [3, 2-b]pyridindiyl, and pyrrolo [3, 2-c]pyridindiyl), quinolindiyl, quinoxalindiyl, quinazolindiyl, thiadiazolopyrimidindiyl (including all isomeric forms, e.g., [1, 2, 5]thiadiazolo [3,4-d]pyrimidindiyl and thiadiazolo [4, 5-d]pyrimidindiyl), and thienopyridindiyl (including all isomeric forms, e.g., thieno [2, 3-b]pyridindiyl, thieno [2, 3-c]pyridindiyl, thieno [3, 2-b]pyridindiyl, and thieno [3, 2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1, 5-phenanthrolindiyl, 1, 6-phenanthrolindiyl, 1, 7-phenanthrolindiyl, 1, 9-phenanthrolindiyl, and 2, 10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1, 4-dihydrobenzo [d][1, 3]oxazinyl, 3, 4-dihydrobenzo [c][1, 2]-oxazinyl, and 3, 4-dihydrobenzo [d][1, 2]oxazinyl), dihydrobenzothienyl, dihydroisobenzofuranyl, dihydrobenzo [c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1, 4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1, 3, 5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. Heterocyclylene groups are bonded to the rest of a molecule through the non-aromatic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1, 4-dihydrobenzo [d][1, 3]oxazindiyl, 3, 4-dihydrobenzo [c][1, 2]oxazindiyl, and 3, 4-dihydrobenzo [d][1, 2]oxazindiyl), dihydrobenzothiendiyl, dihydroisobenzofurandiyl, dihydrobenzo [c]thiendiyl, dihydrofurdiyl, dihydroisoindoldiyl, dihydropyrandiyl, dihydropyrazoldiyl, dihydropyrazindiyl, dihydropyridindiyl, dihydropyrimidindiyl, dihydropyrroldiyl, dioxolandiyl, 1, 4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydroindoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydrothiendiyl, thiamorpholindiyl, thiazolidindiyl, thiochromandiyl, tetrahydroquinolindiyl, and 1, 3, 5-trithiandiyl. In certain embodiments, the heterocyclylene is optionally substituted with one or more substituents Q as described herein.

The term “halogen,” “halide,” or “halo” refers to fluoro, chloro, bromo, and/or iodo.

The term “optionally substituted” is intended to mean that a group or substituent, such as an alkyl, heteroalkyl, alkylene, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, or heterocyclylene group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a} C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O) NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center (s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P) sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P) sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers to the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) a diastereomer, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of a diastereomer, a mixture of two or more diastereomers, a tautomer, a mixture of two or more tautomers, or an isotopic variant of the compound referenced therein.”

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; (ii) a pharmaceutically acceptable salt, solvate, or hydrate of the compound referenced therein; or (iii) a pharmaceutically acceptable salt, solvate, or hydrate of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, provided herein is a compound of Formula (I):

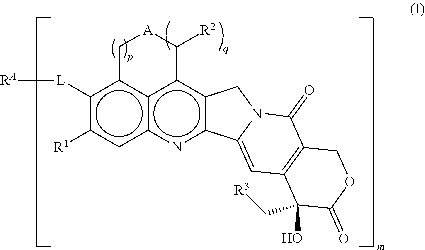

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each A is independently O, S, S(O), S(O₂), or N(R⁴);
each L is independently a linker;
each R¹, R², and R³ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1d}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1d})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O) NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O) NR^{1b}R^{1c}, —NR^{1a}C(O)SR^{1d}, —NR^{1a}C(NR^{1d})NR^{1b}R^{1c}, —NR^{1d}C(S)R^{1d}, —NR^{1a}C(S)OR^{1d}, —NR^{1a}C(S)NR^{1b}R^{1c}, —NR^{1d}S(O)R^{1d}, —NR^{1a}S(O)₂R^{1d}, —NR^{1d}S(O)NR^{1b}R^{1c}, —NR^{1a}S(O)₂NR^{1b}R^{1c}, —SR^{1a}, —S(O)R^{1d}, —S(O)₂R^{1a}, —S(O)NR^{1b}R^{1c}, or —S(O)₂NR^{1b}R^{1c};
each R⁴ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
each R^{1d}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
R^{A} is an antibody or an antigen-binding fragment thereof;
m is an integer of 1, 2, 3, 4, 5, 6, 7, 8, 9, 10, 11, 12, 13, 14, 15, or 16; and
each p and q is independently an integer of 0, 1, or 2; with the proviso that the total of p and q is greater than 0;
wherein each alkyl, heteroalkyl, alkenyl, alkynyl, cycloalkyl, aryl, aralkyl, heteroaryl, and heterocyclyl is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b} R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O) OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR'S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};

wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S) NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S) NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In another embodiment, provided herein is a compound of Formula (II):

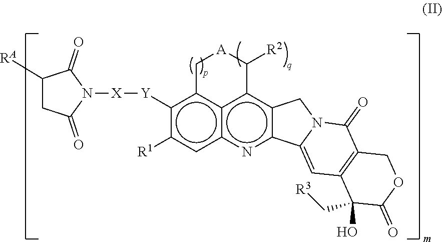

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each X is independently C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
each Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene; and
R¹, R², R³, R^{A}, A, m, p, and q are each as defined herein;
wherein each alkylene, heteroalkylene, alkenylene, heteroalkenylene, alkynylene, heteroalkynylene, cycloalkylene, arylene, heteroarylene, and heterocyclylene is optionally substituted with one or more substituents Q.

In certain embodiments, in Formula (I) or (II), each p is an integer of 0 and each q is an integer of 1. In certain embodiments, in Formula (I) or (II), each p is an integer of 1 and each q is an integer of 0.

In certain embodiments, in Formula (I) or (II), each p is an integer of 0 and each q is an integer of 2. In certain embodiments, in Formula (I) or (II), each p and q is an integer of 1. In certain embodiments, in Formula (I) or (II), each p is an integer of 2 and each q is an integer of 0.

In yet another embodiment, provided herein is a compound of Formula (III):

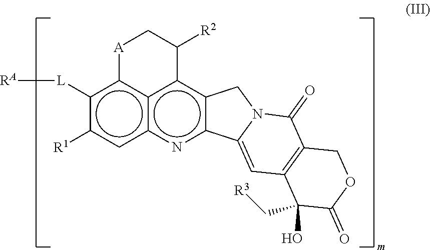

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, A, L, and m are each as defined herein.

In yet another embodiment, provided herein is a compound of Formula (IV):

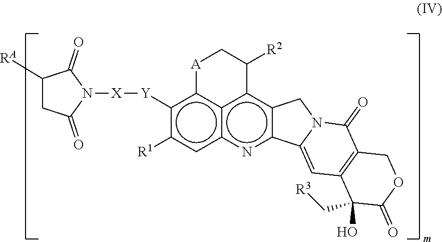

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, A, X, Y, and m are each as defined herein.

In yet another embodiment, provided herein is a compound of Formula (V):

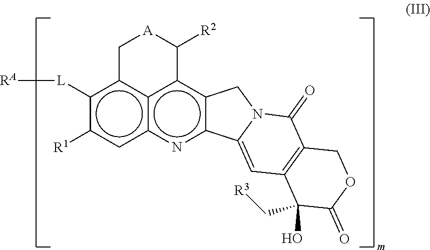

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, A, L, and m are each as defined herein.

In yet another embodiment, provided herein is a compound of Formula (VI):

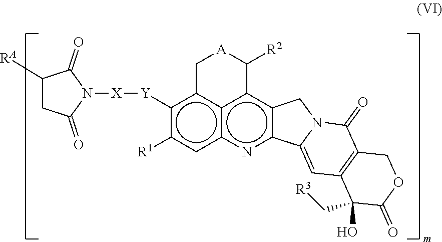

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, A, X, Y, and m are each as defined herein.

In certain embodiments, in any one of Formulae (I) to (VI), each A is O. In certain embodiments, in any one of Formulae (I) to (VI), each A is S. In certain embodiments, in any one of Formulae (I) to (VI), each A is S(O). In certain embodiments, in any one of Formulae (I) to (VI), each A is S(O₂). In certain embodiments, in any one of Formulae (I) to (VI), each A is independently N(R⁴), wherein R⁴ is as defined herein. In certain embodiments, in any one of Formulae (I) to (VI), each A is independently N(R⁴), wherein R⁴ is (i) hydrogen; (ii) C₁₋₆ alkyl or C₁₋₆ heteroalkyl, each optionally substituted with one or more substituents Q; or (iii) —C(O)R^{1a}, wherein R^{1a} is as defined herein. In certain embodiments, in any one of Formulae (I) to (VI), each A is independently N(H), N(CH₃), or N(C(O)CH₃). In certain embodiments, in any one of Formulae (I) to (VI), each A is N(H).

In yet another embodiment, provided herein is a compound of Formula (VII):

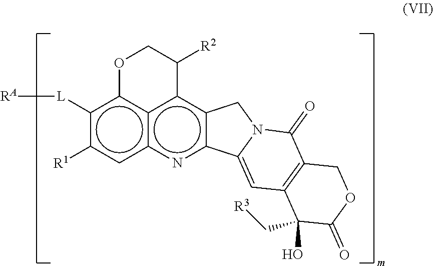

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, L, and m are each as defined herein.

In yet another embodiment, provided herein is a compound of Formula (VIII):

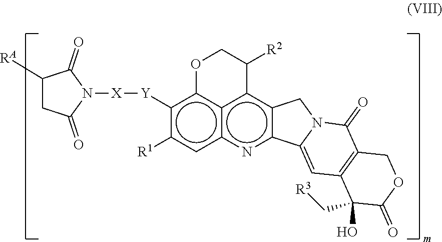

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, X, Y, and m are each as defined herein.

In yet another embodiment, provided herein is a compound of Formula (TX):

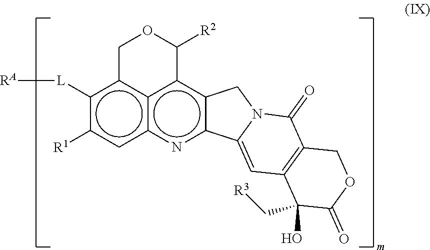

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, L, and m are each as defined herein.

In yet another embodiment, provided herein is a compound of Formula (X):

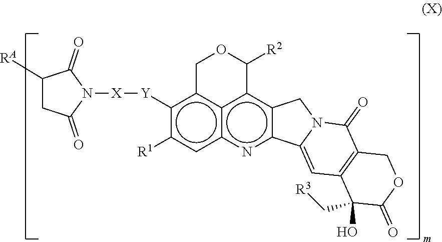

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, X, Y, and m are each as defined herein.

In yet another embodiment, provided herein is a compound of Formula (XI):

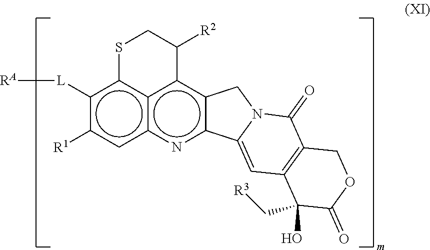

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, L, and m are each as defined herein.

In yet another embodiment, provided herein is a compound of Formula (XII):

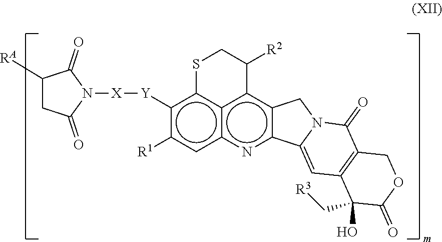

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, X, Y, and m are each as defined herein.

In still another embodiment, provided herein is a compound of Formula (XIII):

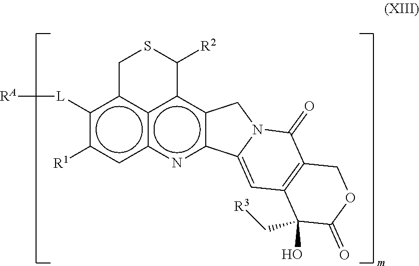

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, L, and m are each as defined herein.

In still another embodiment, provided herein is a compound of Formula (XIV):

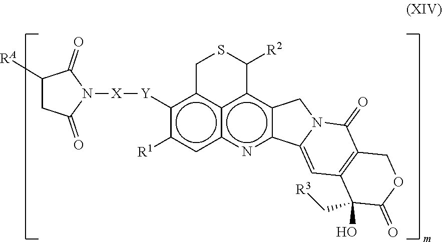

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{A}, X, Y, and m are each as defined herein.

In certain embodiments, in any one of Formulae (I) to (XIV), each R¹ is independently hydrogen, deuterium, or halo. In certain embodiments, in any one of Formulae (I) to (XIV), each R¹ is independently hydrogen, deuterium, or fluoro. In certain embodiments, in any one of Formulae (I) to (XIV), each R¹ is independently hydrogen or fluoro. In certain embodiments, in any one of Formulae (I) to (XIV), each R¹ is hydrogen. In certain embodiments, in any one of Formulae (I) to (XIV), each R¹ is fluoro.

In certain embodiments, in any one of Formulae (I) to (XIV), each R² is independently (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein. In certain embodiments, in any one of Formulae (I) to (XIV), each R² is independently hydrogen, amino, methylamino, acetamido, or hydroxyacetamido. In certain embodiments, in any one of Formulae (I) to (XIV), each R² is independently hydrogen or amino. In certain embodiments, in any one of Formulae (I) to (XIV), each R² is hydrogen. In certain embodiments, in any one of Formulae (I) to (XIV), each R² is amino.

In certain embodiments, in any one of Formulae (I) to (XIV), each R³ is independently C₁₋₆ alkyl, C₁₋₆ heteroalkyl, or C₂₋₆ alkenyl, each optionally substituted with one or more substituents Q. In certain embodiments, in any one of Formulae (I) to (XIV), each R³ is independently methyl, fluoromethyl, methoxymethyl, or ethenyl. In certain embodiments, in any one of Formulae (I) to (XIV), each R³ is methyl.

In certain embodiments, in any one of Formulae (I) to (XIV),
each R¹ is independently hydrogen, deuterium, or halo;
each R² is independently (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein; and
each R³ is independently C₁₋₆ alkyl, C₁₋₆ heteroalkyl, or C₂₋₆ alkenyl, each optionally substituted with one or more substituents Q.

In certain embodiments, in any one of Formulae (I) to (XIV),
each R¹ is independently hydrogen, deuterium, or fluoro;
each R² is independently hydrogen, amino, methylamino, acetamido, or hydroxyacetamido; and
each R³ is independently methyl, fluoromethyl, hydroxymethyl, or ethenyl.

In certain embodiments, in any one of Formulae (I) to (XIV),
each R¹ is independently hydrogen or fluoro;
each R² is independently hydrogen or amino; and
each R³ is independently methyl.

In certain embodiments, in any one of Formulae (I) to (XIV),
each R¹ is independently hydrogen or fluoro;
each R² is independently hydrogen; and
each R³ is independently methyl.

In one embodiment, provided herein is a compound of Formula (XV):

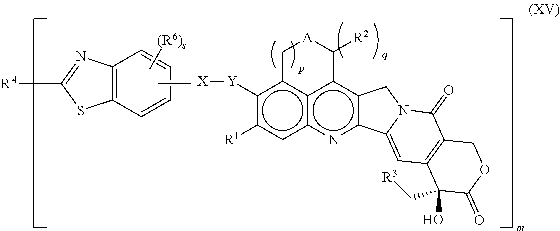

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
each R⁶ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋ₛ aralkyl, heteroaryl, or heterocyclyl, each optionally substituted with one or more substituents Q; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S) NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1c}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O) R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each s is independently an integer of 0, 1, 2, or 3; and
R¹, R², R³, R^{1a}, R^{1b}, R^{1c}, R^{1d}, R^{A}, A, X, Y, m, p, and q are each as defined herein.

In certain embodiments, in Formula (XV), each R⁶ is independently deuterium or halo. In certain embodiments, in Formula (XV), each R⁶ is independently deuterium or fluoro. In certain embodiments, in Formula (XV), each R⁶ is independently halo. In certain embodiments, in Formula (XV), each R⁶ is fluoro.

In certain embodiments, in Formula (XV), each s is independently an integer of 0 or 1. In certain embodiments, in Formula (XV), each s is an integer of 0. In certain embodiments, in Formula (XV), each s is an integer of 1.

In another embodiment, provided herein is a compound of Formula (XVI):

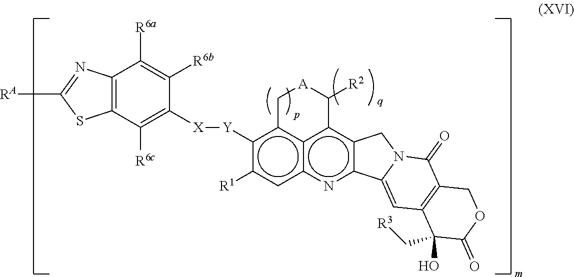

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein each R^{6a}, R^{6b}, and R^{6c} is independently hydrogen or R⁶; and R¹, R², R³, R⁶, R^{A}, A, X, Y, m, p, and q are each as defined herein.

In certain embodiments, in Formula (XV) or (XVI), each p is an integer of 0 and each q is an integer of 1. In certain embodiments, in Formula (XV) or (XVI), each p is an integer of 1 and each q is an integer of 0.

In certain embodiments, in Formula (XV) or (XVI), each p is an integer of 0 and each q is an integer of 2. In certain embodiments, in Formula (XV) or (XVI), each p and q is an integer of 1. In certain embodiments, in Formula (XV) or (XVI), each p is an integer of 2 and each q is an integer of 0.

In yet another embodiment, provided herein is a compound of Formula (XVII):

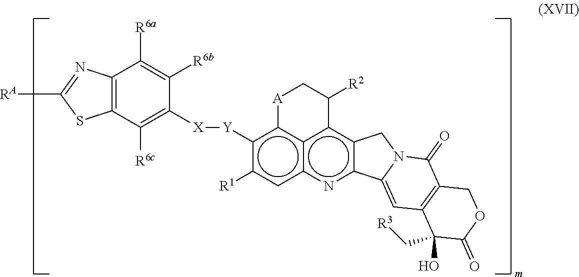

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, A, X, Y, and m are each as defined herein.

In yet another embodiment, provided herein is a compound of Formula (XVIII):

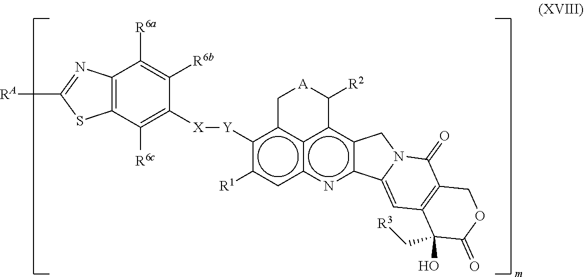

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, A, X, Y, and m are each as defined herein.

In certain embodiments, in any one of Formulae (XV) to (XVIII), each A is O. In certain embodiments, in any one of Formulae (XV) to (XVIII), each A is S. In certain embodiments, in any one of Formulae (XV) to (XVIII), each A is S(O). In certain embodiments, in any one of Formulae (XV) to (XVIII), each A is S(O₂). In certain embodiments, in any one of Formulae (XV) to (XVIII), each A is independently N (R⁴), wherein R⁴ is as defined herein. In certain embodiments, in any one of Formulae (XV) to (XVIII), each A is independently N(R⁴), wherein R⁴ is (i) hydrogen; (ii) C₁₋₆ alkyl or C₁₋₆ heteroalkyl, each optionally substituted with one or more substituents Q; or (iii) —C(O)R^{1a}, wherein R^{1a} is as defined herein. In certain embodiments, in any one of Formulae (XV) to (XVIII), each A is independently N(H), N(CH₃), or N(C(O) CH₃). In certain embodiments, in any one of Formulae (XV) to (XVIII), each A is N (H).

In yet another embodiment, provided herein is a compound of Formula (XIX):

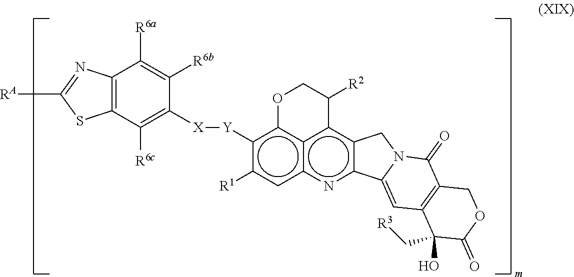

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, X, Y, and m are each as defined herein.

In yet another embodiment, provided herein is a compound of Formula (XX):

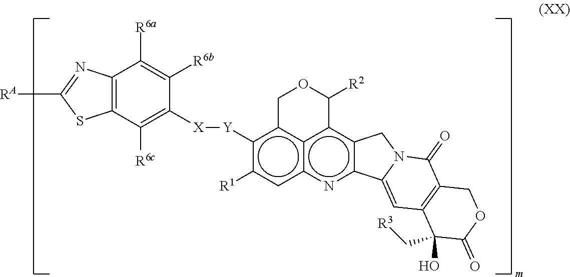

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, X, Y, and m are each as defined herein.

In yet another embodiment, provided herein is a compound of Formula (XXI):

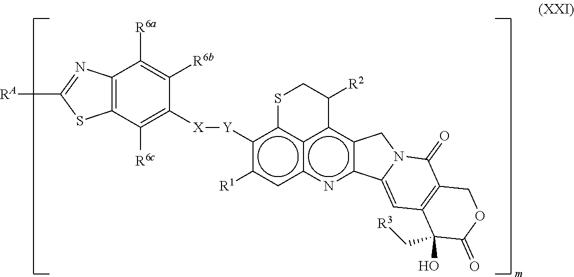

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, X, Y, and m are each as defined herein.

In still another embodiment, provided herein is a compound of Formula (XXII):

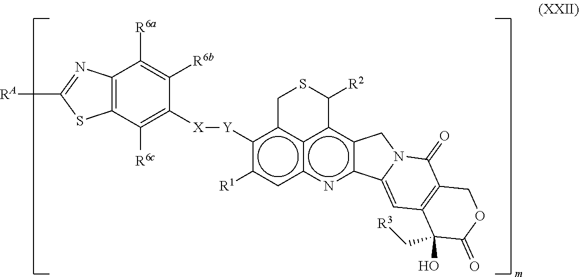

or a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R², R³, R^{6a}, R^{6b}, R^{6c}, R^{A}, X, Y, and m are each as defined herein.

In certain embodiments, in any one of Formulae (XV) to (XXII), each R¹ is independently hydrogen, deuterium, or halo. In certain embodiments, in any one of Formulae (XV) to (XXII), each R¹ is independently hydrogen, deuterium, or fluoro. In certain embodiments, in any one of Formulae (XV) to (XXII), each R¹ is independently hydrogen or fluoro. In certain embodiments, in any one of Formulae (XV) to (XXII), each R¹ is hydrogen. In certain embodiments, in any one of Formulae (XV) to (XXII), each R¹ is fluoro.

In certain embodiments, in any one of Formulae (XV) to (XXII), each R² is independently (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein. In certain embodiments, in any one of Formulae (XV) to (XXII), each R² is independently hydrogen, amino, methylamino, acetamido, or hydroxyacetamido. In certain embodiments, in any one of Formulae (XV) to (XXII), each R² is independently hydrogen or amino. In certain embodiments, in any one of Formulae (XV) to (XXII), each R² is hydrogen. In certain embodiments, in any one of Formulae (XV) to (XXII), each R² is amino.

In certain embodiments, in any one of Formulae (XV) to (XXII), each R³ is independently C₁₋₆ alkyl, C₁₋₆ heteroalkyl, or C₂₋₆ alkenyl, each optionally substituted with one or more substituents Q. In certain embodiments, in any one of Formulae (XV) to (XXII), each R³ is independently methyl, fluoromethyl, methoxymethyl, or ethenyl. In certain embodiments, in any one of Formulae (XV) to (XXII), each R³ is methyl.

In certain embodiments, in any one of Formulae (XVI) to (XXII), each R⁶, is independently hydrogen or deuterium. In certain embodiments, in any one of Formulae (XVI) to (XXII), each R⁶, is hydrogen.

In certain embodiments, in any one of Formulae (XVI) to (XXII), each R^{6b} is independently hydrogen, deuterium, or halo. In certain embodiments, in any one of Formulae (XVI) to (XXII), each R^{6b} is independently hydrogen, deuterium, or fluoro. In certain embodiments, in any one of Formulae (XVI) to (XXII), each R^{6b} is independently hydrogen or fluoro. In certain embodiments, in any one of Formulae (XVI) to (XXII), each R^{6b} is hydrogen. In certain embodiments, in any one of Formulae (XVI) to (XXII), each R^{6b} is fluoro.

In certain embodiments, in any one of Formulae (XVI) to (XXII), each R^{6c} is independently hydrogen or deuterium. In certain embodiments, in any one of Formulae (XVI) to (XXII), each R^{6c} is hydrogen.

In certain embodiments, in any one of Formulae (XVI) to (XXII),
each R¹ is independently hydrogen, deuterium, or halo;
each R² is independently (i) hydrogen or deuterium; (ii) heterocyclyl, optionally substituted with one or more substituents Q; or (iii) —NR^{1b}R^{1c} or —NR^{1b}C(O)R^{1d}; wherein R^{1a}, R^{1b}, R^{1c}, and R^{1d} are each as defined herein;
each R³ is independently C₁₋₆ alkyl, C₁₋₆ heteroalkyl, or C₂₋₆ alkenyl, each optionally substituted with one or more substituents Q;
each R^{6a} and R^{6c} is independently hydrogen or deuterium; and
each R^{6b} is independently hydrogen or halo.

In certain embodiments, in any one of Formulae (XVI) to (XXII),
each R¹ is independently hydrogen, deuterium, or fluoro;
each R² is independently hydrogen, amino, methylamino, acetamido, or hydroxyacetamido;
each R³ is independently methyl, fluoromethyl, hydroxymethyl, or ethenyl;
each R^{6a} and R^{6c} is hydrogen; and
each R^{6b} is independently hydrogen or fluoro.

In certain embodiments, in any one of Formulae (XVI) to (XXII),
each R¹ is independently hydrogen or fluoro;
each R² is independently hydrogen or amino;
each R³ is methyl;
each R^{6a} and R^{6c} is hydrogen; and
each R^{6b} is independently hydrogen or fluoro.

In certain embodiments, in any one of Formulae (XVI) to (XXII),
each R¹ is independently hydrogen or fluoro;
each R² is hydrogen;
each R³ is methyl;
each R^{6a} and R^{6c} is hydrogen; and
each R^{6b} is independently hydrogen or fluoro.

In one embodiment, R^{A} is a full length or intact monoclonal antibody. In another embodiment, R^{A} is a monoclonal antibody or an antigen-binding fragment thereof. In yet another embodiment, R^{A} is a single domain antibody or an antigen-binding fragment thereof. In yet another embodiment, R^{A} is a V_{H}H antibody. In still another embodiment, R^{A} is a V_{NAR} antibody.

In one embodiment, R^{A} is a human, humanized, or chimeric antibody, or an antigen-binding fragment thereof. In another embodiment, R^{A} is a human antibody or an antigen-binding fragment thereof. In yet another embodiment, R^{A} is a humanized antibody or an antigen-binding fragment thereof. In still another embodiment, R^{A} is a chimeric antibody or an antigen-binding fragment thereof.

In one embodiment, R^{A} is an IgA, IgD, IgE, IgG, or IgM antibody, or an antigen-binding fragment thereof. In another embodiment, R^{A} is an IgA antibody or an antigen-binding fragment thereof. In yet another embodiment, R^{A} is an IgD antibody or an antigen-binding fragment thereof. In yet another embodiment, R^{A} is an IgE antibody or an antigen-binding fragment thereof. In yet another embodiment, R^{A} is an IgG antibody or an antigen-binding fragment thereof. In still another embodiment, R^{A} is an IgM antibody or an antigen-binding fragment thereof.

In one embodiment, R^{A} is an IgA1, IgA2, IgG1, IgG2, IgG3, or IgG4 antibody, or an antigen-binding fragment thereof. In another embodiment, R^{A} is an IgA1 or IgA2, or an antigen-binding fragment thereof. In yet another embodiment, R^{A} is an IgA1 or an antigen-binding fragment thereof. In yet another embodiment, R^{A} is an IgA2 or an antigen-binding fragment thereof. In yet another embodiment, R^{A} is an IgG1, IgG2, IgG3, or IgG4 antibody, or an antigen-binding fragment thereof. In yet another embodiment, R^{A} is an IgG1 antibody or an antigen-binding fragment thereof. In yet another embodiment, R^{A} is an IgG2 antibody or an antigen-binding fragment thereof. In yet another embodiment, R^{A} is an IgG3 antibody or an antigen-binding fragment thereof. In still another embodiment, R^{A} is an IgG4 antibody or an antigen-binding fragment thereof.

In one embodiment, R^{A} is a single-chain variable fragment (scFv), Fab, Fab′, F (ab)₂, F (ab′)₂, Fv, diabody, triabody, tetrabody, or minibody of an antibody provided herein. In another embodiment, R^{A} is an scFv of an antibody provided herein. In yet another embodiment, R^{A} is a Fab of an antibody provided herein. In yet another embodiment, R^{A} is a Fab′ of an antibody provided herein. In yet another embodiment, R^{A} is a F(ab)₂ of an antibody provided herein. In yet another embodiment, R^{A} is a F (ab′)₂ of an antibody provided herein. In yet another embodiment, R^{A} is a Fv of an antibody provided herein. In yet another embodiment, R^{A} is a diabody of an antibody provided herein. In yet another embodiment, R^{A} is a triabody of an antibody provided herein. In yet another embodiment, R^{A} is a tetrabody of an antibody provided herein. In still another embodiment, R^{A} is a minibody of an antibody provided herein.

In one embodiment, R^{A} is an afucosylated antibody or an antigen-binding fragment thereof.

In one embodiment, R^{A} is a recombinant antibody or an antigen-binding fragment thereof. In another embodiment, R^{A} is a purified antibody or an antigen-binding fragment thereof. In yet another embodiment, R^{A} is an isolated antibody or an antigen-binding fragment thereof.

In certain embodiments, R^{A} is an antibody or an antigen-binding fragment thereof, wherein the antibody specifically binds to AXL, B7 homolog 3 (B7H3), B-cell maturation antigen (BCMA), cadherin 3, cadherin 6, CanAg, carbonic anhydrase VI (CA6), C4-4A, carcinoembryonic antigen-related cell adhesion molecules (CEACAMs), CD19, CD20, CD22, CD25, CD30, CD33, CD37, CD44, CD56, CD70, CD74, CD79b, CD123, CD138, CD142, CD352, cripto 1 growth factor, delta-like 3 (DLL3), ectonucleotide pyrophosphatase/phosphor-diesterase family member 3 (ENPP3), endothelin receptor type B (ETBR), EPH receptor A2 (EPHA2), ephrin A4, epidermal growth factor receptor (EGFR), FMS-like tyrosine kinase 3 (FLT3), fibroblast growth factor receptor 2 (FGFR2), fibroblast growth factor receptor 3 (FGFR3), folate receptor 1 (FOLR1 or FRα), guanylate cyclase 2C (GCC), hepatocyte growth factor receptor (HGFR), HER2, HER3, integrin αV, KIT, Lewis Y antigen, LIV-1, lymphocyte antigen 6E (LY6E), lysosomal-associated membrane protein 1 (LAMP-1), mesothelin (MSLN), mucin 1 (MUC1), mucin-16 (MUC16), nectin-4, notch receptor 3 (NOTCH3), osteoactivin, prostate-specific membrane antigen (PSMA), six transmembrane epithelial antigen of the prostate 1 (STEAP1), SLAM family member 7 (SLAMF7), SLITRK6, ST2, sodium-dependent phosphate transport protein 2B (SLC34A2), T-cell immunoglobulin and mucin domain 1 (TIM-1), transferrin receptor protein 1, tumor-associated calcium signal transducer 2 (TROP2), or tyrosine-protein kinase-like 7 (PTK7).

In certain embodiments, R^{A} is an anti-B7H3 antibody, anti-CD20 antibody, anti-FOLR1 antibody, anti-HER2 antibody, anti-MSLN antibody, anti-TROP2 antibody, or an antigen-binding fragment thereof. In certain embodiments, R^{A} is an anti-B7H3 antibody or an antigen-binding fragment thereof. In certain embodiments, R^{A} is an anti-CD20 antibody or an antigen-binding fragment thereof. In certain embodiments, R^{A} is an anti-FOLR1 antibody or an antigen-binding fragment thereof. In certain embodiments, R^{A} is an anti-HER2 antibody or an antigen-binding fragment thereof. In certain embodiments, R^{A} is an anti-MSLN antibody or an antigen-binding fragment thereof. In certain embodiments, R^{A} is an anti-TROP2 antibody or an antigen-binding fragment thereof.

In one embodiment, R^{A} is an anti-B7H3 single domain antibody or an antigen-binding fragment thereof, wherein the antibody comprises (i) a chain complementarity determining region 1 (CDR1) of SEQ ID NO: 1; (ii) a chain complementarity determining region 2 (CDR2) of SEQ ID NO: 2; and (iii) a chain complementarity determining region 3 (CDR3) of SEQ ID NO: 3. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-B7H3 single domain antibody or an antigen-binding fragment thereof, wherein the antibody comprises a variable region of SEQ ID NO: 4. In yet another embodiment, R^{A} is an anti-B7H3 single domain antibody or an antigen-binding fragment thereof, wherein the antibody comprises an amino acid sequence of SEQ ID NO: 5.

In one embodiment, R^{A} is an anti-B7H3 single domain antibody or an antigen-binding fragment thereof, wherein the antibody comprises (i) a CDR1 of SEQ ID NO: 6; (ii) a CDR2 of SEQ ID NO: 7; and (iii) a CDR3 of SEQ ID NO: 8. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-B7H3 single domain antibody or an antigen-binding fragment thereof, wherein the antibody comprises a variable region of SEQ ID NO: 9. In yet another embodiment, R^{A} is an anti-B7H3 single domain antibody or an antigen-binding fragment thereof, wherein the antibody comprises an amino acid sequence of SEQ ID NO: 10.

In one embodiment, R^{A} is an anti-CD20 antibody or an antigen-binding fragment thereof, wherein the antibody comprises (i) a light chain complementarity determining region 1 (CDRL1) of SEQ ID NO: 11; (ii) a light chain complementarity determining region 2 (CDRL2) of SEQ ID NO: 12; (iii) a light chain complementarity determining region 3 (CDRL3) of SEQ ID NO: 13; (iv) a heavy chain complementarity determining region 1 (CDRH1) of SEQ ID NO: 14; (v) a heavy chain complementarity determining region 2 (CDRH2) of SEQ ID NO: 15; and (vi) a heavy chain complementarity determining region 3 (CDRH3) of SEQ ID NO: 16. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-CD20 antibody or an antigen-binding fragment thereof, wherein the antibody comprises (i) a light chain variable region of SEQ ID NO: 17; and (ii) a heavy chain variable region of SEQ ID NO: 18. In yet another embodiment, R^{A} is an anti-CD20 antibody or an antigen-binding fragment thereof, wherein the antibody comprises (i) a light chain of SEQ ID NO: 19; and (ii) a heavy chain of SEQ ID NO: 20.

In one embodiment, R^{A} is an anti-FOLR1 antibody or an antigen-binding fragment thereof, wherein the antibody comprises (i) a CDRL1 of SEQ ID NO: 21; (ii) a CDRL2 of SEQ ID NO: 22; (iii) a CDRL3 of SEQ ID NO: 23; (iv) a CDRH1 of SEQ ID NO: 24; (v) a CDRH2 of SEQ ID NO: 25; and (vi) a CDRH3 of SEQ ID NO: 26. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-FOLR1 antibody or an antigen-binding fragment thereof, wherein the antibody comprises (i) a light chain variable region of SEQ ID NO: 27; and (ii) a heavy chain variable region of SEQ ID NO: 28. In yet another embodiment, R^{A} is an anti-FOLR1 antibody or an antigen-binding fragment thereof, wherein the antibody comprises (i) a light chain of SEQ ID NO: 29; and (ii) a heavy chain of SEQ ID NO: 30.

In one embodiment, R^{A} is an anti-HER2 antibody or an antigen-binding fragment thereof, wherein the antibody comprises (i) a CDRL1 of SEQ ID NO: 31; (ii) a CDRL2 of SEQ ID NO: 32; (iii) a CDRL3 of SEQ ID NO: 33; (iv) a CDRH1 of SEQ ID NO: 34; (v) a CDRH2 of SEQ ID NO: 35; and (vi) a CDRH3 of SEQ ID NO: 36. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-HER2 antibody or an antigen-binding fragment thereof, wherein the antibody comprises (i) a light chain variable region of SEQ ID NO: 37; and (ii) a heavy chain variable region of SEQ ID NO: 38. In yet another embodiment, R^{A} is an anti-HER2 antibody or an antigen-binding fragment thereof, wherein the antibody comprises (i) a light chain of SEQ ID NO: 39; and (ii) a heavy chain of SEQ ID NO: 40.

In one embodiment, R^{A} is an anti-MSLN single domain antibody or an antigen-binding fragment thereof, wherein the antibody comprises (i) a CDR1 of SEQ ID NO: 41; (ii) a CDR2 of SEQ ID NO: 42; and (iii) a CDR3 of SEQ ID NO: 43. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-MSLN single domain antibody or an antigen-binding fragment thereof, wherein the antibody comprises a variable region of SEQ ID NO: 44. In yet another embodiment, R^{A} is an anti-MSLN single domain antibody or an antigen-binding fragment thereof, wherein the antibody comprises an amino acid sequence of SEQ ID NO: 45.

In one embodiment, R^{A} is an anti-MSLN single domain antibody or an antigen-binding fragment thereof, wherein the antibody comprises (i) a CDR1 of SEQ ID NO: 46; (ii) a CDR2 of SEQ ID NO: 47; and (iii) a CDR3 of GRY. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-MSLN single domain antibody or an antigen-binding fragment thereof, wherein the antibody comprises a variable region of SEQ ID NO: 48. In yet another embodiment, R^{A} is an anti-MSLN single domain antibody or an antigen-binding fragment thereof, wherein the antibody comprises an amino acid sequence of SEQ ID NO: 49.

Additional anti-MSLN single domain antibodies and antigen-binding fragments thereof include those disclosed in WO 2019/246003 A1, the disclosure of which is incorporated herein by reference in its entirety.

In one embodiment, R^{A} is an anti-TROP2 antibody or an antigen-binding fragment thereof, wherein the antibody comprises (i) a CDRL1 of SEQ ID NO: 50; (ii) a CDRL2 of SEQ ID NO: 51; (iii) a CDRL3 of SEQ ID NO: 52; (iv) a CDRH1 of SEQ ID NO: 53; (v) a CDRH2 of SEQ ID NO: 54; and (vi) a CDRH3 of SEQ ID NO: 55. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-TROP2 antibody or an antigen-binding fragment thereof, wherein the antibody comprises (i) a light chain variable region of SEQ ID NO: 56; and (ii) a heavy chain variable region of SEQ ID NO: 57. In yet another embodiment, R^{A} is an anti-TROP2 antibody or an antigen-binding fragment thereof, wherein the antibody comprises (i) a light chain of SEQ ID NO: 58; and (ii) a heavy chain of SEQ ID NO: 59.

In one embodiment, R^{A} is an anti-CD79b antibody or an antigen-binding fragment thereof, wherein the antibody comprises (i) a CDRL1 of SEQ ID NO: 60; (ii) a CDRL2 of SEQ ID NO: 61; (iii) a CDRL3 of SEQ ID NO: 62; (iv) a CDRH1 of SEQ ID NO: 63; (v) a CDRH2 of SEQ ID NO: 64; and (vi) a CDRH3 of SEQ ID NO: 65. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-TROP2 antibody or an antigen-binding fragment thereof, wherein the antibody comprises (i) a light chain variable region of SEQ ID NO: 66; and (ii) a heavy chain variable region of SEQ ID NO: 67. In yet another embodiment, R^{A} is an anti-TROP2 antibody or an antigen-binding fragment thereof, wherein the antibody comprises (i) a light chain of SEQ ID NO: 68; and (ii) a heavy chain of SEQ ID NO: 69.

In one embodiment, R^{A} is an anti-CD79b antibody or an antigen-binding fragment thereof, wherein the antibody comprises (i) a CDRL1 of SEQ ID NO: 70; (ii) a CDRL2 of SEQ ID NO: 71; (iii) a CDRL3 of SEQ ID NO: 72; (iv) a CDRH1 of SEQ ID NO: 73; (v) a CDRH2 of SEQ ID NO: 74; and (vi) a CDRH3 of SEQ ID NO: 75. In certain embodiments, the CDRs provided herein are defined according to the IMGT or Kabat numbering system. In certain embodiments, the CDRs provided herein are defined according to the IMGT numbering system. In certain embodiments, the CDRs provided herein are defined according to the Kabat numbering system. In another embodiment, R^{A} is an anti-TROP2 antibody or an antigen-binding fragment thereof, wherein the antibody comprises (i) a light chain variable region of SEQ ID NO: 76; and (ii) a heavy chain variable region of SEQ ID NO: 77. In yet another embodiment, R^{A} is an anti-TROP2 antibody or an antigen-binding fragment thereof, wherein the antibody comprises (i) a light chain of SEQ ID NO: 78; and (ii) a heavy chain of SEQ ID NO: 79.

In one embodiment, L is a cleavable linker. In another embodiment, L is a non-cleavable linker.

In certain embodiments, L is a cleavable linker that is sensitive to an acidic pH. In certain embodiments, L is a cleavable linker comprising a reducible disulfide. In certain embodiments, L is a linker cleavable by glutathione. In certain embodiments, L is a linker cleavable by an enzyme. In certain embodiments, L is a linker cleavable by a protease. In certain embodiments, L is a linker cleavable by a lysosomal protease. In certain embodiments, L is a linker cleavable by cathepsin B. In certain embodiments, L is a linker cleavable by a glycosidase. In certain embodiments, L is a linker cleavable by a β-glycosidase. In certain embodiments, L is a linker cleavable by a galactosidase. In certain embodiments, L is a linker cleavable by a β-galactosidase. In certain embodiments, L is a linker cleavable by a glucuronidase. In certain embodiments, L is a linker cleavable by a β-glucuronidase. In certain embodiments, L is a linker cleavable by a phosphatase. Exemplary linkers suitable for a compound provided herein include, but are not limited to, those disclosed in Beck et al., Nat. Rev. Drug Discov. 2017, 16, 317-37; Bargh et al., Chem. Soc. Rev. 2019, 48, 4361-74; the disclosure of each of which is incorporated herein by reference in its entirety.

In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, C₂₋₅₀ heteroalkynylene, C₃₋₁₀ cycloalkylene, C₆₋₁₄ arylene, heteroarylene, or heterocyclylene, each of which is optionally substituted with one or more substituents Q. In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, or C₂₋₅₀ heteroalkynylene, each of which is optionally substituted with one or more substituents Q. In certain embodiments, L is C₁₋₅₀ alkylene or C₁₋₅₀ heteroalkylene, each of which is optionally substituted with one or more substituents Q.

In certain embodiments, L is C₁₋₅₀ alkylene, optionally substituted with one or substituents Q. In certain embodiments, L is C₁₋₄₀ alkylene, optionally substituted with one or more substituents Q. In certain embodiments, L is C₁₋₃₀ alkylene, optionally substituted with one or more substituents Q. In certain embodiments, L is C₁₋₂₀ alkylene, optionally substituted with one or more substituents Q. In certain embodiments, L is C₂₋₁₆ alkylene, optionally substituted with one or more substituents Q. In certain embodiments, L is C₂₋₁₂ alkylene, optionally substituted with one or more substituents Q. In certain embodiments, L is C₂₋₆ alkylene, optionally substituted with one or more substituents Q. In certain embodiments, L is C₁₋₂₀ alkylene, optionally substituted with one or two oxo. In certain embodiments, L is C₂₋₁₆ alkylene, optionally substituted with one or two oxo. In certain embodiments, L is C₂₋₁₂ alkylene, optionally substituted with one or two oxo. In certain embodiments, L is C₂₋₆ alkylene, optionally substituted with one or two oxo. In certain embodiments, L is —(CH₂)ᵤ—, optionally substituted with one or two oxo; wherein u is an integer of 1, 2, 3, 4, 5, 6, 7, 8, 9, 10, 11, 12, 13, 14, 15, or 16. In certain embodiments, L is —(CH₂)ᵤ—, optionally substituted with one or two oxo; wherein u is an integer of 2, 3, 4, 5, 6, 7, 8, 9, 10, 11, or 12. In certain embodiments, L is —(CH₂)ᵤ—, optionally substituted with one or two oxo; wherein r is an integer of 2, 3, 4, 5, or 6.

In certain embodiments, L is C₁₋₅₀ heteroalkylene, optionally substituted with one or more substituents Q. In certain embodiments, L is C₁₋₄₀ heteroalkylene, optionally substituted with one or more substituents Q. In certain embodiments, L is C₁₋₃₀ heteroalkylene, optionally substituted with one or more substituents Q. In certain embodiments, L is C₁₋₂₀ heteroalkylene, optionally substituted with one or more substituents Q. In certain embodiments, L is C₂₋₁₆ heteroalkylene, optionally substituted with one or more substituents Q. In certain embodiments, L is C₂₋₁₂ heteroalkylene, optionally substituted with one or more substituents Q. In certain embodiments, L is C₂₋₆ heteroalkylene, optionally substituted with one or more substituents Q. In certain embodiments, L is C₁₋₂₀ heteroalkylene, optionally substituted with one, two, or three oxo. In certain embodiments, L is C₂₋₁₆ heteroalkylene, optionally substituted with one, two, or three oxo. In certain embodiments, L is C₂₋₁₂ heteroalkylene, optionally substituted with one, two, or three oxo. In certain embodiments, L is C₂₋₆ heteroalkylene, optionally substituted with one, two, or three oxo.

In certain embodiments, L is C₂₋₅₀ heteroalkylene comprising an ethyleneoxy (—CH₂CH₂O—) group, optionally substituted with one or more substituents Q. In certain embodiments, L is C₂₋₄₀ heteroalkylene comprising an ethyleneoxy group, optionally substituted with one or more substituents Q. In certain embodiments, L is C₂₋₃₀ heteroalkylene comprising an ethyleneoxy group, optionally substituted with one or more substituents Q. In certain embodiments, L is C₂₋₂₀ heteroalkylene comprising an ethyleneoxy group, optionally substituted with one or more substituents Q. In certain embodiments, L is C₂₋₁₄ heteroalkylene comprising an ethyleneoxy group, optionally substituted with one or more substituents Q. In certain embodiments, L is C₂₋₁₀ heteroalkylene comprising an ethyleneoxy group, optionally substituted with one or more substituents Q. In certain embodiments, L is C₂₋₆ heteroalkylene comprising an ethyleneoxy group, optionally substituted with one or more substituents Q.

In certain embodiments, L is C₃₋₅₀ heteroalkylene comprising a propyleneoxy (—CH₂CH₂CH₂O—) group, optionally substituted with one or more substituents Q. In certain embodiments, L is C₃₋₄₀ heteroalkylene comprising a propyleneoxy group, optionally substituted with one or more substituents Q. In certain embodiments, L is C₃₋₃₀ heteroalkylene comprising a propyleneoxy group, optionally substituted with one or more substituents Q. In certain embodiments, L is C₃₋₂₀ heteroalkylene comprising a propyleneoxy group, optionally substituted with one or more substituents Q. In certain embodiments, L is C₃₋₁₄ heteroalkylene comprising a propyleneoxy group, optionally substituted with one or more substituents Q. In certain embodiments, L is C₃₋₁₀ heteroalkylene comprising a propyleneoxy group, optionally substituted with one or more substituents Q.

In certain embodiments, L is C₁₋₅₀ alkylene, C₁₋₅₀ heteroalkylene, C₂₋₅₀ alkenylene, C₂₋₅₀ heteroalkenylene, C₂₋₅₀ alkynylene, or C₂₋₅₀ heteroalkynylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, heteroalkylene, alkenylene, heteroalkenylene, alkynylene, heteroalkynylene, cycloalkylene, arylene, heteroarylene, and heterocyclylene are each optionally substituted with one or more substituents Q. In certain embodiments, L is C₁₋₅₀ alkylene or C₁₋₅₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, heteroalkylene, cycloalkylene, arylene, heteroarylene, and heterocyclylene are each optionally substituted with one or more substituents Q.

In certain embodiments, L is C₁₋₅₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₄₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₃₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q.

In certain embodiments, L is C₁₋₅₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₄₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₃₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one, two, three, or four methylene groups are each independently replaced by a divalent group; wherein each divalent group is independently cyclohexanediyl, phendiyl, triazoldiyl, or 2, 5-dioxopyrrolidindiyl. In certain embodiments, L is C₁₋₂₀ alkylene, wherein one, two, three, or four methylene groups are each independently replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₂₀ alkylene, wherein one or two methylene groups are each independently replaced by a divalent group; wherein each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein one or two methylene groups are each independently replaced by a divalent group; wherein each divalent group is independently cyclohexanediyl, phendiyl, triazoldiyl, or 2, 5-dioxopyrrolidindiyl. In certain embodiments, L is C₁₋₂₀ alkylene, wherein one or two methylene groups are each independently replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₂₀ alkylene, wherein a methylene group is replaced by a divalent group; wherein the divalent group is C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, L is C₁₋₂₀ alkylene, wherein a methylene group is replaced by a divalent group; wherein the divalent group is cyclohexanediyl, phendiyl, triazoldiyl, or 2, 5-dioxopyrrolidindiyl. In certain embodiments, L is C₁₋₂₀ alkylene, wherein a methylene group is replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₅₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₄₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₃₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one or more methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q.

In certain embodiments, L is C₁₋₅₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₄₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₃₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one, two, three, or four methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one, two, three, or four methylene groups are each independently replaced by a divalent group; wherein each divalent group is independently cyclohexanediyl, phendiyl, triazoldiyl, or 2, 5-dioxopyrrolidindiyl. In certain embodiments, L is C₁₋₂₀ heteroalkylene, wherein one, two, three, or four methylene groups are each independently replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₂₀ heteroalkylene, wherein one or two methylene groups are each independently replaced by a divalent group; wherein each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein one or two methylene groups are each independently replaced by a divalent group; wherein each divalent group is independently cyclohexanediyl, phendiyl, triazoldiyl, or 2, 5-dioxopyrrolidindiyl. In certain embodiments, L is C₁₋₂₀ heteroalkylene, wherein one or two methylene groups are each independently replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₂₀ heteroalkylene, wherein a methylene group is replaced by a divalent group; wherein the divalent group is C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, L is C₁₋₂₀ heteroalkylene, wherein a methylene group is replaced by a divalent group; wherein the divalent group is cyclohexanediyl, phendiyl, triazoldiyl, or 2, 5-dioxopyrrolidindiyl. In certain embodiments, L is C₁₋₂₀ heteroalkylene, wherein a methylene group is replaced by a divalent group; wherein each divalent group is independently cyclohexane-1, 4-diyl, phen-1, 3-diyl, phen-1, 4-diyl, 1, 2, 3-triazol-1, 4-diyl, or 2, 5-dioxopyrrolidin-1, 3-diyl.

In certain embodiments, L is C₁₋₄₀ alkylene-C₃₋₁₀ cycloalkylene, C₁₋₄₀ heteroalkylene-C₃₋₁₀ cycloalkylene, C₁₋₄₀ alkylene-C₆₋₁₄ arylene, C₁₋₄₀ heteroalkylene-C₆₋₁₄ arylene, C₁₋₄₀ alkylene-heteroarylene, C₁₋₄₀ heteroalkylene-heteroarylene, C₁₋₄₀ alkylene-heterocyclylene, C₁₋₄₀ heteroalkylene-heterocyclylene, or heteroarylene-heterocyclylene, where each alkylene, heteroalkylene, cycloalkylene, arylene, heteroarylene, and heterocyclylene is optionally substituted with one or more substituents Q. In certain embodiments, L is C₁₋₄₀ alkylene-C₃₋₁₀ cycloalkylene, where the alkylene and cycloalkylene are each optionally substituted with one or more substituents Q. In certain embodiments, L is C₁₋₄₀ heteroalkylene-C₃₋₁₀ cycloalkylene, where the heteroalkylene and cycloalkylene are each optionally substituted with one or more substituents Q. In certain embodiments, L is C₁₋₄₀ alkylene-C₆₋₁₄ arylene, where the alkylene and arylene are each optionally substituted with one or more substituents Q. In certain embodiments, L is C₁₋₄₀ heteroalkylene-C₆₋₁₄ arylene, where the heteroalkylene and arylene are each optionally substituted with one or more substituents Q. In certain embodiments, L is C₁₋₄₀ alkylene-heteroarylene, where the alkylene and heteroarylene are each optionally substituted with one or more substituents Q. In certain embodiments, L is C₁₋₄₀ heteroalkylene-heteroarylene, where the heteroalkylene and heteroarylene are each optionally substituted with one or more substituents Q. In certain embodiments, L is C₁₋₄₀ alkylene-heterocyclylene, where the alkylene and heterocyclylene are each optionally substituted with one or more substituents Q. In certain embodiments, L is C₁₋₄₀ heteroalkylene-heterocyclylene, where the heteroalkylene and heterocyclylene are each optionally substituted with one or more substituents Q. In certain embodiments, L is C₁₋₄₀ heteroarylene-heterocyclylene, where the heteroarylene and heterocyclylene are each optionally substituted with one or more substituents Q.

In certain embodiments, L is:

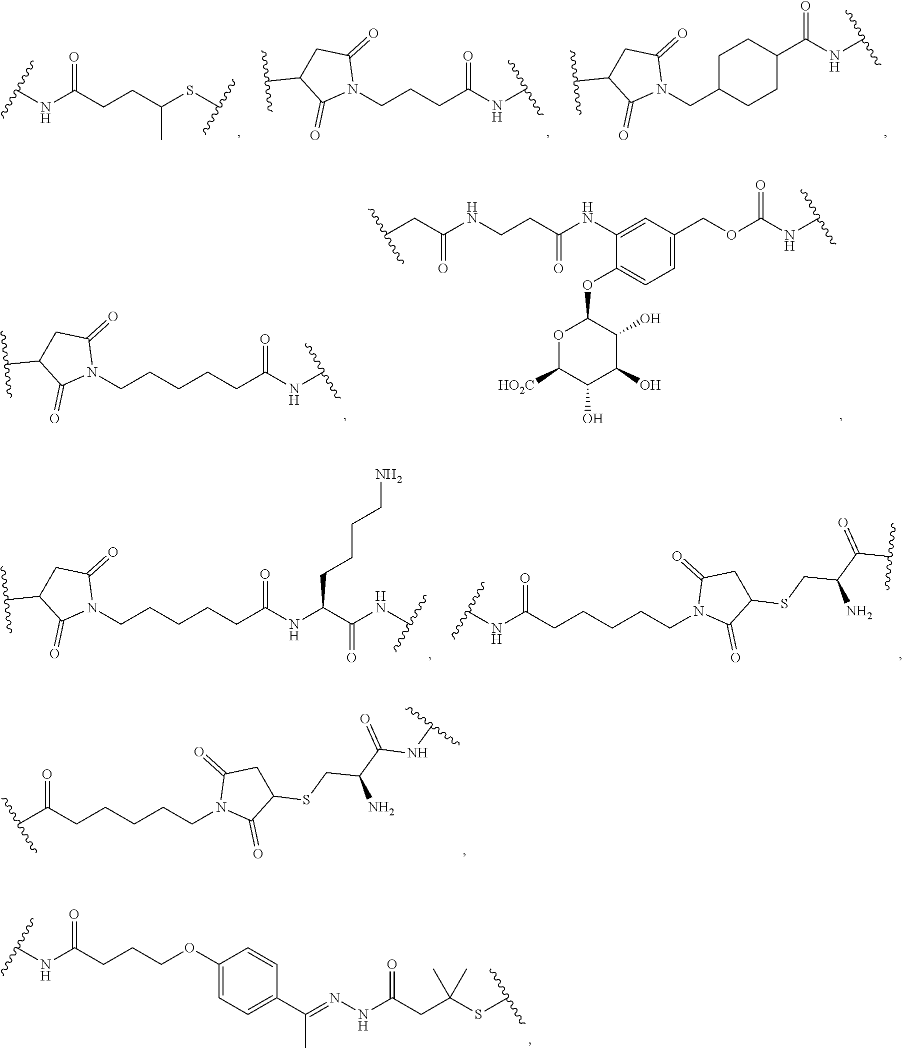

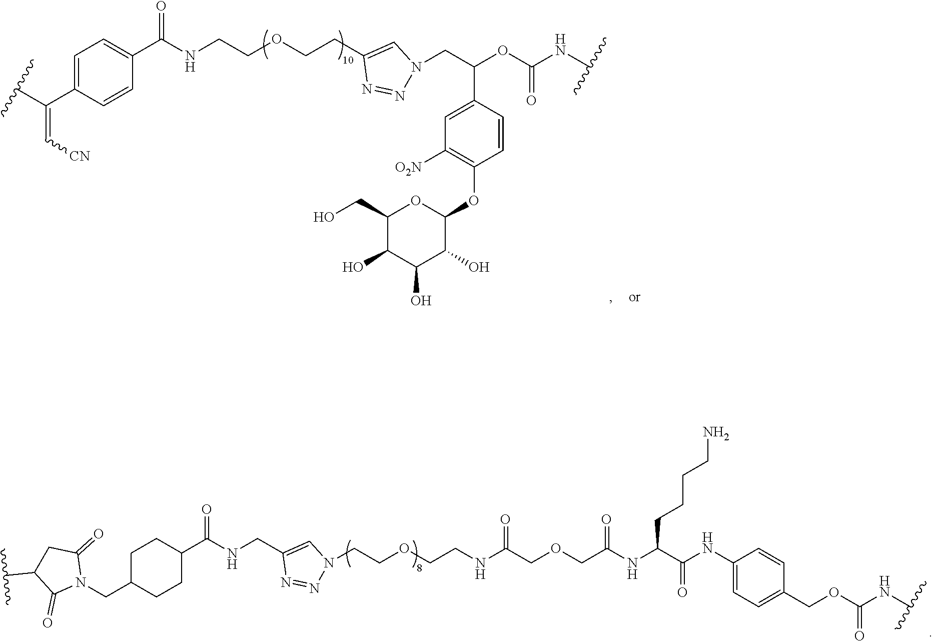

In certain embodiments, L is:

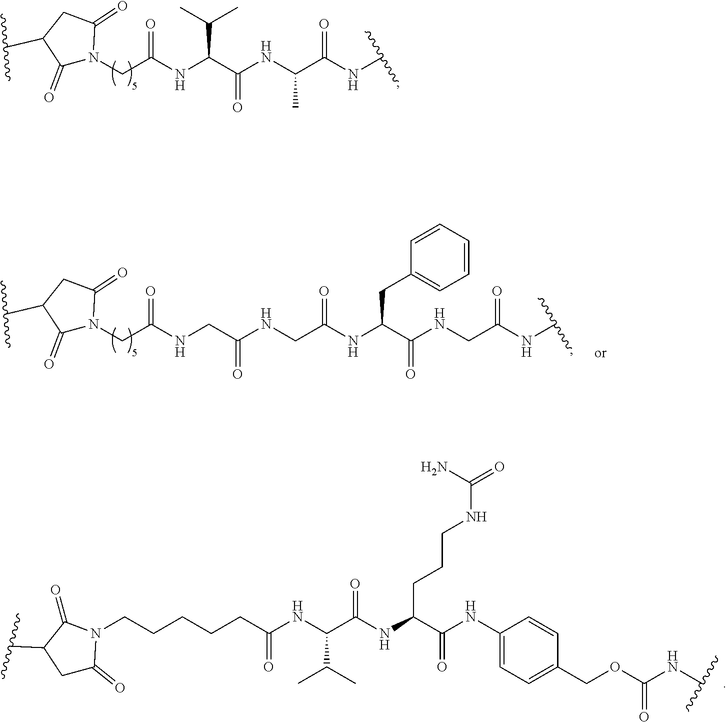

In certain embodiments, L is:

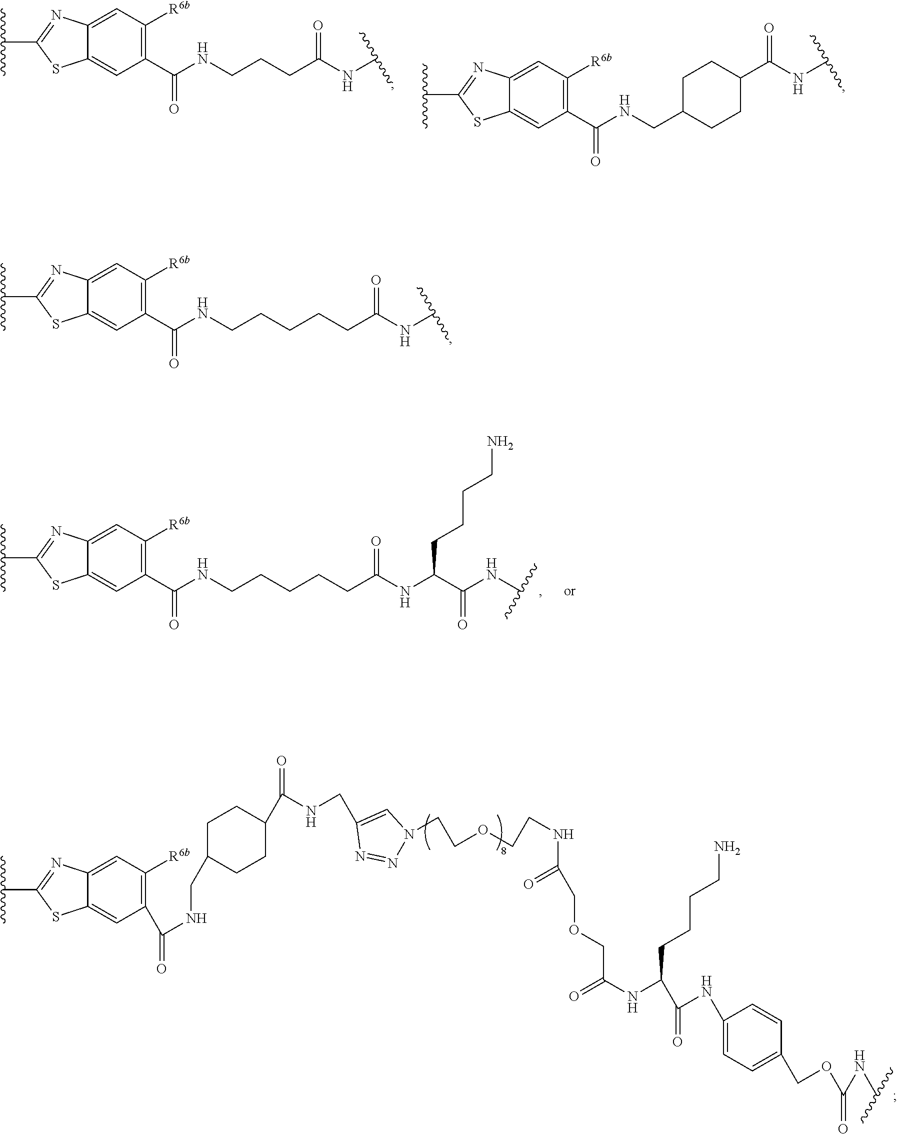

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

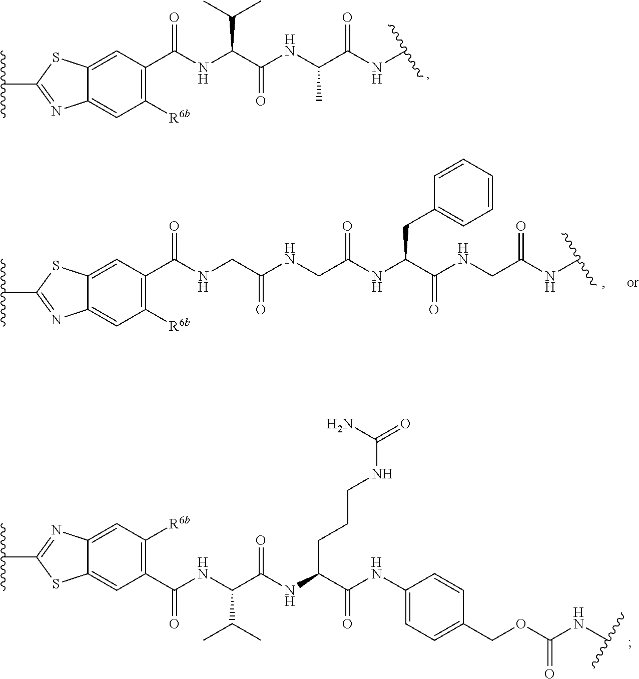

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

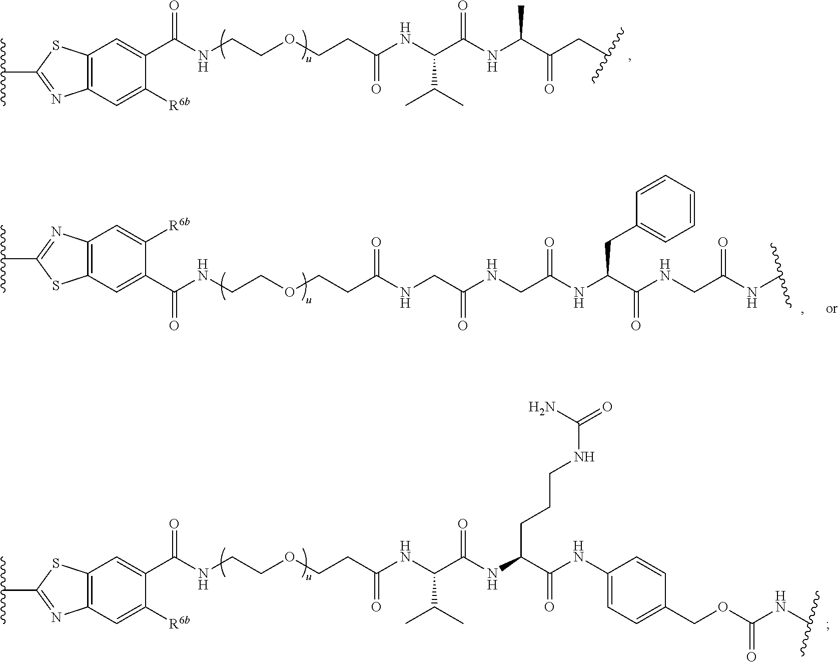

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro; and in one embodiment, each u is independently an integer of 1, 2, 3, 4, 5, 6, 7, or 8; in another embodiment, each u is independently an integer of 4 or 8.

In certain embodiments. L is:

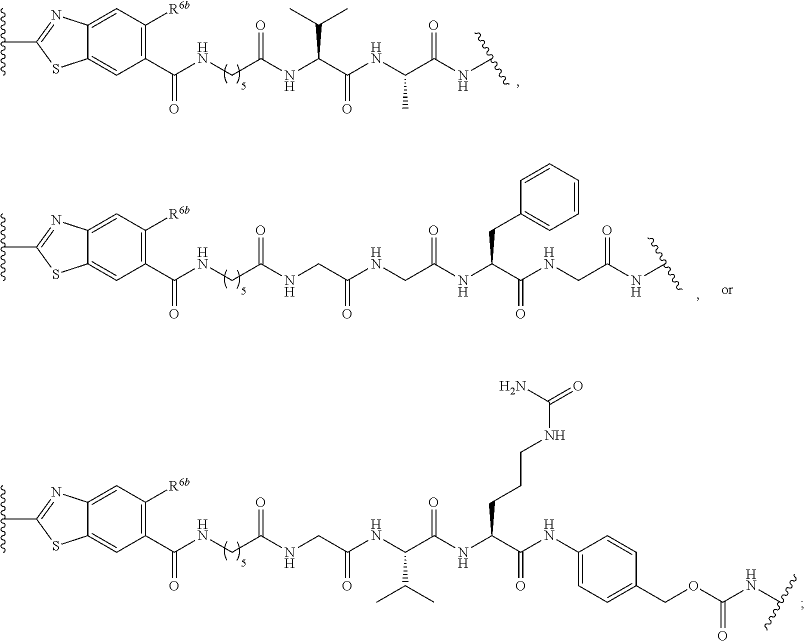

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

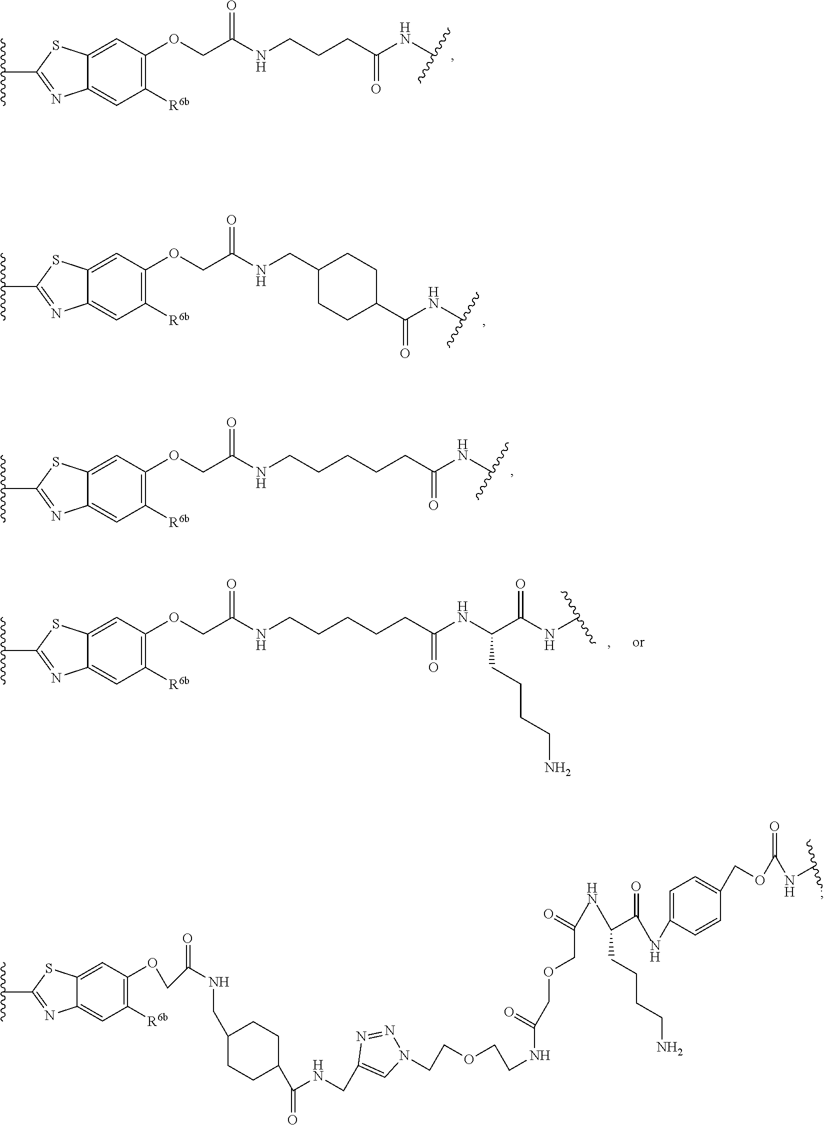

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

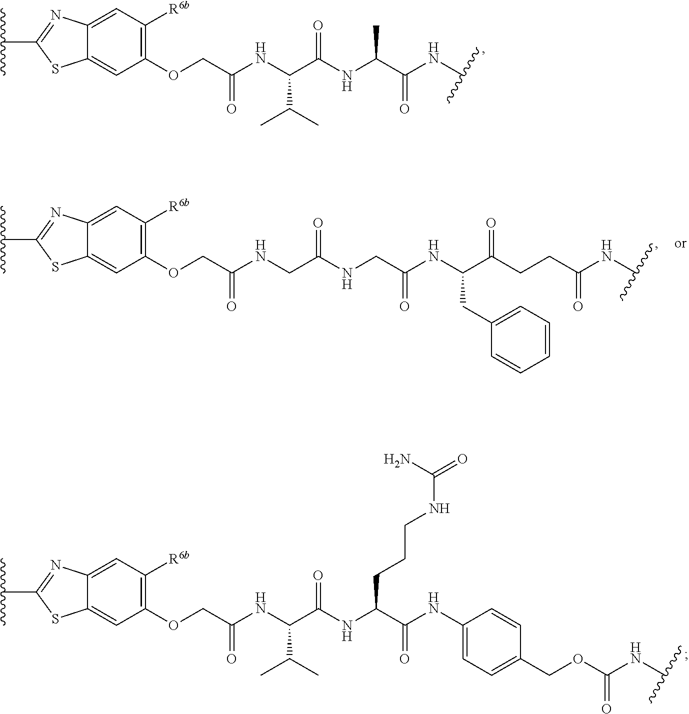

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

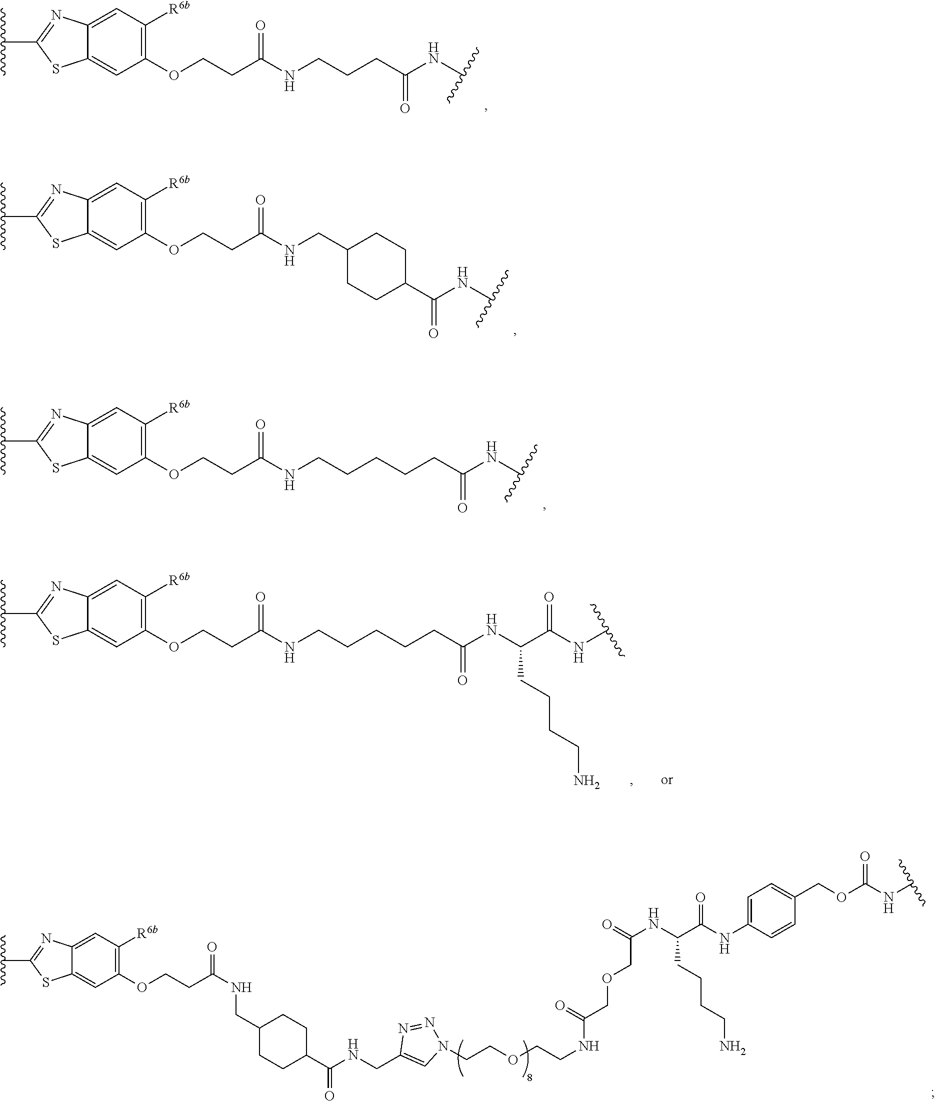

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

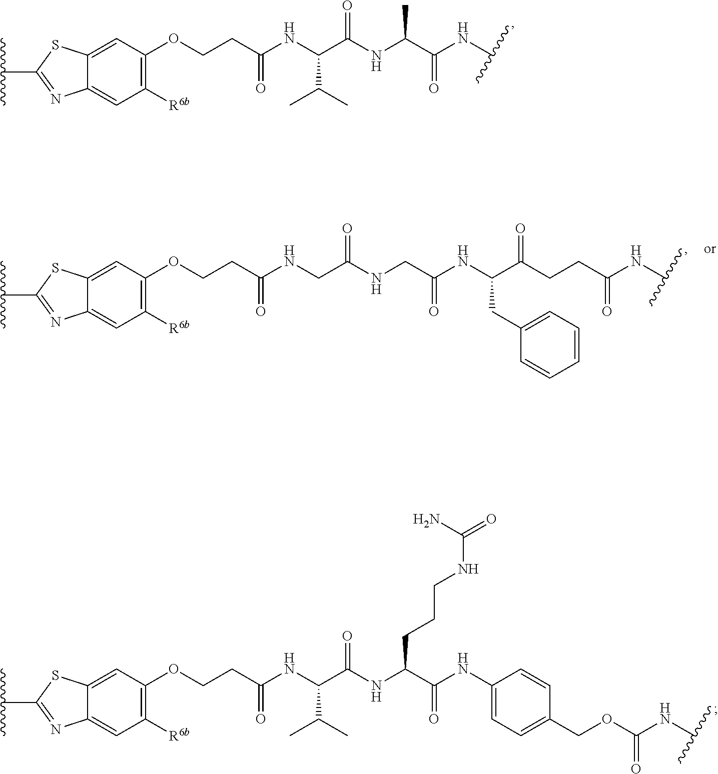

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro.

In certain embodiments, L is:

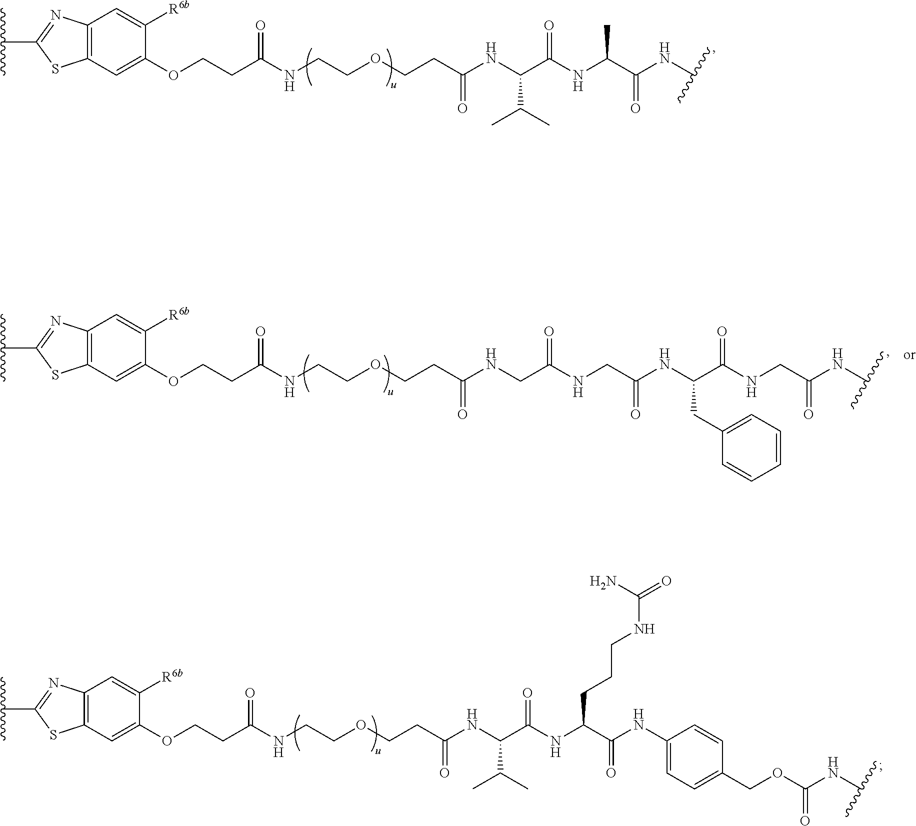

wherein, in one embodiment, each R^{6b} is independently hydrogen or halo; in another embodiment, each R^{6b} is independently hydrogen or fluoro; in yet another embodiment, each R^{6b} is hydrogen; in still another embodiment, each R^{6b} is fluoro; and in one embodiment, each u is independently an integer of 1, 2, 3, 4, 5, 6, 7, or 8; in another embodiment, each u is independently an integer of 4 or 8.

In certain embodiments, each

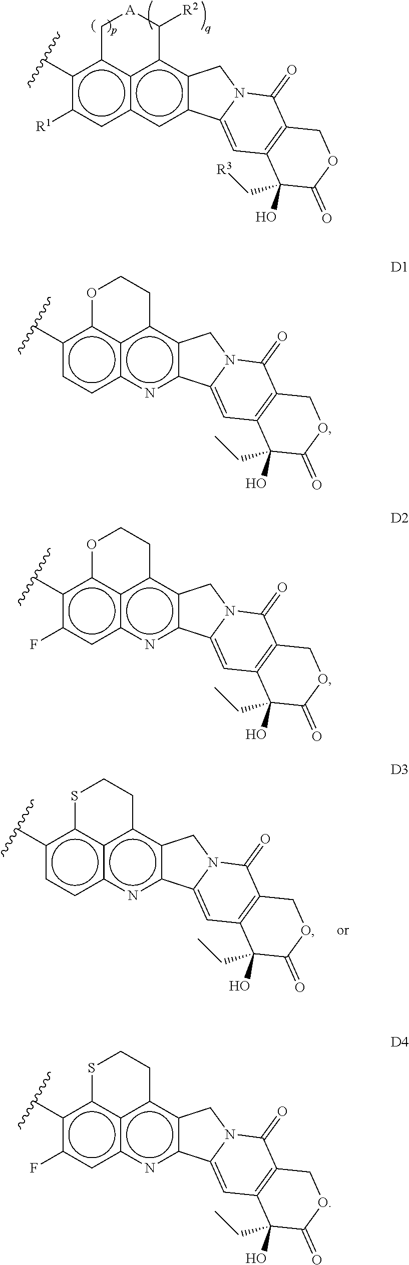

moiety is independently.

In certain embodiments, each

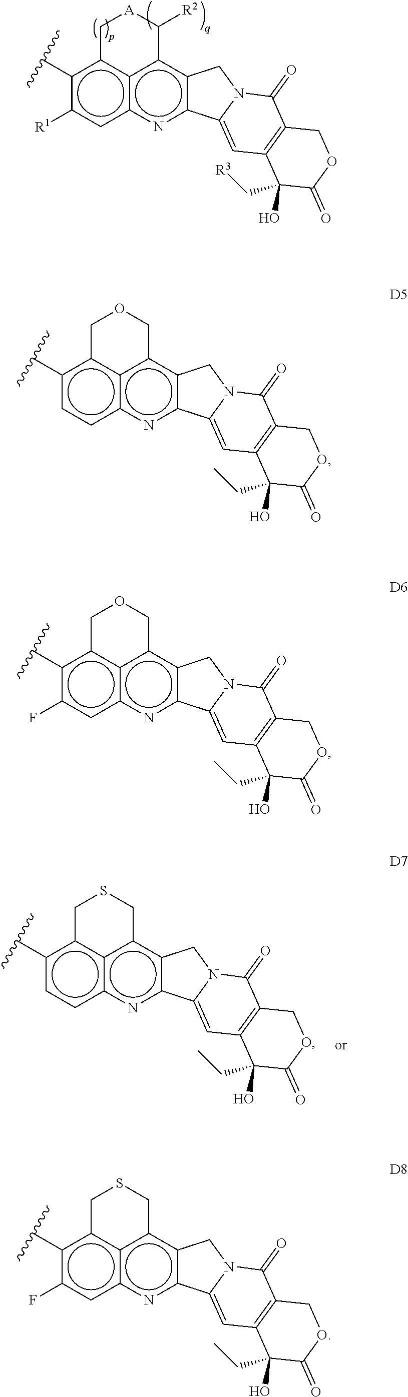

moiety is independently

In one embodiment, provided herein is a compound of Formula (IA):

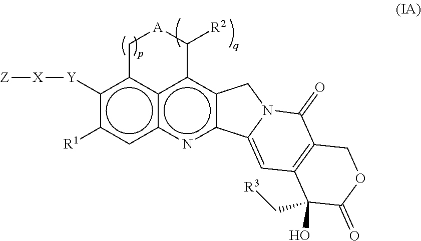

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein:
A is O, S, S(O), S(O₂, or N(R⁴);
X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
Y is a bond, C₁₋₆ alkylene, C₁₋₁₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene;
Z is

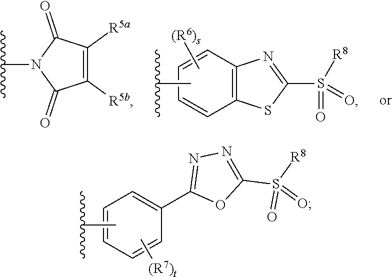

each R¹, R², R³, R^{5a}, and R^{5b} is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O) SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1a}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁴ is (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
each R⁶ and R⁷ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R¹, —S(O)NR^{1b}R^{1c}, or —S(O)₂NR^{1b}R^{1c};
each R¹ is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ is aralkyl, heteroaryl, or heterocyclyl;
p and q are each independently an integer of 0, 1, or 2; with the proviso that the total of p and q is greater than 0;
s is an integer of 0, 1, 2, or 3; and
t is an integer of 0, 1, 2, 3, or 4;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OP(O)(OR^{c})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O) OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃_10 cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S) NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S) NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, Z is

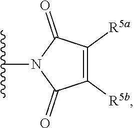

wherein R^{5a} and R^{5b} are each as defined herein.

In another embodiment, provided herein is a compound of Formula (IIA):

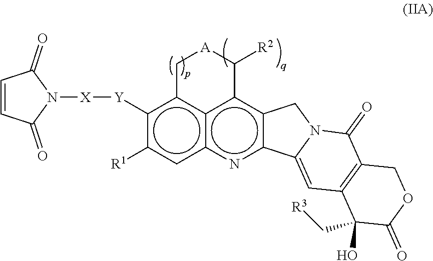

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, A, X, Y, p, and q are each as defined herein.

In certain embodiments, in Formula (IA) or (IIA), p is an integer of 0 and q is an integer of 1. In certain embodiments, in Formula (IA) or (IIA), p is an integer of 1 and q is an integer of 0.

In certain embodiments, in Formula (IA) or (IIA), p is an integer of 0 and q is an integer of 2. In certain embodiments, in Formula (IA) or (IIA), p and q are each an integer of 1. In certain embodiments, in Formula (IA) or (IIA), p is an integer of 2 and q is an integer of 0.

In another embodiment, provided herein is a compound of Formula (TITA):

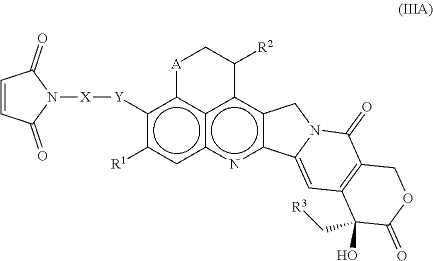

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, A, X, and Y are each as defined herein.

In yet another embodiment, provided herein is a compound of Formula (IVA):

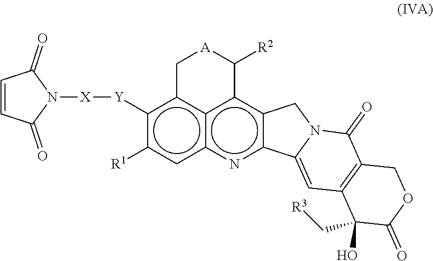

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, A, X, and Y are each as defined herein.

In certain embodiments, in any one of Formulae (IA) to (IVA), A is O. In certain embodiments, in any one of Formulae (IA) to (IVA), A is S. In certain embodiments, in any one of Formulae (IA) to (IVA), A is S(O). In certain embodiments, in any one of Formulae (IA) to (IVA), A is S(O₂). In certain embodiments, in any one of Formulae (IA) to (IVA), A is N(R⁴), wherein R⁴ is as defined herein. In certain embodiments, in any one of Formulae (IA) to (IVA), A is N(R⁴), wherein R⁴ is (i) hydrogen; (ii) C₁₋₆ alkyl or C₁₋₆ heteroalkyl, each optionally substituted with one or more substituents Q; or (iii) —C(O)R^{1a}, wherein R^{1a} is as defined herein. In certain embodiments, in any one of Formulae (IA) to (IVA), A is N(H), N(CH₃), or N(C(O)CH₃). In certain embodiments, in any one of Formulae (IA) to (IVA), A is N(H).

In yet another embodiment, provided herein is a compound of Formula (VA):

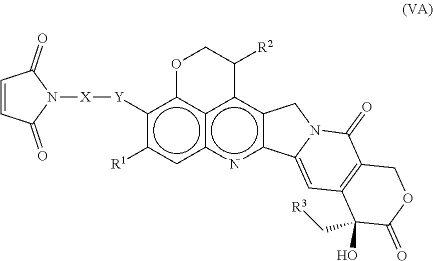

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, X, and Y are each as defined herein.

In yet another embodiment, provided herein is a compound of Formula (VIA):

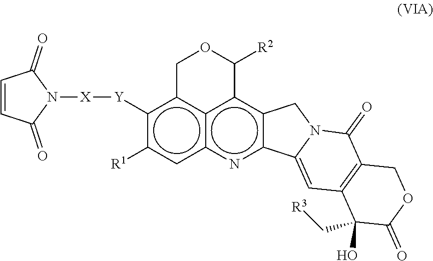

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, X, and Y are each as defined herein.

In still another embodiment, provided herein is a compound of Formula (VIIA):

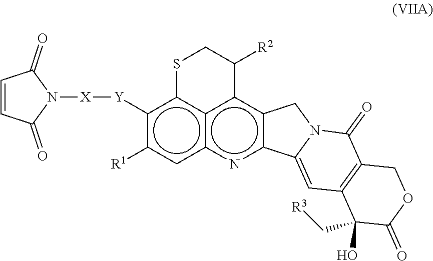

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, X, and Y are each as defined herein.

In still another embodiment, provided herein is a compound of Formula (VIIIA):

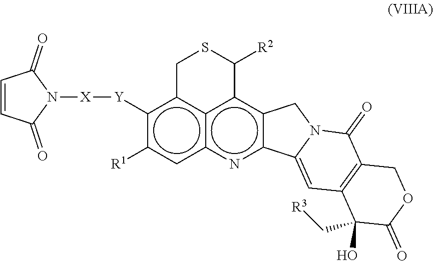

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, X, and Y are each as defined herein.

In certain embodiments, in any one of Formulae (IA) to (VIIIA), R¹ is hydrogen, deuterium, or halo. In certain embodiments, in any one of Formulae (IA) to (VIIIA), R¹ is hydrogen, deuterium, or fluoro. In certain embodiments, in any one of Formulae (IA) to (VIIIA), R¹ is hydrogen or fluoro. In certain embodiments, in any one of Formulae (IA) to (VIIIA), R¹ is hydrogen. In certain embodiments, in any one of Formulae (IA) to (VIIIA), R¹ is fluoro.

In certain embodiments, in any one of Formulae (IA) to (VIIIA), R² is (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein. In certain embodiments, in any one of Formulae (IA) to (VIIIA), R² is hydrogen, amino, methylamino, acetamido, or hydroxyacetamido. In certain embodiments, in any one of Formulae (IA) to (VIIIA), R² is hydrogen or amino. In certain embodiments, in any one of Formulae (IA) to (VIIIA), R² is hydrogen. In certain embodiments, in any one of Formulae (IA) to (VIIIA), R² is amino.

In certain embodiments, in any one of Formulae (IA) to (VIIIA), R³ is C₁₋₆ alkyl, C₁₋₆ heteroalkyl, or C₂₋₆ alkenyl, each optionally substituted with one or more substituents Q. In certain embodiments, in any one of Formulae (IA) to (VIIIA), R³ is methyl, fluoromethyl, methoxymethyl, or ethenyl. In certain embodiments, in any one of Formulae (IA) to (VIIIA), R³ is methyl.

In certain embodiments, in any one of Formulae (IA) to (VIIIA),
R¹ is hydrogen, deuterium, or halo;
R² is (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein; and
R³ is C₁₋₆ alkyl, C₁₋₆ heteroalkyl, or C₂₋₆ alkenyl, each optionally substituted with one or more substituents Q.

In certain embodiments, in any one of Formulae (IA) to (VIIIA),
R¹ is hydrogen, deuterium, or fluoro;
R² is hydrogen, amino, methylamino, acetamido, or hydroxyacetamido; and
R³ is methyl, fluoromethyl, hydroxymethyl, or ethenyl.

In certain embodiments, in any one of Formulae (IA) to (VIIIA),
R¹ is hydrogen or fluoro;
R² is hydrogen or amino; and
R³ is methyl.

In certain embodiments, in any one of Formulae (IA) to (VIIIA),
R¹ is hydrogen or fluoro;
R² is hydrogen; and
R³ is methyl.

In one embodiment, provided herein is a compound of Formula (IXA):

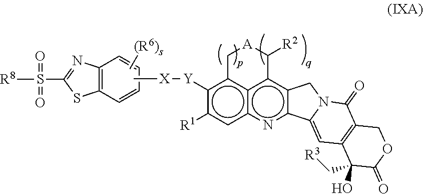

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁶, R⁸, A, X, Y, p, q, and s are each as defined herein.

In certain embodiments, in Formula (IXA), each R⁶ is independently deuterium or halo. In certain embodiments, in Formula (IXA), each R⁶ is independently deuterium or fluoro. In certain embodiments, in Formula (IXA), each R⁶ is independently halo. In certain embodiments, in Formula (IXA), each R⁶ is fluoro.

In certain embodiments, in Formula (IXA), s is an integer of 0 or 1. In certain embodiments, in Formula (IXA), s is an integer of 0. In certain embodiments, in Formula (IXA), s is an integer of 1.

In another embodiment, provided herein is a compound of Formula (XA):

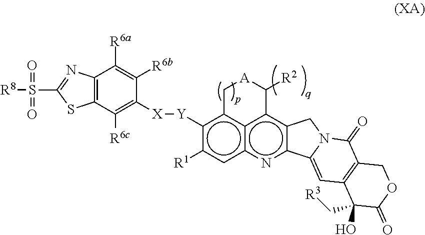

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R^{6a}, R^{6b}, and R^{6c} are each independently hydrogen or R⁶; and R¹, R², R³, R⁶, R⁸, A, X, Y, p, and q are each as defined herein.

In certain embodiments, in Formula (IXA) or (XA), p is an integer of 0 and q is an integer of 1. In certain embodiments, in Formula (IXA) or (XA), p is an integer of 1 and q is an integer of 0.

In certain embodiments, in Formula (IXA) or (XA), p is an integer of 0 and q is an integer of 2. In certain embodiments, in Formula (IXA) or (XA), p and q are each an integer of 1. In certain embodiments, in Formula (IXA) or (XA), p is an integer of 2 and q is an integer of 0.

In yet another embodiment, provided herein is a compound of Formula (XIA):

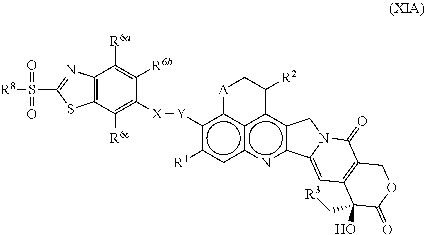

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}, R^{6c}, A, X, and Y are each as defined herein.

In yet another embodiment, provided herein is a compound of Formula (XIIA):

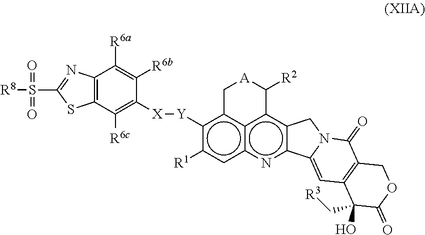

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}R⁶%, X, and Y are each as defined herein.

In certain embodiments, in any one of Formulae (IXA) to (XIIA), A is O. In certain embodiments, in any one of Formulae (IXA) to (XIIA), A is S. In certain embodiments, in any one of Formulae (IXA) to (XIIA), A is S(O). In certain embodiments, in any one of Formulae (IXA) to (XIIA), A is S(O₂). In certain embodiments, in any one of Formulae (IXA) to (XIIA), A is N(R⁴), wherein R⁴ is as defined herein. In certain embodiments, in any one of Formulae (IXA) to (XIIA), A is N(R⁴), wherein R⁴ is (i) hydrogen; (ii) C₁₋₆ alkyl or C₁₋₆ heteroalkyl, each optionally substituted with one or more substituents Q; or (iii) —C(O)R^{1a}, wherein R^{1a} is as defined herein. In certain embodiments, in any one of Formulae (IXA) to (XIIA), A is N(H), N(CH₃), or N(C(O)CH₃). In certain embodiments, in any one of Formulae (IXA) to (XIIA), A is N(H).

In yet another embodiment, provided herein is a compound of Formula (XIIIA):

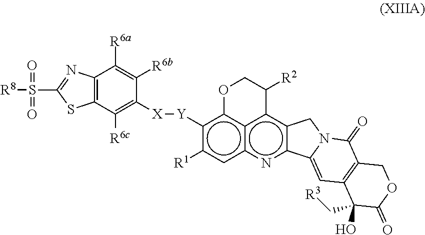

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}, R^{6c} X, and Y are each as defined herein.

In yet another embodiment, provided herein is a compound of Formula (XIVA):

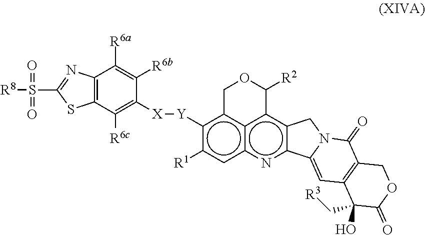

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}, R^{6c}, X, and Y are each as defined herein.

In still another embodiment, provided herein is a compound of Formula (XVA):

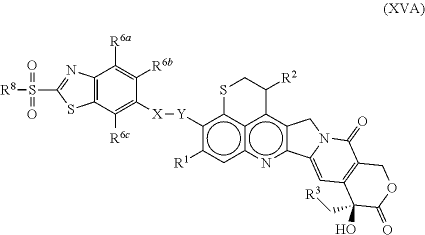

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}, R^{6c}, X, and Y are each as defined herein.

In still another embodiment, provided herein is a compound of Formula (XVIA):

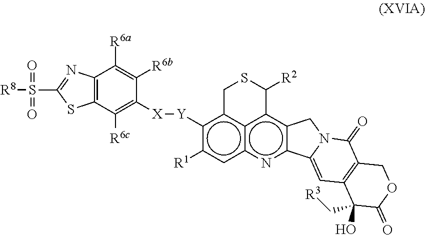

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein R¹, R², R³, R⁸, R^{6a}, R^{6b}, R^{6c} X, and Y are each as defined herein.

In certain embodiments, in any one of Formulae (IXA) to (XVIA), R¹ is hydrogen, deuterium, or halo. In certain embodiments, in any one of Formulae (IXA) to (XVIA), R¹ is hydrogen, deuterium, or fluoro. In certain embodiments, in any one of Formulae (IXA) to (XVIA), R¹ is hydrogen or fluoro. In certain embodiments, in any one of Formulae (IXA) to (XVIA), R¹ is hydrogen. In certain embodiments, in any one of Formulae (IXA) to (XVIA), R¹ is fluoro.

In certain embodiments, in any one of Formulae (IXA) to (XVIA), R² is (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein. In certain embodiments, in any one of Formulae (IXA) to (XVIA), R² is hydrogen, amino, methylamino, acetamido, or hydroxyacetamido. In certain embodiments, in any one of Formulae (IXA) to (XVIA), R² is hydrogen or amino. In certain embodiments, in any one of Formulae (IXA) to (XVIA), R² is hydrogen. In certain embodiments, in any one of Formulae (IXA) to (XVIA), R² is amino.

In certain embodiments, in any one of Formulae (IXA) to (XVIA), R³ is C₁₋₆ alkyl, C₁₋₆ heteroalkyl, or C₂₋₆ alkenyl, each optionally substituted with one or more substituents Q. In certain embodiments, in any one of Formulae (IXA) to (XVIA), R 3 is methyl, fluoromethyl, methoxymethyl, or ethenyl. In certain embodiments, in any one of Formulae (IXA) to (XVIA), R³ is methyl.

In certain embodiments, in any one of Formulae (IXA) to (XVIA), R¹ is C₁₋₆ alkyl, C₁₋₆ heteroalkyl, or C₃₋₁₀ cycloalkyl, each optionally substituted with one or more substituents Q. In certain embodiments, in any one of Formulae (IXA) to (XVIA), R¹ is C₁₋₆ alkyl, optionally substituted with one or more substituents Q. In certain embodiments, in any one of Formulae (IXA) to (XVIA), R⁸ is methyl. In certain embodiments, in any one of Formulae (IXA) to (XVIA), R⁸ is C₁₋₆ heteroalkyl, optionally substituted with one or more substituents Q. In certain embodiments, in any one of Formulae (IXA) to (XVIA), R⁸ is trifluoromethyl. In certain embodiments, in any one of Formulae (IXA) to (XVIA), R⁸ is C₃₋₁₀ cycloalkyl, optionally substituted with one or more substituents Q. In certain embodiments, in any one of Formulae (IXA) to (XVIA), R⁸ is monocyclic C₃₋₁₀ cycloalkyl, optionally substituted with one or more substituents Q. In certain embodiments, in any one of Formulae (IXA) to (XVIA), R⁸ is cyclopropyl or 1-fluorocyclopropyl. In certain embodiments, in any one of Formulae (IXA) to (XVIA), R⁸ is cyclopropyl.

In certain embodiments, in any one of Formulae (XA) to (XVIA), R^{6a} is hydrogen or deuterium. In certain embodiments, in any one of Formulae (XA) to (XVIA), R^{6a} is hydrogen.

In certain embodiments, in any one of Formulae (XA) to (XVIA), R^{6b} is hydrogen, deuterium, or halo. In certain embodiments, in any one of Formulae (XA) to (XVIA), R^{6b} is hydrogen, deuterium, or fluoro. In certain embodiments, in any one of Formulae (XA) to (XVIA), R^{6b} is hydrogen or fluoro. In certain embodiments, in any one of Formulae (XA) to (XVIA), R^{6b} is hydrogen. In certain embodiments, in any one of Formulae (XA) to (XVIA), R^{6b} is fluoro.

In certain embodiments, in any one of Formulae (XA) to (XVIA), R^{6c} is hydrogen or deuterium. In certain embodiments, in any one of Formulae (XA) to (XVIA), R^{6c} is hydrogen.

In certain embodiments, in any one of Formulae (XA) to (XVIA),
R¹ is hydrogen, deuterium, or halo;
R² is (i) hydrogen or deuterium; (ii) heterocyclyl, optionally substituted with one or more substituents Q; or (iii) —NR^{1b}R^{1c} or —NR^{1a}C(O)R^{1d}; wherein R^{1a}, R^{1b}, R^{1c}, and R^{1d} are each as defined herein; and
R³ is C₁₋₆ alkyl, C₁₋₆ heteroalkyl, or C₂₋₆ alkenyl, each optionally substituted with one or more substituents Q;
R⁸ is C₁₋₆ alkyl, C₁₋₆ heteroalkyl, or C₃₋₁₀ cycloalkyl, each optionally substituted with one or more substituents Q;
R^{6a} and R^{6c} are each independently hydrogen or deuterium; and
R^{6b} is hydrogen, deuterium, or halo.

In certain embodiments, in any one of Formulae (XA) to (XVIA),
R¹ is hydrogen, deuterium, or fluoro;
R² is hydrogen, amino, methylamino, acetamido, or hydroxyacetamido; and
R³ is methyl, fluoromethyl, hydroxymethyl, or ethenyl;
R⁸ is methyl, fluoromethyl, cyclopropyl, or 1-fluorocyclopropyl;
R^{6a} and R^{6c} are each hydrogen; and
R^{6b} is hydrogen or fluoro.

In certain embodiments, in any one of Formulae (XA) to (XVIA),
R¹ is hydrogen or fluoro;
R² is hydrogen or amino;
R³ is methyl;
R⁸ is methyl or cyclopropyl;
R^{6a} and R^{6c} are each hydrogen; and
R^{6b} is hydrogen or fluoro.

In certain embodiments, in any one of Formulae (XA) to (XVIA),
R⁸ is hydrogen or fluoro;
R² is hydrogen;
R³ is methyl;
R¹ is methyl or cyclopropyl;
R^{6a} and R^{6c} are each hydrogen; and
R^{6b} is hydrogen or fluoro.

The groups, R¹, R², R³, R⁴, R⁶, R⁸, R^{5a}, R^{5b}, R^{6a}, R^{6b}, R^{6c}, A, X, Y, Z, m, p, q, and s in formulae described herein, including Formulae (I) to (XXII) and (IA) to (XVIA), are further defined in the embodiments described herein. All combinations of the embodiments provided herein for such groups are within the scope of this disclosure.

In certain embodiments, each R¹ is hydrogen. In certain embodiments, each R¹ is deuterium. In certain embodiments, each R¹ is cyano. In certain embodiments, each R¹ is independently halo. In certain embodiments, each R¹ is independently fluoro or chloro. In certain embodiments, each R¹ is fluoro. In certain embodiments, each R¹ is nitro. In certain embodiments, each R¹ is independently C₁₋₆ alkyl, optionally substituted with one or more substituents Q. In certain embodiments, each R¹ is independently methyl. In certain embodiments, each R¹ is independently C₁₋₆ heteroalkyl, optionally substituted with one or more substituents Q. In certain embodiments, each R¹ is independently C₂₋₆ alkenyl, optionally substituted with one or more substituents Q. In certain embodiments, each R¹ is independently C₂₋₆ alkynyl, optionally substituted with one or more substituents Q. In certain embodiments, each R¹ is independently C₃₋₁₀ cycloalkyl, optionally substituted with one or more substituents Q. In certain embodiments, each R¹ is independently C₆₋₁₄ aryl, optionally substituted with one or more substituents Q. In certain embodiments, each R¹ is independently C₇₋₁₅ aralkyl, optionally substituted with one or more substituents Q. In certain embodiments, each R¹ is independently heteroaryl, optionally substituted with one or more substituents Q. In certain embodiments, each R¹ is independently heterocyclyl, optionally substituted with one or more substituents Q. In certain embodiments, each R¹ is independently hydrogen or fluoro.

In certain embodiments, each R¹ is independently —C(O)R^{1a}, wherein R^{1a} is as defined herein. In certain embodiments, each R¹ is independently —C(O)OR^{1a}, wherein R^{1a} is as defined herein. In certain embodiments, each R¹ is independently —C (O)NR^{1b}R^{1c}, wherein R^{1b} and R^{1c} are each as defined herein. In certain embodiments, each R¹ is independently —C(O)SR^{1a}, wherein R^{1a} is as defined herein. In certain embodiments, each R¹ is independently —C(NR^{1a})NR^{1b}R^{1c}, wherein R^{1a}, R^{1b}, and R^{1c} are each as defined herein. In certain embodiments, each R¹ is independently —C(S)R^{1a}, wherein R^{1a} is as defined herein. In certain embodiments, each R¹ is independently —C(S)OR^{1a}, wherein R^{1a} is as defined herein. In certain embodiments, each R¹ is independently —C(S)NR^{1b}R^{1c}, wherein R^{1b} and R^{1c} are each as defined herein. In certain embodiments, each R¹ is independently —OR^{1a}, wherein R^{1a} is as defined herein. In certain embodiments, each R¹ is independently —OC(O)R^{1a}, wherein R^{1a} is as defined herein. In certain embodiments, each R¹ is independently —OC(O)OR^{1a}, wherein R^{1a} is as defined herein. In certain embodiments, each R¹ is independently —OC(O)NR^{1b}R^{1c}, wherein R^{1b} and R^{1c} are each as defined herein. In certain embodiments, each R¹ is independently —OC(O)SR^{1a}, wherein R^{1d} is as defined herein. In certain embodiments, each R¹ is independently —OC(NR^{1a})NR^{1b}R^{1c}, wherein R^{1d}, R^{1b}, and R^{1c} are each as defined herein. In certain embodiments, each R¹ is independently —OC(S)R^{1d}, wherein R^{1a} is as defined herein. In certain embodiments, each R¹ is independently —OC(S)OR^{1a}, wherein R^{1a} is as defined herein. In certain embodiments, each R¹ is independently —OC(S)NR^{1b}R^{1c}, wherein R^{1b} and R^{1c} are each as defined herein. In certain embodiments, each R¹ is independently —OS(O)R^{1d} wherein R^{1d} is as defined herein. In certain embodiments, each R¹ is independently-OS(O)₂R^{1a}, wherein R^{1d} is as defined herein. In certain embodiments, each R¹ is independently —OS(O)NR^{1b}R^{1c}, wherein R^{1b} and R^{1c} are each as defined herein. In certain embodiments, each R¹ is independently —OS(O)₂NR^{1b}R^{1c}, wherein R^{1b} and R^{1c} are each as defined herein. In certain embodiments, each R¹ is independently —NR^{1b}R^{1c}, wherein R^{1b} and R^{1c} are each as defined herein. In certain embodiments, each R¹ is independently —NR^{1a}C(O)R^{1d}, wherein R^{1a} and R^{1d} are each as defined herein. In certain embodiments, each R¹ is independently —NR^{1a}C(O)OR^{1d}, wherein R^{1a} and R^{1d} are each as defined herein. In certain embodiments, each R¹ is independently —NR^{1a}C(O)NR^{1b}R^{1c}, wherein R^{1d}, R^{1b}, and R^{1c} are each as defined herein. In certain embodiments, each R¹ is independently —NR^{1a}C(O)SR^{1d}, wherein R^{1a} and R^{1d} are each as defined herein. In certain embodiments, each R¹ is independently —NR^{1a}C(NR^{1d})NR^{1b}R^{1c}, wherein R^{1d}, R^{1b}, R^{1c}, and R^{1d} are each as defined herein. In certain embodiments, each R¹ is independently —NR^{1a}C(S)R^{1d}, wherein R^{1a} and R^{1d} are each as defined herein. In certain embodiments, each R¹ is independently —N^{1a}C(S)OR^{1d}, wherein R^{1a} and R^{1d} are each as defined herein. In certain embodiments, each R¹ is independently —NR^{1a}C(S)NR^{1b}R^{1c}, wherein R^{1a}, R^{1b}, and R^{1c} are each as defined herein. In certain embodiments, each R¹ is independently —NR^{1a}S(O)R^{1d}, wherein R^{1a} and R^{1d} are each as defined herein. In certain embodiments, each R¹ is independently —NR^{1a}S(O)₂R^{1d}, wherein R^{1a} and R^{1d} are each as defined herein. In certain embodiments, each R¹ is independently —NR^{1a}S(O)NR^{1b}R^{1c}, wherein R^{1d}, R^{1b}, and R^{1c} are each as defined herein. In certain embodiments, each R¹ is independently —NR^{1a}S(O)₂NR^{1b}R^{1c}, wherein R^{1a}, R^{1b}, and R^{1c} are each as defined herein. In certain embodiments, each R¹ is independently —SR^{1a}, wherein R^{1d} is as defined herein. In certain embodiments, each R¹ is independently —S(O)R^{1a}, wherein R^{1d} is as defined herein. In certain embodiments, each R¹ is independently —S(O)₂R^{1a}, wherein R^{1d} is as defined herein. In certain embodiments, each R¹ is independently —S(O)NR^{1b}R^{1c}, wherein R^{1b} and R^{1c} are each as defined herein. In certain embodiments, each R¹ is independently —S(O)₂NR^{1b}R^{1c}, wherein R^{1b} and R^{1c} are each as defined herein.

In certain embodiments, each R² is hydrogen. In certain embodiments, each R² is deuterium. In certain embodiments, each R² is cyano. In certain embodiments, each R² is independently halo. In certain embodiments, each R² is independently fluoro or chloro. In certain embodiments, each R² is fluoro. In certain embodiments, each R² is nitro. In certain embodiments, each R² is independently C₁₋₆ alkyl, optionally substituted with one or more substituents Q. In certain embodiments, each R² is methyl. In certain embodiments, each R² is independently C₁₋₆ heteroalkyl, optionally substituted with one or more substituents Q. In certain embodiments, each R² is independently C₂₋₆ alkenyl, optionally substituted with one or more substituents Q. In certain embodiments, each R² is independently C₂₋₆ alkynyl, optionally substituted with one or more substituents Q. In certain embodiments, each R² is independently C₃₋₁₀ cycloalkyl, optionally substituted with one or more substituents Q. In certain embodiments, each R² is independently C₆₋₁₄ aryl, optionally substituted with one or more substituents Q. In certain embodiments, each R² is independently C₇₋₁₅ aralkyl, optionally substituted with one or more substituents Q. In certain embodiments, each R² is independently heteroaryl, optionally substituted with one or more substituents Q. In certain embodiments, each R² is independently heterocyclyl, optionally substituted with one or more substituents Q.

In certain embodiments, each R² is independently —C(O)R^{1a}, wherein R^{1d} is as defined herein. In certain embodiments, each R² is independently —C(O)OR^{1d}, wherein R^{1d} is as defined herein. In certain embodiments, each R² is independently —C(O)NR^{1b}R^{1c}, wherein R^{1b} and R^{1c} are each as defined herein. In certain embodiments, each R² is independently —C(O)SR^{1a}, wherein R^{1a} is as defined herein. In certain embodiments, each R² is independently —C(NR^{1a})NR^{1b}R^{1c}, wherein R^{1a}, R^{1b}, and R^{1c} are each as defined herein. In certain embodiments, each R² is independently —C(S)R^{1a}, wherein R^{1a} is as defined herein. In certain embodiments, each R² is independently —C(S)OR^{1a}, wherein R^{1d} is as defined herein. In certain embodiments, each R² is independently —C(S)NR^{1b}R^{1c}, wherein R^{1b} and R^{1c} are each as defined herein. In certain embodiments, each R² is independently —OR^{1a}, wherein R^{1a} is as defined herein. In certain embodiments, each R² is independently —OC(O)R^{1a}, wherein R^{1a} is as defined herein. In certain embodiments, each R² is independently —OC(O)OR^{1d}, wherein R^{1d} is as defined herein. In certain embodiments, each R² is independently —OC(O)NR^{1b}R^{1c}, wherein R^{1b} and R^{1c} are each as defined herein. In certain embodiments, each R² is independently —OC(O)SR^{1d}, wherein R^{1d} is as defined herein. In certain embodiments, each R² is independently —OC(NR^{1d})NR^{1b}R^{1c}, wherein R^{1d}, R^{1b}, and R^{1c} are each as defined herein. In certain embodiments, each R² is independently —OC(S)R^{1d}, wherein R^{1a} is as defined herein. In certain embodiments, each R² is independently —OC(S)OR^{1d}, wherein R^{1d} is as defined herein. In certain embodiments, each R² is independently —OC(S)NR^{1b}R^{1c}, wherein R^{1b} and R^{1c} are each as defined herein. In certain embodiments, each R² is independently —OS(O)R^{1d}, wherein R^{1d} is as defined herein. In certain embodiments, each R² is independently —OS(O)₂R^{1a}, wherein R^{1a} is as defined herein. In certain embodiments, each R² is independently —OS(O)NR^{1b}R^{1c}, wherein R^{1b} and R^{1c} are each as defined herein. In certain embodiments, each R² is independently —OS(O)₂NR^{1b}R^{1c}, wherein R^{1b} and R^{1c} are each as defined herein. In certain embodiments, each R² is independently —NR^{1b}R^{1c}, wherein R^{1b} and R^{1c} are each as defined herein. In certain embodiments, each R² is independently amino (—NH₂) or methylamino (—NHCH₃). In certain embodiments, each R² is independently —NR^{1a}C(O)R^{1d}, wherein R^{1a} and R^{1d} are each as defined herein. In certain embodiments, each R² is independently acetamido or hydroxyacetamido. In certain embodiments, each R² is independently —NR^{1a}C(O)OR^{1d}, wherein R^{1a} and R^{1d} are each as defined herein. In certain embodiments, each R² is independently —NR^{1a}C(O)NR^{1b}R^{1c}, wherein R^{1d}, R^{1b}, and R^{1c} are each as defined herein. In certain embodiments, each R² is independently —NR^{1a}C(O)SR^{1d} wherein R^{1a} and R^{1d} are each as defined herein. In certain embodiments, each R² is independently —NR^{1d}C(NR^{1d})NR^{1b}R^{1c}, wherein R^{1d}, R^{1b}, R^{1c}, and R^{1d} are each as defined herein. In certain embodiments, each R² is independently —NR^{1a}C(S)R^{1d}, wherein R^{1a} and R^{1d} are each as defined herein. In certain embodiments, each R² is independently —NR^{1a}C(S)OR^{1d}, wherein R^{1a} and R^{1d} are each as defined herein. In certain embodiments, each R² is independently —NR^{1a}C(S)NR^{1b}R^{1c}, wherein R^{1a}, Rib, and R^{1c} are each as defined herein. In certain embodiments, each R² is independently —NR^{1d}S(O)R^{1d}, wherein R^{1a} and R^{1d} are each as defined herein. In certain embodiments, each R² is independently —NR^{1a}S(O)₂R^{1d}, wherein R^{1a} and R^{1d} are each as defined herein. In certain embodiments, each R² is independently —NR^{1a}S(O)NR^{1b}R^{1c}, wherein R^{1d}, R^{1b}, and R^{1c} are each as defined herein. In certain embodiments, each R² is independently —NR^{1a}S(O)₂NR^{1b}R^{1c}, wherein R^{1d}, R^{1b}, and R^{1c} are each as defined herein. In certain embodiments, each R² is independently —SR^{1a}, wherein R^{1d} is as defined herein. In certain embodiments, each R² is independently —S(O)R^{1d}, wherein R^{1a} is as defined herein. In certain embodiments, each R² is independently —S(O)₂R^{1a}, wherein R^{1a} is as defined herein. In certain embodiments, each R² is independently —S(O)NR^{1b}R^{1c}, wherein R^{1b} and R^{1c} are each as defined herein. In certain embodiments, each R² is independently —S(O)₂NR^{1b}R^{1c}, wherein R^{1b} and R^{1c} are each as defined herein.

In certain embodiments, each R³ is hydrogen. In certain embodiments, each R³ is deuterium. In certain embodiments, each R³ is cyano. In certain embodiments, each R³ is independently halo. In certain embodiments, each R³ is independently fluoro or chloro. In certain embodiments, each R³ is fluoro. In certain embodiments, each R³ is nitro. In certain embodiments, each R³ is independently C₁₋₆ alkyl, optionally substituted with one or more substituents Q. In certain embodiments, each R³ is independently methyl, fluoromethyl, or methoxymethyl. In certain embodiments, each R³ is methyl. In certain embodiments, each R³ is independently C₁₋₆ heteroalkyl, optionally substituted with one or more substituents Q. In certain embodiments, each R³ is independently C₂₋₆ alkenyl, optionally substituted with one or more substituents Q. In certain embodiments, each R³ is ethenyl. In certain embodiments, each R³ is independently C₂₋₆ alkynyl, optionally substituted with one or more substituents Q. In certain embodiments, each R³ is independently C₃₋₁₀ cycloalkyl, optionally substituted with one or more substituents Q. In certain embodiments, each R³ is independently C₆₋₁₄ aryl, optionally substituted with one or more substituents Q. In certain embodiments, each R³ is independently C₇₋₁₅ aralkyl, optionally substituted with one or more substituents Q. In certain embodiments, each R³ is independently heteroaryl, optionally substituted with one or more substituents Q. In certain embodiments, each R³ is independently heterocyclyl, optionally substituted with one or more substituents Q.

In certain embodiments, each R³ is independently —C(O)R^{1a}, wherein R^{1a} is as defined herein. In certain embodiments, each R³ is independently —C(O)OR^{1a}, wherein R^{1a} is as defined herein. In certain embodiments, each R³ is independently —C(O)NR^{1b}R^{1c}, wherein R^{1b} and R^{1c} are each as defined herein. In certain embodiments, each R³ is independently —C(O)SR^{1a}, wherein R^{1a} is as defined herein. In certain embodiments, each R³ is independently —C(NR^{1a})NR^{1b}R^{1c}, wherein R^{1a}, R^{1b}, and R^{1c} are each as defined herein. In certain embodiments, each R³ is independently —C(S)R^{1d}, wherein R^{1d} is as defined herein. In certain embodiments, each R³ is independently —C(S)OR^{1d}, wherein R^{1d} is as defined herein. In certain embodiments, each R³ is independently —C(S)NR^{1b}R^{1c}, wherein R^{1b} and R^{1c} are each as defined herein. In certain embodiments, each R³ is independently —OR^{1a}, wherein R^{1d} is as defined herein. In certain embodiments, each R³ is independently —OC(O)R^{1d}, wherein R^{1d} is as defined herein. In certain embodiments, each R³ is independently —OC(O)OR^{1d}, wherein R^{1d} is as defined herein. In certain embodiments, each R³ is independently —OC(O)NR^{1b}R^{1c}, wherein R^{1b} and R^{1c} are each as defined herein. In certain embodiments, each R³ is independently —OC(O)SR^{1d}, wherein R^{1d} is as defined herein. In certain embodiments, each R³ is independently —OC(NR^{1d})NR^{1b}R^{1c}, wherein R^{1d}, R^{1b}, and R^{1c} are each as defined herein. In certain embodiments, each R³ is independently —OC(S)R^{1d}, wherein R^{1d} is as defined herein. In certain embodiments, each R³ is independently —OC(S)OR^{1d}, wherein R^{1d} is as defined herein. In certain embodiments, each R³ is independently —OC(S)NR^{1b}R^{1c}, wherein R^{1b} and R^{1c} are each as defined herein. In certain embodiments, each R³ is independently —OS(O)R^{1d}, wherein R^{1d} is as defined herein. In certain embodiments, each R³ is independently —OS(O)₃R^{1a}, wherein R^{1d} is as defined herein. In certain embodiments, each R³ is independently —OS(O)NR^{1b}R^{1c}, wherein R^{1b} and R^{1c} are each as defined herein. In certain embodiments, each R³ is independently —OS(O)₂NR^{1b}R^{1c}, wherein R^{1b} and R^{1c} are each as defined herein. In certain embodiments, each R³ is independently —NR^{1b}R^{1c}, wherein R^{1b} and R^{1c} are each as defined herein. In certain embodiments, each R³ is independently —NR^{1a}C(O)R^{1d}, wherein R^{1a} and R^{1d} are each as defined herein. In certain embodiments, each R³ is independently —NR^{1a}C(O)OR^{1d}, wherein R^{1a} and R^{1d} are each as defined herein. In certain embodiments, each R³ is independently —NR^{1a}C(O)NR^{1b}R^{1c}, wherein R^{1d}, R^{1b}, and R^{1c} are each as defined herein. In certain embodiments, each R³ is independently —NR^{1a}C(O)SR^{1d}, wherein R^{1a} and R^{1d} are each as defined herein. In certain embodiments, each R³ is independently —NR^{1a}C(NR^{1d})NR^{1b}R^{1c} wherein R^{1d}, R^{1b}, R^{1c}, and R^{1d} are each as defined herein. In certain embodiments, each R³ is independently —NR^{1a}C(S)R^{1d}, wherein R^{1a} and R^{1d} are each as defined herein. In certain embodiments, each R³ is independently —NR^{1a}C(S)OR^{1d}, wherein R^{1a} and R^{1d} are each as defined herein. In certain embodiments, each R³ is independently —NR^{1a}C(S)NR^{1b}R^{1c}, wherein R^{1a}, R^{1b}, and R^{1c} are each as defined herein. In certain embodiments, each R³ is independently —NR^{1a}S(O)R^{1d}, wherein R^{1a} and R^{1d} are each as defined herein. In certain embodiments, each R³ is independently —NR^{1a}S(O)₂R^{1d}, wherein R^{1a} and R^{1d} are each as defined herein. In certain embodiments, each R³ is independently —NR^{1a}S(O)NR^{1b}R^{1c}, wherein R^{1d}, R^{1b}, and R^{1c} are each as defined herein. In certain embodiments, each R³ is independently —NR^{1a}S(O)₂NR^{1b}R^{1c}, wherein R^{1a}, R^{1b}, and R^{1c} are each as defined herein. In certain embodiments, each R³ is independently —SR^{1a}, wherein R^{1d} is as defined herein. In certain embodiments, each R³ is independently —S(O)R^{1a}, wherein R^{1d} is as defined herein. In certain embodiments, each R³ is independently —S(O)₂R^{1a}, wherein R^{1d} is as defined herein. In certain embodiments, each R³ is independently —S(O)NR^{1b}R^{1c}, wherein R^{1b} and R^{1c} are each as defined herein. In certain embodiments, each R³ is independently —S(O)₂NR^{1b}R^{1c}, wherein R^{1b} and R^{1c} are each as defined herein.

In certain embodiments, each R⁴ is hydrogen. In certain embodiments, each R⁴ is independently C₁₋₆ alkyl, optionally substituted with one or more substituents Q. In certain embodiments, each R⁴ is methyl. In certain embodiments, each R⁴ is independently C₁₋₆ heteroalkyl, optionally substituted with one or more substituents Q. In certain embodiments, each R⁴ is independently C₂₋₆ alkenyl, optionally substituted with one or more substituents Q. In certain embodiments, each R⁴ is independently C₂₋₆ alkynyl, optionally substituted with one or more substituents Q. In certain embodiments, each R⁴ is independently C₃₋₁₀ cycloalkyl, optionally substituted with one or more substituents Q. In certain embodiments, each R⁴ is independently C₆₋₁₄ aryl, optionally substituted with one or more substituents Q. In certain embodiments, each R⁴ is independently C₇₋₁₅ aralkyl, optionally substituted with one or more substituents Q. In certain embodiments, each R⁴ is independently heteroaryl, optionally substituted with one or more substituents Q. In certain embodiments, each R⁴ is independently heterocyclyl, optionally substituted with one or more substituents Q.

In certain embodiments, each R⁴ is independently —C(O)R^{1a}, wherein R^{1d} is as defined herein. In certain embodiments, each R⁴ is acetyl. In certain embodiments, each R⁴ is independently —C(O)OR^{1d}, wherein R^{1d} is as defined herein. In certain embodiments, each R⁴ is independently —C(O)NR^{1b}R^{1c}, wherein R^{1b} and R^{1c} are each as defined herein. In certain embodiments, each R⁴ is independently —C(O)SR^{1d}, wherein R^{1a} is as defined herein. In certain embodiments, each R⁴ is independently —C(NR^{1a})NR^{1b}R^{1c}, wherein R^{1a}, R^{1b}, and R^{1c} are each as defined herein. In certain embodiments, each R⁴ is independently —C(S)R^{1d}, wherein R^{1d} is as defined herein. In certain embodiments, each R⁴ is independently —C(S)OR^{1a}, wherein R^{1a} is as defined herein. In certain embodiments, each R⁴ is independently —C(S)NR^{1b}R^{1c}, wherein R^{1b} and R^{1c} are each as defined herein. In certain embodiments, each R⁴ is independently —S(O)R^{1d}, wherein R^{1d} is as defined herein. In certain embodiments, each R⁴ is independently —S(O)₂R^{1a}, wherein R^{1d} is as defined herein. In certain embodiments, each R⁴ is independently —S(O)NR^{1b}R^{1c}, wherein R^{1b} and R^{1c} are each as defined herein. In certain embodiments, each R⁴ is independently —S(O)₂NR^{1b}R^{1c}, wherein R^{1b} and R^{1c} are each as defined herein.

In certain embodiments, each R⁶ is deuterium. In certain embodiments, each R⁶ is cyano. In certain embodiments, each R⁶ is independently halo. In certain embodiments, each R⁶ is independently fluoro or chloro. In certain embodiments, each R⁶ is fluoro. In certain embodiments, each R⁶ is nitro. In certain embodiments, each R⁶ is independently C₁₋₆ alkyl, optionally substituted with one or more substituents Q. In certain embodiments, each R⁶ is independently methyl or trifluoromethyl. In certain embodiments, each R⁶ is methyl. In certain embodiments, each R⁶ is independently C₁₋₆ heteroalkyl, optionally substituted with one or more substituents Q. In certain embodiments, each R⁶ is independently C₂₋₆ alkenyl, optionally substituted with one or more substituents Q. In certain embodiments, each R⁶ is independently C₂₋₆ alkynyl, optionally substituted with one or more substituents Q. In certain embodiments, each R⁶ is independently C₃₋₁₀ cycloalkyl, optionally substituted with one or more substituents Q. In certain embodiments, each R⁶ is independently C₆₋₁₄ aryl, optionally substituted with one or more substituents Q. In certain embodiments, each R⁶ is independently C₇₋₁₅ aralkyl, optionally substituted with one or more substituents Q. In certain embodiments, each R⁶ is independently heteroaryl, optionally substituted with one or more substituents Q. In certain embodiments, each R⁶ is independently heterocyclyl, optionally substituted with one or more substituents Q.

In certain embodiments, each R⁶ is independently —C(O)R^{1d}, wherein R^{1d} is as defined herein. In certain embodiments, each R⁶ is independently —C(O)OR^{1d}, wherein R^{1d} is as defined herein. In certain embodiments, each R⁶ is independently —C(O)NR^{1b}R^{1c}, wherein R^{1b} and R^{1c} are each as defined herein. In certain embodiments, each R⁶ is independently —C(O)SR^{1d}, wherein R^{1d} is as defined herein. In certain embodiments, each R⁶ is independently —C(NR^{1d})NR^{1b}R^{1c} wherein R^{1d}, R^{1b}, and R^{1c} are each as defined herein. In certain embodiments, each R⁶ is independently —C(S)R^{1d}, wherein R^{1d} is as defined herein. In certain embodiments, each R⁶ is independently —C(S)OR, wherein R^{1d} is as defined herein. In certain embodiments, each R⁶ is independently —C(S)NR^{1b}R^{1c}, wherein R^{1b} and R^{1c} are each as defined herein. In certain embodiments, each R⁶ is independently —OR^{1a}, wherein R^{1a} is as defined herein. In certain embodiments, each R⁶ is independently —OC(O)R^{1a}, wherein R^{1a} is as defined herein. In certain embodiments, each R⁶ is independently —OC(O)OR^{1a}, wherein R^{1d} is as defined herein. In certain embodiments, each R⁶ is independently —OC(O)NR^{1b}R^{1c}, wherein R^{1b} and R^{1c} are each as defined herein. In certain embodiments, each R⁶ is independently —OC(O)SR^{1a}, wherein R^{1a} is as defined herein. In certain embodiments, each R⁶ is independently —OC(NR^{1a})NR^{1b}R^{1c}, wherein R^{1d}, R^{1b}, and R^{1c} are each as defined herein. In certain embodiments, each R⁶ is independently —OC(S)R^{1d}, wherein R^{1d} is as defined herein. In certain embodiments, each R⁶ is independently —OC(S)OR^{1a}, wherein R^{1d} is as defined herein. In certain embodiments, each R⁶ is independently —OC(S)NR^{1b}R^{1c}, wherein R^{1b} and R^{1c} are each as defined herein. In certain embodiments, each R⁶ is independently —OS(O)R^{1d}, wherein R^{1d} is as defined herein. In certain embodiments, each R⁶ is independently —OS(O)₆R^{1d}, wherein R^{1d} is as defined herein. In certain embodiments, each R⁶ is independently —OS(O)NR^{1b}R^{1c}, wherein R^{1b} and R^{1c} are each as defined herein. In certain embodiments, each R⁶ is independently —OS(O)₂NR^{1b}R^{1c}, wherein R^{1b} and R^{1c} are each as defined herein. In certain embodiments, each R⁶ is independently —NR^{1b}R^{1c}, wherein R^{1b} and R^{1c} are each as defined herein. In certain embodiments, each R⁶ is independently —NR^{1a}C(O)R^{1d}, wherein R^{1a} and R^{1d} are each as defined herein. In certain embodiments, each R⁶ is independently —NR^{1a}C(O)OR^{1d}, wherein R^{1a} and R^{1d} are each as defined herein. In certain embodiments, each R⁶ is independently —NR^{1a}C(O)NR^{1b}R^{1c}, wherein R^{1d}, R^{1b}, and R^{1c} are each as defined herein. In certain embodiments, each R⁶ is independently —NR^{1a}C(O)SR^{1d}, wherein R^{1a} and R^{1d} are each as defined herein. In certain embodiments, each R⁶ is independently —NR^{1a}C(NR^{d})NR^{1b}R^{1c}, wherein R^{1a}, R^{1b}, R^{1c}, and R^{1d} are each as defined herein. In certain embodiments, each R⁶ is independently —NR^{1a}C(S)R^{1d}, wherein R^{1a} and R^{1d} are each as defined herein. In certain embodiments, each R⁶ is independently —NR^{1a}C(S)OR^{1d}, wherein R^{1a} and R^{1d} are each as defined herein. In certain embodiments, each R⁶ is independently —NR^{1a}C(S)NR^{1b}R^{1c}, wherein R^{1d}, R^{1b}, and R^{1c} are each as defined herein. In certain embodiments, each R⁶ is independently —NR^{1a}S(O)R^{1d}, wherein R^{1a} and R^{1d} are each as defined herein. In certain embodiments, each R⁶ is independently —NR^{1a}S(O)₂R^{1d}, wherein R^{1a} and R^{1d} are each as defined herein. In certain embodiments, each R⁶ is independently —NR^{1a}S(O)NR^{1b}R^{1c}, wherein R^{1a}, R^{1b}, and R^{1c} are each as defined herein. In certain embodiments, each R⁶ is independently —NR^{1a}S(O)₂NR^{1b}R^{1c}, wherein R^{1a}, R^{1b}, and R^{1c} are each as defined herein. In certain embodiments, each R⁶ is independently —SR^{1a}, wherein R^{1d} is as defined herein. In certain embodiments, each R⁶ is independently —S(O)R^{1d}, wherein R^{1a} is as defined herein. In certain embodiments, each R⁶ is independently —S(O)₂R^{1a}, wherein R^{1a} is as defined herein. In certain embodiments, each R⁶ is independently —S(O)NR^{1b}R^{1c}, wherein R^{1b} and R^{1c} are each as defined herein. In certain embodiments, each R⁶ is independently —S(O)₂NR^{1b}R^{1c}, wherein R^{1b} and R^{1c} are each as defined herein.

In certain embodiments, R⁸ is hydrogen. In certain embodiments, R⁸ is deuterium. In certain embodiments, R⁸ is C₁₋₆ alkyl, optionally substituted with one or more substituents Q. In certain embodiments, R⁸ is methyl or trifluoromethyl. In certain embodiments, R⁸ is methyl. In certain embodiments, R⁸ is C₁₋₆ heteroalkyl, optionally substituted with one or more substituents Q. In certain embodiments, R⁸ is C₂₋₆ alkenyl, optionally substituted with one or more substituents Q. In certain embodiments, R⁸ is C₂₋₆ alkynyl, optionally substituted with one or more substituents Q. In certain embodiments, R⁸ is C₃₋₁₀ cycloalkyl, optionally substituted with one or more substituents Q. In certain embodiments, R⁸ is monocyclic C₃₋₁₀ cycloalkyl, optionally substituted with one or more substituents Q. In certain embodiments, R⁸ is cyclopropyl, cyclobutyl, cyclopentyl, cyclohexyl, or cycloheptyl, each optionally substituted with one or more substituents Q. In certain embodiments, R⁸ is cyclopropyl or 1-fluorocyclopropyl. In certain embodiments, R⁸ is cyclopropyl. In certain embodiments, R⁸ is bicyclic C₄₋₁₀ cycloalkyl, optionally substituted with one or more substituents Q. In certain embodiments, R⁸ is bridged, fused, or spiro C₄₋₁₀ cycloalkyl, each optionally substituted with one or more substituents Q. In certain embodiments, each R⁸ is independently C₆₋₁₄ aryl, optionally substituted with one or more substituents Q. In certain embodiments, each R⁸ is independently C₇₋₁₅ aralkyl, optionally substituted with one or more substituents Q. In certain embodiments, each R⁸ is independently heteroaryl, optionally substituted with one or more substituents Q. In certain embodiments, each R⁸ is independently heterocyclyl, optionally substituted with one or more substituents Q. In certain embodiments, each R⁸ is independently monocyclic heterocyclyl, optionally substituted with one or more substituents Q. In certain embodiments, each R⁸ is independently 3-, 4-, 5-, 6-, or 7-membered heterocyclyl, each optionally substituted with one or more substituents Q. In certain embodiments, each R⁸ is independently bicyclic heterocyclyl, optionally substituted with one or more substituents Q. In certain embodiments, each R⁸ is independently bridged, fused, or spiro heterocyclyl, each optionally substituted with one or more substituents Q.

In certain embodiments, each R^{5a} is hydrogen. In certain embodiments, each R^{5a} is deuterium. In certain embodiments, each R^{5a} is cyano. In certain embodiments, each R^{5a} is independently halo. In certain embodiments, each R^{5a} is independently fluoro or chloro. In certain embodiments, each R^{5a} is fluoro. In certain embodiments, each R^{5a} is nitro. In certain embodiments, each R^{5a} is independently C₁₋₆ alkyl, optionally substituted with one or more substituents Q. In certain embodiments, each R^{5a} is independently methyl or trifluoromethyl. In certain embodiments, each R^{5a} is methyl. In certain embodiments, each R^{5a} is independently C₁₋₆ heteroalkyl, optionally substituted with one or more substituents Q. In certain embodiments, each R^{5a} is independently C₂₋₆ alkenyl, optionally substituted with one or more substituents Q. In certain embodiments, each R^{5a} is independently C₂₋₆ alkynyl, optionally substituted with one or more substituents Q. In certain embodiments, each R^{5a} is independently C₃₋₁₀ cycloalkyl, optionally substituted with one or more substituents Q. In certain embodiments, each R^{5a} is independently C₆₋₁₄ aryl, optionally substituted with one or more substituents Q. In certain embodiments, each R^{5a} is independently C₇₋₁₅ aralkyl, optionally substituted with one or more substituents Q. In certain embodiments, each R⁵, is independently heteroaryl, optionally substituted with one or more substituents Q. In certain embodiments, each R⁵, is independently heterocyclyl, optionally substituted with one or more substituents Q.

In certain embodiments, each R⁵, is independently —C(O)R^{1a}, wherein R^{1a} is as defined herein. In certain embodiments, each R⁵, is independently —C(O)OR^{1a}, wherein R^{1a} is as defined herein. In certain embodiments, each R⁵, is independently —C(O)NR^{1b}R^{1c}, wherein R^{1b} and R^{1c} are each as defined herein. In certain embodiments, each R⁵, is independently —C(O)SR^{1a}, wherein R^{1a} is as defined herein. In certain embodiments, each R⁵, is independently —C(NR^{1a})NR^{1b}R^{1c}, wherein R^{1a}, R^{1b}, and R^{1c} are each as defined herein. In certain embodiments, each R^{5a} is independently —C(S)R^{1d}, wherein R^{1d} is as defined herein. In certain embodiments, each R^{5a} is independently —C(S)OR^{1d}, wherein R^{1d} is as defined herein. In certain embodiments, each R^{5a} is independently —C(S)NR^{1b}R^{1c}, wherein R^{1b} and R^{1c} are each as defined herein. In certain embodiments, each R^{5a} is independently —OR^{1a}, wherein R^{1d} is as defined herein. In certain embodiments, each R^{5a} is independently —OC(O)R^{1d}, wherein R^{1d} is as defined herein. In certain embodiments, each R^{5a} is independently —OC(O)OR^{1d}, wherein R^{1d} is as defined herein. In certain embodiments, each R^{5a} is independently —OC(O)NR^{1b}R^{1c}, wherein R^{1b} and R^{1c} are each as defined herein. In certain embodiments, each R^{5a} is independently —OC(O)SR^{1d}, wherein R^{1d} is as defined herein. In certain embodiments, each R^{5a} is independently —OC(NR^{1d})NR^{1b}R^{1c}, wherein R^{1d}, R^{1b}, and R^{1c} are each as defined herein. In certain embodiments, each R^{5a} is independently —OC(S)R^{1d}, wherein R^{1d} is as defined herein. In certain embodiments, each R^{5a} is independently —OC(S)OR^{1d}, wherein R^{1d} is as defined herein. In certain embodiments, each R^{5a} is independently —OC(S)NR^{1b}R^{1c} wherein R^{1b} and R^{1c} are each as defined herein. In certain embodiments, each R^{5a} is independently —OS(O)R^{1d}, wherein R^{1d} is as defined herein. In certain embodiments, each R^{5a} is independently —OS(O)_{5d}R^{1a}, wherein R^{1d} is as defined herein. In certain embodiments, each R^{5a} is independently —OS(O)NR^{1b}R^{1c}, wherein R^{1b} and R^{1c} are each as defined herein. In certain embodiments, each R^{5a} is independently —OS(O)₂NR^{1b}R^{1c}, wherein R^{1b} and R^{1c} are each as defined herein. In certain embodiments, each R^{5a} is independently —NR^{1b}R^{1c}, wherein R^{1b} and R^{1c} are each as defined herein. In certain embodiments, each R^{5a} is independently —NR^{1d}C(O)R^{1d}, wherein R^{1a} and R^{1d} are each as defined herein. In certain embodiments, each R^{5a} is independently —NR^{1a} C(O)OR^{1d}, wherein R^{1a} and R^{1d} are each as defined herein. In certain embodiments, each R^{5a} is independently —NR^{1d}C(O)NR^{1b}R^{1c}, wherein R^{1d}, R^{1b}, and R^{1c} are each as defined herein. In certain embodiments, each R^{5a} is independently —NR^{1d}C(O)SR^{1d} wherein R^{1a} and R^{1d} are each as defined herein. In certain embodiments, each R^{5d} is independently —NR^{1d}C(NR^{1d})NR^{1b}R^{1c}, wherein R^{1d}, R^{1b}, R^{1c}, and R^{1d} are each as defined herein. In certain embodiments, each R^{5a} is independently —NR^{1d}C(S)R^{1d}, wherein R^{1a} and R^{1d} are each as defined herein. In certain embodiments, each R^{5a} is independently —NR^{1d}C(S)OR^{1d}, wherein R^{1a} and R^{1d} are each as defined herein. In certain embodiments, each R^{5a} is independently —NR^{1d}C(S)NR^{1b}R^{1c}, wherein R^{1d}, Rib, and R^{1c} are each as defined herein. In certain embodiments, each R^{5a} is independently —NR^{1d}S(O)R^{1d}, wherein R^{1a} and R^{1d} are each as defined herein. In certain embodiments, each R^{5a} is independently —NR^{1d}S(O)₂R^{1d}, wherein R^{1a} and R^{1d} are each as defined herein. In certain embodiments, each R^{5a} is independently —NR^{1d}S(O)NR^{1b}R^{1c}, wherein R^{1d}, R^{1b}, and R^{1c} are each as defined herein. In certain embodiments, each R^{5d} is independently —NR^{1d}S(O)₂NR^{1b}R^{1c}, wherein R^{1d}, R^{1b}, and R^{1c} are each as defined herein. In certain embodiments, each R^{5a} is independently —SR^{1d}, wherein R^{1d} is as defined herein. In certain embodiments, each R^{5a} is independently —S(O)R^{1d}, wherein R^{1a} is as defined herein. In certain embodiments, each R^{5a} is independently —S(O)₂R^{1a}, wherein R^{1a} is as defined herein. In certain embodiments, each R^{5b} is independently —S(O)NR^{1b}R^{1c}, wherein R^{1b} and R^{1c} are each as defined herein. In certain embodiments, each R^{5b} is independently —S(O)₂NR^{1b}R^{1c}, wherein R^{1b} and R^{1c} are each as defined herein.

In certain embodiments, each R^{5b} is hydrogen. In certain embodiments, each R^{5b} is deuterium. In certain embodiments, each R^{5b} is cyano. In certain embodiments, each R^{5b} is independently halo. In certain embodiments, each R^{5b} is independently fluoro or chloro. In certain embodiments, each R^{5b} is fluoro. In certain embodiments, each R^{5b} is nitro. In certain embodiments, each R^{5b} is independently C₁₋₆ alkyl, optionally substituted with one or more substituents Q. In certain embodiments, each R^{5b} is independently methyl or trifluoromethyl. In certain embodiments, each R^{5b} is methyl. In certain embodiments, each R^{5b} is independently C₁₋₆ heteroalkyl, optionally substituted with one or more substituents Q. In certain embodiments, each R^{5b} is independently C₂₋₆ alkenyl, optionally substituted with one or more substituents Q. In certain embodiments, each R^{5b} is independently C₂₋₆ alkynyl, optionally substituted with one or more substituents Q. In certain embodiments, each R^{5b} is independently C₃₋₁₀ cycloalkyl, optionally substituted with one or more substituents Q. In certain embodiments, each R^{5b} is independently C₆₋₁₄ aryl, optionally substituted with one or more substituents Q. In certain embodiments, each R^{5b} is independently C₇₋₁₅ aralkyl, optionally substituted with one or more substituents Q. In certain embodiments, each R^{5b} is independently heteroaryl, optionally substituted with one or more substituents Q. In certain embodiments, each R^{5b} is independently heterocyclyl, optionally substituted with one or more substituents Q.

In certain embodiments, each R^{5b} is independently —C(O)R^{1a}, wherein R^{1a} is as defined herein. In certain embodiments, each R^{5b} is independently —C(O)OR^{1a}, wherein R^{1a} is as defined herein. In certain embodiments, each R^{5b} is independently —C(O)NR^{1b}R^{1c}, wherein R^{1b} and R^{1c} are each as defined herein. In certain embodiments, each R^{5b} is independently —C(O)SR^{1a}, wherein R^{1a} is as defined herein. In certain embodiments, each R^{5b} is independently —C(NR^{1d})NR^{1b}R^{1c}, wherein R^{1d}, R^{1b}, and R^{1c} are each as defined herein. In certain embodiments, each R^{5b} is independently —C(S)R^{1a}, wherein R^{1a} is as defined herein. In certain embodiments, each R^{5b} is independently —C(S)OR^{1a}, wherein R^{1d} is as defined herein. In certain embodiments, each R^{5b} is independently —C(S)NR^{1b}R^{1c}, wherein R^{1b} and R^{1c} are each as defined herein. In certain embodiments, each R^{5b} is independently —OR^{1a}, wherein R^{1d} is as defined herein. In certain embodiments, each R^{5b} is independently —OC(O)R^{1d}, wherein R^{1a} is as defined herein. In certain embodiments, each R^{5b} is independently —OC(O)OR^{1a}, wherein R^{1a} is as defined herein. In certain embodiments, each R^{5b} is independently —OC(O)NR^{1b}R^{1c}, wherein R^{1b} and R^{1c} are each as defined herein. In certain embodiments, each R^{5b} is independently —OC(O)SR^{1a}, wherein R^{1a} is as defined herein. In certain embodiments, each R^{5b} is independently —OC(NR^{1a})NR^{1b}R^{1c}, wherein R^{1a}, R^{1b}, and R^{1c} are each as defined herein. In certain embodiments, each R^{5b} is independently —OC(S)R^{1a}, wherein R^{1a} is as defined herein. In certain embodiments, each R^{5b} is independently —OC(S)OR^{1d}, wherein R^{1d} is as defined herein. In certain embodiments, each R^{5b} is independently —OC(S)NR^{1b}R^{1c} wherein R^{1b} and R^{1c} are each as defined herein. In certain embodiments, each R^{5b} is independently —OS(O)R^{1d}, wherein R^{1d} is as defined herein. In certain embodiments, each R^{5b} is independently —OS(O)_{5b}R^{1a}, wherein R^{1d} is as defined herein. In certain embodiments, each R^{5b} is independently —OS(O)NR^{1b}R^{1c}, wherein R^{1b} and R^{1c} are each as defined herein. In certain embodiments, each R^{5b} is independently —OS(O)₂NR^{1b}R^{1c}, wherein R^{1b} and R^{1c} are each as defined herein. In certain embodiments, each R^{5b} is independently —NR^{1b}R^{1c}, wherein R^{1b} and R^{1c} are each as defined herein. In certain embodiments, each R^{5b} is independently —NR^{1a}C(O)R^{1d}, wherein R^{1a} and R^{1d} are each as defined herein. In certain embodiments, each R^{5b} is independently —NR^{1a} C(O)OR^{1d}, wherein R^{1a} and R^{1d} are each as defined herein. In certain embodiments, each R^{5b} is independently —NR^{1a}C(O)NR^{1b}R^{1c}, wherein R^{1d}, R^{1b}, and R^{1c} are each as defined herein. In certain embodiments, each R^{5b} is independently —NR^{1a}C(O)SR^{1d} wherein R^{1a} and R^{1d} are each as defined herein. In certain embodiments, each R^{5b} is independently —NR^{1d}C(NR^{1d})NR^{1b}R^{1c}, wherein R^{1d}, R^{1b}, R^{1c}, and R^{1d} are each as defined herein. In certain embodiments, each R^{5b} is independently —NR^{1a}C(S)R^{1d}, wherein R^{1a} and R^{1d} are each as defined herein. In certain embodiments, each R^{5b} is independently —NR^{1a}C(S)OR^{1d}, wherein R^{1a} and R^{1d} are each as defined herein. In certain embodiments, each R^{5b} is independently —NR^{1a}C(S)NR^{1b}R^{1c}, wherein R^{1d}, Rib, and R^{1c} are each as defined herein. In certain embodiments, each R^{5b} is independently —NR^{1a}S(O)R^{1d}, wherein R^{1a} and R^{1d} are each as defined herein. In certain embodiments, each R^{5b} is independently —NR^{1a}S(O)₂R^{1d}, wherein R^{1a} and R^{1d} are each as defined herein. In certain embodiments, each R^{5b} is independently —NR^{1a}S(O)NR^{1b}R^{1c}, wherein R^{1d}, R^{1b}, and R^{1c} are each as defined herein. In certain embodiments, each R^{5b} is independently —NR^{1a}S(O)₂NR^{1b}R^{1c}, wherein R^{1d}, R^{1b}, and R^{1c} are each as defined herein. In certain embodiments, each R^{5b} is independently —SR^{1a}, wherein R^{1d} is as defined herein. In certain embodiments, each R^{5b} is independently —S(O)R^{1d}, wherein R^{1a} is as defined herein. In certain embodiments, each R^{5b} is independently —S(O)₂R^{1a}, wherein R^{1a} is as defined herein. In certain embodiments, each R^{5b} is independently —S(O)NR^{1b}R^{1c}, wherein R^{1b} and R^{1c} are each as defined herein. In certain embodiments, each R^{5b} is independently —S(O)₂NR^{1b}R^{1c}, wherein R^{1b} and R^{1c} are each as defined herein.

In certain embodiments, each R^{6a} is hydrogen. In certain embodiments, each R^{6a} is deuterium. In certain embodiments, each R^{6a} is cyano. In certain embodiments, each R^{6a} is independently halo. In certain embodiments, each R^{6a} is independently fluoro or chloro. In certain embodiments, each R^{6a} is fluoro. In certain embodiments, each R^{6a} is nitro. In certain embodiments, each R^{6a} is independently C₁₋₆ alkyl, optionally substituted with one or more substituents Q. In certain embodiments, each R^{6a} is independently methyl or trifluoromethyl. In certain embodiments, each R^{6a} is methyl. In certain embodiments, each R^{6a} is independently C₁₋₆ heteroalkyl, optionally substituted with one or more substituents Q. In certain embodiments, each R^{6a} is independently C₂₋₆ alkenyl, optionally substituted with one or more substituents Q. In certain embodiments, each R^{6a} is independently C₂₋₆ alkynyl, optionally substituted with one or more substituents Q. In certain embodiments, each R^{6a} is independently C₃₋₁₀ cycloalkyl, optionally substituted with one or more substituents Q. In certain embodiments, each R^{6a} is independently C₆₋₁₄ aryl, optionally substituted with one or more substituents Q. In certain embodiments, each R^{6a} is independently C₇₋₁₅ aralkyl, optionally substituted with one or more substituents Q. In certain embodiments, each R^{6a} is independently heteroaryl, optionally substituted with one or more substituents Q. In certain embodiments, each R^{6a} is independently heterocyclyl, optionally substituted with one or more substituents Q.

In certain embodiments, each R^{6a} is independently —C(O)R^{1d}, wherein R^{1a} is as defined herein. In certain embodiments, each R^{6a} is independently —C(O)OR^{1a}, wherein R^{1a} is as defined herein. In certain embodiments, each R^{6a} is independently —C(O)NR^{1b}R^{1c}, wherein R^{1b} and R^{1c} are each as defined herein. In certain embodiments, each R^{6a} is independently —C(O)SR^{1a}, wherein R^{1d} is as defined herein. In certain embodiments, each R^{6a} is independently —C(NR^{1d})NR^{1b}R^{1c}, wherein R^{1d}, R^{1b}, and R^{1c} are each as defined herein. In certain embodiments, each R^{6a} is independently —C(S)R^{1d}, wherein R^{1d} is as defined herein. In certain embodiments, each R^{6a} is independently —C(S)OR^{1d}, wherein R^{1d} is as defined herein. In certain embodiments, each R^{6a} is independently —C(S)NR^{1b}R^{1c}, wherein R^{1b} and R^{1c} are each as defined herein. In certain embodiments, each R^{6a} is independently —OR¹, wherein R^{1d} is as defined herein. In certain embodiments, each R^{6d} is independently —OC(O)R^{1d}, wherein R^{1d} is as defined herein. In certain embodiments, each R^{6d} is independently —OC(O)OR^{1a}, wherein R^{1d} is as defined herein. In certain embodiments, each R^{6a} is independently —OC(O)NR^{1b}R^{1c}, wherein R^{1b} and R^{1c} are each as defined herein. In certain embodiments, each R^{6d} is independently —OC(O)SR^{1a}, wherein R^{1d} is as defined herein. In certain embodiments, each R^{6d} is independently —OC(NR^{1d})NR^{1b}R^{1c}, wherein R^{1d}, R^{1b}, and R^{1c} are each as defined herein. In certain embodiments, each R^{6d} is independently —OC(S)R^{1d}, wherein R^{1d} is as defined herein. In certain embodiments, each R^{6d} is independently —OC(S)OR^{1d}, wherein R^{1d} is as defined herein. In certain embodiments, each R^{6d} is independently —OC(S)NR^{1b}R^{1c} wherein R^{1b} and R^{1c} are each as defined herein. In certain embodiments, each R^{6d} is independently —OS(O)R^{1d}, wherein R^{1d} is as defined herein. In certain embodiments, each R^{6d} is independently —OS(O)₆ₐR^{1a}, wherein R^{1d} is as defined herein. In certain embodiments, each R^{6d} is independently —OS(O)NR^{1b}R^{1c}, wherein R^{1b} and R^{1c} are each as defined herein. In certain embodiments, each R^{6d} is independently —OS(O)₂NR^{1b}R^{1c}, wherein R^{1b} and R^{1c} are each as defined herein. In certain embodiments, each R^{6d} is independently —NR^{1b}R^{1c}, wherein R^{1b} and R^{1c} are each as defined herein. In certain embodiments, each R^{6d} is independently —NR^{1d}C(O)R^{1d}, wherein R^{1a} and R^{1d} are each as defined herein. In certain embodiments, each R^{6d} is independently —NR^{1a} C(O)OR^{1d}, wherein R^{1a} and R^{1d} are each as defined herein. In certain embodiments, each R^{6d} is independently —NR^{1d}C(O)NR^{1b}R^{1c}, wherein R^{1d}, R^{1b}, and R^{1c} are each as defined herein. In certain embodiments, each R^{6d} is independently —NR^{1d}C(O)SR^{1d} wherein R^{1a} and R^{1d} are each as defined herein. In certain embodiments, each R^{6d} is independently —NR^{1d}C(NR^{1d})NR^{1b}R^{1c}, wherein R^{1d}, R^{1b}, R^{1c}, and R^{1d} are each as defined herein. In certain embodiments, each R^{6d} is independently —NR^{1d}C(S)R^{1d}, wherein R^{1a} and R^{1d} are each as defined herein. In certain embodiments, each R^{6d} is independently —NR^{1d}C(S)OR^{1d}, wherein R^{1a} and R^{1d} are each as defined herein. In certain embodiments, each R^{6d} is independently —NR^{1d}C(S)NR^{1b}R^{1c}, wherein R^{1d}, Rib, and R^{1c} are each as defined herein. In certain embodiments, each R^{6d} is independently —NR^{1d}S(O)R^{1d}, wherein R^{1a} and R^{1d} are each as defined herein. In certain embodiments, each R^{6d} is independently —NR^{1d}S(O)₂R^{1d}, wherein R^{1a} and R^{1d} are each as defined herein. In certain embodiments, each R^{6d} is independently —NR^{1d}S(O)NR^{1b}R^{1c}, wherein R^{1d}, R^{1b}, and R^{1c} are each as defined herein. In certain embodiments, each R^{6d} is independently —NR^{1d}S(O)₂NR^{1b}R^{1c}, wherein R^{1d}, R^{1b}, and R^{1c} are each as defined herein. In certain embodiments, each R^{6d} is independently —SR¹, wherein R^{1d} is as defined herein. In certain embodiments, each R⁶, is independently —S(O)R^{1a}, wherein R^{1a} is as defined herein. In certain embodiments, each R⁶, is independently —S(O)₂R^{1a}, wherein R^{1d} is as defined herein. In certain embodiments, each R^{6a} is independently —S(O)NR^{1b}R^{1c}, wherein R^{1b} and R^{1c} are each as defined herein. In certain embodiments, each R^{6a} is independently —S(O)₂NR^{1b}R^{1c}, wherein R^{1b} and R^{1c} are each as defined herein.

In certain embodiments, each R^{6b} is hydrogen. In certain embodiments, each R^{6b} is deuterium. In certain embodiments, each R^{6b} is cyano. In certain embodiments, each R^{6b} is independently halo. In certain embodiments, each R^{6b} is independently fluoro or chloro. In certain embodiments, each R^{6b} is fluoro. In certain embodiments, each R^{6b} is nitro. In certain embodiments, each R^{6b} is independently C₁₋₆ alkyl, optionally substituted with one or more substituents Q. In certain embodiments, each R^{6b} is independently methyl or trifluoromethyl. In certain embodiments, each R^{6b} is methyl. In certain embodiments, each R^{6b} is independently C₁₋₆ heteroalkyl, optionally substituted with one or more substituents Q. In certain embodiments, each R^{6b} is independently C₂₋₆ alkenyl, optionally substituted with one or more substituents Q. In certain embodiments, each R^{6b} is independently C₂₋₆ alkynyl, optionally substituted with one or more substituents Q. In certain embodiments, each R^{6b} is independently C₃₋₁₀ cycloalkyl, optionally substituted with one or more substituents Q. In certain embodiments, each R^{6b} is independently C₆₋₁₄ aryl, optionally substituted with one or more substituents Q. In certain embodiments, each R^{6b} is independently C₇₋₁₅ aralkyl, optionally substituted with one or more substituents Q. In certain embodiments, each R^{6b} is independently heteroaryl, optionally substituted with one or more substituents Q. In certain embodiments, each R^{6b} is independently heterocyclyl, optionally substituted with one or more substituents Q.

In certain embodiments, each R^{6b} is independently —C(O)R^{1d}, wherein R^{1d} is as defined herein. In certain embodiments, each R^{6b} is independently —C(O)OR^{1d}, wherein R^{1d} is as defined herein. In certain embodiments, each R^{6b} is independently —C(O)NR^{1b}R^{1c}, wherein R^{1b} and R^{1c} are each as defined herein. In certain embodiments, each R^{6b} is independently —C(O)SR^{1d}, wherein R^{1d} is as defined herein. In certain embodiments, each R^{6b} is independently —C(NR^{1d})NR^{1b}R^{1c}, wherein R^{1d}, R^{1b}, and R^{1c} are each as defined herein. In certain embodiments, each R^{6b} is independently —C(S)R^{1d}, wherein R^{1d} is as defined herein. In certain embodiments, each R^{6b} is independently —C(S)OR^{1d}, wherein R^{1d} is as defined herein. In certain embodiments, each R^{6b} is independently —C(S)NR^{1b}R^{1c} wherein R^{1b} and R^{1c} are each as defined herein. In certain embodiments, each R^{6b} is independently —OR^{1a}, wherein R^{1d} is as defined herein. In certain embodiments, each R^{6b} is independently —OC(O)R^{1d}, wherein R^{1d} is as defined herein. In certain embodiments, each R^{6b} is independently —OC(O)OR^{1a}, wherein R^{1d} is as defined herein. In certain embodiments, each R^{6b} is independently —OC(O)NR^{1b}R^{1c} wherein R^{1b} and R^{1c} are each as defined herein. In certain embodiments, each R^{6b} is independently —OC(O)SR^{1a}, wherein R^{1d} is as defined herein. In certain embodiments, each R^{6b} is independently —OC(NR^{1d})NR^{1b}R^{1c}, wherein R^{1d}, R^{1b}, and R^{1c} are each as defined herein. In certain embodiments, each R^{6b} is independently —OC(S)R^{1d}, wherein R^{1d} is as defined herein. In certain embodiments, each R^{6b} is independently —OC(S)OR^{1d}, wherein R^{1d} is as defined herein. In certain embodiments, each R^{6b} is independently —OC(S)NR^{1b}R^{1c} wherein R^{1b} and R^{1c} are each as defined herein. In certain embodiments, each R^{6b} is independently —OS(O)R^{1d}, wherein R^{1d} is as defined herein. In certain embodiments, each R^{6b} is independently —OS(O)_{6b}R^{1d}, wherein R^{1d} is as defined herein. In certain embodiments, each R^{6b} is independently —OS(O)NR^{1b}R^{1c}, wherein R^{1b} and R^{1c} are each as defined herein. In certain embodiments, each R^{6b} is independently —OS(O)₂NR^{1b}R^{1c}, wherein R^{1b} and R^{1c} are each as defined herein. In certain embodiments, each R^{6b} is independently —NR^{1b}R^{1c}, wherein R^{1b} and R^{1c} are each as defined herein. In certain embodiments, each R^{6b} is independently —NR^{1d}C(O)R^{1d}, wherein R^{1a} and R^{1d} are each as defined herein. In certain embodiments, each R^{6b} is independently —NR^{1a} C(O)OR^{1d}, wherein R^{1a} and R^{1d} are each as defined herein. In certain embodiments, each R^{6b} is independently —NR^{1d}C(O)NR^{1b}R^{1c}, wherein R^{1d}, R^{1b}, and R^{1c} are each as defined herein. In certain embodiments, each R^{6b} is independently —NR^{1d}C(O)SR^{1d} wherein R^{1a} and R^{1d} are each as defined herein. In certain embodiments, each R^{6b} is independently —NR^{1d}C(NR^{1d})NR^{1b}R^{1c}, wherein R^{1a}, R^{1b}, R^{1c}, and R^{1d} are each as defined herein. In certain embodiments, each R^{6b} is independently —NR^{1d}C(S)R^{1d}, wherein R^{1a} and R^{1d} are each as defined herein. In certain embodiments, each R^{6b} is independently —NR^{1d}C(S)OR^{1d}, wherein R^{1a} and R^{1d} are each as defined herein. In certain embodiments, each R^{6b} is independently —NR^{1d}C(S)NR^{1b}R^{1c}, wherein R^{1d}, Rib, and R^{1c} are each as defined herein. In certain embodiments, each R^{6b} is independently —NR^{1d}S(O)R^{1d}, wherein R^{1a} and R^{1d} are each as defined herein. In certain embodiments, each R^{6b} is independently —NR^{1d}S(O)₂R^{1d}, wherein R^{1a} and R^{1d} are each as defined herein. In certain embodiments, each R^{6b} is independently —NR^{1d}S(O)NR^{1b}R^{1c}, wherein R^{1d}, R^{1b}, and R^{1c} are each as defined herein. In certain embodiments, each R^{6b} is independently —NR^{1d}S(O)₂NR^{1b}R^{1c} wherein R^{1d}, R^{1b}, and R^{1c} are each as defined herein. In certain embodiments, each R^{6b} is independently —SR^{1a}, wherein R^{1a} is as defined herein. In certain embodiments, each R^{6b} is independently —S(O)R^{1a}, wherein R^{1a} is as defined herein. In certain embodiments, each R^{6b} is independently —S(O)₂R^{1a}, wherein R^{1a} is as defined herein. In certain embodiments, each R^{6b} is independently —S(O)NR^{1b}R^{1c}, wherein R^{1b} and R^{1c} are each as defined herein. In certain embodiments, each R^{6b} is independently —S(O)₂NR^{1b}R^{1c}, wherein R^{1b} and R^{1c} are each as defined herein.

In certain embodiments, each R^{6c} is hydrogen. In certain embodiments, each R^{1c} is deuterium. In certain embodiments, each R^{6c} is cyano. In certain embodiments, each R^{6c} is independently halo. In certain embodiments, each R^{6c} is independently fluoro or chloro. In certain embodiments, each R^{6c} is fluoro. In certain embodiments, each R^{6c} is nitro. In certain embodiments, each R^{6c} is independently C₁₋₆ alkyl, optionally substituted with one or more substituents Q. In certain embodiments, each R^{6c} is independently methyl or trifluoromethyl. In certain embodiments, each R^{6c} is methyl. In certain embodiments, each R^{6c} is independently C₁₋₆ heteroalkyl, optionally substituted with one or more substituents Q. In certain embodiments, each R^{6c} is independently C₂₋₆ alkenyl, optionally substituted with one or more substituents Q. In certain embodiments, each R^{6c} is independently C₂₋₆ alkynyl, optionally substituted with one or more substituents Q. In certain embodiments, each R^{6c} is independently C₃₋₁₀ cycloalkyl, optionally substituted with one or more substituents Q. In certain embodiments, each R^{6c} is independently C₆₋₁₄ aryl, optionally substituted with one or more substituents Q. In certain embodiments, each R^{6c} is independently C₇₋₁₅ aralkyl, optionally substituted with one or more substituents Q. In certain embodiments, each R^{6c} is independently heteroaryl, optionally substituted with one or more substituents Q. In certain embodiments, each R^{6c} is independently heterocyclyl, optionally substituted with one or more substituents Q.

In certain embodiments, each R^{6c} is independently —C(O)R^{1a}, wherein R^{1d} is as defined herein. In certain embodiments, each R^{6c} is independently —C(O)OR^{1d}, wherein R^{1d} is as defined herein. In certain embodiments, each R^{6c} is independently —C(O)NR^{1b}R^{1c}, wherein R^{1b} and R^{1c} are each as defined herein. In certain embodiments, each R^{6c} is independently —C(O)SR^{1d}, wherein R^{1d} is as defined herein. In certain embodiments, each R^{6c} is independently —C(NR^{1d})NR^{1b}R^{1c}, wherein R^{1d}, R^{1b}, and R^{1c} are each as defined herein. In certain embodiments, each R^{6c} is independently —C(S)R^{1d}, wherein R^{1d} is as defined herein. In certain embodiments, each R^{6c} is independently —C(S)OR^{1d}, wherein R^{1d} is as defined herein. In certain embodiments, each R^{6c} is independently —C(S)NR^{1b}R^{1c}, wherein R^{1b} and R^{1c} are each as defined herein. In certain embodiments, each R^{6c} is independently —OR^{1a}, wherein R^{1a} is as defined herein. In certain embodiments, each R^{6c} is independently —OC(O)R^{1a}, wherein R^{1a} is as defined herein. In certain embodiments, each R^{6c} is independently —OC(O)OR^{1a}, wherein R^{1a} is as defined herein. In certain embodiments, each R^{6c} is independently —OC(O)NR^{1b}R^{1c}, wherein R^{1b} and R^{1c} are each as defined herein. In certain embodiments, each R^{6c} is independently —OC(O)SR^{1a}, wherein R^{1a} is as defined herein. In certain embodiments, each R^{6c} is independently —OC(NR^{1d})NR^{1b}R^{1c}, wherein R^{1d}, R^{1b}, and R^{1c} are each as defined herein. In certain embodiments, each R^{6c} is independently —OC(S)R^{1a}, wherein R^{1d} is as defined herein. In certain embodiments, each R^{6c} is independently —OC(S)OR^{1d}, wherein R^{1d} is as defined herein. In certain embodiments, each R^{6c} is independently —OC(S)NR^{1b}R^{1c} wherein R^{1b} and R^{1c} are each as defined herein. In certain embodiments, each R^{6c} is independently —OS(O)R^{1d}, wherein R^{1d} is as defined herein. In certain embodiments, each R^{6c} is independently —OS(O)_{6c}R^{1a}, wherein R^{1d} is as defined herein. In certain embodiments, each R^{6c} is independently —OS(O)NR^{1b}R^{1c}, wherein R^{1b} and R^{1c} are each as defined herein. In certain embodiments, each R^{6c} is independently —OS(O)₂NR^{1b}R^{1c}, wherein R^{1b} and R^{1c} are each as defined herein. In certain embodiments, each R^{6c} is independently —NR^{1b}R^{1c}, wherein R^{1b} and R^{1c} are each as defined herein. In certain embodiments, each R^{6c} is independently —NR^{1a}C(O)R^{1d}, wherein R^{1a} and R^{1d} are each as defined herein. In certain embodiments, each R^{6c} is independently —NR^{1a} C(O)OR^{1d}, wherein R^{1a} and R^{1d} are each as defined herein. In certain embodiments, each R^{6c} is independently —NR^{1a}C(O)NR^{1b}R^{1c}, wherein R^{1d}, R^{1b}, and Ric are each as defined herein. In certain embodiments, each R^{6c} is independently —NR^{1a}C(O)SR^{1d} wherein R^{1a} and R^{1d} are each as defined herein. In certain embodiments, each R^{1c} is independently —NR^{1a}C(NR^{1d})NR^{1b}R^{1c}, wherein R^{1d}, R^{1b}, R^{1c}, and R^{1d} are each as defined herein. In certain embodiments, each R^{6c} is independently —NR^{1a}C(S)R^{1d}, wherein R^{1a} and R^{1d} are each as defined herein. In certain embodiments, each R^{6c} is independently —NR^{1a}C(S)OR^{1d}, wherein R^{1a} and R^{1d} are each as defined herein. In certain embodiments, each R^{6c} is independently —NR^{1a}C(S)NR^{1b}R^{1c}, wherein R^{1d}, Rib, and R^{1c} are each as defined herein. In certain embodiments, each R^{6c} is independently —NR^{1a}S(O)R^{1d}, wherein R^{1a} and R^{1d} are each as defined herein. In certain embodiments, each R^{6c} is independently —NR^{1a}S(O)₂R^{1d}, wherein R^{1a} and R^{1d} are each as defined herein. In certain embodiments, each R^{6c} is independently —NR^{1a}S(O)NR^{1b}R^{1c}, wherein R^{1d}, R^{1b}, and R^{1c} are each as defined herein. In certain embodiments, each R^{1c} is independently —NR^{1a}S(O)₂NR^{1b}R^{1c}, wherein R^{1a}, R^{1b}, and R^{1c} are each as defined herein. In certain embodiments, each R^{6c} is independently —SR^{1a}, wherein R^{1a} is as defined herein. In certain embodiments, each R^{6c} is independently —S(O)R^{1a}, wherein R^{1a} is as defined herein. In certain embodiments, each R^{6c} is independently —S(O)₂R^{1a}, wherein R^{1a} is as defined herein. In certain embodiments, each R^{6c} is independently —S(O)NR^{1b}R^{1c}, wherein R^{1b} and R^{1c} are each as defined herein. In certain embodiments, each R^{6c} is independently —S(O)₂NR^{1b}R^{1c}, wherein R^{1b} and R^{1c} are each as defined herein.

In certain embodiments, each A is O. In certain embodiments, each A is S. In certain embodiments, each A is S(O). In certain embodiments, each A is S(O₂). In certain embodiments, each A is independently N(R⁴), wherein R⁴ is as defined herein. In certain embodiments, each A is independently N(R⁴), wherein R⁴ is (i) hydrogen; (ii) C₁₋₆ alkyl or C₁₋₆ heteroalkyl, each optionally substituted with one or more substituents Q; or (iii) —C(O)R^{1a}, wherein R^{1a} is as defined herein. In certain embodiments, each A is independently N(H), N(CH₃), or N(C(O)CH₃). In certain embodiments, each A is N(H).

In certain embodiments, each X is independently C₁₋₄₀ alkylene, optionally substituted with one or more substituents Q. In certain embodiments, each X is independently C₁₋₃₀ alkylene, optionally substituted with one or more substituents Q. In certain embodiments, each X is independently C₁₋₂₀ alkylene, optionally substituted with one or more substituents Q. In certain embodiments, each X is independently C₁₋₄₀ heteroalkylene, optionally substituted with one or more substituents Q. In certain embodiments, each X is independently C₁₋₃₀ heteroalkylene, optionally substituted with one or more substituents Q. In certain embodiments, each X is independently C₁₋₂₀ heteroalkylene, optionally substituted with one or more substituents Q. In certain embodiments, each X is independently C₂₋₄₀ alkenylene, optionally substituted with one or more substituents Q. In certain embodiments, each X is independently C₂₋₄₀ heteroalkenylene, optionally substituted with one or more substituents Q. In certain embodiments, each X is independently C₂₋₄₀ alkynylene, optionally substituted with one or more substituents Q. In certain embodiments, each X is independently C₂₋₄₀ heteroalkynylene, optionally substituted with one or more substituents Q. In certain embodiments, each X is independently C₃₋₁₀ cycloalkylene, optionally substituted with one or more substituents Q. In certain embodiments, each X is independently C₆₋₁₄ arylene, optionally substituted with one or more substituents Q. In certain embodiments, each X is independently heteroarylene, optionally substituted with one or more substituents Q. In certain embodiments, each X is independently heterocyclylene, optionally substituted with one or more substituents Q.

In certain embodiments, each X is independently C₁₋₄₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, each X is independently C₁₋₃₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q. In certain embodiments, each X is independently C₁₋₂₀ alkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkylene, cycloalkylene, arylene, heteroarylene, and heterocyclylene are each optionally substituted with one or more substituents Q.

In certain embodiments, each X is independently C₁₋₄₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, each X is independently C₁₋₃₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q. In certain embodiments, each X is independently C₁₋₂₀ heteroalkylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkylene, cycloalkylene, arylene, heteroarylene, and heterocyclylene are each optionally substituted with one or more substituents Q.

In certain embodiments, each X is independently C₂₋₄₀ alkenylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkenylene, cycloalkylene, arylene, heteroarylene, and heterocyclylene are each optionally substituted with one or more substituents Q. In certain embodiments, each X is independently C₂₋₄₀ heteroalkenylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkenylene, cycloalkylene, arylene, heteroarylene, and heterocyclylene are each optionally substituted with one or more substituents Q. In certain embodiments, each X is independently C₂₋₄₀ alkynylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the alkynylene, cycloalkylene, arylene, heteroarylene, and heterocyclylene are each optionally substituted with one or more substituents Q. In certain embodiments, each X is independently C₂₋₄₀ heteroalkynylene, wherein one or more methylene groups are each independently and optionally replaced by a divalent group, and each divalent group is independently C₃₋₁₀ cycloalkylene, C₆₋₁₄ arylene, heteroarylene, or heterocyclylene; and wherein the heteroalkynylene, cycloalkylene, arylene, heteroarylene, and heterocyclylene are each optionally substituted with one or more substituents Q.

In certain embodiments, each X is independently:

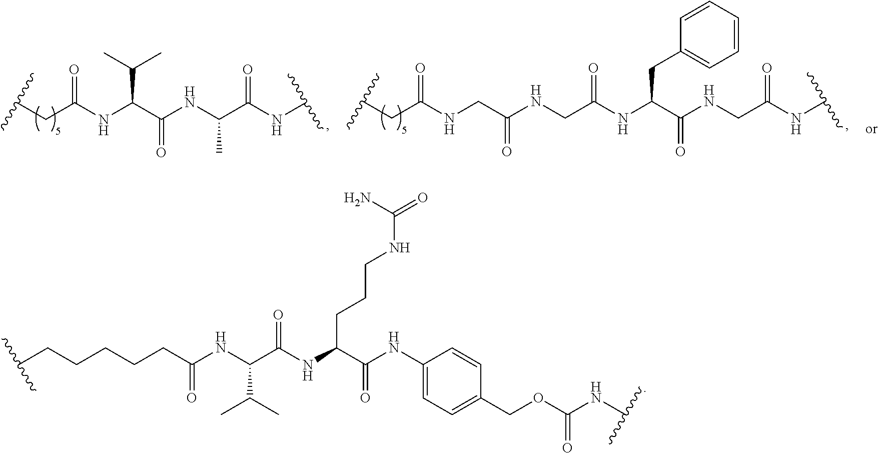

In certain embodiments, each is independently:

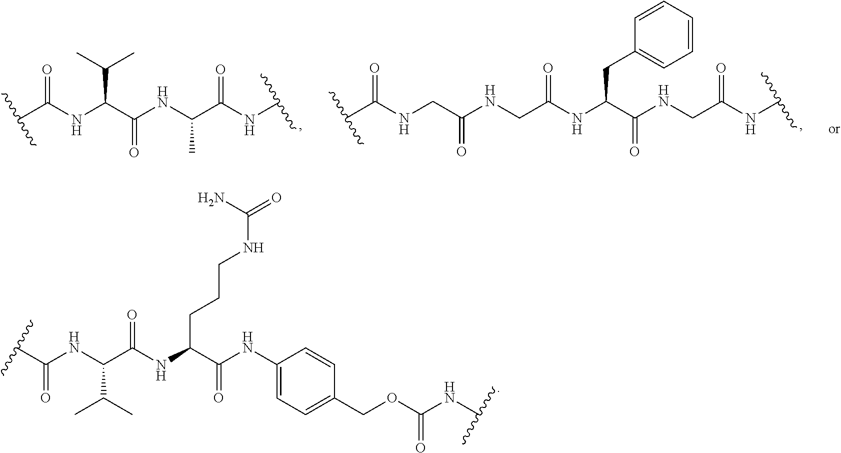 [SMILES: *NC(=O)[C@H](C)NC(=O)[C@@H](NC(*)=O)C(C)C ; *NC(=O)CNC(=O)[C@H](Cc1ccccc1)NC(=O)CNC(=O)CNC(*)=O ; *NC(=O)OCc1ccc(NC(=O)[C@H](CCCNC(N)=O)NC(=O)[C@@H](NC(*)=O)C(C)C)cc1]

In certain embodiments, each X is independently:

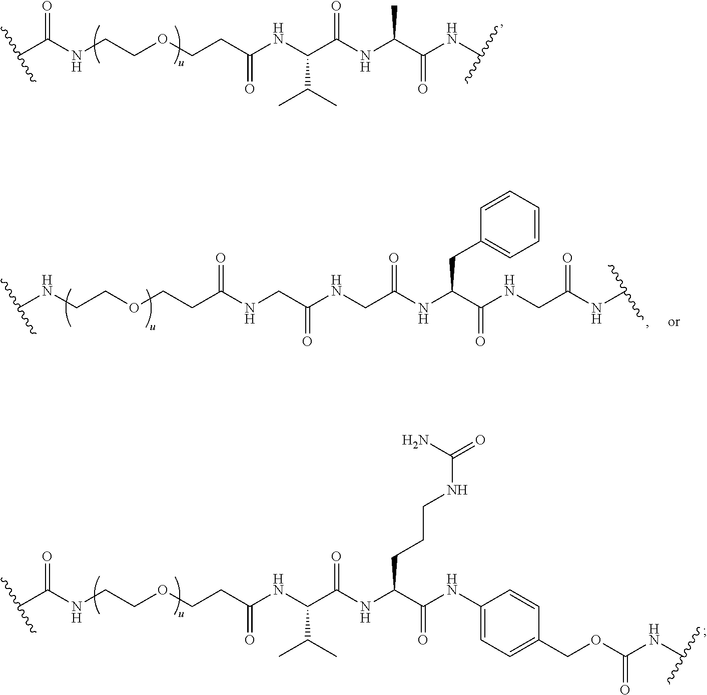

wherein, in one embodiment, each u is independently an integer of 1, 2, 3, 4, 5, 6, 7, or 8; in another embodiment, each u is independently an integer of 4 or 8.

In certain embodiments, each X is independently:

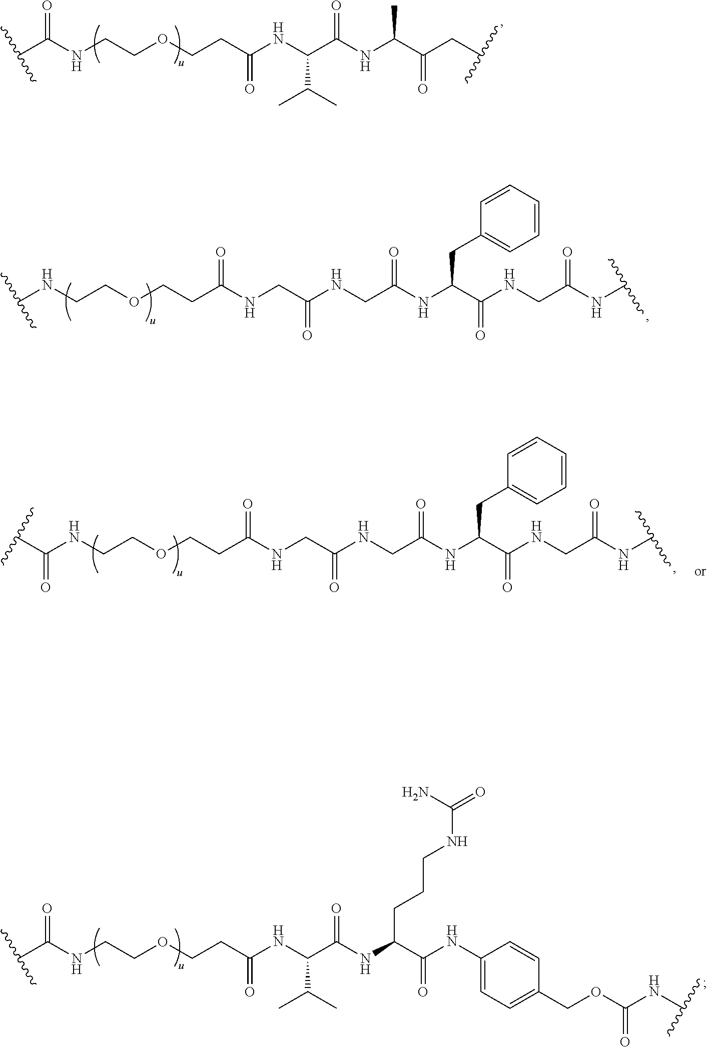

wherein, in one embodiment, each u is independently an integer of 1, 2, 3, 4, 5, 6, 7, or 8; in another embodiment, each u is independently an integer of 4 or 8.

In certain embodiments, each X is independently:

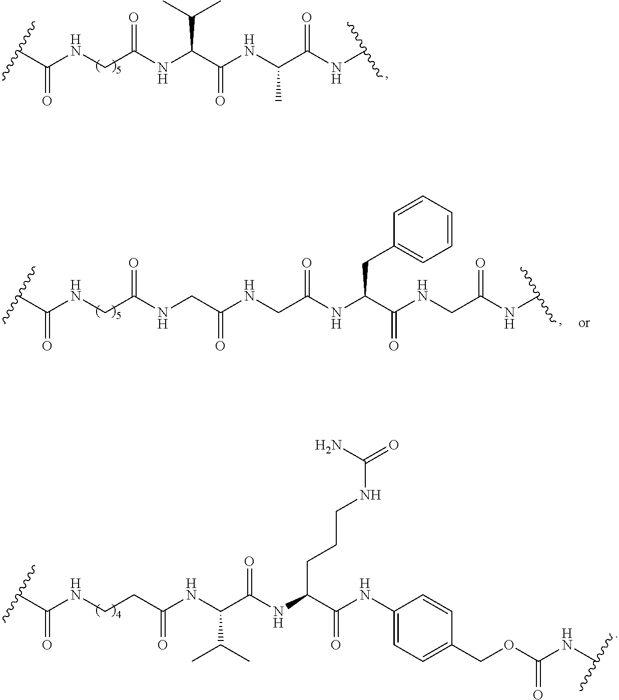

In certain embodiments, each X is independently:

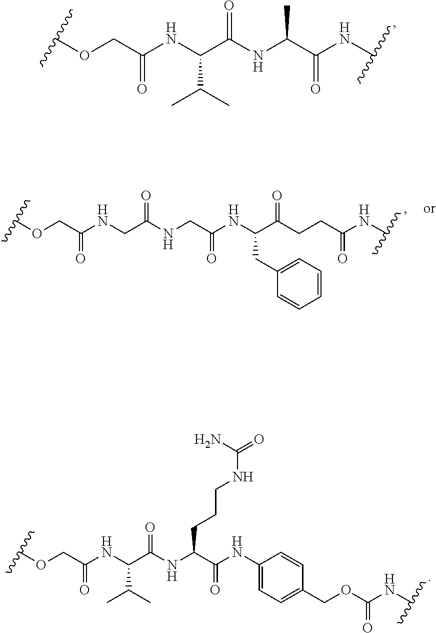 [SMILES: *NC(=O)[C@H](C)NC(=O)[C@@H](NC(=O)CO*)C(C)C ; *NC(=O)CCC(=O)[C@H](Cc1ccccc1)NC(=O)CNC(=O)CNC(=O)CO* ; *NC(=O)OCc1ccc(NC(=O)[C@H](CCCNC(N)=O)NC(=O)[C@@H](NC(=O)CO*)C(C)C)cc1]

In certain embodiments, each X is independently:

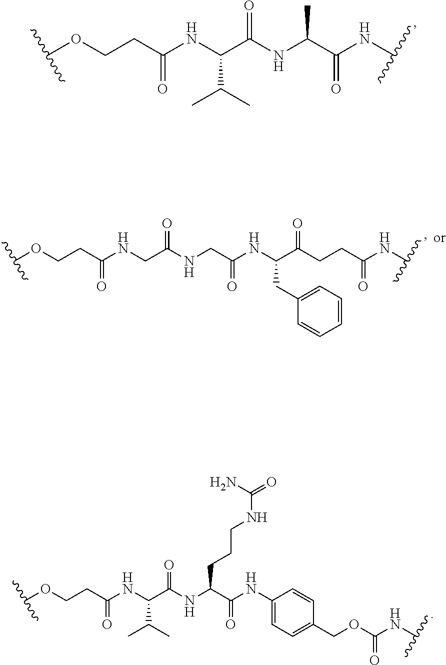 [SMILES: *NC(=O)[C@H](C)NC(=O)[C@@H](NC(=O)CCO*)C(C)C ; *NC(=O)CCC(=O)[C@H](Cc1ccccc1)NC(=O)CNC(=O)CNC(=O)CCO* ; *NC(=O)OCc1ccc(NC(=O)[C@H](CCCNC(N)=O)NC(=O)[C@@H](NC(=O)CCO*)C(C)C)cc1]

In certain embodiments, each X is independently:

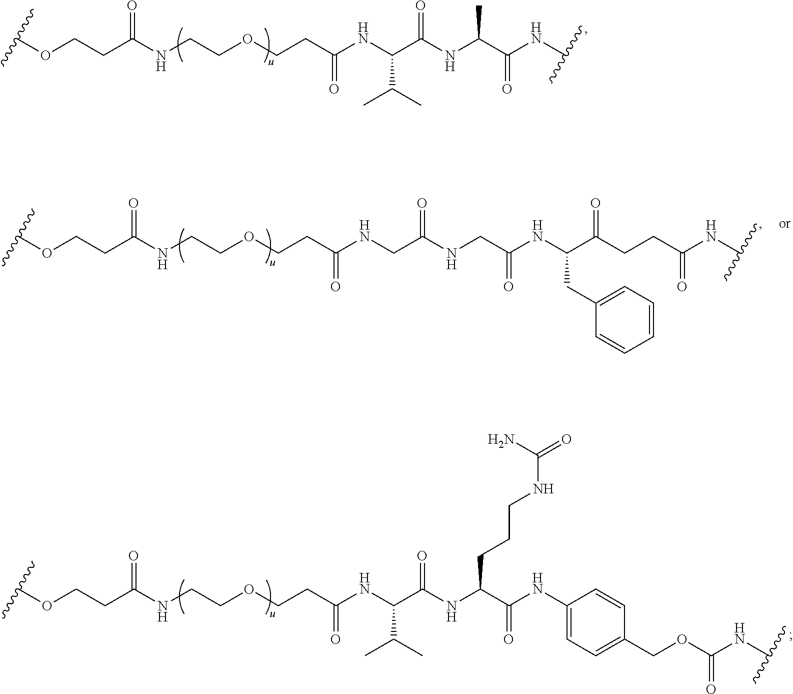

wherein, in one embodiment, each u is independently an integer of 1, 2, 3, 4, 5, 6, 7, or 8; in another embodiment, each u is independently an integer of 4 or 8.

In certain embodiments, each Y is a bond. In certain embodiments, each Y is independently C₁₋₆ alkylene, optionally substituted with one or more substituents Q. In certain embodiments, each Y is independently —(CH₂)—, wherein v is an integer of 1, 2, 3, 4, 5, or 6. In certain embodiments, each Y is independently ethanediyl, propanediyl, or butanediyl. In certain embodiments, each Y is independently ethane-1, 1-diyl, propane-1, 3-diyl, or 2-methylpropane-1, 3-diyl. In certain embodiments, each Y is independently C₁₋₆ heteroalkylene, optionally substituted with one or more substituents Q. In certain embodiments, each Y is —C(O)NH— or —OC(O) NH—. In certain embodiments, each Y is independently C₂₋₆ alkenylene, optionally substituted with one or more substituents Q. In certain embodiments, each Y is independently C₂₋₆ alkynylene, optionally substituted with one or more substituents Q. In certain embodiments, each Y is independently C₃₋₁₀ cycloalkylene, optionally substituted with one or more substituents Q. In certain embodiments, each Y is independently C₆₋₁₄ arylene, optionally substituted with one or more substituents Q. In certain embodiments, each Y is independently C₇₋₁₅ is aralkylene, optionally substituted with one or more substituents Q. In certain embodiments, each Y is independently heteroarylene, optionally substituted with one or more substituents Q. In certain embodiments, each Y is independently heterocyclylene, optionally substituted with one or more substituents Q.

In certain embodiments, Z is

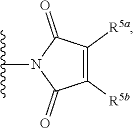

wherein R^{5a} and R^{5b} are each as defined herein. In certain embodiments, Z is

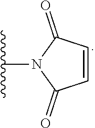 [SMILES: *N1C(=O)C=CC1=O]

In certain embodiments, Z is

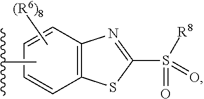

wherein each R⁶, R⁸, and s is as defined herein. In certain embodiments, Z is

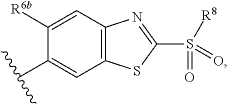

wherein R⁸ and R^{6b} are each as defined herein. In certain embodiments, Z is

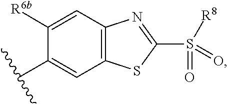

wherein R⁸ is C₁₋₆ alkyl, C₁₋₆ heteroalkyl, or C₃₋₁₀ cycloalkyl, each optionally substituted with one or more substituents Q; and R^{6b} is hydrogen or halo. In certain embodiments, Z is

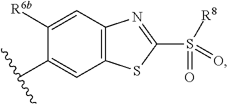

wherein R⁸ is methyl, cyclopropyl, or 1-fluorocyclopropyl; and R^{6b} is hydrogen or fluoro. In certain embodiments, Z is

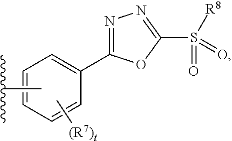

wherein
R⁷, R⁸, and t are each as defined herein.

In certain embodiments, m is an integer of 1, 2, 3, 4, 5, 6, 7, 8, 9, 10, 11, or 12. In certain embodiments, m is an integer of 1, 2, 3, 4, 5, 6, 7, 8, 9, or 10. In certain embodiments, m is an integer of 1, 2, 3, 4, 5, 6, 7, or 8. In certain embodiments, m is an integer of 1. In certain embodiments, m is an integer of 2. In certain embodiments, m is an integer of 3. In certain embodiments, m is an integer of 4. In certain embodiments, m is an integer of 5. In certain embodiments, m is an integer of 6. In certain embodiments, m is an integer of 7. In certain embodiments, m is an integer of 8. In certain embodiments, m is an integer of 9. In certain embodiments, m is an integer of 10. In certain embodiments, m is an integer of 11. In certain embodiments, m is an integer of 12. In certain embodiments, m is an integer of 11. In certain embodiments, m is an integer of 13. In certain embodiments, m is an integer of 11. In certain embodiments, m is an integer of 14. In certain embodiments, m is an integer of 11. In certain embodiments, m is an integer of 15. In certain embodiments, m is an integer of 11. In certain embodiments, m is an integer of 16.

In certain embodiments, each p is an integer of 0. In certain embodiments, each p is an integer of 1. In certain embodiments, each p is an integer of 2.

In certain embodiments, each q is an integer of 0. In certain embodiments, each q is an integer of 1. In certain embodiments, each q is an integer of 2.

In certain embodiments, each s is an integer of 0. In certain embodiments, each s is an integer of 1. In certain embodiments, each s is an integer of 2. In certain embodiments, each s is an integer of 3.

In one embodiment, provided herein is a compound of:

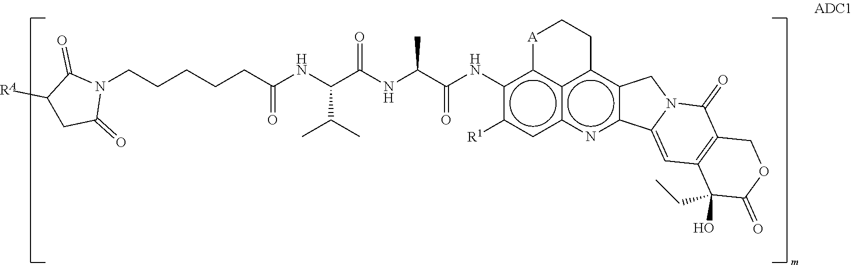

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In another embodiment, provided herein is a compound of:

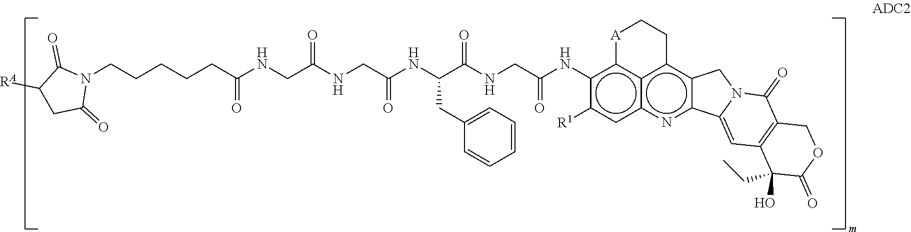

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment provided herein is a compound of:

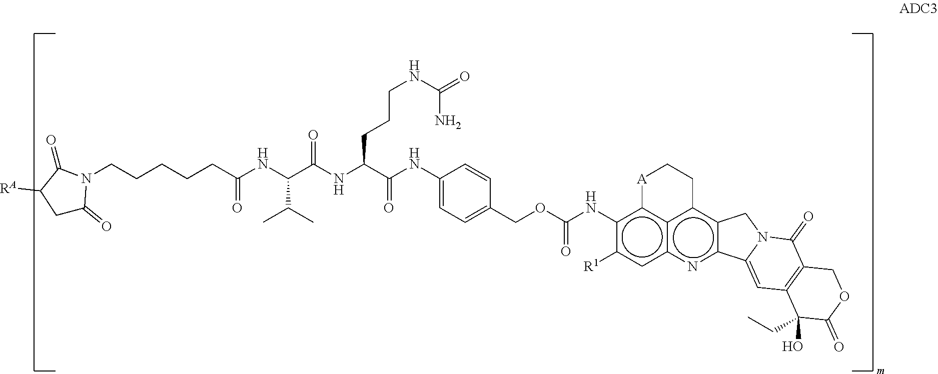

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

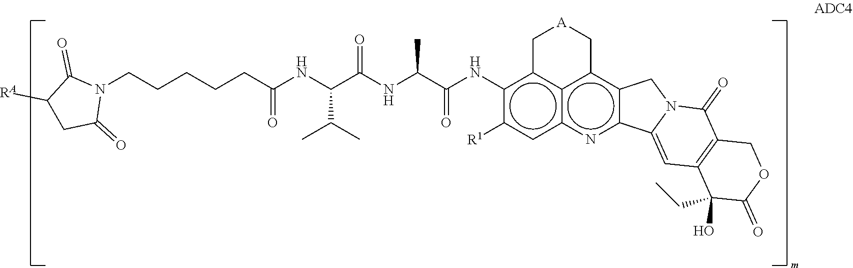

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

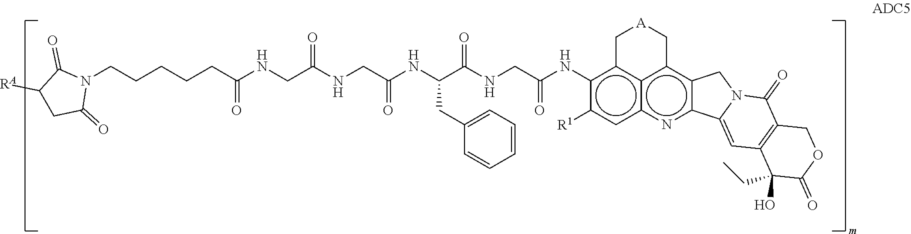

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In still another embodiment, provided herein is a compound of:

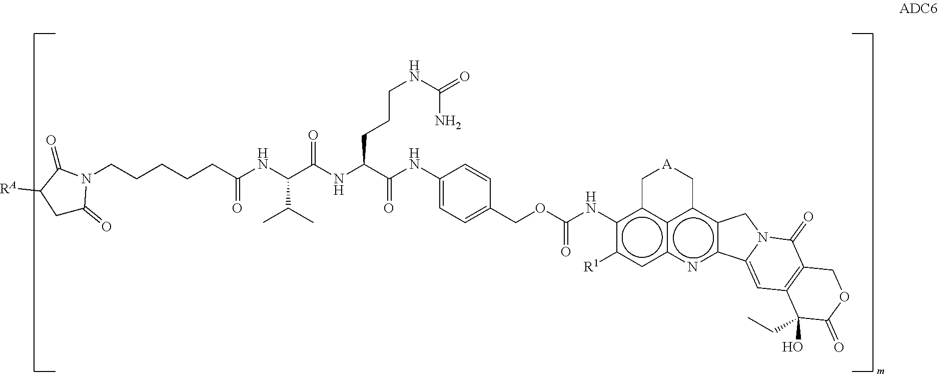

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, 0 or S; and m is as defined herein, in one embodiment, an integer of 2, 3, 4, 5, 6, 7, or 8; and wherein each linker is independently connected covalently to a thiol group of a cysteine residue of the antibody.

In one embodiment, provided herein is a compound of:

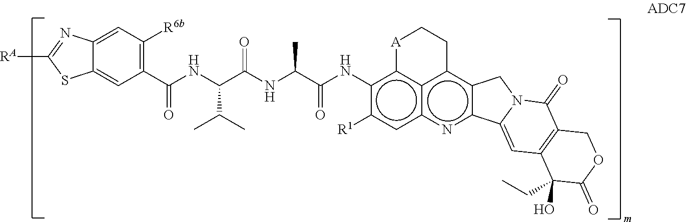

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In another embodiment, provided herein is a compound of:

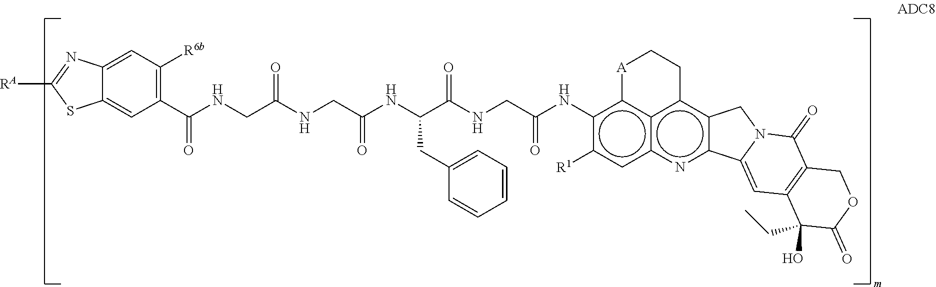

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

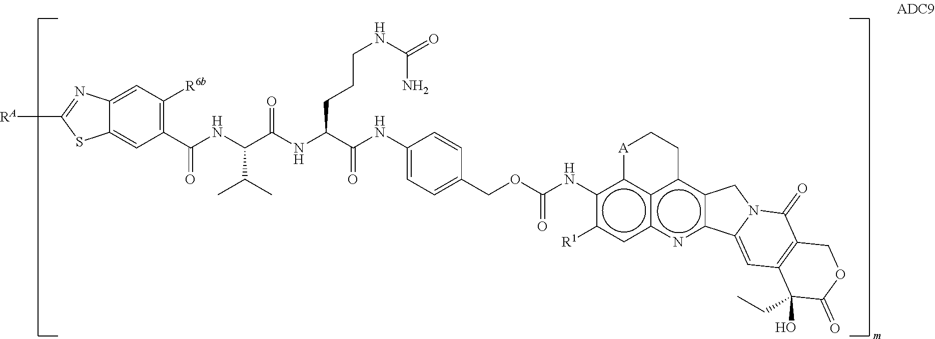

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

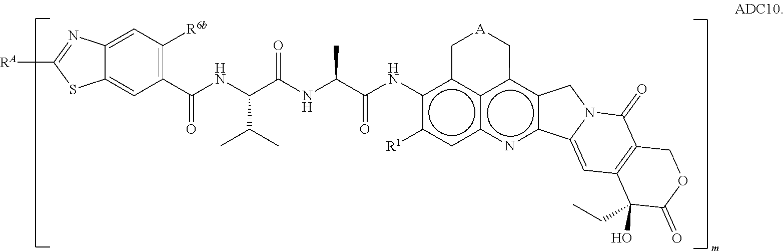

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

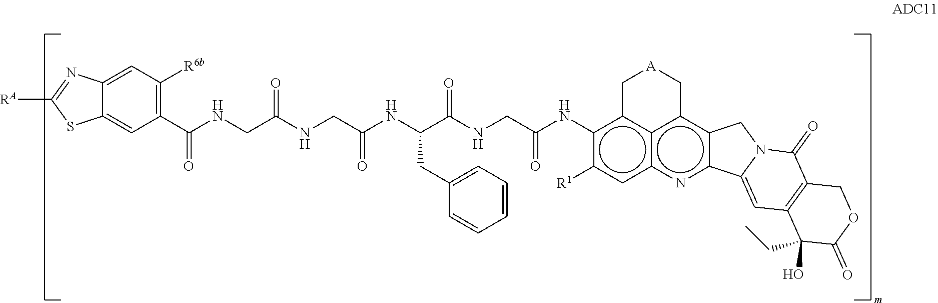

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In still another embodiment, provided herein is a compound of:

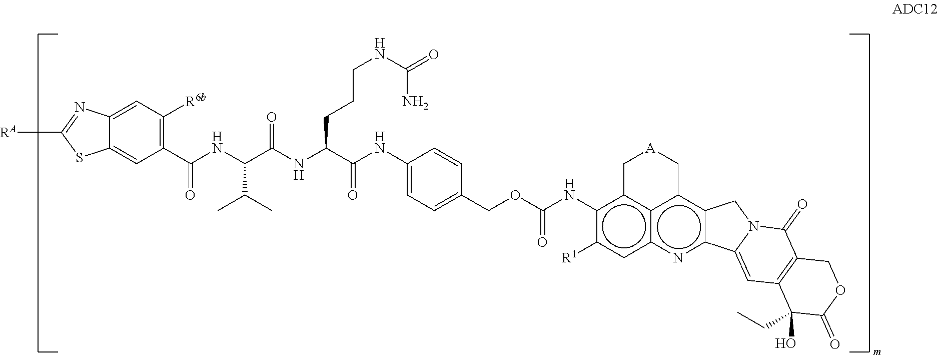

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In one embodiment, provided herein is a compound of:

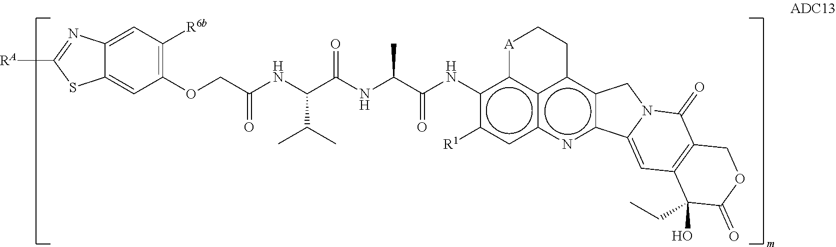

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In another embodiment, provided herein is a compound of

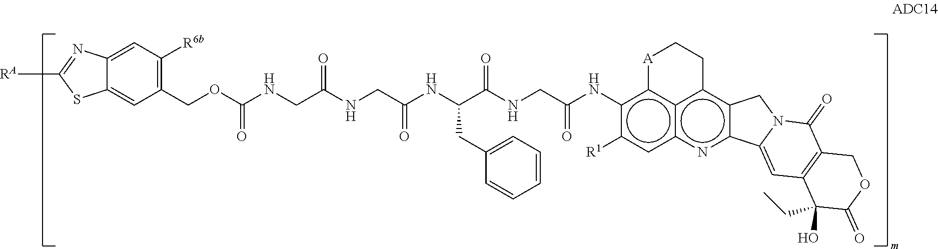

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

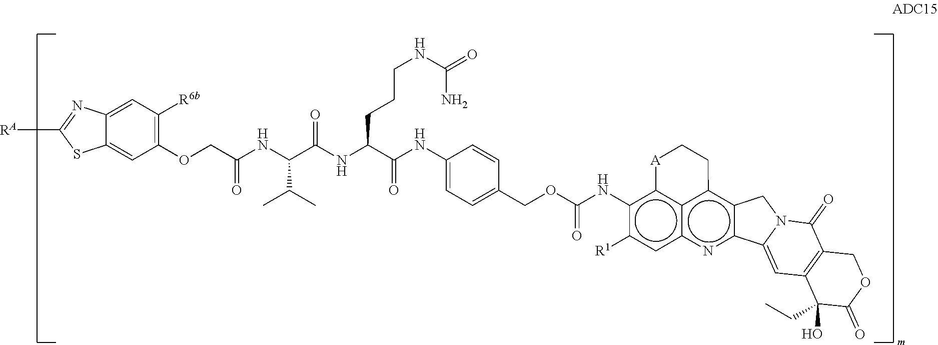

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

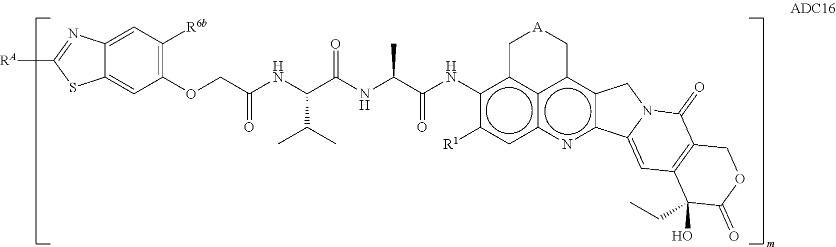

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

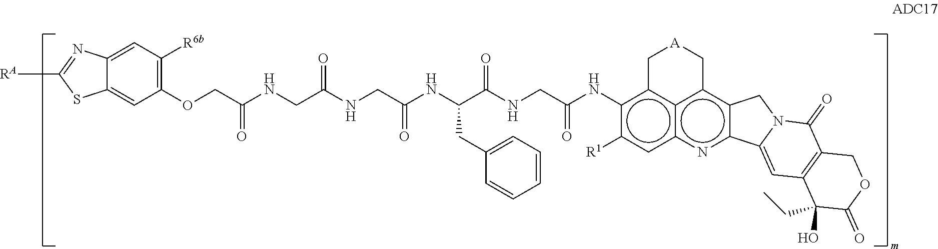

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In still another embodiment, provided herein is a compound of:

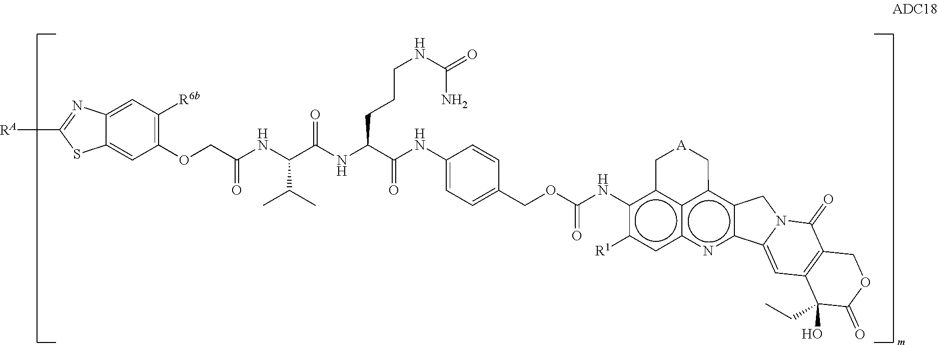

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In one embodiment, provided herein is a compound of:

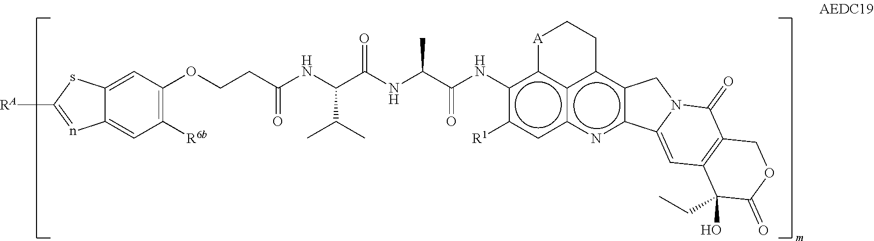

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In another embodiment, provided herein is a compound of:

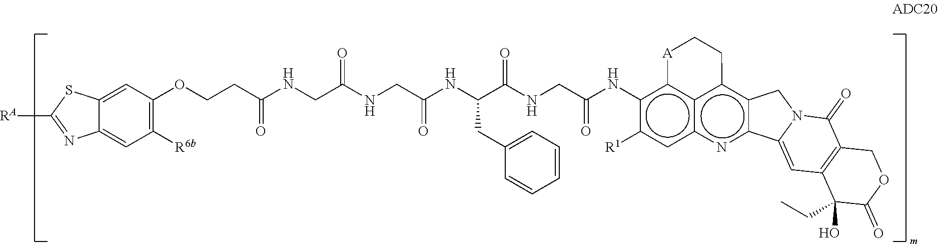

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

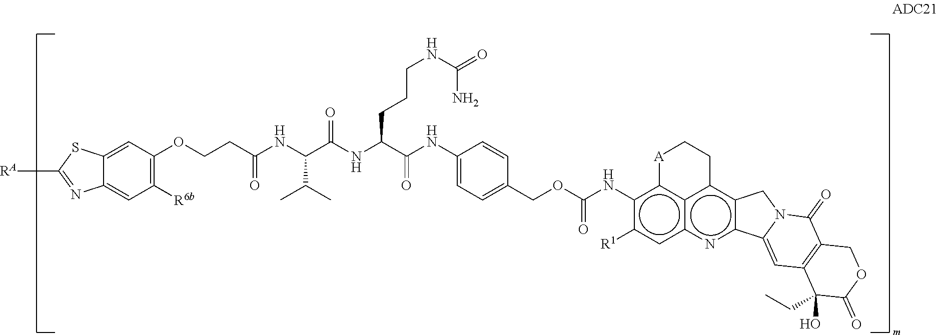

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

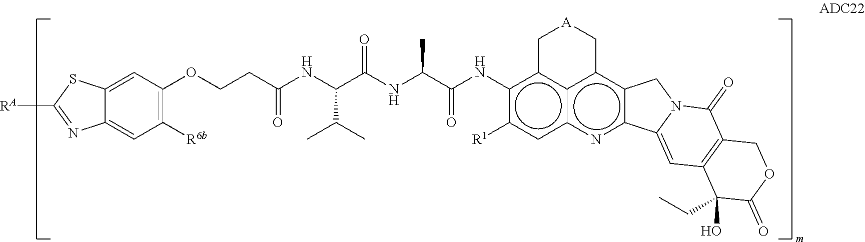

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

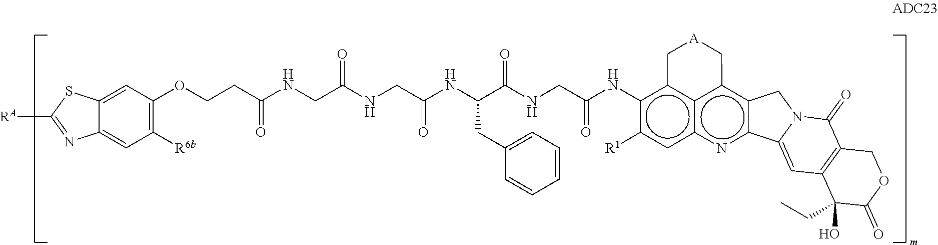

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In still another embodiment, provided herein is a compound of:

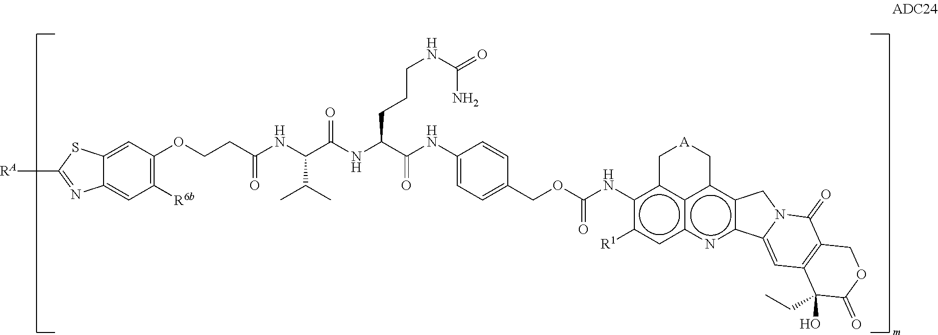

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In one embodiment, provided herein is a compound of:

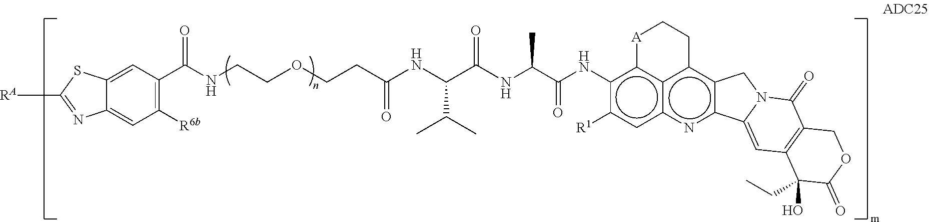

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is an integer of 1, 2, 3, 4, 5, 6, 7, or 8, in one embodiment, an integer of 4 or 8; and wherein each linker is independently connected covalently to a thiol group of a cysteine residue of the antibody.

In another embodiment, provided herein is a compound of:

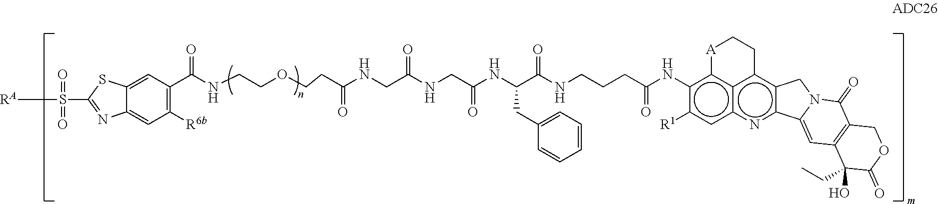

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

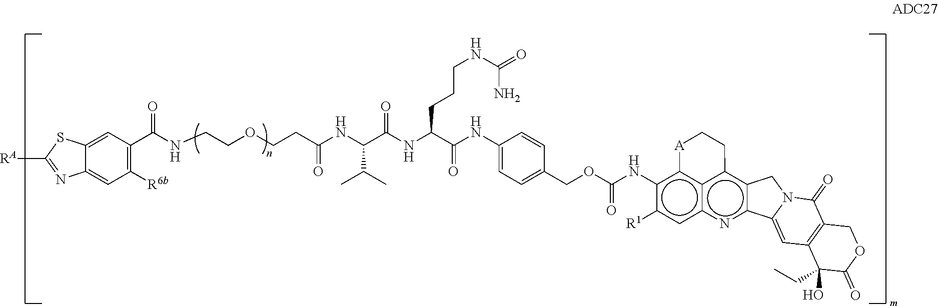

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

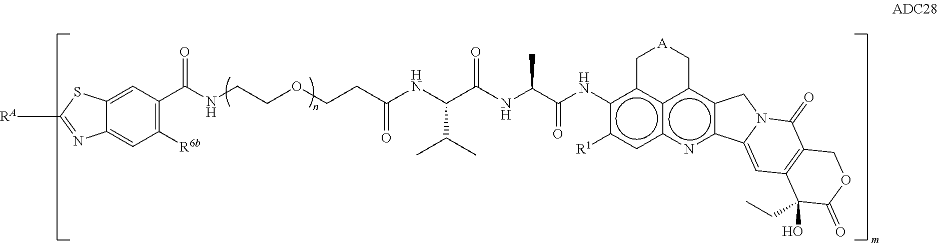

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

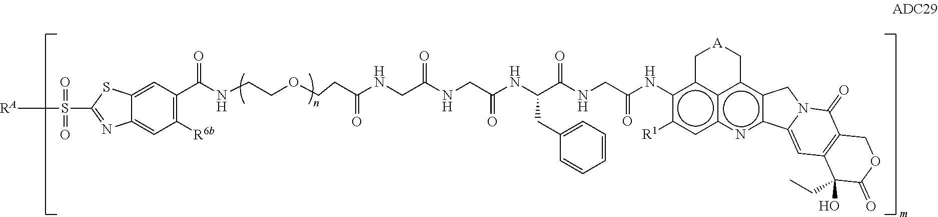

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In still another embodiment, provided herein is a compound of:

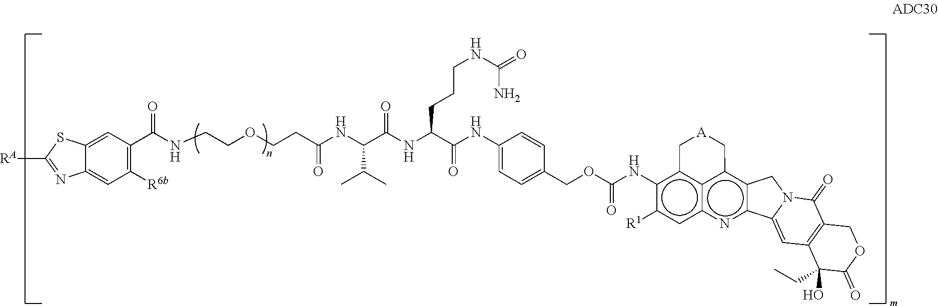

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In one embodiment, provided herein is a compound of:

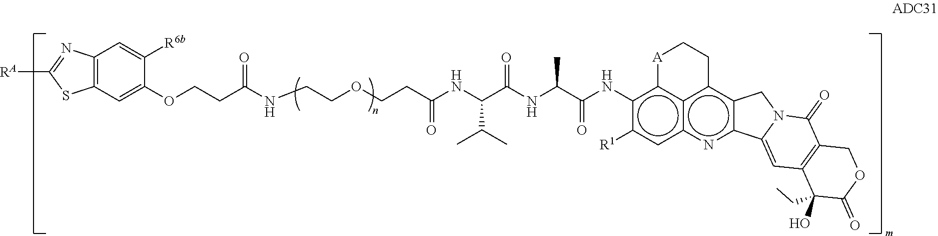

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of 4 or 8; and wherein each linker is independently connected covalently to a thiol group of a cysteine residue of the antibody.

In another embodiment, provided herein is a compound of:

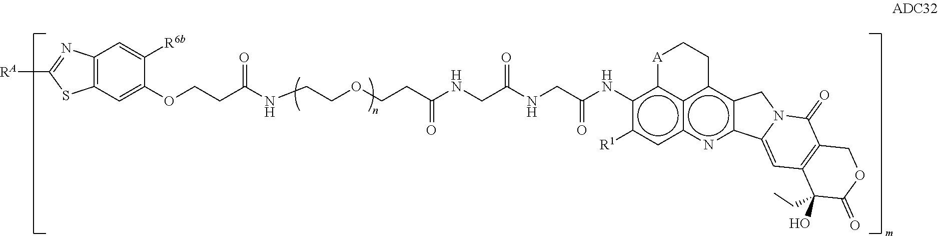

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

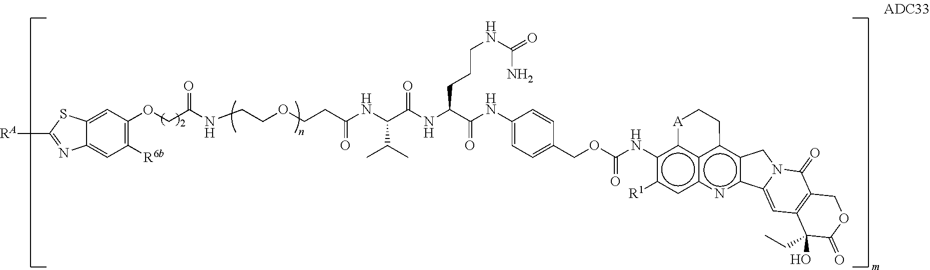

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

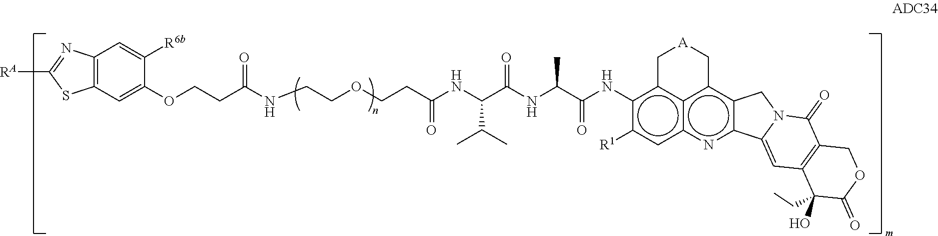

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In yet another embodiment, provided herein is a compound of:

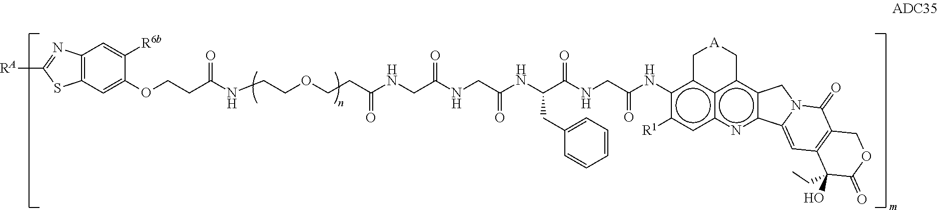

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In still another embodiment, provided herein is a compound of:

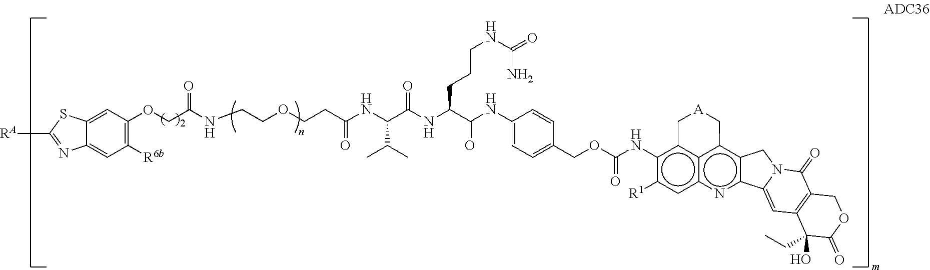

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; m is as defined herein, in one embodiment, an integer of 2, 3, 4, 5, 6, 7, or 8; and each u is as defined herein, in one embodiment, an integer of an integer of 4 or 8; and wherein each linker is independently connected covalently to a thiol group of a cysteine residue of the antibody.

In one embodiment, provided herein is a compound of:

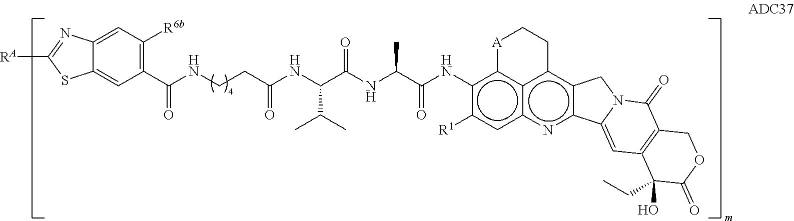

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In another embodiment, provided herein is a compound of:

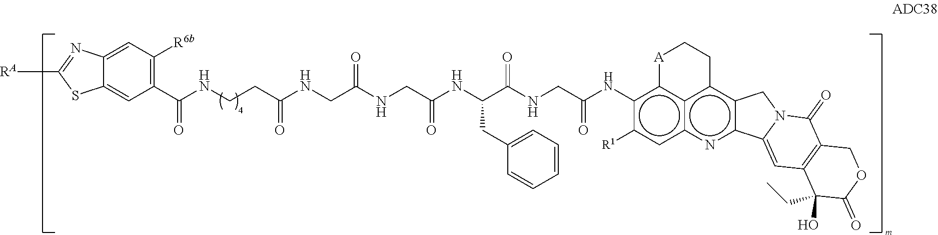

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

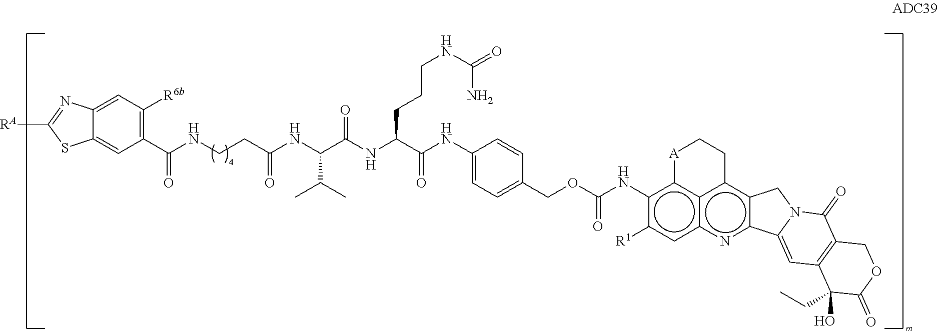

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

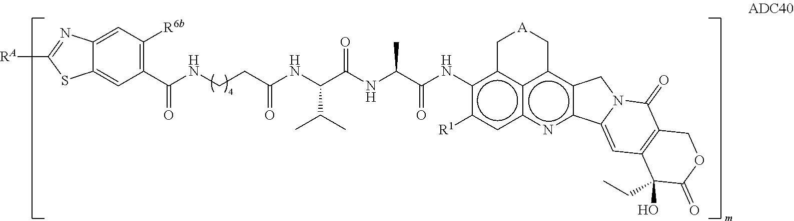

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In yet another embodiment, provided herein is a compound of:

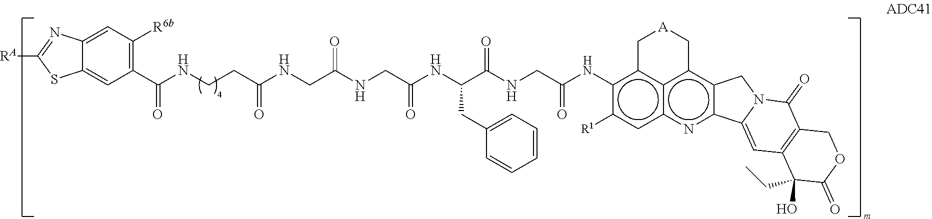

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In still another embodiment, provided herein is a compound of:

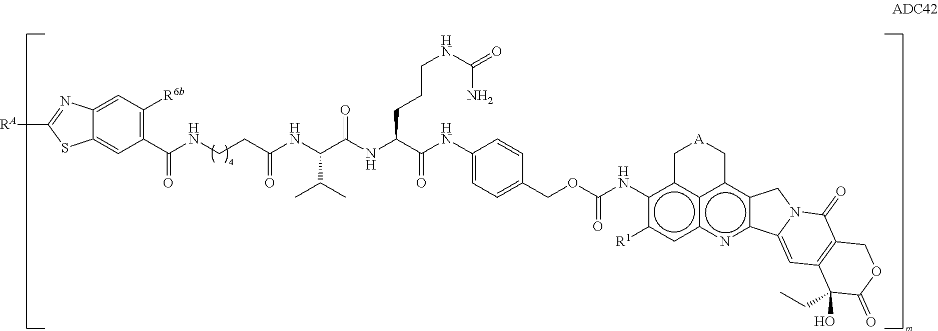

or a diastereomer, a mixture of two or more diastereomers, a tautomer, a mixture of two or more tautomers, or an isotopic variant thereof; or a pharmaceutically acceptable salt, solvate, or hydrate thereof; wherein each R¹ is as defined herein, in one embodiment, hydrogen or fluoro; each R^{6b} is as defined herein, in one embodiment, hydrogen or fluoro; R^{A} is an antibody as defined herein, in one embodiment, an anti-B7H3 antibody, anti-CD20 antibody, anti-FOLR1 antibody, anti-HER2 antibody, anti-MSLN antibody, or anti-TROP2 antibody; each A is as defined herein, in one embodiment, O or S; and m is as defined herein, in one embodiment, an integer of 2, 3, 4, 5, 6, 7, or 8; and wherein each linker is independently connected covalently to a thiol group of a cysteine residue of the antibody.

In certain embodiments, an ADC compound provided herein in a composition (e.g., a pharmaceutical composition) has a drug-antibody ratio (DAR) ranging from about 0.5 to about 12, from about 1 to about 10, from about 2 to about 8, or from about 3 to about 5. In certain embodiments, an ADC compound provided herein in a composition (e.g., a pharmaceutical composition) has a DAR ranging from about 0.5 to about 12. In certain embodiments, an ADC compound provided herein in a composition (e.g., a pharmaceutical composition) has a DAR ranging from about 1 to about 10. In certain embodiments, an ADC compound provided herein in a composition (e.g., a pharmaceutical composition) has a DAR ranging from about 2 to about 8. In certain embodiments, an ADC compound provided herein in a composition (e.g., a pharmaceutical composition) has a DAR ranging from about 3 to about 5. In certain embodiments, an ADC compound provided herein in a composition (e.g., a pharmaceutical composition) has a DAR of about 2, about 2.5, about 3, about 3.5, about 4, about 4.5, about 5, about 5.5, about 6, about 6.5, about 7, about 7.5, or about 8.

In one embodiment, provided herein is:
6-(2, 5-dioxo-2, 5-dihydro-1H-pyrrol-1-yl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-hexanamide A1;
6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-1-oxopropan-2-yl) amino)-3-methyl-1-oxobutan-2-yl)-hexanamide A2;
6-(2, 5-dioxo-2, 5-dihydro-1H-pyrrol-1-yl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl) amino)-1-oxo-3-phenylpropan-2-yl) amino)-2-oxoethyl)-amino)-2-oxoethyl) hexanamide A3;
6-(2, 5-dioxo-2, 5-dihydro-1H-pyrrol-1-yl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl) amino)-1-oxo-3-phenylpropan-2-yl) amino)-2-oxoethyl) amino)-2-oxoethyl) hexanamide A4;
4-((S)-2-((S)-2-(6-(2, 5-dioxo-2, 5-dihydro-1H-pyrrol-1-yl) hexanamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate A5;
4-((S)-2-((S)-2-(6-(2, 5-dioxo-2, 5-dihydro-1H-pyrrol-1-yl) hexanamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-carbamate A6;
6-(2, 5-dioxo-2, 5-dihydro-1H-pyrrol-1-yl)-N—((S)-1-(((S)-1-(((S)-9-ethyl-9-hydroxy-10, 13-dioxo-9, 10, 13, 15-tetrahydro-1H, 3H, 12H-pyrano [3, 4, 5-de]pyrano [3′, 4′: 6, 7]-indolizino [1,2-b]quinolin-4-yl) amino)-1-oxopropan-2-yl) amino)-3-methyl-1-oxobutan-2-yl)-hexanamide A7; or
6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-9, 10, 13, 15-tetrahydro-1H, 3H, 12H-pyrano [3, 4, 5-de]pyrano [3′, 4′: 6, 7]-indolizino [1,2-b]quinolin-4-yl) amino)-1-oxopropan-2-yl) amino)-3-methyl-1-oxobutan-2-yl)-hexanamide A8;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

In another embodiment, provided herein is:
6-(2, 5-dioxo-2, 5-dihydro-1H-pyrrol-1-yl)-N—((S)-1-(((S)-1-(((S)-9-ethyl-9-hydroxy-10, 13-dioxo-1, 2, 9, 10, 13, 15-hexahydro-12H-pyrano [3′, 4′: 6, 7]indolizino [1,2-b]-thiopyrano [4, 3, 2-de]quinolin-4-yl) amino)-1-oxopropan-2-yl) amino)-3-methyl-1-oxobutan-2-yl)-hexanamide B1;
6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]thio-pyrano [4, 3, 2-de]quinolin-4-yl) amino)-1-oxopropan-2-yl) amino)-3-methyl-1-oxobutan-2-yl)-hexanamide B2;
6-(2, 5-dioxo-2, 5-dihydro-1H-pyrrol-1-yl)-N-(2-((2-(((S)-1-((2-(((S)-9-ethyl-9-hydroxy-10, 13-dioxo-1, 2, 9, 10, 13, 15-hexahydro-12H-pyrano [3′, 4′: 6, 7]indolizino [1,2-b]thio-pyrano [4, 3, 2-de]quinolin-4-yl) amino)-2-oxoethyl) amino)-1-oxo-3-phenylpropan-2-yl) amino)-2-oxoethyl) amino)-2-oxoethyl) hexanamide B3;
6-(2, 5-dioxo-2, 5-dihydro-1H-pyrrol-1-yl)-N-(2-((2-(((S)-1-((2-(((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]-thiopyrano [4, 3, 2-de]quinolin-4-yl) amino)-2-oxoethyl) amino)-1-oxo-3-phenylpropan-2-yl)-amino)-2-oxoethyl) amino)-2-oxoethyl) hexanamide B4;
4-((S)-2-((S)-2-(6-(2, 5-dioxo-2, 5-dihydro-1H-pyrrol-1-yl) hexanamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [3′, 4′: 6, 7]indolizino [1,2-b]thiopyrano [4, 3, 2-de]quinolin-4-yl) carbamate B5;
4-((S)-2-((S)-2-(6-(2, 5-dioxo-2, 5-dihydro-1H-pyrrol-1-yl) hexanamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]thiopyrano [4, 3, 2-de]quinolin-4-yl) carbamate B6;
6-(2, 5-dioxo-2, 5-dihydro-1H-pyrrol-1-yl)-N—((S)-1-(((S)-1-(((S)-9-ethyl-9-hydroxy-10, 13-dioxo-9, 10, 13, 15-tetrahydro-1H, 3H, 12H-pyrano [3′, 4′: 6, 7]indolizino [1,2-b]-thiopyrano [3, 4, 5-de]quinolin-4-yl) amino)-1-oxopropan-2-yl) amino)-3-methyl-1-oxobutan-2-yl)-hexanamide B7; or
6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-9, 10, 13, 15-tetrahydro-1H, 3H, 12H-pyrano [3′, 4′: 6, 7]indolizino [1,2-b]-thiopyrano [3, 4, 5-de]quinolin-4-yl) amino)-1-oxopropan-2-yl) amino)-3-methyl-1-oxobutan-2-yl) hexanamide B8;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

In yet another embodiment, provided herein is:
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-2-(methylsulfonyl) benzo [d]thiazole-6-carboxamide C1;
N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-2-(methylsulfonyl) benzo [d]-thiazole-6-carboxamide C2;
4-((S)-2-((S)-3-methyl-2-(2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C3;
2-(cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-amino)-1-oxopropan-2-yl) amino)-3-methyl-1-oxobutan-2-yl) benzo [d]thiazole-6-carboxamide C4;
2-(cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylprop an-2-yl) amino)-2-oxoethyl) amino)-2-oxo-ethyl) benzo [d]thiazole-6-carboxamide C5;
4-((S)-2-((S)-2-(2-(cyclopropylsulfonyl) benzo [d]thiazole-6-carboxamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C6;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-2-(methylsulfonyl) benzo [d]thiazole-6-carboxamide C7;
N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-2-(methyl-sulfonyl) benzo [d]thiazole-6-carboxamide C8;
4-((S)-2-((S)-3-methyl-2-(2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C9;
2-(cyclopropylsulfonyl)-N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-amino)-1-oxopropan-2-yl) amino)-3-methyl-1-oxobutan-2-yl) benzo [d]thiazole-6-carboxamide C10;
2-(cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl) benzo [d]thiazole-6-carboxamide C11;
4-((S)-2-((S)-2-(2-(cyclopropylsulfonyl) benzo [d]thiazole-6-carboxamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C12;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-5-fluoro-2-(methylsulfonyl) benzo [d]-thiazole-6-carboxamide C13;
N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-5-fluoro-2-(methylsulfonyl)-benzo [d]thiazole-6-carboxamide C14;
4-((S)-2-((S)-3-methyl-2-(5-fluoro-2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C15;
2-(cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-5-fluorobenzo [d]thiazole-6-carboxamide C16;
2-(cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylprop an-2-yl) amino)-2-oxoethyl) amino)-2-oxoethyl)-5-fluorobenzo [d]thiazole-6-carboxamide C17;
4-((S)-2-((S)-2-(2-(cyclopropylsulfonyl)-5-fluorobenzo [d]thiazole-6-carboxamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]-quinolin-4-yl) carbamate C18;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl)-5-fluoro-2-(methylsulfonyl) benzo [d]-thiazole-6-carboxamide C19;
N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-2-oxo-ethyl) amino)-1-oxo-3-phenylpropan-2-yl) amino)-2-oxoethyl) amino)-2-oxoethyl)-5-fluoro-2-(methylsulfonyl) benzo [d]thiazole-6-carboxamide C20;
4-((S)-2-((S)-3-methyl-2-(5-fluoro-2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C21;
2-(cyclopropylsulfonyl)-N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-5-fluorobenzo [d]-thiazole-6-carboxamide C22;
2-(cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′. 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl)-5-fluorobenzo [d]thiazole-6-carboxamide C23;
4-((S)-2-((S)-2-(2-(cyclopropylsulfonyl)-5-fluorobenzo [d]thiazole-6-carboxamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C24;
(S)—N—((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(2-((2-(methylsulfonyl) benzo [d]thiazol-6-yl) oxy) acetamido) butanamide C25;
(S)—N-(2-(((S)-9-ethyl-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(2-((2-(methylsulfonyl) benzo [d]thiazol-6-yl) oxy) acetamido) acetamido) acetamido)-3-phenyl-propanamide C26;
4-((S)-2-((S)-3-methyl-2-(2-((2-(methylsulfonyl) benzo [d]thiazol-6-yl) oxy)-acetamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C27;
(S)—N—((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(3-((2-(methylsulfonyl) benzo [d]thiazol-6-yl) oxy) propanamido) butanamide C28;
(S)—N-(2-(((S)-9-ethyl-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenyl-propanamide C29;
4-((S)-2-((S)-3-methyl-2-(3-((2-(methylsulfonyl) benzo [d]thiazol-6-yl) oxy)-propanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C30;
N-((17S,20S)-21-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluoro-2-(methylsulfonyl)-benzo [d]thiazole-6-carboxamide C31;
N-((17S,20S)-21-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-17-isopropyl-20-methyl-15, 18, 21-trioxo-3, 6, 9, 12-tetraoxa-16, 19-diazahenicosyl)-5-fluoro-2-(methylsulfonyl) benzo [d]thiazole-6-carboxamide C32;
2-(cyclopropylsulfonyl)-N-((17S,20S)-21-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C33;
2-(cyclopropylsulfonyl)-N-((17S,20S)-21-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C34;
N-((29S,32S)-33-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C35;
N-((29S,32S)-33-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C36;
2-(cyclopropylsulfonyl)-N-((29S,32S)-33-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diaza-tritriacontyl)-5-fluorobenzo [d]thiazole-6-carboxamide C37;
2-(cyclopropylsulfonyl)-N-((29S,32S)-33-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluorobenzo [d]thiazole-6-carboxamide C38;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl)-1-(3-((2-(methylsulfonyl) benzo [d]thiazol-6-yl) oxy)-propanamido)-3, 6, 9, 12-tetraoxapentadecan-15-amide C39;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxo-propan-2-yl) amino)-3-methyl-1-oxobutan-2-yl)-1-(3-((2-(methylsulfonyl) benzo [d]thiazol-6-yl) oxy) propanamido)-3, 6, 9, 12-tetraoxapentadecan-15-amide C40;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-1-(3-((2-(methylsulfonyl) benzo [d]thiazol-6-yl) oxy)-propanamido)-3, 6, 9, 12, 15, 18, 21, 24-octaoxaheptacosan-27-amide C41;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxo-propan-2-yl) amino)-3-methyl-1-oxobutan-2-yl)-1-(3-((2-(methylsulfonyl) benzo [d]thiazol-6-yl)-oxy) propanamido)-3, 6, 9, 12, 15, 18, 21, 24-octaoxaheptacosan-27-amide C42;
N-(6-(((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl) amino)-6-oxohexyl)-2-(methylsulfonyl)-benzo [d]thiazole-6-carboxamide C43;
N—((S)-5-benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(methylsulfonyl) benzo [d]thiazole-6-carboxamide C44;
4-((S)-2-((S)-3-methyl-2-(6-(2-(methylsulfonyl) benzo [d]thiazole-6-carboxamido)-hexanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) carbamate C45;
2-(cyclopropylsulfonyl)-N-(6-(((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) amino)-6-oxohexyl)-benzo [d]thiazole-6-carboxamide C46;
N—((S)-5-benzyl-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl) benzo [d]-thiazole-6-carboxamide C47;
4-((S)-2-((S)-2-(6-(2-(cyclopropylsulfonyl) benzo [d]thiazole-6-carboxamido)-hexanamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C48;
2-(cyclopropylsulfonyl)-N-(6-(((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) amino)-6-oxohexyl)-5-fluoro-benzo [d]thiazole-6-carboxamide C49;
N—((S)-5-benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl)-5-fluorobenzo [d]thiazole-6-carboxamide C50;
4-((S)-2-((S)-2-(6-(2-(cyclopropylsulfonyl)-5-fluorobenzo [d]thiazole-6-carboxamido)-hexanamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]-quinolin-4-yl) carbamate C51;
(S)—N-(2-(((S)-9-ethyl-5-fluoro-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenyl-propanamide C52; or
N—((S)-5-benzyl-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-16, 19, 22, 25-tetraoxa-3, 6, 9, 12-tetraazaheptacosan-27-yl)-2-(methylsulfonyl) benzo [d]thiazole-6-carboxamide C53;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

In yet another embodiment, provided herein is a trastuzumab ADC selected from ADC-A1 to ADC-A8, ADC-B1 to ADC-B8, and ADC-C1 to ADC-C53. In one embodiment, trastuzumab is an anti-HER2 antibody comprising (i) a CDRL1 of SEQ ID NO: 31; (ii) a CDRL2 of SEQ ID NO: 32; (iii) a CDRL3 of SEQ ID NO: 33; (iv) a CDRH1 of SEQ ID NO: 34; (v) a CDRH2 of SEQ ID NO: 35; and (vi) a CDRH3 of SEQ ID NO: 36. In another embodiment, trastuzumab is an anti-HER2 antibody trastuzumab is an anti-HER2 antibody comprising a light chain variable region of SEQ ID NO: 37; and a heavy chain variable region of SEQ ID NO: 38. In yet another embodiment, trastuzumab is an anti-HER2 antibody comprising a light chain of SEQ ID NO: 39; and a heavy chain of SEQ ID NO: 40.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers, unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

A compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2, 2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1, 2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1, 5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1, 3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound provided herein, e.g., a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In still another embodiment, the pharmaceutical composition provided herein is formulated in a dosage form for subcutaneous administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient (s) (e.g., a compound provided herein) sufficient to produce the desired therapeutic effect, in association with the required pharmaceutical excipient (s). Examples of a unit-dosage form include, but are not limited to, an ampoule and syringe. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### Methods of Use

In one embodiment, provided herein is a method of treating, preventing, or ameliorating one or more symptoms of a proliferative disease in a subject, comprising administering to the subject in need thereof a therapeutically effective amount of a compound provided herein, e.g., a compound of Formula (I), or a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the proliferative disease is cancer. In certain embodiments, the cancer is a solid tumor. In certain embodiments, the cancer is a hematologic malignancy.

In certain embodiments, the cancer is breast cancer, colorectal cancer, epidermoid carcinoma, gastric cancer, leukemia, lung cancer, lymphoma, melanoma, oral cancer, ovarian cancer, or pancreatic cancer. In certain embodiments, the cancer is breast cancer, B cell leukemia, Burkitt's lymphoma, colorectal cancer, epidermoid carcinoma, gastric cancer, lung cancer, melanoma, oral cancer, ovarian cancer, pancreatic cancer, T cell leukemia, or T cell lymphoma.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is resectable. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In certain embodiments, the therapeutically effective amount of a compound provided herein is ranging from about 0.1 mg/kg every four weeks to about 20 mg/kg every week, from about 0.2 mg/kg every four weeks to about 10 mg/kg every week, from about 0.5 mg/kg every four weeks to about 5 mg/kg every week, or from about 1 mg/kg every four weeks to about 2 mg/kg every week.

In one embodiment, the therapeutically effective amount of a compound provided herein is ranging from about 0.1 mg/kg every four weeks to about 20 mg/kg every week. In another embodiment, the therapeutically effective amount of a compound provided herein is ranging from about 0.2 mg/kg every four weeks to about 10 mg/kg every week. In yet another embodiment, the therapeutically effective amount of a compound provided herein is ranging from about 0.5 mg/kg every four weeks to about 5 mg/kg every week. In yet another embodiment, the therapeutically effective amount of a compound provided herein is ranging from about 1 mg/kg every four weeks to about 2 mg/kg every week. In still another embodiment, the therapeutically effective amount of a compound provided herein is about 1, about 1.5, about 2, about 2.5, about 3, about 3.5, about 4, about 4.5, or about 5 mg/kg every three weeks.

Depending on the disorder, disease, or condition to be treated and the subject's condition, a compound provided herein may be administered by parenteral (e.g., intramuscular, intraperitoneal, intravenous, CIV, intracisternal injection or infusion, subcutaneous injection, or implant) routes of administration. A compound provided herein may be formulated in suitable dosage unit with a pharmaceutically acceptable excipient, carrier, adjuvant, or vehicle, appropriate for the route of administration.

In one embodiment, a compound provided herein is administered parenterally. In another embodiment, a compound provided herein is administered intravenously. In yet another embodiment, a compound provided herein is administered intramuscularly. In still another embodiment, a compound provided herein is administered subcutaneously.

A compound provided herein can be delivered as a single dose such as, e.g., a single bolus injection, or over time such as, e.g., continuous infusion over time or divided bolus doses over time. A compound provided herein can be administered repetitively if necessary, for example, until the subject experiences stable disease or regression, or until the patient experiences disease progression or unacceptable toxicity.

A compound provided herein can be administered once a week, once every two weeks, once every three weeks, once every four weeks, once every five weeks, or once every six weeks. In one embodiment, a compound provided herein is administered once a week. In another embodiment, a compound provided herein is administered once every two weeks. In yet another embodiment, a compound provided herein is administered once every two weeks. In yet another embodiment, a compound provided herein is administered once every three weeks. In yet another embodiment, a compound provided herein is administered once every four weeks. In yet another embodiment, a compound provided herein is administered once every five weeks. In yet another embodiment, a compound provided herein is administered once every six weeks.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 3 weeks, 4 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 3 weeks, 4 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered intravenously. Thus, in accordance with certain embodiments, a compound provided herein is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration intravenously. In another embodiment, a compound provided herein is administered by one mode of administration, e.g., intravenously, whereas the second agent (an anticancer agent) is administered by another mode of administration, e.g., orally.

In one embodiment, provided herein is a method of inhibiting the growth of a cell, comprising contacting the cell with a compound provided herein, e.g., a compound of Formula (I), or an enantiomer, a mixture of enantiomers, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is a human cell. In certain embodiments, the cell is a human cancerous cell.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); mmol (millimoles); h (hour or hours); min (minute or minutes); AcCl (acetyl chloride); ACN (acetonitrile); Ac (acetyl); Ac₂O (acetic anhydride); Bn (benzyl); tBu (tert-butyl); DCC(N, N′-dicyclohexylcarbodiimide); DCM (dichloromethane); DIPEA (N, N-diisopropylethylamine); DMF (dimethylformamide); DMSO (dimethyl sulfoxide); Et (ethyl); EtOAc (ethyl acetate); EtOH (ethanol); HATU (1-[bis(dimethyl-amino) methylene]-1H-1, 2, 3-triazolo[4, 5-b]pyridinium 3-oxide hexafluoro-phosphate); HOSu (1-hydroxypyrrolidine-2, 5-dione); MCPBA (meta-chloroperoxybenzoic acid); MeOH (methanol); PE (petroleum ether); PPA (polyphosphoric acid); PPTS (pyridinium p-toluene-sulfonate); TEA (triethylamine); TFA (trifluoro-acetic acid); THF (tetrahydrofuran); TCEP (tris (2-carboxyethyl)-phosphine); HPLC (high-performance liquid chromatography); LCMS (liquid chromatography-mass spectrometry); MS (mass spectrometry); and NMR (nuclear magnetic resonance).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Synthesis of 6-(2, 5-dioxo-2, 5-dihydro-1H-pyrrol-1-yl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) hexanamide A1

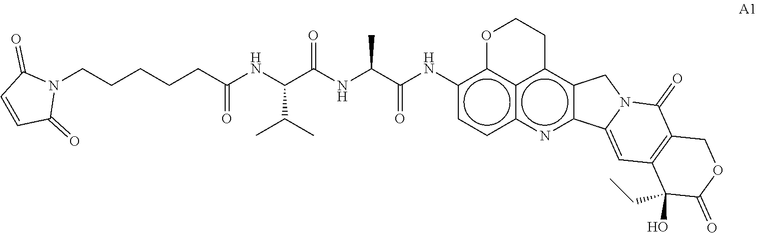

Compound A1 was synthesized as shown in Scheme 1,

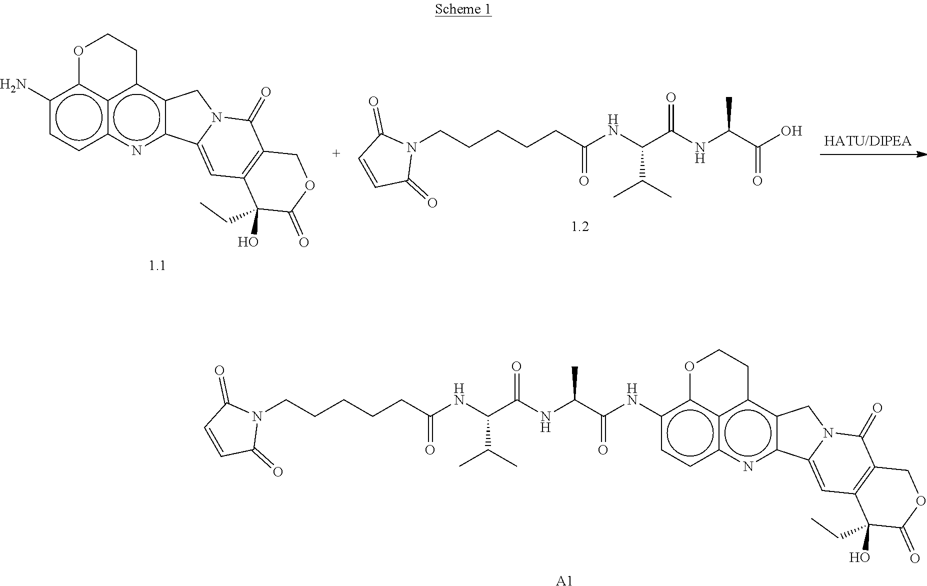

(S)-4-Amino-9-ethyl-9-hydroxy-1, 9, 12, 15-tetrahydro-13H-pyrano [4, 3, 2-de]-pyrano [3′, 4′: 6, 7]indolizino [1,2-b]quinoline-10, 13 (2H)-dione 1.1 was prepared as described in U.S. Pat. No. 4,939,255 or Sugimori et al., J. Med. Chem. 1998, 41, 2308-18, the disclosure of each of which is incorporated herein by reference in its entirety. To a solution of compound 1.1 (67.3 mg, 0.166 mmol) in DMF (4.8 mL) under argon were added (6-(2, 5-dioxo-2, 5-dihydro-1H-pyrrol-1-yl) hexanoyl)-L-valyl-L-alanine 1.2 (52.4 mg, 0.174 mmol), HATU (94.6 mg, 0.249 mmol), and DIPEA (21.4 mg, 0.166 mmol). After stirring at room temperature for 25 h, the reaction mixture was purified by column chromatography (SiO₂, MeOH/DCM) to afford compound A1 (45 mg) in 35% yield. ¹H NMR (400 MHz, DMSO-d₆) δ 9.48 (s, 1H), 8.40 (s, 1H), 8.32 (d, J=7.0 Hz, 1H), 7.89-7.76 (m, 1H), 7.68 (d, J=9.2 Hz, 1H), 7.27 (s, 1H), 6.95 (s, 2H), 6.49 (s, 1H), 5.39 (s, 2H), 5.22 (s, 2H), 4.56 (t, J=7.0 Hz, 1H), 4.49 (t, J=5.8 Hz, 2H), 4.23-4.13 (m, 1H), 2.10 (ddq, J=21.5, 14.2, 7.1 Hz, 2H), 2.01-1.88 (m, 1H), 1.82 (dq, J=14.0, 7.0 Hz, 2H), 1.43 (dt, J=15.0, 8.3 Hz, 5H), 1.31 (d, J=7.1 Hz, 4H), 1.28-1.08 (m, 14H), 0.90-0.74 (m, 12H); MS (ESI) m/z: 769.3 [M+H]⁺.

### Example 2

### Synthesis of 6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-1, 2, 9, 10, 13, 15-hexahydro-12H-pyrano-[4, 3, 2-de]pyrano [3′, 4′: 6, 7]-indolizino [1,2-b]quinolin-4-yl) amino)-1-oxopropan-2-yl) amino)-3-methyl-1-oxobutan-2-yl)-hexanamide A2

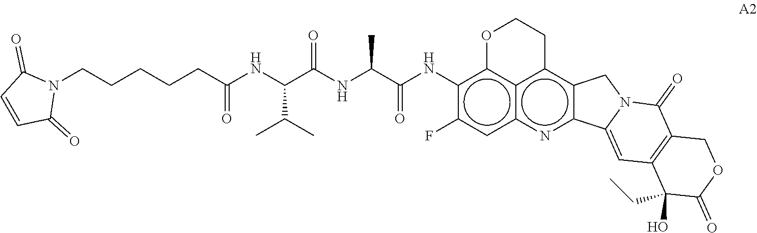

Compound A2 was synthesized as shown in Scheme 2.

Preparation of 5, 7-difluoro-8-nitrochroman-4-one 2.2. To a solution of 5, 7-difluorochroman-4-one 2.1 (104.5 g, 0.568 mol) in conc. H₂SO₄ (627 mL) at 0-5° C. was added potassium nitrate (62.8 g, 0.622 mol) in portions over 2 h. After stirring at 0-5° C. for 1 h, the reaction solution was added dropwise to ice water and extracted with EtOAc. The combined organic layers were washed with brine, dried over anhydrous Na₂SO₄, filtered, concentrated, and purified by column chromatography (SiO₂, EtOAc/PE) to afford compound 2.2 (94.5 g) in 73% yield.

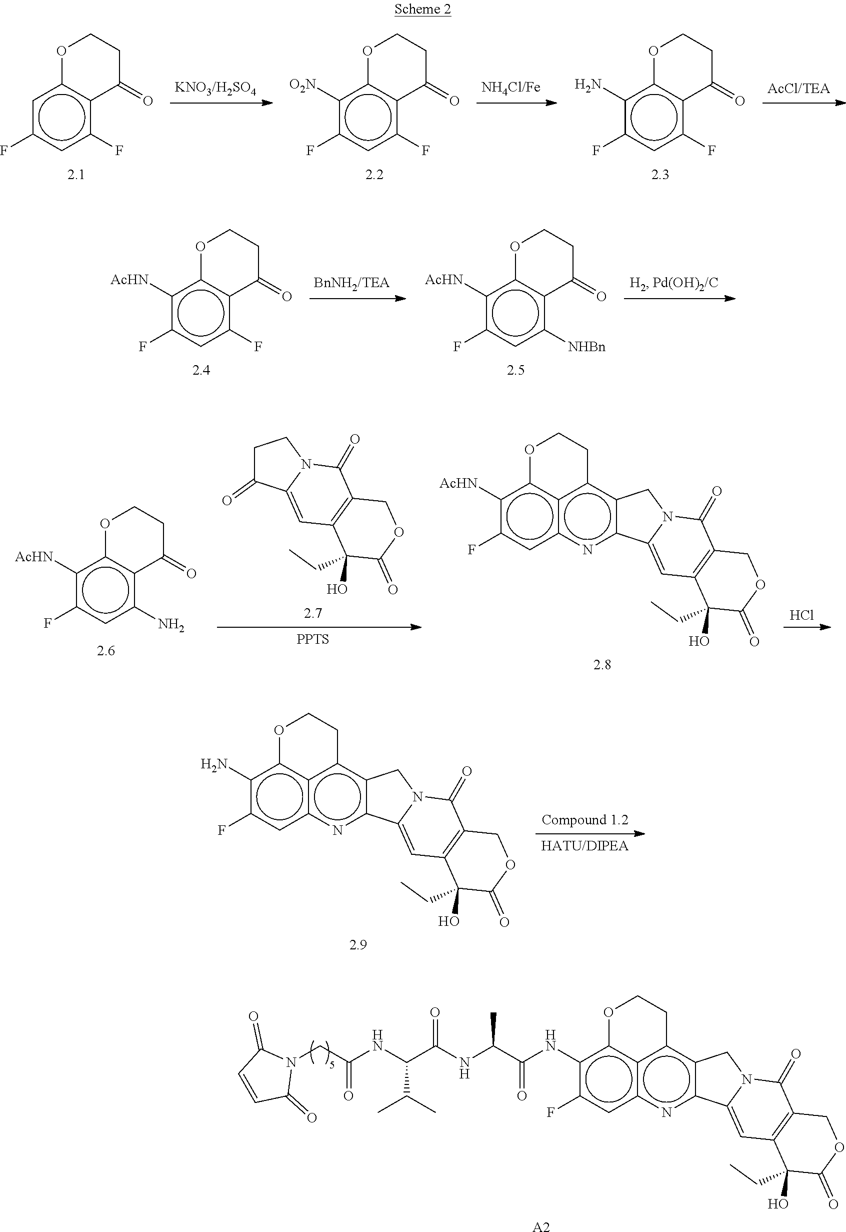

Preparation of 8-amino-5, 7-difluorochroman-4-one 2.3. To a solution of compound 2.2 (80 g, 0.349 mol) in EtOH (2.13 L) and water (267 mL) were added NH₄Cl (56.6 g, 1.06 mol) and iron powder (157.5 g) under argon. After stirring at 80° C. for 2 h, the reaction mixture was filtered, concentrated, and purified by column chromatography (SiO₂, EtOAc/PE) to afford compound 2.3 (42 g) in 60% yield.

Preparation of N-(5, 7-difluoro-4-oxochroman-8-yl) acetamide 2.4. To a solution of compound 2.3 (42 g, 0.211 mol) in DCM (1.68 L) was added TEA (97.4 g, 0.963 mol) under argon, followed by dropwise addition of acetyl chloride (66.2 g, 0.844 mol) at 0-5° C. After stirring at room temperature for 20 h, the reaction mixture was concentrated, treated with saturated saline (500 mL), filtered, and dried to afford compound 2.4 (28 g) in 55% yield.

Preparation of N-(5-(benzylamino)-7-fluoro-4-oxochroman-8-yl) acetamide 2.5. To a solution of compound 2.4 (28 g, 0.116 mol) in toluene (560 mL) were added TEA (35.2 g, 0.348 mol) and benzylamine (19.3 g, 0.18 mol) under argon. After stirring at 100° C. for 3 h, the reaction mixture was concentrated, diluted with EtOAc (400 mL), stirred at room temperature for 30 min, filtered, concentrated, and purified by column chromatography (SiO₂, MeOH/DCM) to afford compound 2.5 (18 g) in 47% yield.

Preparation of N-(5-amino-7-fluoro-4-oxochroman-8-yl) acetamide 2.6. To a solution of compound 2.5 (1.7 g, 5.18 mmol) in MeOH (70 mL) was added Pd(OH)₂/C (0.11 g) under N₂. After stirring under H₂ at room temperature for 48 h, the reaction mixture was filtered, concentrated, and purified by column chromatography (SiO₂, MeOH/DCM) to afford compound 2.6 (1.1 g) in 89% yield.

Preparation of (S)—N-(9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) acetamide 2.8. To a solution of compound 2.6 (130 mg, 0.546 mmol) in toluene (26 mL) were added (S)-4-ethyl-4-hydroxy-7, 8-dihydro-1H-pyrano [3,4-f]indolizine-3, 6, 10 (4H)-trione 2.7 (144 mg, 0.547 mmol) and PPTS (144 mg, 0.573 mmol) under argon. After refluxing for 4 h, the reaction mixture was concentrated and purified by column chromatography (SiO₂, MeOH/DCM) to afford compound 2.8 (60 mg) in 24% yield.

Preparation of (S)-4-amino-9-ethyl-5-fluoro-9-hydroxy-1, 9, 12, 15-tetrahydro-13H-pyrano [4, 3, 2-de]pyrano [3′, 4′: 6, 7]indolizino [1,2-b]quinoline-10, 13 (2H)-dione 2.9. A solution of compound 2.8 (60 mg, 0.129 mmol) in 6M HCl (9 mL) was refluxed for 1.5 h. The reaction mixture was then concentrated and purified by column chromatography (SiO₂, MeOH/DCM) to afford compound 2.9 (50 mg) in 92% yield.

Preparation of 6-(2, 5-dioxo-2, 5-dihydro-1H-pyrrol-1-yl)-N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano-[3′, 4′: 6, 7]-indolizino [1,2-b]quinolin-4-yl) amino)-1-oxopropan-2-yl) amino)-3-methyl-1-oxo-butan-2-yl)-hexanamide A2. To a solution of compound 2.9 (70.3 mg, 0.166 mmol) in DMF (4.8 mL) under argon were added (6-(2, 5-dioxo-2, 5-dihydro-1H-pyrrol-1-yl) hexanoyl)-L-valyl-L-alanine 1.2 (52.4 mg, 0.174 mmol), HATU (94.6 mg, 0.249 mmol), and DIPEA (21.4 mg, 0.166 mmol). After stirring at room temperature for 25 h, the reaction mixture was purified by column chromatography (SiO₂, MeOH/DCM) to afford compound A2 (48 mg) in 37% yield. MS (ESI) m/z: 787.3 [M+H]⁺.

### Example 3

### Synthesis of 6-(2, 5-dioxo-2, 5-dihydro-1H-pyrrol-1-yl)-N—((S)-1-(((S)-1-(((S)-9-ethyl-9-hydroxy-10, 13-dioxo-9, 10, 13, 15-tetrahydro-1H, 3H, 12H-pyrano-[3, 4, 5-de]pyrano [3′, 4′: 6, 7]indolizino [1,2-b]quinolin-4-yl) amino)-1-oxopropan-2-yl) amino)-3-methyl-1-oxobutan-2-yl) hexanamide A7

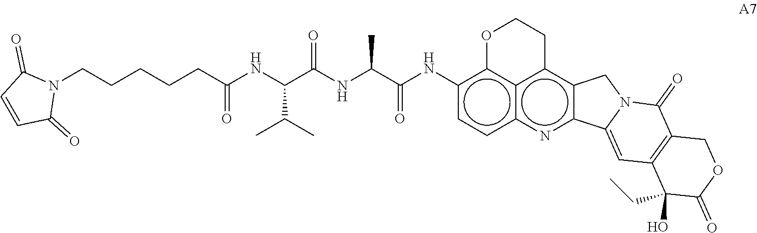

Compound A7 is synthesized as shown in Scheme 3.

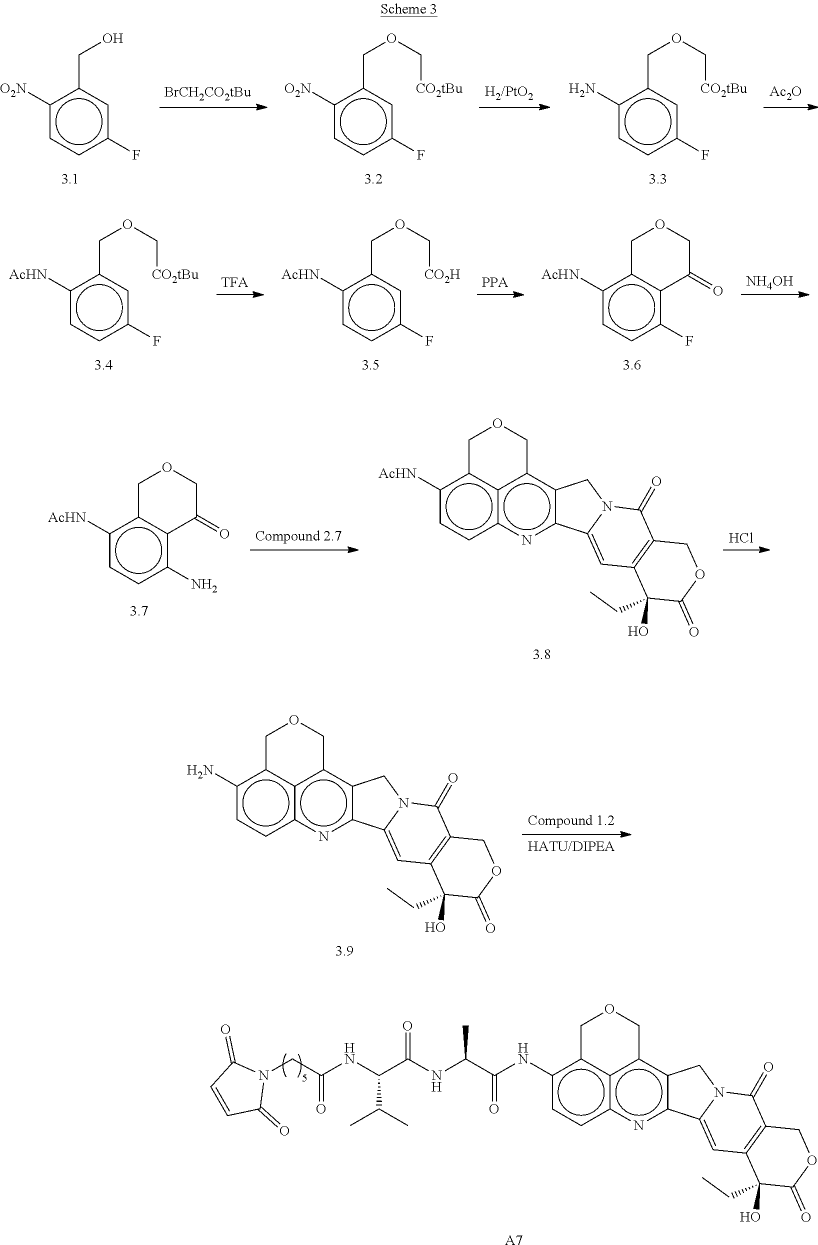

### Example 4

### Synthesis of N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-2-oxoethyl) amino)-1-oxo-3-phenylpropan-2-yl) amino)-2-oxoethyl) amino)-2-oxoethyl)-2-(methylsulfonyl)-benzo [d]thiazole-6-carboxamide

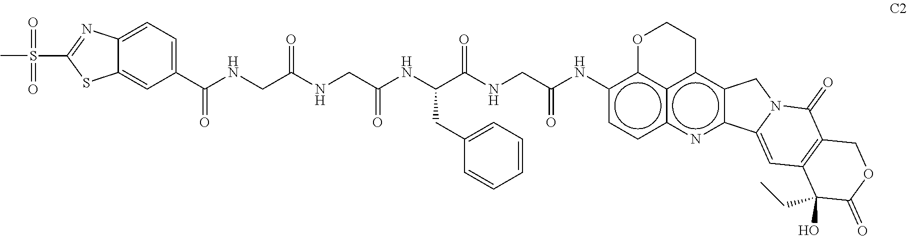

Compound C2 was synthesized as shown in Scheme 4.

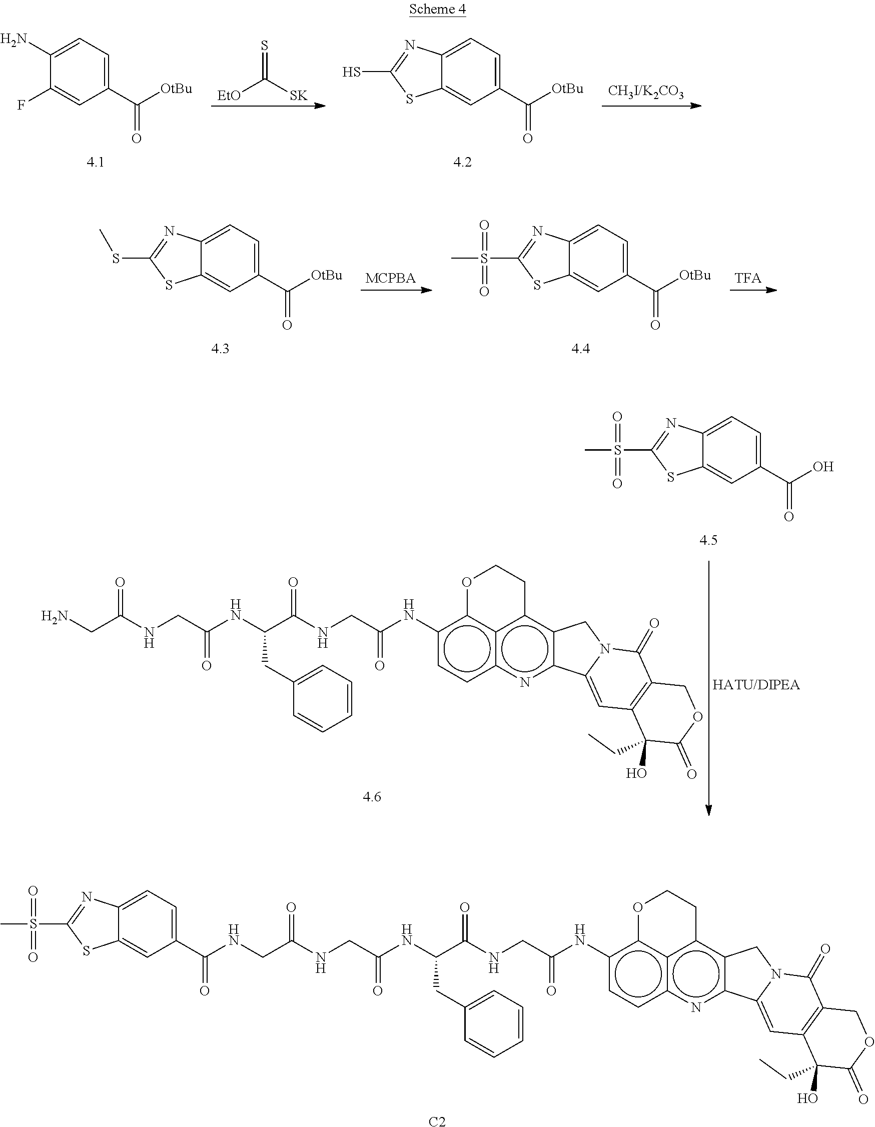

Preparation of tert-butyl 2-mercaptobenzo [d]thiazole-6-carboxylate 4.2. To a solution of tert-butyl 4-amino-3-fluorobenzoate 4.1 (5 g, 23.7 mmol) in DMF (107 mL) was added potassium ethyl xanthate (6.5 g, 33.2 mmol) under argon. After stirring at 120-130° C. for 12 h, the reaction mixture was filtered, diluted with water (200 mL), and adjusted to pH 2 with HCl. The resulting precipitates were collected by filtration to afford compound 4.2 (4.7 g) in 75% yield.

Preparation of tert-butyl 2-(methylthio) benzo [d]thiazole-6-carboxylate 4.3. To a solution of compound 4.2 (0.8 g, 2.99 mmol) in THF (32 mL) was added K₂CO₃ (1.03 g, 7.45 mmol), followed by dropwise addition of methyl iodide (0.85 g, 5.99 mmol) at 0-5° C. After stirring at room temperature for 2 h, the reaction mixture was filtered, concentrated, and purified by column chromatography (SiO₂, EtOAc/PE) to afford compound 4.3 (0.5 g) in 60% yield.

Preparation of tert-butyl 2-(methylsulfonyl) benzo [d]thiazole-6-carboxylate 4.4. To a solution of compound 4.3 (0.5 g, 1.78 mmol) in DCM (20 mL) was added 85% MCPBA (1.5 g, 7.39 mmol). After stirring at room temperature for 16 h, the reaction mixture was filtered, concentrated, and purified by column chromatography (SiO₂, EtOAc/PE) to afford compound 4.4 (0.45 g) in 81% yield.

Preparation of 2-(methylsulfonyl) benzo [d]thiazole-6-carboxylic acid 4.5. To a solution of compound 4.4 (0.45 g, 1.44 mmol) in DMC (4.5 mL) was added TFA (4.5 mL). After stirring at room temperature for 2 h, the reaction mixture was concentrated and purified by column chromatography (SiO₂, MeOH/DCM) to afford compound 4.5 (0.3 g) in 81% yield. ¹H NMR (600 MHz, DMSO-d₆) δ 13.46 (s, 1H), 9.01 (s, 1H), 8.35 (d, J=8.6 Hz, 1H), 8.22 (dd, J=8.6, 1.6 Hz, 1H), 3.63 (s, 3H).

Preparation of N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxo-ethyl)-2-(methylsulfonyl)-benzo [d]thiazole-6-carboxamide C2. To a solution of (S)-2-(2-(2-aminoacetamido) acetamido)-N-(2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxo-ethyl)-3-phenylpropanamide 4.6 (120 mg, 0.166 mmol) in DMF (4.8 mL) under argon were compound 4.5 (44.8 mg, 0.174 mmol), HATU (94.6 mg, 0.249 mmol), and DIPEA (21.4 mg, 0.166 mmol). After stirring at room temperature for 25 h, the reaction mixture was purified by column chromatography (SiO₂, MeOH/DCM) to afford compound C2 (45 mg) in 28% yield. ¹H NMR (600 MHz, DMSO-d₆) δ 9.55 (s, 1H), 9.17 (t, J=5.8 Hz, 1H), 8.85 (d, J=1.7 Hz, 1H), 8.57 (t, J=5.8 Hz, 1H), 8.41 (d, J=9.2 Hz, 1H), 8.37-8.22 (m, 3H), 8.19 (dd, J=8.6, 1.7 Hz, 1H), 7.78-7.62 (m, 1H), 7.36-7.28 (m, 3H), 7.25 (t, J=7.5 Hz, 2H), 7.18 (t, J=7.3 Hz, 1H), 6.53 (s, 1H), 5.76 (s, 2H), 5.44 (s, 2H), 5.26 (s, 2H), 4.58 (td, J=9.2, 4.1 Hz, 1H), 4.53 (t, J=5.9 Hz, 2H), 4.13 (q, J=5.2 Hz, 2H), 4.02 (t, J=4.8 Hz, 2H), 3.96 (d, J=5.8 Hz, 2H), 3.87-3.78 (m, 2H), 3.60 (s, 4H), 3.17 (d, J=5.2 Hz, 6H), 2.96-2.81 (m, 1H), 2.08-1.94 (m, 1H), 1.88 (ddp, J=21.4, 14.4, 7.3 Hz, 2H), 0.87 (t, J=18.9, 7.2 Hz, 3H); ¹³C NMR (151 MHz, DMSO-d₆) δ 172.93, 172.21, 170.10, 169.59, 169.16, 168.44, 166.26, 157.31, 154.01, 151.69, 150.50, 146.27, 145.14, 141.69, 138.35, 136.47, 135.97, 133.88, 129.62, 128.59, 127.28, 126.78, 126.01, 125.47, 124.90, 123.90, 122.59, 121.10, 119.35, 116.82, 97.14, 72.86, 65.73, 65.64, 54.60, 49.70, 43.39, 42.47, 42.41, 40.52, 38.09, 30.77, 25.86, 8.24; MS (ESI) m/z: 962.3 [M+H]⁺.

### Example 5

### Synthesis of N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6,7]indolizino [1,2-b]quinolin-4-yl) amino)-2-oxoethyl) amino)-1-oxo-3-phenylpropan-2-yl) amino)-2-oxoethyl) amino)-2-oxoethyl)-2-(methylsulfonyl) benzo [d]thiazole-6-carboxamide C8

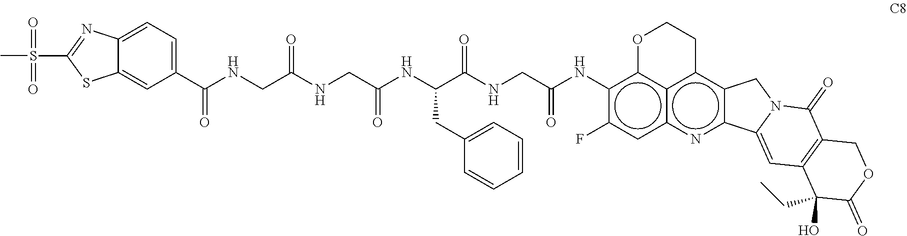

Compound C8 was synthesized as shown in Scheme 5.

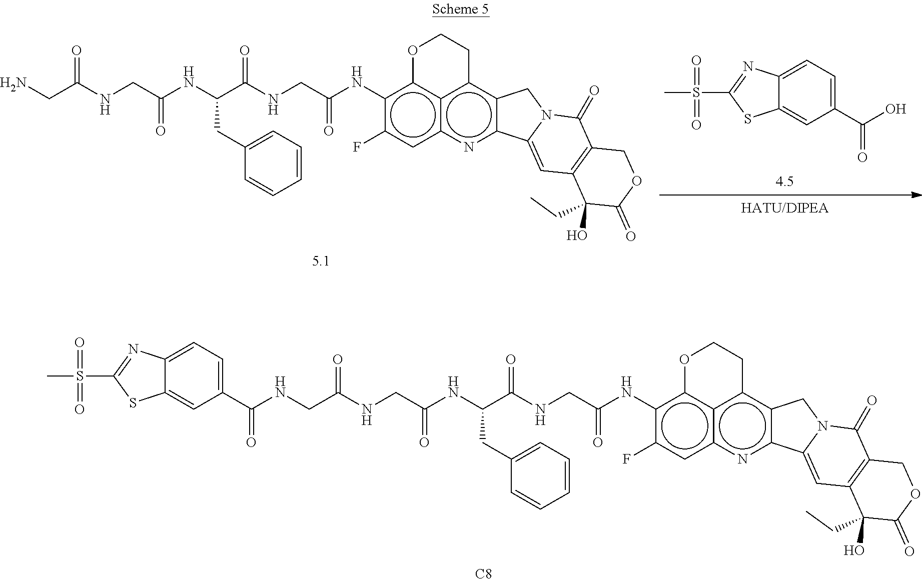

To a solution of (S)-2-(2-(2-aminoacetamido) acetamido)-N-(2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl)-3-phenylpropanamide 5.1 (120 mg, 0.162 mmol) in DMF (4.8 mL) under argon were added 2-(methylsulfonyl) benzo [d]thiazole-6-carboxylic acid 4.5 (43.7 mg, 0.170 mmol), HATU (92.3 mg, 0.243 mmol), and DIPEA (20.9 mg, 0.162 mmol). After stirring at room temperature for 25 h, the reaction mixture was purified by column chromatography (SiO₂, MeOH/DCM) to afford compound C8 (33 mg) in 21% yield. ¹H NMR (600 MHz, DMSO-d₆) δ 9.70 (s, 1H), 9.15 (t, J=5.8 Hz, 1H), 8.85 (d, J=1.7 Hz, 1H), 8.48 (t, broad, 1H), 8.31 (d, J=8.5 Hz, 1H), 8.29-8.13 (m, 3H), 7.56 (d, J=11.1 Hz, 1H), 7.33 (s, 1H), 7.30-7.21 (m, 4H), 7.19-7.13 (m, 1H), 6.53 (s, 1H), 5.44 (s, 2H), 5.26 (s, 2H), 4.58 (td, J=9.1, 4.2 Hz, 1H), 4.53 (t, J=6.0 Hz, 2H), 4.10-3.97 (m, 1H), 3.95 (d, J=5.7 Hz, 2H), 3.81 (dd, J=16.6, 6.0 Hz, 1H), 3.73-3.52 (m, 5H), 3.19-3.02 (m, 2H), 2.87 (dd, J=13.9, 9.9 Hz, 1H), 1.87 (dhept, J=21.4, 7.3 Hz, 2H), 1.43-1.10 (m, 17H), 0.88 (t, J=7.3 Hz, 3H); MS (ESI) m/z: 981.2 [M+H]⁺.

### Example 6

### Synthesis of 2-(cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano-[3′, 4′: 6, 7]indolizino [1,2-b]quinolin-4-yl)-amino)-1-oxopropan-2-yl) amino)-3-methyl-1-oxobutan-2-yl)-5-fluorobenzo [d]thiazole-6-carboxamide C16

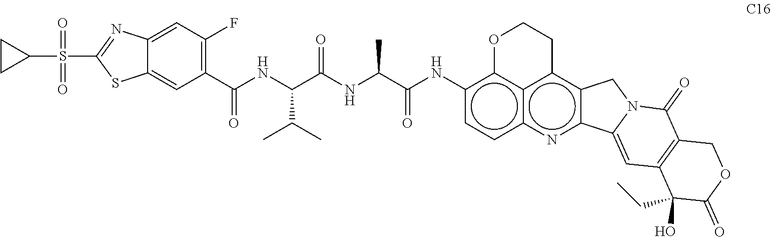

Compound C16 is synthesized as shown in Scheme 6.

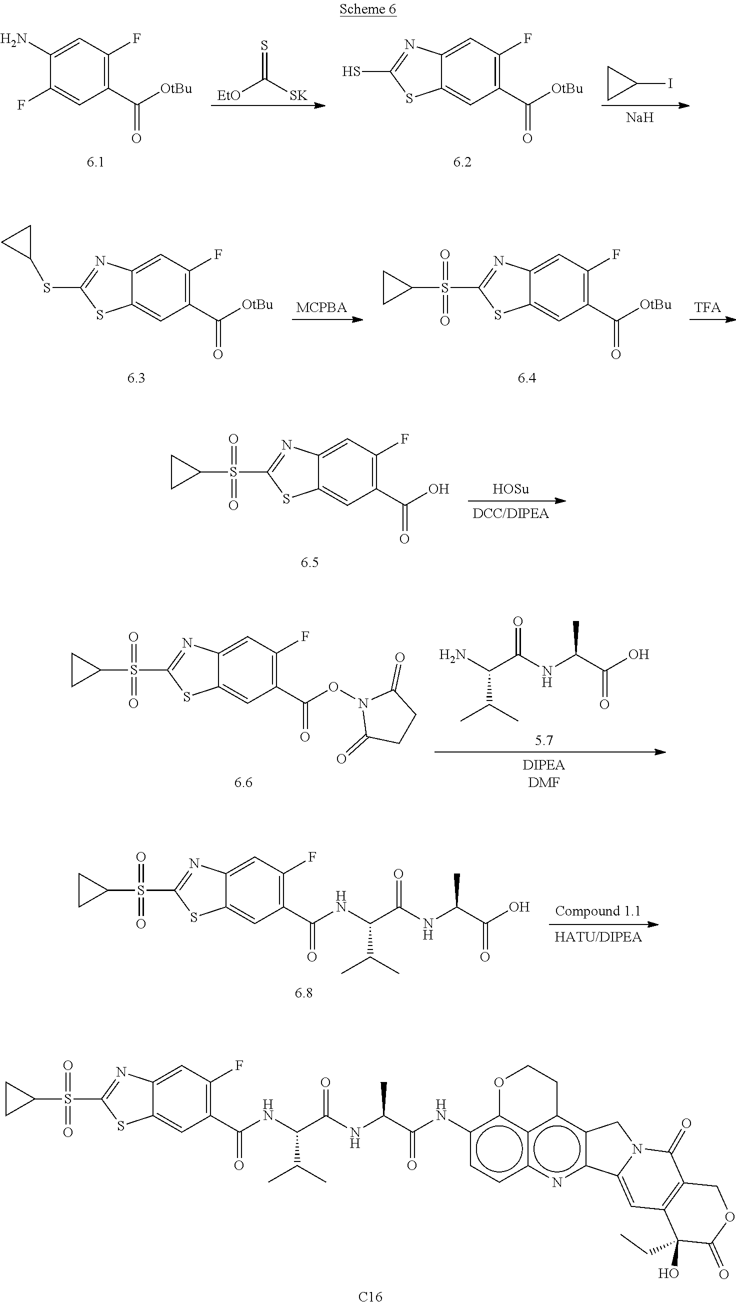

### Example 7

### Synthesis of (S)—N-(2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenyl-propanamide C29

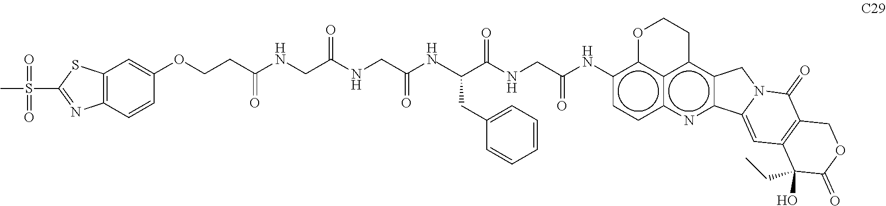

Compound C29 was synthesized as shown in Schemes 7a and 7b.

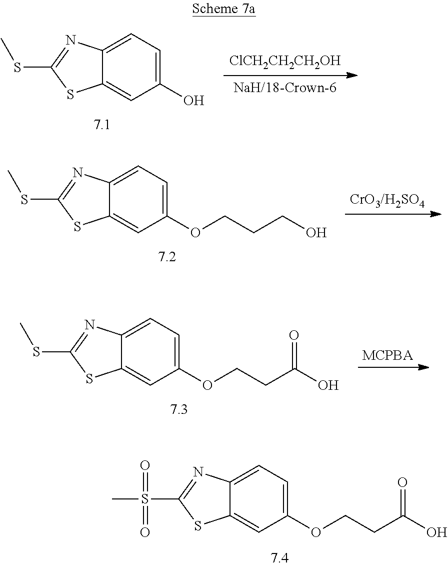

Preparation of 3-((2-(methylthio) benzo [d]thiazol-6-yl) oxy) propan-1-ol 7.2. To a mixture of NaH (60%) (1.16 g, 29 mmol) in dry DMF (10 mL) under argon was added 2-(methylthio) benzo [d]thiazol-6-ol 7.1 (5 g, 27.6 mmol). After the mixture was stirred for 20 min, 18-crown-6 (0.55 g, 2.08 mmol) and 3-chloro-1-propanol (3.89 g, 41.1 mmol) were added subsequently. The reaction mixture was stirred at 80° C. for 48 h, and then poured into water (200 mL) and extracted with EtOAc. The combined organic layers were washed with brine, dried over anhydrous Na₂SO₄, concentrated, and purified by column chromatography (SiO₂, EtOAc/PE) to afford compound 7.2 (1.4 g) in 21% yield.

Preparation of 3-((2-(methylthio) benzo [d]thiazol-6-yl) oxy) propanoic acid 7.3. To a solution of CrO₃ (1.04 g, 10.4 mmol) in water (1.5 mL) at 0-5° C. was added 98% conc. H₂SO₄ (1.6 ml, 16.3 mmol) slowly. After stirring at 0-5° C. for 30 min, the mixture was added to a solution of compound 7.2 (1.4 g, 5.85 mmol) in acetone (58 mL) at 0-5° C. The reaction mixture was stirred at 0-5° C. for 30 min, and then diluted with water (120 mL) and extracted with EtOAc. The combined organic layers were washed with brine, dried over anhydrous Na₂SO₄, and concentrated to afford compound 7.3 (1 g), which was used directly in the next step without further purification.

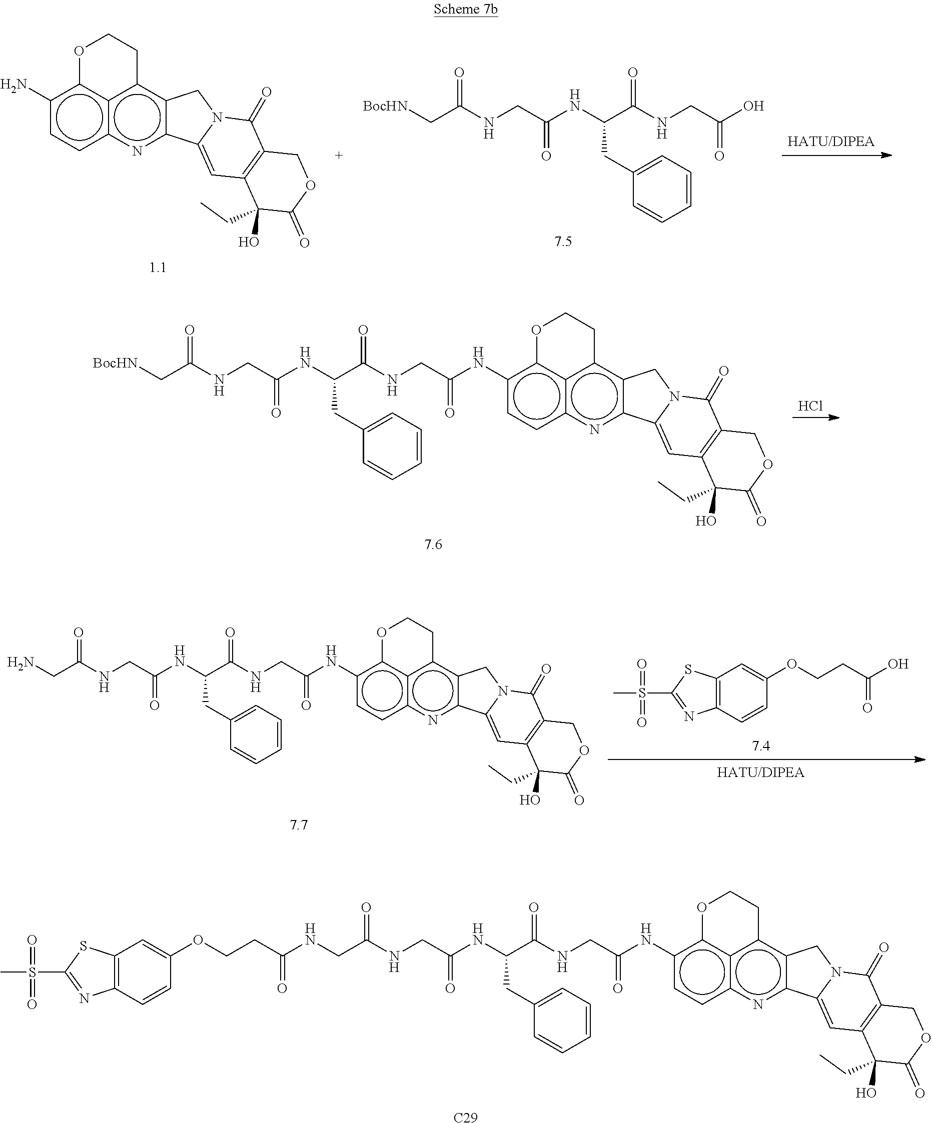

Preparation of 3-((2-(methylsulfonyl) benzo [d]thiazol-6-yl) oxy) propanoic acid 7.4. To a solution of compound 7.3 (1 g, 3.95 mmol) in DCM (40 mL) was added MCPBA (3 g, 17.4 mmol). After stirring at room temperature for 12 h, the reaction mixture was filtered, concentrated, and purified by column chromatography (SiO₂, MeOH/DCM) to afford compound 7.4 (0.5 g) in 44% yield. ¹H NMR (600 MHz, DMSO-d₆) δ 8.13 (d, J=9.0 Hz, 1H), 7.92 (d, J=2.5 Hz, 1H), 7.30 (dd, J=9.0, 2.6 Hz, 1H), 4.30 (t, J=6.0 Hz, 2H), 3.55 (s, 3H), 2.78 (t, J=6.0 Hz, 2H).

Preparation of tert-butyl (2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-amino)-2-oxoethyl) amino)-1-oxo-3-phenylpropan-2-yl) amino)-2-oxoethyl) amino)-2-oxoethyl)-carbamate 7.6. To a solution of compound 1.1 (250 mg, 0.617 mmol) in DMF (10 mL) under argon were added (tert-butoxycarbonyl) glycylglycyl-L-phenylalanylglycine 7.5 (283 mg, 0.648 mmol), HATU (352 mg, 0.926 mmol), and DIPEA (80 mg, 0.617 mmol). After stirring at room temperature for 20 h, the reaction mixture was poured into water (20 mL) and extracted with EtOAc (5×20 mL). The combined organic layers were washed with brine, dried over anhydrous Na₂SO₄, concentrated, and purified by column chromatography (SiO₂, MeOH/DCM) to afford compound 7.6 (250 mg) in 49% yield.

Preparation of (S)-2-(2-(2-aminoacetamido) acetamido)-N-(2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl)-3-phenylpropanamide 7.7. To a mixture of compound 7.6 (250 mg, 0.303 mmol) in EtOAc (12.5 mL) was added 6M HCl in EtOAc (5 mL). After stirring at room temperature for 12 h, the reaction mixture was concentrated and purified by trituration with EtOAc/PE to afford compound 7.7 (115 mg) in 52% yield.

Preparation of (S)—N-(2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxo-ethyl)-2-(2-(2-(3-((2-(methylsulfonyl) benzo [d]thiazol-6-yl) oxy) propanamido) acetamido)-acetamido)-3-phenyl-propanamide C29. To a solution of compound 7.7 (120 mg, 0.166 mmol) in DMF (4.8 mL) under argon were added compound 7.4 (52.4 mg, 0.174 mmol), HATU (94.6 mg, 0.249 mmol), and DIPEA (21.4 mg, 0.166 mmol). After stirring at room temperature for 25 h, the reaction mixture was purified by column chromatography (SiO₂, MeOH/DCM) to afford compound C29 (40 mg) in 24% yield. MS (ESI) m/z: 1007.3 [M+H]⁺.

### Example 8

### Synthesis of 2-(cyclopropylsulfonyl)-N-((17S,20S)-21-(((S)-9-ethyl-9-hydroxy-10, 13-dioxo-1, 2, 9, 10, 13, 15-hexahydro-12H-pyrano [4, 3, 2-de]pyrano [3′, 4′: 6,7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C33

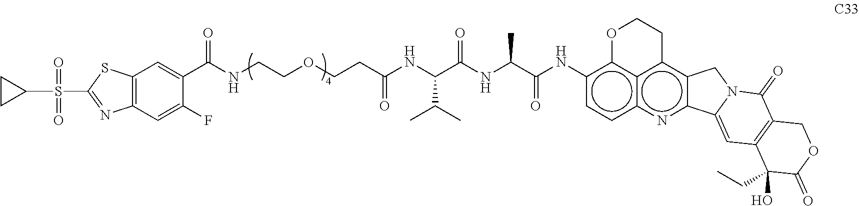

Compound C33 is synthesized as shown in Scheme 8.

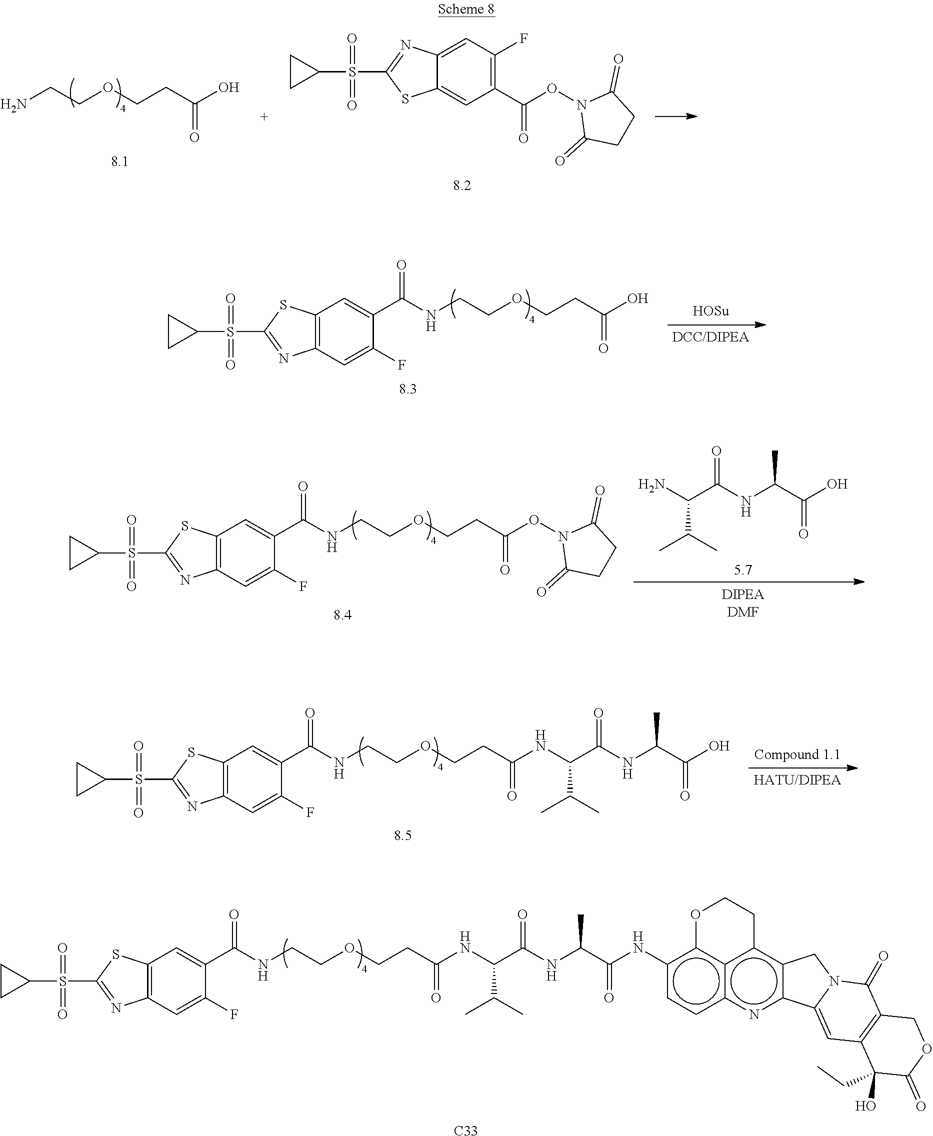

### Example 9

Synthesis of N—((S)-1-(((S)-1-(((S)-9-ethyl-9-hydroxy-10, 13-dioxo-1, 2, 9, 10, 13, 15-hexahydro-12H-pyrano[4, 3, 2-de] pyrano[3′, 4′: 6,7] indolizino[1, 2-b] quinolin-4-yl)amino)-1-oxopropan-2-yl)amino)-3-methyl-1-oxobutan-2-yl)-1-(3-((2-(methylsulfonyl)benzo[d] thiazol-6-yl)oxy)-propanamido)-3, 6, 9, 12-tetraoxapentadecan-15-amide C39

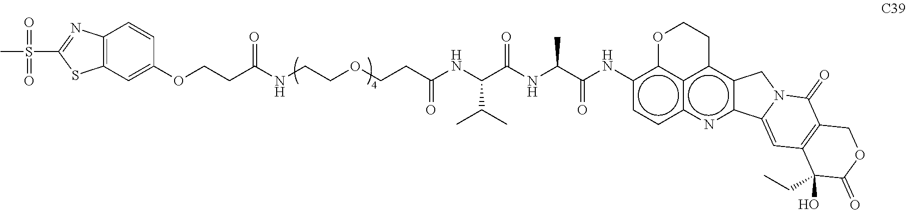

Compound C39 is synthesized as shown in Scheme 9.

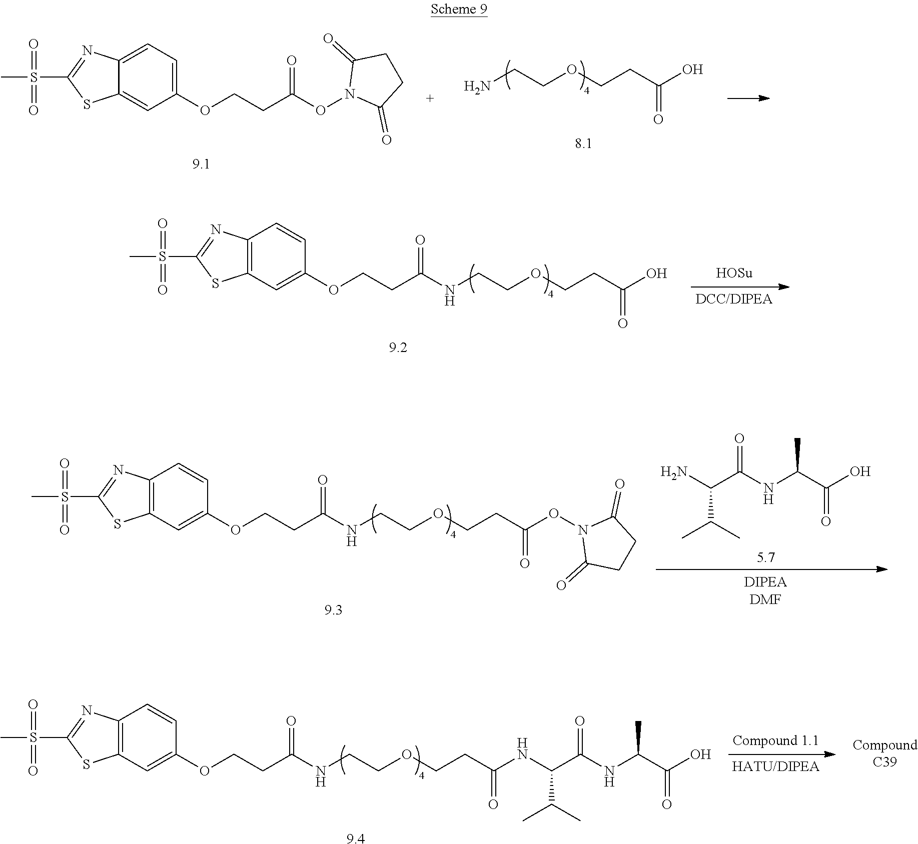

### Example 10

### Synthesis of (S)—N-(2-(((S)-9-ethyl-5-fluoro-9-hydroxy-10, 13-dioxo-1, 2, 9, 10, 13, 15-hexahydro-12H-pyrano-[4, 3, 2-de]pyrano [3′, 4′: 6, 7]indolizino [1, 2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenylpropanamide C52

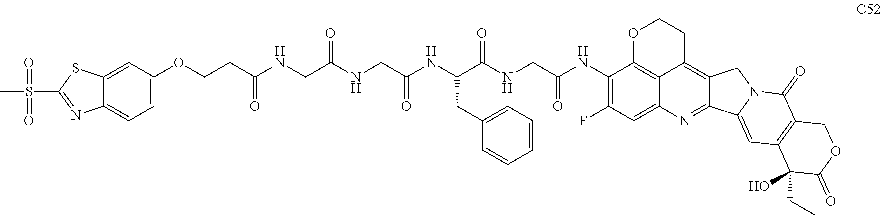

Compound C52 was synthesized as shown in Scheme 10.

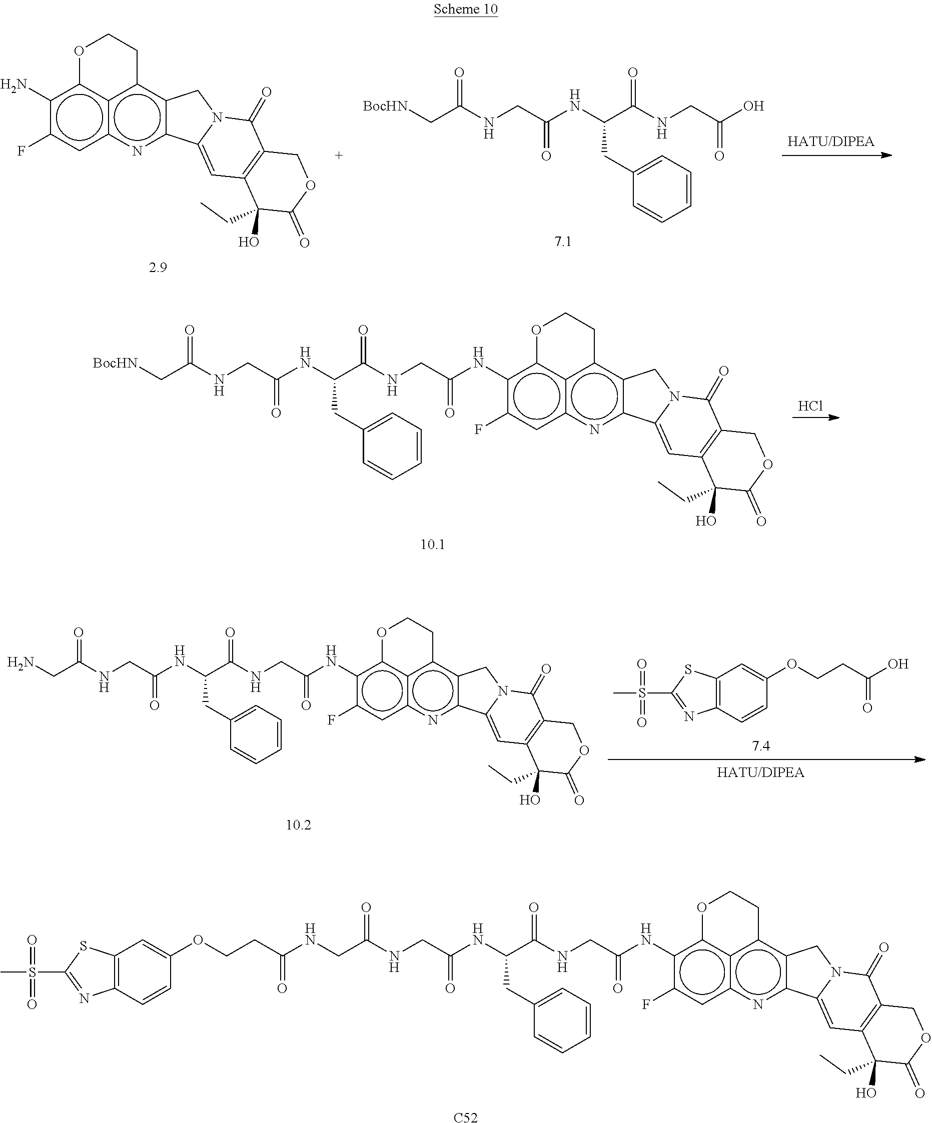

Preparation of tert-butyl (2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl) carbamate 10.1. To a solution of (S)-4-amino-9-ethyl-5-fluoro-9-hydroxy-1, 9, 12, 15-tetrahydro-13H-pyrano [4, 3, 2-de]pyrano [3′, 4′: 6, 7]indolizino [1,2-b]quinoline-10, 13 (2H)-dione 2.9 (250 mg, 0.59 mmol) in DMF (10 mL) under argon were added (tert-butoxycarbonyl) glycyl-glycyl-L-phenylalanylglycine 7.1 (271 mg, 0.62 mmol), HATU (337 mg, 0.886 mmol), and DIPEA (76 mg, 0.59 mmol). After stirring at room temperature for 20 h, the reaction mixture was poured into water (20 mL) and extracted with EtOAc (5×20 mL). The combined organic layers were washed with brine, dried over anhydrous Na₂SO₄, concentrated, and purified by column chromatography (SiO₂, MeOH/DCM) to afford compound 10.1 (250 mg) in 50% yield.

Preparation of (S)-2-(2-(2-aminoacetamido) acetamido)-N-(2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl)-3-phenylpropanamide 10.2. To a mixture of compound 10.1 (250 mg, 0.297 mmol) in EtOAc (12.5 mL) was added 6M HCl in EtOAc (5 mL). After stirring at room temperature for 12 h, the reaction mixture was concentrated and purified by trituration with EtOAc/PE to afford compound 10.2 (95 mg) in 43% yield.

Preparation of (S)—N-(2-(((S)-9-ethyl-5-fluoro-9-hydroxy-10, 13-dioxo-1, 2, 9, 10, 13, 15-hexahydro-12H-pyrano-[4, 3, 2-de]pyrano [3′, 4′: 6, 7]indolizino [1, 2-b]quinolin-4-yl) amino)-2-oxoethyl)-2-(2-(2-(3-((2-(methylsulfonyl) benzo [d]thiazol-6-yl) oxy) propanamido)-acetamido) acetamido)-3-phenylpropanamide C52. To a solution of compound 10.2 (120 mg, 0.162 mmol) in DMF (4.8 mL) under argon were added 3-((2-(methylsulfonyl) benzo [d]thiazol-6-yl) oxy) propanoic acid 7.4 (51 mg, 0.17 mmol), HATU (92 mg, 0.243 mmol), and DIPEA (21 mg, 0.162 mmol). After stirring at room temperature for 25 h, the reaction mixture was purified by column chromatography (SiO₂, MeOH/DCM) to afford compound C52 (35 mg) in 21% yield. MS (ESI) m/z: 1025.3 [M+H]⁺.

The following compound was prepared similarly according to the synthetic procedures or methodologies exemplified herein.

6-(2, 5-Dioxo-2, 5-dihydro-1H-pyrrol-1-yl)-N—((S)-1-(((S)-1-(((S)-9-ethyl-9-hydroxy-10, 13-dioxo-1, 2, 9, 10, 13, 15-hexahydro-12H-pyrano [3′, 4′: 6, 7]indolizino [1,2-b]thio-pyrano [4, 3, 2-de]quinolin-4-yl) amino)-1-oxopropan-2-yl) amino)-3-methyl-1-oxobutan-2-yl)-hexanamide B1. MS (ESI) m/z: 785.3 [M+H]⁺.

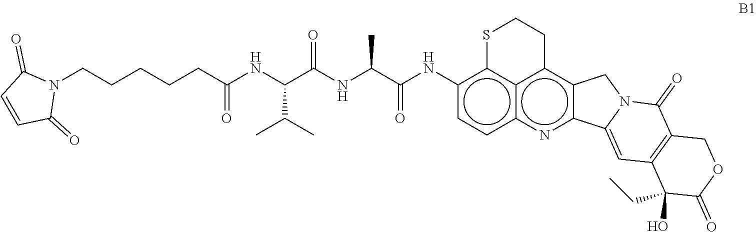

N—((S)-5-Benzyl-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-16, 19, 22, 25-tetraoxa-3, 6, 9, 12-tetraazaheptacosan-27-yl)-2-(methylsulfonyl) benzo [d]thiazole-6-carboxamide C53. MS (ESI) m/z: 1210.4 [M+H]⁺.

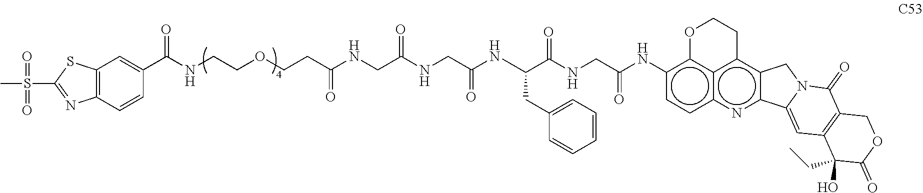

The following compounds are prepared similarly according to the synthetic procedures or methodologies exemplified herein.

6-(2, 5-Dioxo-2, 5-dihydro-1H-pyrrol-1-yl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl) amino)-1-oxo-3-phenylpropan-2-yl) amino)-2-oxoethyl) amino)-2-oxoethyl) hexanamide A3.

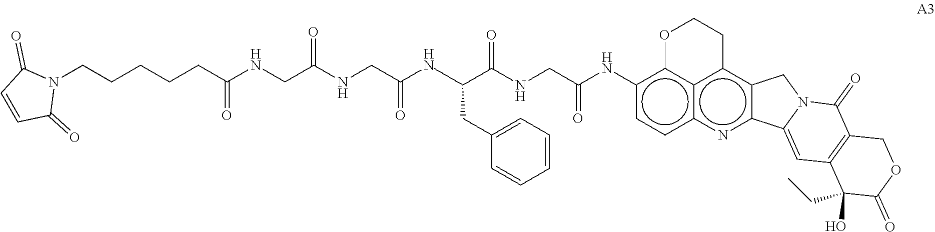

6-(2, 5-Dioxo-2, 5-dihydro-1H-pyrrol-1-yl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) amino)-2-oxoethyl) amino)-1-oxo-3-phenylpropan-2-yl) amino)-2-oxoethyl) amino)-2-oxoethyl) hexanamide A4.

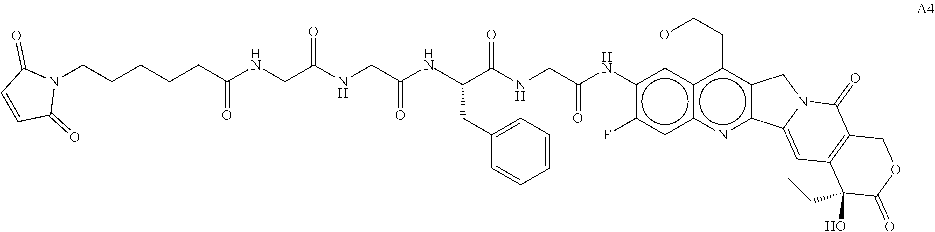

4-((S)-2-((S)-2-(6-(2, 5-Dioxo-2, 5-dihydro-1H-pyrrol-1-yl) hexanamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate A5.

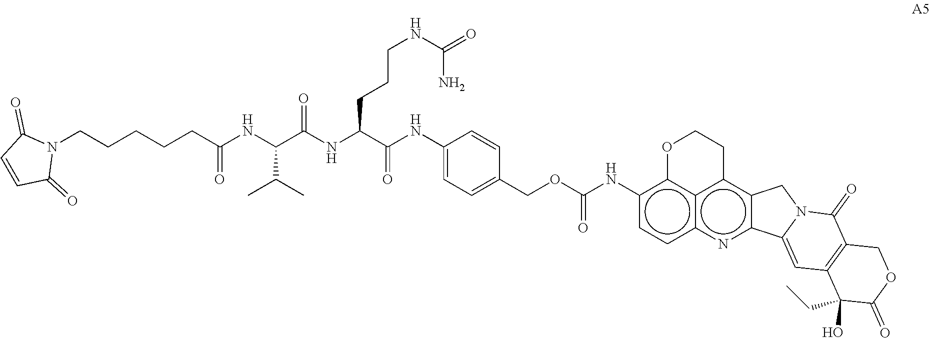

4-((S)-2-((S)-2-(6-(2, 5-Dioxo-2, 5-dihydro-1H-pyrrol-1-yl) hexanamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7] indolizino [1, 2-b]quinolin-4-yl)-carbamate A6.

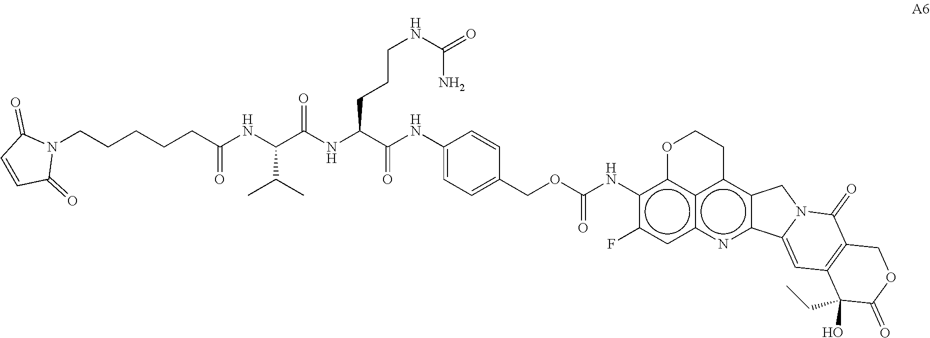

6-(2, 5-Dioxo-2, 5-dihydro-1H-pyrrol-1-yl)-N—((S)-1-(((S)-1-(((S)-9-ethyl-5-fluoro-9-hydroxy-10, 13-dioxo-9, 10, 13, 15-tetrahydro-1H, 3H, 12H-pyrano [3, 4, 5-de]pyrano [3′, 4′: 6, 7]-indolizino [1,2-b]quinolin-4-yl) amino)-1-oxopropan-2-yl) amino)-3-methyl-1-oxobutan-2-yl)-hexanamide A8.

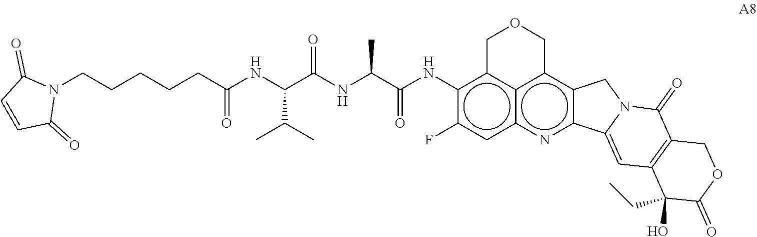

6-(2, 5-Dioxo-2, 5-dihydro-1H-pyrrol-1-yl)-N—((S)-1-(((S)-1-(((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]thio-pyrano [4, 3, 2-de]quinolin-4-yl) amino)-1-oxopropan-2-yl) amino)-3-methyl-1-oxobutan-2-yl)-hexanamide B2.

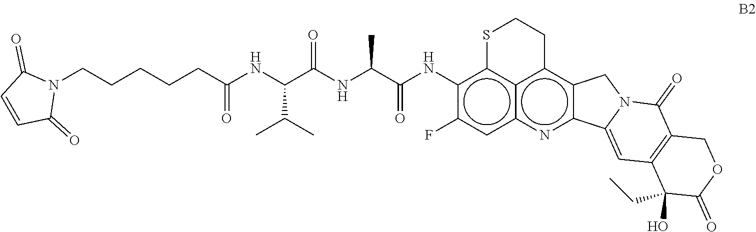

6-(2, 5-Dioxo-2, 5-dihydro-1H-pyrrol-1-yl)-N-(2-((2-(((S)-1-((2-(((S)-9-ethyl-9-hydroxy-10, 13-dioxo-1, 2, 9, 10, 13, 15-hexahydro-12H-pyrano [3′, 4′: 6, 7]indolizino [1,2-b]thio-pyrano [4, 3, 2-de]quinolin-4-yl) amino)-2-oxoethyl) amino)-1-oxo-3-phenylpropan-2-yl) amino)-2-oxoethyl) amino)-2-oxoethyl) hexanamide B3.

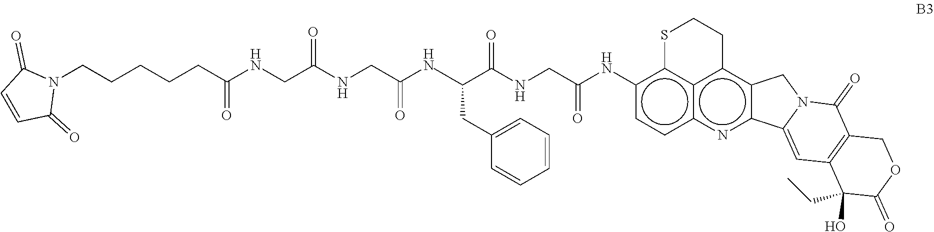

6-(2, 5-Dioxo-2, 5-dihydro-1H-pyrrol-1-yl)-N-(2-((2-(((S)-1-((2-(((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]-thiopyrano [4, 3, 2-de]quinolin-4-yl) amino)-2-oxoethyl) amino)-1-oxo-3-phenylpropan-2-yl)-amino)-2-oxoethyl) amino)-2-oxoethyl) hexanamide B4.

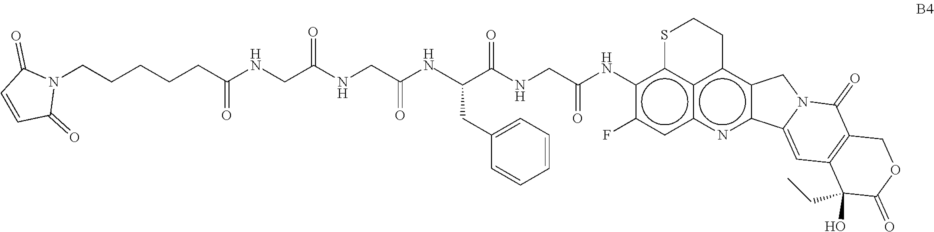

4-((S)-2-((S)-2-(6-(2, 5-Dioxo-2, 5-dihydro-1H-pyrrol-1-yl) hexanamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [3′, 4′: 6, 7]indolizino [1,2-b]thiopyrano [4, 3, 2-de]quinolin-4-yl) carbamate B5.

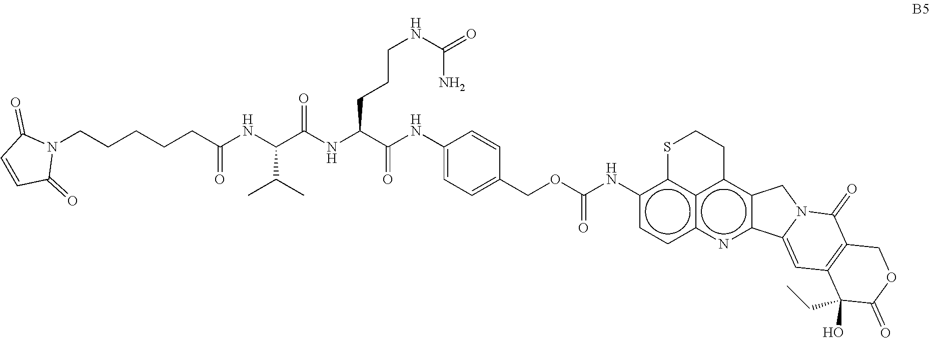

4-((S)-2-((S)-2-(6-(2, 5-Dioxo-2, 5-dihydro-1H-pyrrol-1-yl) hexanamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [3′, 4′: 6, 7]indolizino [1,2-b]thiopyrano [4, 3, 2-de]quinolin-4-yl) carbamate B6.

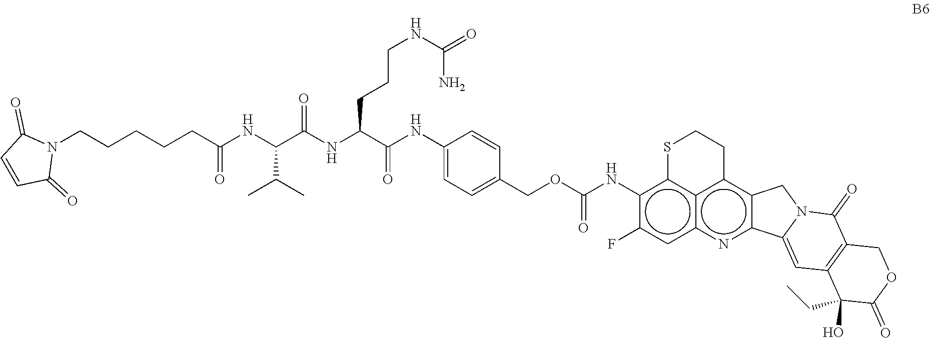

6-(2, 5-Dioxo-2, 5-dihydro-1H-pyrrol-1-yl)-N—((S)-1-(((S)-1-(((S)-9-ethyl-9-hydroxy-10, 13-dioxo-9, 10, 13, 15-tetrahydro-1H, 3H, 12H-pyrano [3′, 4′: 6, 7]indolizino [1,2-b]-thiopyrano [3, 4, 5-de]quinolin-4-yl) amino)-1-oxopropan-2-yl) amino)-3-methyl-1-oxobutan-2-yl)-hexanamide B7.

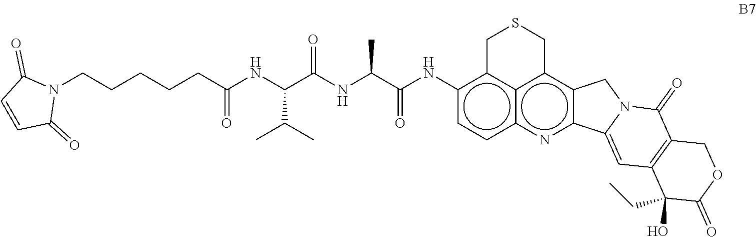

6-(2, 5-Dioxo-2, 5-dihydro-1H-pyrrol-1-yl)-N—((S)-1-(((S)-1-(((S)-9-ethyl-5-fluoro-9-hydroxy-10, 13-dioxo-9, 10, 13, 15-tetrahydro-1H, 3H, 12H-pyrano [3′, 4′: 6, 7]indolizino [1,2-b]-thiopyrano [3, 4, 5-de]quinolin-4-yl) amino)-1-oxopropan-2-yl) amino)-3-methyl-1-oxobutan-2-yl) hexanamide B8.

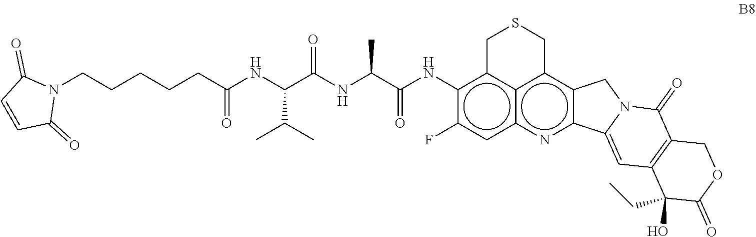

N—((S)-1-(((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-2-(methylsulfonyl) benzo [d]thiazole-6-carboxamide C1.

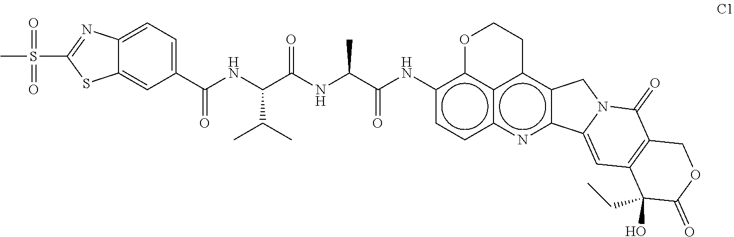

4-((S)-2-((S)-3-Methyl-2-(2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C3.

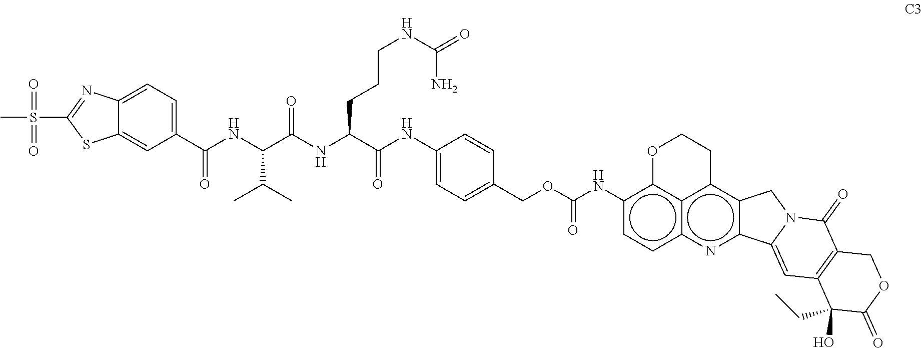

2-(Cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-amino)-1-oxopropan-2-yl) amino)-3-methyl-1-oxobutan-2-yl) benzo [d]thiazole-6-carboxamide C4.

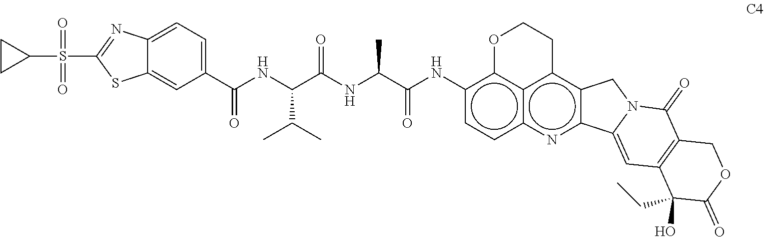

2-(Cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl) benzo [d]thiazole-6-carboxamide C5.

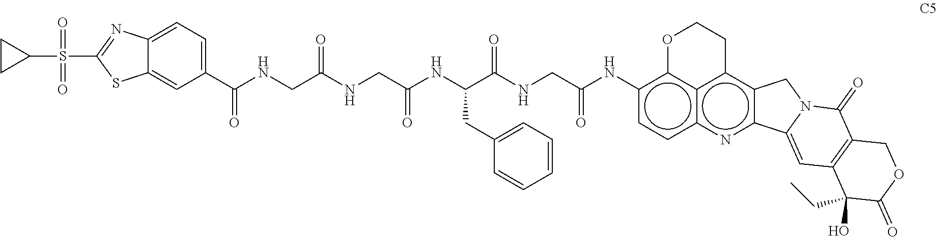

4-((S)-2-((S)-2-(2-(Cyclopropylsulfonyl) benzo [d]thiazole-6-carboxamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C6.

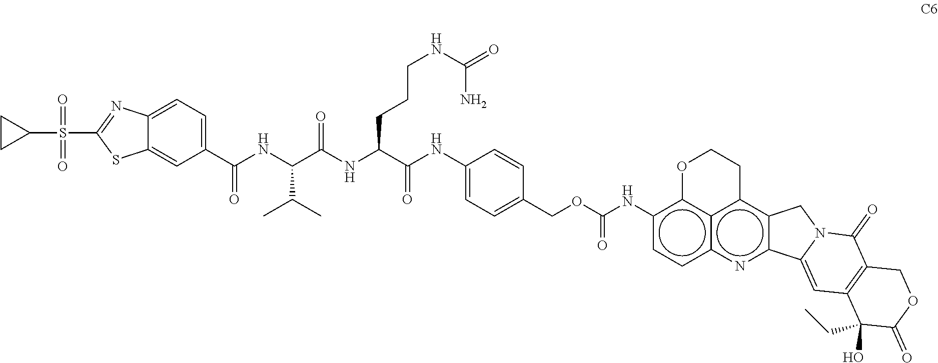

N—((S)-1-(((S)-1-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-1-oxopropan-2-yl) amino)-3-methyl-1-oxobutan-2-yl)-2-(methylsulfonyl) benzo [d]thiazole-6-carboxamide C7.

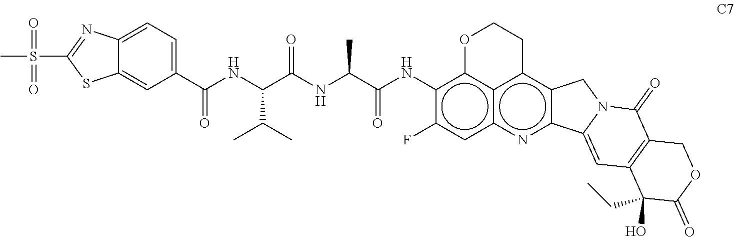

4-((S)-2-((S)-3-Methyl-2-(2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C9.

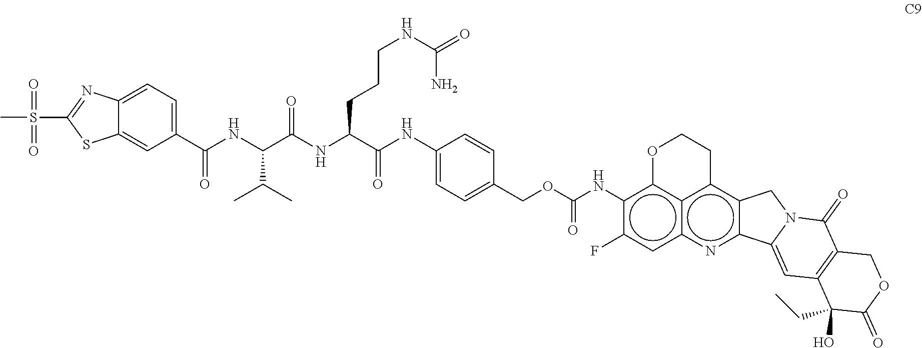

2-(Cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl) benzo [d]thiazole-6-carboxamide C11.

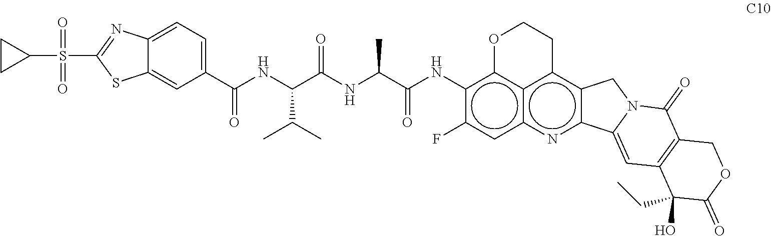

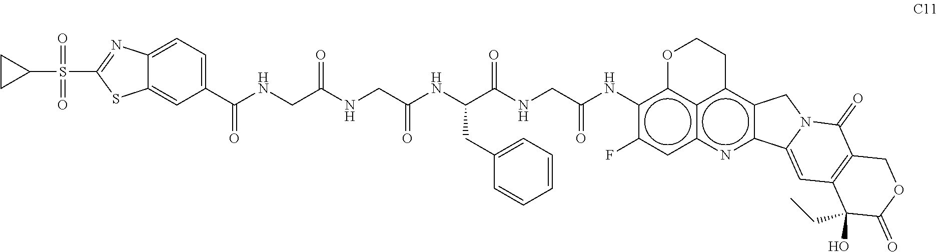

4-((S)-2-((S)-2-(2-(Cyclopropylsulfonyl) benzo [d]thiazole-6-carboxamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′. 4′: 6, 7]indolizino [1,2-b]quinolin-4-yl) carbamate C12.

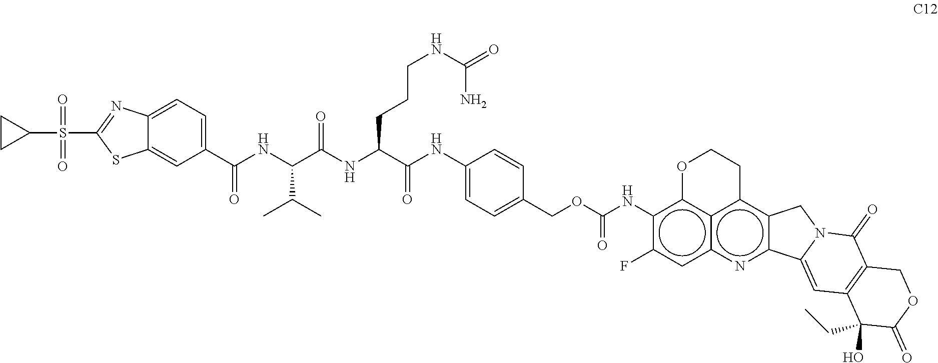

N—((S)-1-(((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-5-fluoro-2-(methylsulfonyl) benzo [d]-thiazole-6-carboxamide C13.

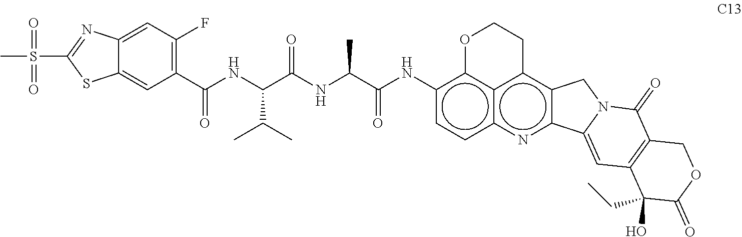

N-(2-((2-(((S)-1-((2-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-5-fluoro-2-(methylsulfonyl) benzo [d]thiazole-6-carboxamide C14.

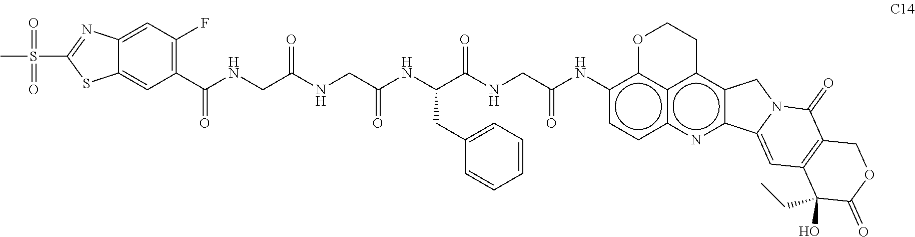

4-((S)-2-((S)-3-Methyl-2-(5-fluoro-2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′. 4′: 6, 7]indolizino [1,2-b]quinolin-4-yl) carbamate C15.

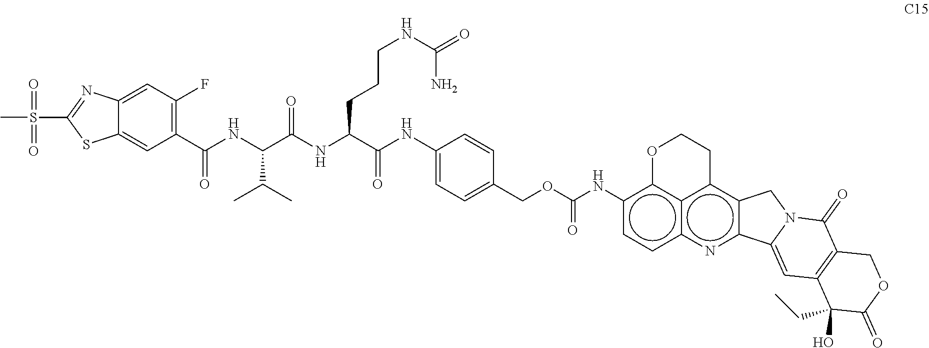

2-(Cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylprop an-2-yl) amino)-2-oxoethyl) amino)-2-oxoethyl)-5-fluorobenzo [d]thiazole-6-carboxamide C17.

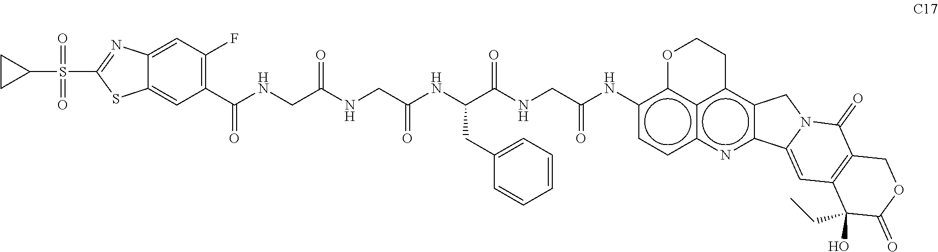

4-((S)-2-((S)-2-(2-(Cyclopropylsulfonyl)-5-fluorobenzo [d]thiazole-6-carboxamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]-quinolin-4-yl) carbamate C18.

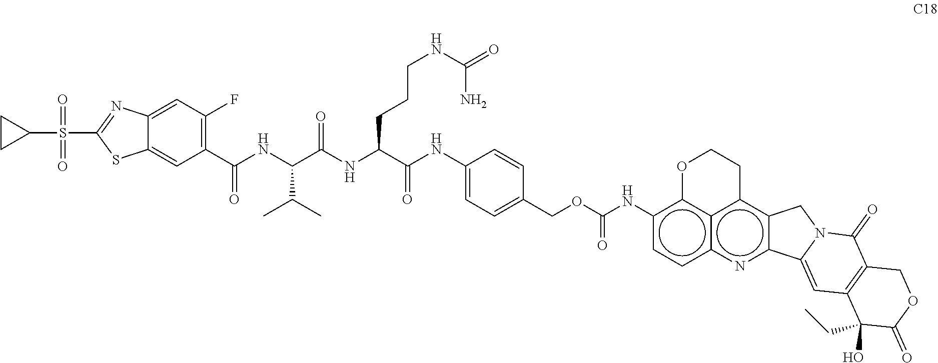

N—((S)-1-(((S)-1-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-1-oxopropan-2-yl) amino)-3-methyl-1-oxobutan-2-yl)-5-fluoro-2-(methylsulfonyl) benzo [d]-thiazole-6-carboxamide C19.

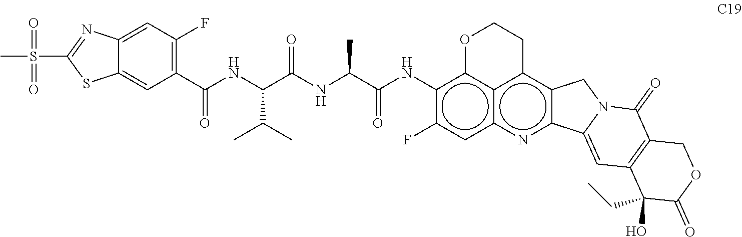

N-(2-((2-(((S)-1-((2-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-2-oxoethyl) amino)-1-oxo-3-phenylpropan-2-yl) amino)-2-oxoethyl) amino)-2-oxoethyl)-5-fluoro-2-(methylsulfonyl) benzo [d]thiazole-6-carboxamide C20.

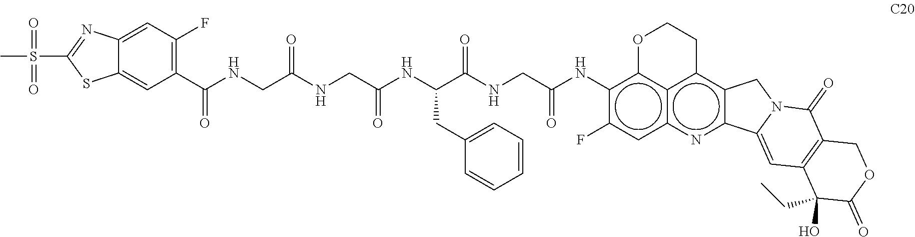

4-((S)-2-((S)-3-Methyl-2-(5-fluoro-2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C21.

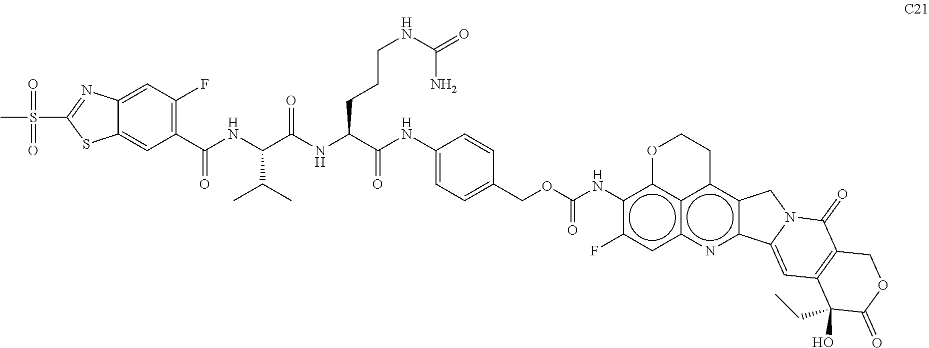

2-(Cyclopropylsulfonyl)-N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-5-fluorobenzo [d]-thiazole-6-carboxamide C22.

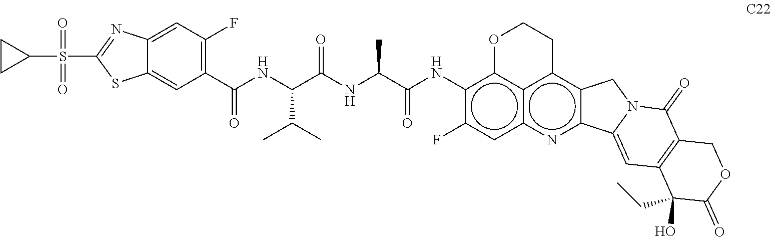

2-(Cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl)-5-fluorobenzo [d]thiazole-6-carboxamide C23.

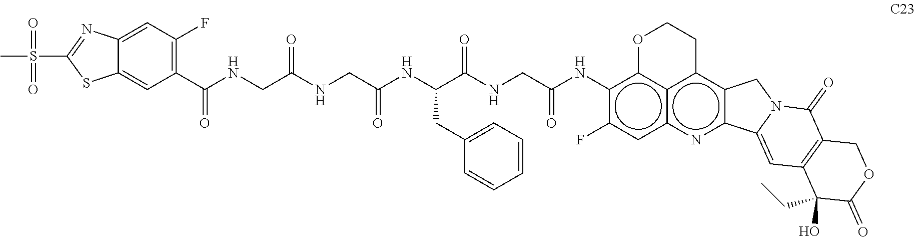

4-((S)-2-((S)-2-(2-(Cyclopropylsulfonyl)-5-fluorobenzo [d]thiazole-6-carboxamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C24.

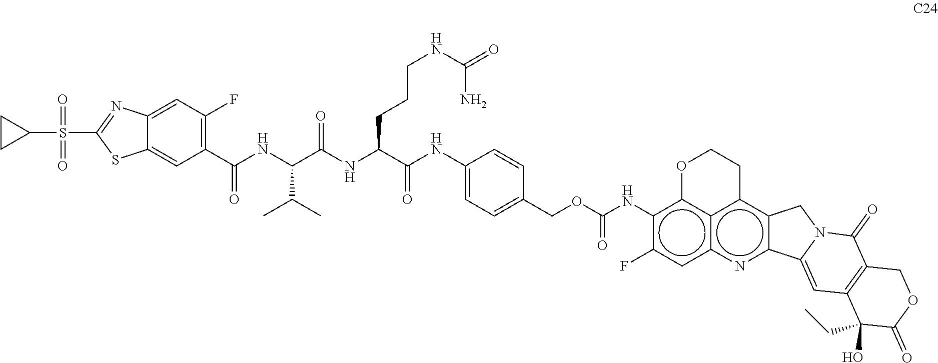

(S)—N—((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(2-((2-(methylsulfonyl) benzo [d]thiazol-6-yl) oxy) acetamido) butanamide C25.

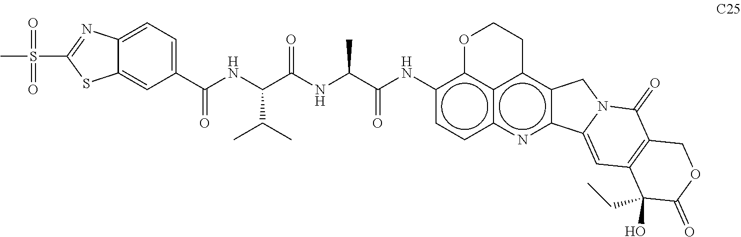

(S)—N-(2-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl)-2-(2-(2-(2-((2-(methylsulfonyl) benzo [d]thiazol-6-yl) oxy) acetamido) acetamido) acetamido)-3-phenyl-propanamide C26.

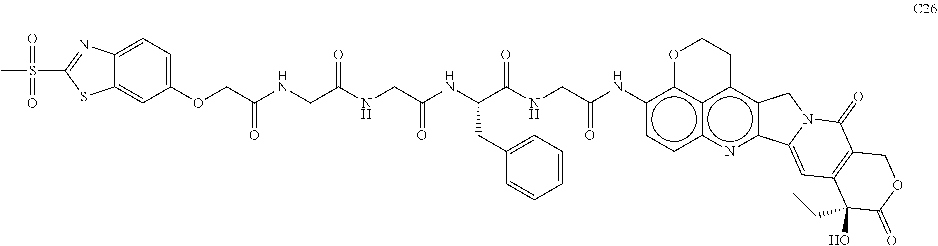

4-((S)-2-((S)-3-Methyl-2-(2-((2-(methylsulfonyl) benzo [d]thiazol-6-yl) oxy)
acetamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C27.

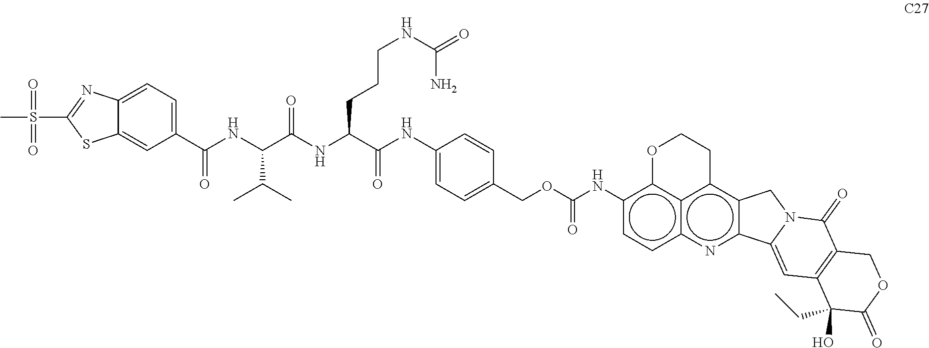

(S)—N—((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(3-((2-(methylsulfonyl) benzo [d]thiazol-6-yl) oxy) propanamido) butanamide C28.

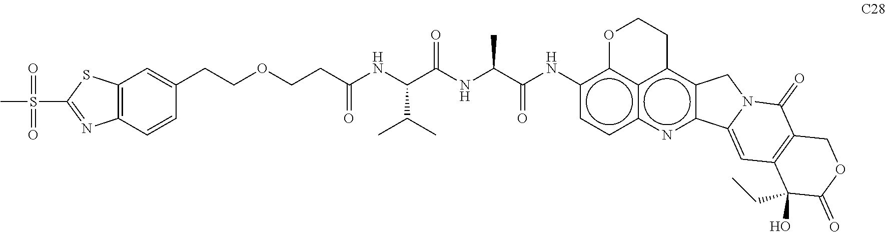

4-((S)-2-((S)-3-Methyl-2-(3-((2-(methylsulfonyl) benzo [d]thiazol-6-yl) oxy)-propanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) carbamate C30.

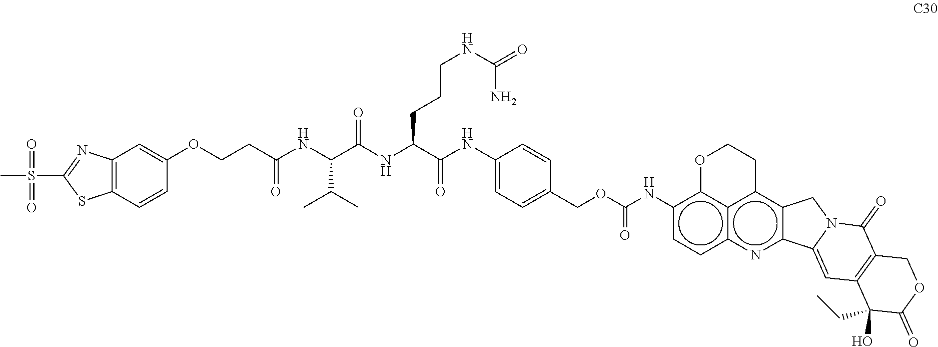

N-((17S,20S)-21-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluoro-2-(methylsulfonyl)-benzo [d]thiazole-6-carboxamide C31.

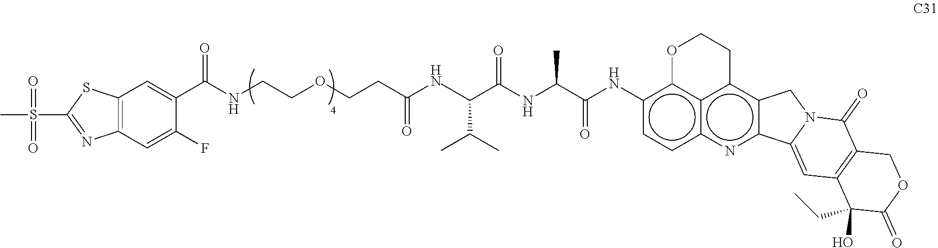

N-((17S,20S)-21-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-17-isopropyl-20-methyl-15, 18, 21-trioxo-3, 6, 9, 12-tetraoxa-16, 19-diazahenicosyl)-5-fluoro-2-(methysulfonyl)benzo[d]thiazole-6-carboxamide C32.

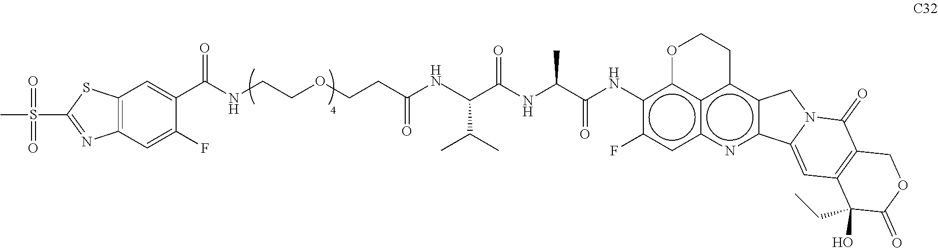

2-(Cyclopropylsulfonyl)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C34.

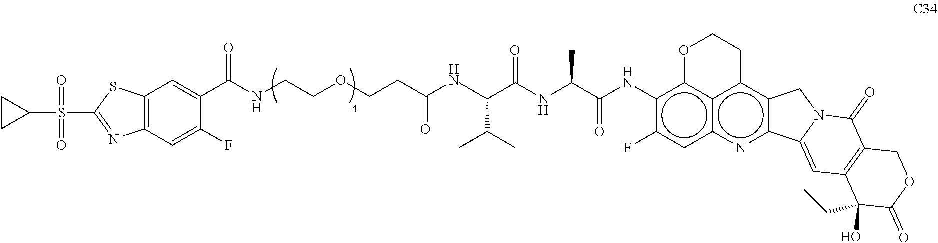

N-((29S,32S)-33-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C35.

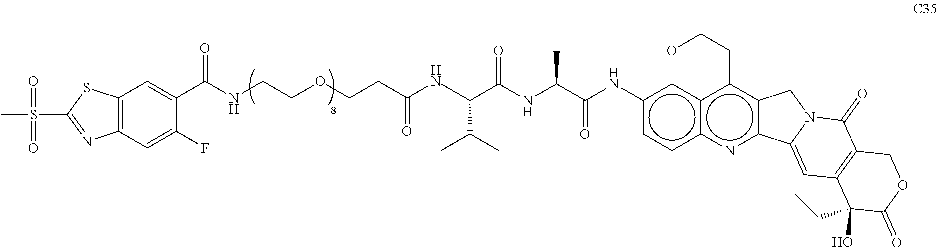

N-((29, 32)-33-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C36.

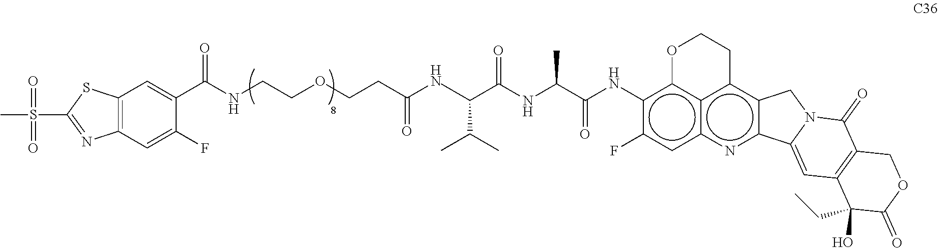

2-(Cyclopropylsulfonyl)-N-((29S,32S)-33-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diaza-tritriacontyl)-5-fluorobenzo [d]thiazole-6-carboxamide C37.

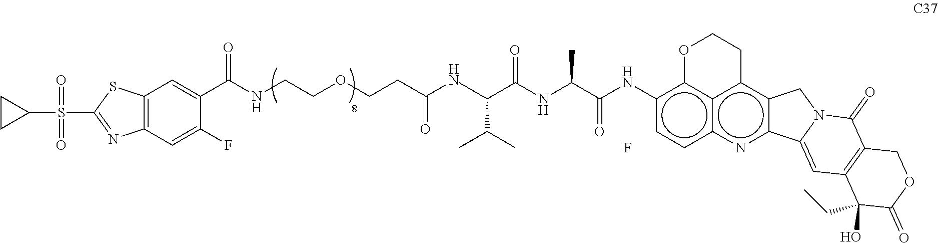

2-(Cyclopropylsulfonyl)-N-((29S,32S)-33-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluorobenzo [d]thiazole-6-carboxamide C38.

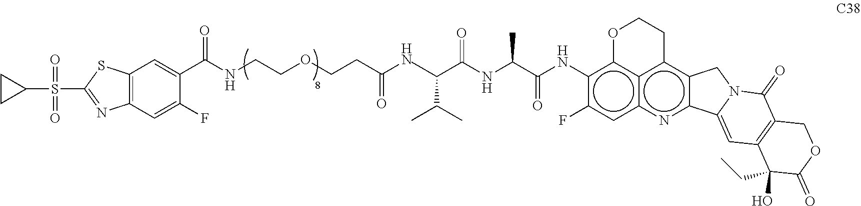

N—((S)-1-(((S)-1-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) amino)-1-oxo-propan-2-yl) amino)-3-methyl-1-oxobutan-2-yl)-1-(3-((2-(methylsulfonyl) benzo [d]thiazol-6-yl) oxy) propanamido)-3, 6, 9, 12-tetraoxapentadecan-15-amide C40.

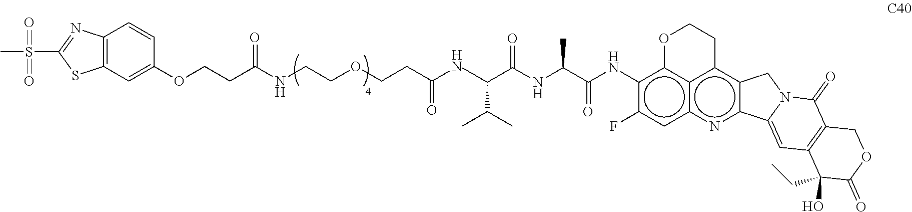

N—((S)-1-(((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-1-(3-((2-(methylsulfonyl) benzo [d]thiazol-6-yl) oxy)-propanamido)-3, 6, 9, 12, 15, 18, 21, 24-octaoxaheptacosan-27-amide C41.

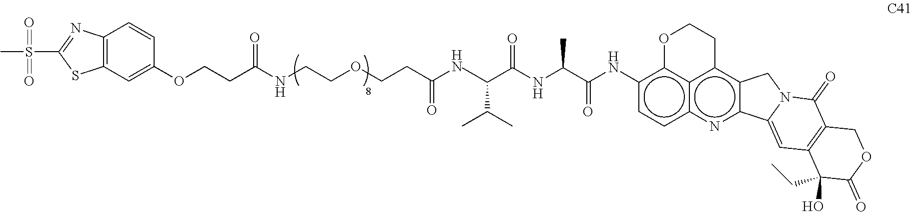

N—((S)-1-(((S)-1-(((S)-9-Ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxo-propan-2-yl) amino)-3-methyl-1-oxobutan-2-yl)-1-(3-((2-(methylsulfonyl) benzo [d]thiazol-6-yl)-oxy) propanamido)-3, 6, 9, 12, 15, 18, 21, 24-octaoxaheptacosan-27-amide C42.

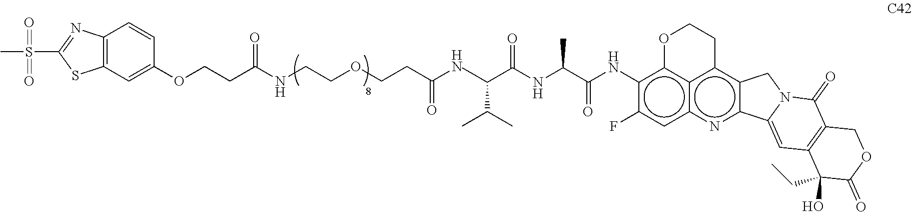

N-(6-(((S)-1-(((S)-1-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl) amino)-6-oxohexyl)-2-(methylsulfonyl)-benzo [d]thiazole-6-carboxamide C43.

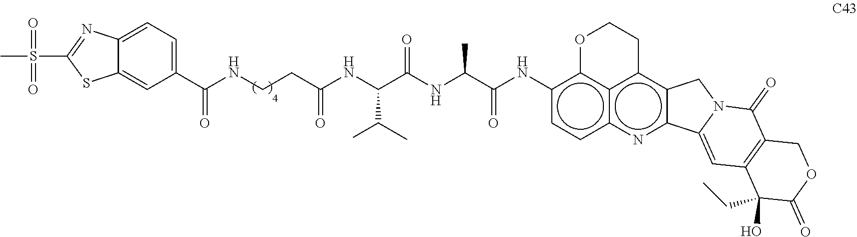

N—((S)-5-Benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(methylsulfonyl) benzo [d]thiazole-6-carboxamide C44.

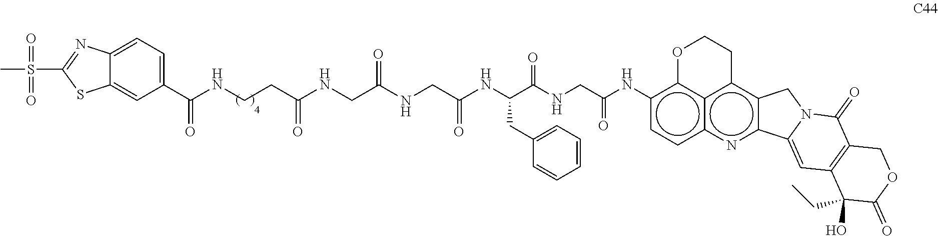

4-((S)-2-((S)-3-Methyl-2-(6-(2-(methylsulfonyl) benzo [d]thiazole-6-carboxamido) hexanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) carbamate C45.

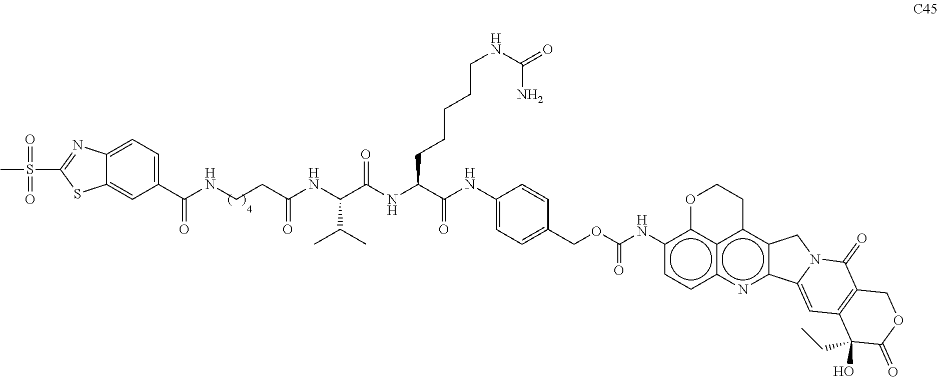

2-(Cyclopropylsulfonyl)-N-(6-(((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) amino)-6-oxohexyl)-benzo [d]thiazole-6-carboxamide C46.

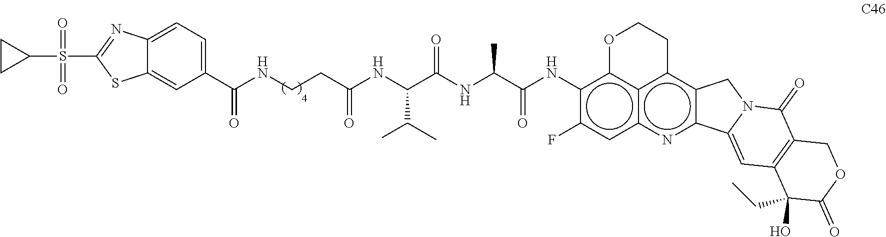

N—((S)-5-Benzyl-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl) benzo [d]-thiazole-6-carboxamide C47.

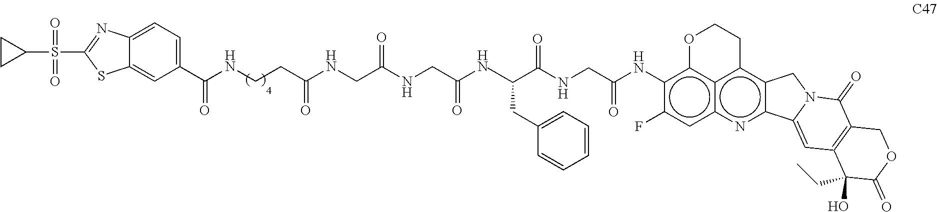

4-((S)-2-((S)-2-(6-(2-(Cyclopropylsulfonyl) benzo [d]thiazole-6-carboxamido)-hexanamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C48.

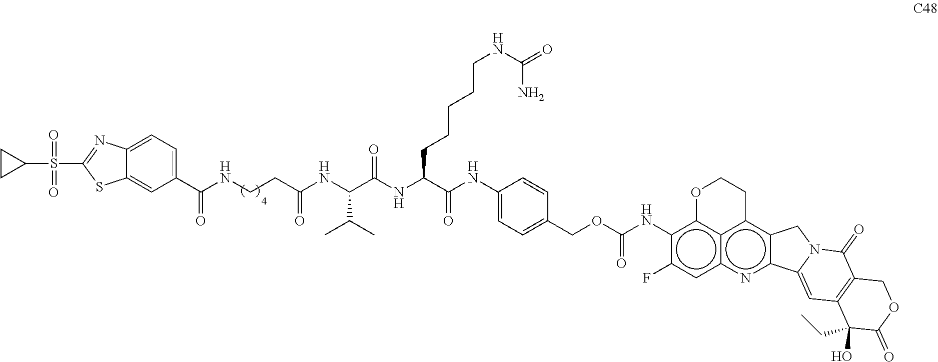

2-(Cyclopropylsulfonyl)-N-(6-(((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl)-amino)-1-oxopropan-2-yl) amino)-3-methyl-1-oxobutan-2-yl) amino)-6-oxohexyl)-5-fluoro-benzo [d]thiazole-6-carboxamide C49.

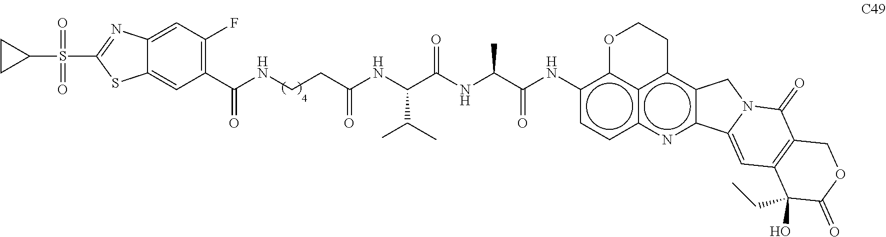

N—((S)-5-Benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl)-5-fluorobenzo [d]thiazole-6-carboxamide C50.

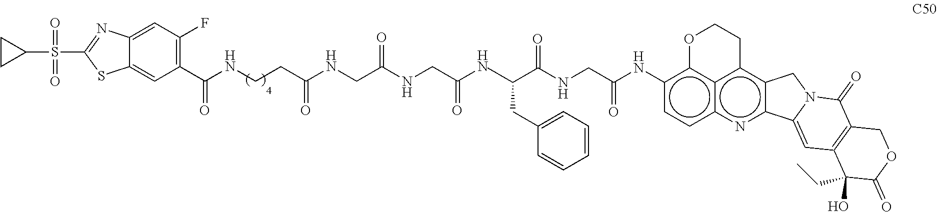

4-((S)-2-((S)-2-(6-(2-(Cyclopropylsulfonyl)-5-fluorobenzo [d]thiazole-6-carboxamido) hexanamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C51.

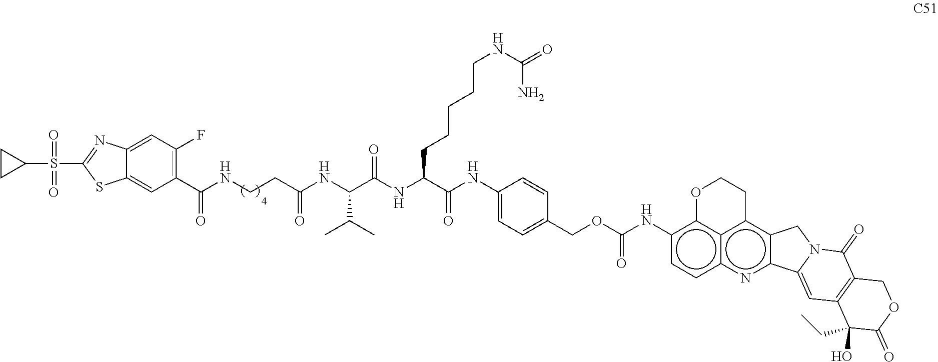

(S)—N-(2-(((S)-9-Ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl)-2-(2-(2-(2-(4-(5-(methylsulfonyl)-1, 3, 4-oxadiazol-2-yl) phenoxy) acetamido) acetamido) acetamido)-3-phenylpropanamide D2.

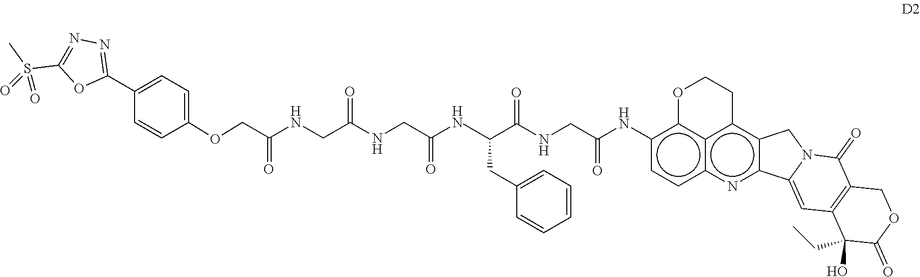

4-((S)-2-((S)-3-Methyl-2-(2-(4-(5-(methylsulfonyl)-1, 3, 4-oxadiazol-2-yl)-phenoxy) acetamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′. 4′: 6, 7]indolizino [1,2-b]-quinolin-4-yl) carbamate D3.

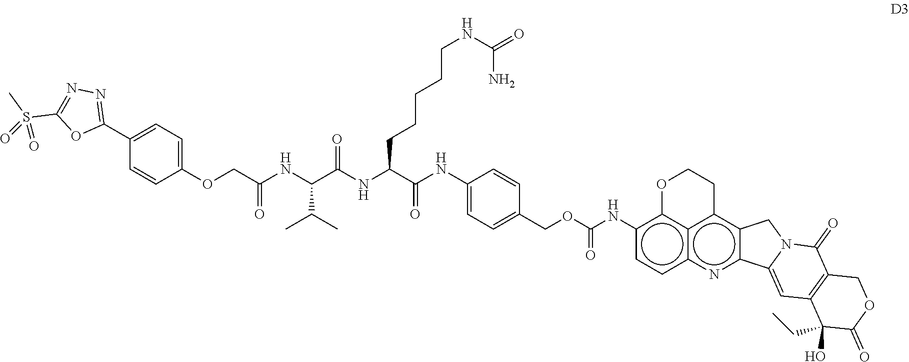

### Example 11

### Antibody-Drug Conjugate Preparation

Monoclonal antibody trastuzumab having a light chain of SEQ ID NO: 39 and a heavy chain of SEQ ID NO: 40 was buffer exchanged into a 20 mM sodium phosphate buffered saline solution and further diluted to 2 mg/mL in the same buffer. Ten molar equivalences of TCEP were added to reduce the interchain disulfide bonds of trastuzumab to generate 8 free cysteines. Twenty molar equivalents of a drug with a linker (drug-linker), e.g., A1, A2, C2, C8, C29, or C52, were added to the reduced trastuzumab solution and incubated for approximately 1 h at room temperature. Excess TCEP and the drug-linker were then removed via a desalting column. The conjugation of the reduced trastuzumab with drug-linker compound A1, A2, C2, C8, C29, and C52 yielded ADC compounds ADC-A1, ADC-A2, ADC-C2, ADC-C8, ADC-C29, and ADC-C52, respectively. The drug-to-antibody ratios (DARs) of ADC compounds ADC-C2, ADC-C8, ADC-C29, and ADC-C52 were determined by LCMS. The results are summarized in Table 1.

TABLE 1
Drug-to-antibody Ratios (DARs) Determination by LCMS

| | Light | Heavy Chain | | |
|---|---|---|---|---|
| Compound | Chain | Major | Minor | DAR |
| Trastuzumab | 23438.33 | 50446.98; 50593.37; 50755.54; 50917.45 | | 0 |
| ADC-A1 | 24207.76 | 52855.35; 52899.61; 53061.82; 53224.05 | | 8 |
| ADC-A2 | 24225.57 | 52910.18; 52953.75; 53115.45; 53277.52 | | 8 |
| ADC-C2 | 24321.22 | 53198.12; 53241.79; 53403.75; 53565.79 | | 8 |
| ADC-C8 | 24339.21 | 53133.57; 53295.77; 53457.8; 53620.13 | 52571.92; 52734.07 | 7.6 |
| ADC-C29 | 24365.57 | 53227.44; 53373.16; 53535.74; 53697.7 | | 8 |
| ADC-C52 | 23438.17 | 53265.25; 53427.61; 53590.14; 53752.04 | 57023.96; 57186.07 | 7.8 |

Similarly, the conjugation of the reduced trastuzumab with drug-linker compounds A3 to A8, B1 to B8, C1, C3 to C7, C9 to C28, C30 to C51, and C53 yields ADC compounds ADC-A3 to ADC-A8, ADC-B1 to ADC-B8, ADC-C1, ADC-C3 to ADC-C7, ADC-C9 to ADC-C28, ADC-C30 to ADC-C51, and ADC-C53, respectively.

### Example B1

### Cell Viability Assays

The human cell lines NCI-N87 (gastric carcinoma), SK-BR-3 (breast adenocarcinoma), and SK—OV-3 (ovarian adenocarcinoma) were seeded at 5,000 cells/well in a culture media in a 96-well flat-bottom plate. Solutions of ADC compounds, ADC-A1, ADC-A2, ADC-C2, ADC-C8, ADC-C29, and ADC-C52, were prepared by a 3-fold serial dilution and added into cells. Trastuzumab deruxtecan (DS-8201) was used as a positive control. The cells were incubated for 4 or 5 days at 37° C. under 5% CO₂. CELLCOUNTING-LITE was then added to each well. The plate was mixed and incubated for 10 min at room temperature. The solution in each cell was transferred to a 96-well white opaque plate for reading. The results are summarized in Table 2.

### Example B2

### Xenograft Mouse Model

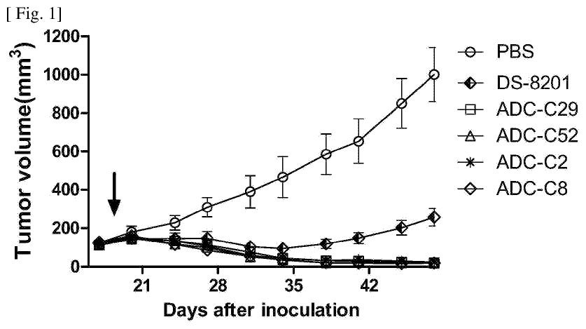

The human breast cancer cell line JIMT-1 (5×10⁶) in 100 μL DPBS was subcutaneously implanted in the right flanks of CB. 17 SCID mice. From around day 15 onwards, all tumors were measured thrice weekly, and the length and width of each mouse tumor were recorded to calculate tumor volumes (volume=length×(width²)×0.5). When the mean tumor volume reached ˜100 mm³, the mice were randomized into dosing groups and treated by a single dose (3 mg/kg) of an ADC compound intraperitoneally. All tumors were measured, and the mice were euthanized individually once their tumor reached a mean tumor measurement of 1.5 cm³. The results are summarized in FIG. 1, which demonstrated that ADC compounds, ADC-C2, ADC-C8, ADC-C29, and ADC-C52, were more potent than trastuzumab deruxtecan (DS-8201) in inhibiting the tumor growth.

TABLE 2
Effects of ADC Compounds on the Viability of Cancerous Cells

| | NCI-N87 | | SK-BR-3 | | SK-OV-3 | |
|---|---|---|---|---|---|---|
| | | Max | | Max | | Max |
| ADC | EC₅₀ | Killing | EC₅₀ | Killing | EC₅₀ | Killing |
| DS-8201 | 0.29 nM | 70% | 0.075 nM | 82% | 0.162 nM | 34% |
| ADC-A1 | 0.35 nM | 62% | 0.076 nM | 76% | 0.091 nM | 67% |
| ADC-A2 | 0.19 nM | 84% | 0.064 nM | 80% | 0.102 nM | 66% |
| ADC-C2 | 0.54 nM | 69% | 0.161 nM | 78% | 0.229 nM | 50% |
| ADC-C8 | 0.15 nM | 84% | 0.057 nM | 87% | 0.102 nM | 34% |
| ADC-C29 | 0.45 nM | 67% | 0.149 nM | 79% | 0.312 nM | 39% |
| ADC-C52 | 0.16 nM | 83% | 0.06 nM | 86% | 0.114 nM | 81% |

Sequences described herein are provided in the sequence table below.

SEQUENCE TABLE

| SEQ ID NO: | Description | Amino Acid Sequence |
|---|---|---|
| 1 | Anti-B7H3-1 CDR1 | INAMG |
| | (Kabat) | |
| 2 | Anti-B7H3-1 CDR2 | GLSSNGDITRQNYAFYVKG |
| | (Kabat) | |
| 3 | Anti-B7H3-1 CDR3 | MPPAST |
| | (Kabat) | |
| 4 | Anti-B7H3-1 Variable | QVQLVESGGGLVQPGGSLRLSCSASGSTSNINAMG |
| | | WYRQAPGKEREFVAGLSSNGDITRQNYAFYVKGR |
| | | FTISRDNSKNTLYLQMNSLRAEDTAVYYCNEMPPA |
| | | STWGQGTQVTVSS |
| 5 | Anti-B7H3-1 | QVQLVESGGGLVQPGGSLRLSCSASGSTSNINAMG |
| | | WYRQAPGKEREFVAGLSSNGDITRQNYAFYVKGR |
| | | FTISRDNSKNTLYLQMNSLRAEDTAVYYCNEMPPA |
| | | STWGQGTQVTVSSDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 6 | Anti-B7H3-2 CDR1 | INAMA |
| | (Kabat) | |
| 7 | Anti-B7H3-2 CDR2 | GVTSSGSIVRENYAFYVKG |
| | (Kabat) | |
| 8 | Anti-B7H3-2 CDR3 | IPPYST |
| | (Kabat) | |
| 9 | Anti-B7H3-2 Variable | EVQLVESGGGLVKPGGSLRLSCAASGSTSSINAMA |
| | | WYRQAPGKQREFVAGVTSSGSIVRENYAFYVKGR |
| | | FTISRDNAKNSLYLQMNSLRAEDTAVYYCNAIPPY |
| | | STWGQGTQVTVSS |
| 10 | Anti-B7H3-2 | EVQLVESGGGLVKPGGSLRLSCAASGSTSSINAMA |
| | | WYRQAPGKQREFVAGVTSSGSIVRENYAFYVKGR |
| | | FTISRDNAKNSLYLQMNSLRAEDTAVYYCNAIPPY |
| | | STWGQGTQVTVSSDKTHTCPPCPAPELLGGPSVFLF |
| | | PPKPKDTLMISRTPEVTCVVVDVSHEDPEVKFNWY |
| | | VDGVEVHNAKTKPREEQYNSTYRVVSVLTVLHQD |
| | | WLNGKEYKCKVSNKALPAPIEKTISKAKGQPREPQ |
| | | VYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| 11 | Anti-CD20 CDRL1 | RASSSVSYIH |
| | (Kabat) | |
| 12 | Anti-CD20 CDRL2 | ATSNLAS |
| | (Kabat) | |
| 13 | Anti-CD20 CDRL3 | QQWTSNPPT |
| | (Kabat) | |
| 14 | Anti-CD20 CDRH1 | SYNMH |
| | (Kabat) | |
| 15 | Anti-CD20 CDRH2 | AIYPGNGDTSYNQKFKG |
| | (Kabat) | |
| 16 | Anti-CD20 CDRH3 | STYYGGDWYFNV |
| | (Kabat) | |
| 17 | Anti-CD20 Light Chain | QIVLSQSPAILSASPGEKVTMTCRASSSVSYIHWFQ |
| | Variable Region | QKPGSSPKPWIYATSNLASGVPVRFSGSGSGTSYSL |
| | | TISRVEAEDAATYYCQQWTSNPPTFGGGTKLEIK |
| 18 | Anti-CD20 Heavy Chain | QVQLQQPGAELVKPGASVKMSCKASGYTFTSYNM |
| | Variable Region | HWVKQTPGRGLEWIGAIYPGNGDTSYNQKFKGKA |
| | | TLTADKSSSTAYMQLSSLTSEDSAVYYCARSTYYG |
| | | GDWYFNVWGAGTTVTVSA |
| 19 | Anti-CD20 Light Chain | QIVLSQSPAILSASPGEKVTMTCRASSSVSYIHWFQ |
| | | QKPGSSPKPWIYATSNLASGVPVRFSGSGSGTSYSL |
| | | TISRVEAEDAATYYCQQWTSNPPTFGGGTKLEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 20 | Anti-CD20 Heavy Chain | QVQLQQPGAELVKPGASVKMSCKASGYTFTSYNM |
| | | HWVKQTPGROLEWIGAIYPGNGDTSYNQKFKGKA |
| | | TLTADKSSSTAYMQLSSLTSEDSAVYYCARSTYYG |
| | | GDWYFNVWGAGTTVTVSAASTKGPSVFPLAPSSK |
| | | STSGGTAALGCLVKDYFPEPVTVSWNSGALTSGVH |
| | | TFPAVLQSSGLYSLSSVVTVPSSSLGTQTYICNVNH |
| | | KPSNTKVDKKAEPKSCDKTHTCPPCPAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 21 | Anti-FOLR1 CDRL1 | RASQSVSFAGTSLMH |
| | (Kabat) | |
| 22 | Anti-FOLR1 CDRL2 | RASNLEA |
| | (Kabat) | |
| 23 | Anti-FOLR1 CDRL3 | QQSREYPYT |
| | (Kabat) | |
| 24 | Anti-FOLR1 CDRH1 | GYFMN |
| | (Kabat) | |
| 25 | Anti-FOLR1 CDRH2 | RIHPYDGDTFYAQKFQG |
| | (Kabat) | |
| 26 | Anti-FOLR1 CDRH3 | YDGSRAMDY |
| | (Kabat) | |
| 27 | Anti-FOLR1 Light Chain | DIVLTQSPASLAVSPGQRATITCRASQSVSFAGTSL |
| | Variable Region | MHWYQQKPGQPPKLLIYRASNLEAGVPARFSGSGS |
| | | GTDFTLTINPVEANDAANYYCQQSREYPYTFGGGT |
| | | KLEIK |
| 28 | Anti-FOLR1 Heavy Chain | QVQLVQSGAEVKKPGASVKVSCKASGYTFTGYFM |
| | Variable Region | NWVRQAPGQGLEWIGRIHPYDGDTFYAQKFQGRV |
| | | TMTRDTSTSTVYMELSSLRSEDTAVYYCTRYDGSR |
| | | AMDYWGQGTTVTVSS |
| 29 | Anti-FOLR1 Light Chain | DIVLTQSPASLAVSPGQRATITCRASQSVSFAGTSL |
| | | MHWYQQKPGQPPKLLIYRASNLEAGVPARFSGSGS |
| | | GTDFTLTINPVEANDAANYYCQQSREYPYTFGGGT |
| | | KLEIKRTVAAPSVFIFPPSDEQLKSGTASVVCLLNNF |
| | | YPREAKVQWKVDNALQSGNSQESVTEQDSKDSTY |
| | | SLSSTLTLSKADYEKHKVYACEVTHQGLSSPVTKS |
| | | FNRGEC |
| 30 | Anti-FOLR1 Heavy Chain | QVQLVQSGAEVKKPGASVKVSCKASGYTFTGYFM |
| | | NWVRQAPGQGLEWIGRIHPYDGDTFYAQKFQGRV |
| | | TMTRDTSTSTVYMELSSLRSEDTAVYYCTRYDGSR |
| | | AMDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPSN |
| | | TKVDKKVEPKSCDKTHTCPPCPAPELLGGPSVFLFP |
| | | PKPKDTLMISRTPEVTCVVVDVSHEDPEVKFNWYV |
| | | DGVEVHNAKTKPREEQYNSTYRVVSVLTVLHQDW |
| | | LNGKEYKCKVSNKALPAPIEKTISKAKGQPREPQV |
| | | YTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| 31 | Anti-HER2 CDRL1 | RASQDVNTAVA |
| | (Kabat) | |
| 32 | Anti-HER2 CDRL2 | SASFLYS |
| | (Kabat) | |
| 33 | Anti-HER2 CDRL3 | QQHYTTPPT |
| | (Kabat) | |
| 34 | Anti-HER2 CDRH1 | DTYIH |
| | (Kabat) | |
| 35 | Anti-HER2 CDRH2 | RIYPTNGYTRYADSVKG |
| | (Kabat) | |
| 36 | Anti-HER2 CDRH3 | WGGDGFYAMDY |
| | (Kabat) | |
| 37 | Anti-HER2 Light Chain | DIQMTQSPSSLSASVGDRVTITCRASQDVNTAVAW |
| | Variable Region | YQQKPGKAPKLLIYSASFLYSGYPSRFSGSRSGTDF |
| | | TLTISSLQPEDFATYYCQQHYTTPPTFGQGTKVEIK |
| 38 | Anti-HER2 Heavy Chain | EVQLVESGGGLVQPGGSLRLSCAASGFNIKDTYIH |
| | Variable Region | WVRQAPGKGLEWVARIYPINGYTRYADSVKGRFT |
| | | ISADTSKNTAYLQMNSLRAEDTAVYYCSRWGGDG |
| | | FYAMDYWGQGTLVTVSS |
| 39 | Anti-HER2 Light Chain | DIQMTQSPSSLSASVGDRVTITCRASQDVNTAVAW |
| | | YQQKPGKAPKLLIYSASFLYSGVPSRFSGSRSGTDF |
| | | TLTISSLQPEDFATYYCQQHYTTPPTFGQGTKVEIK |
| | | RTVAAPSVFIFPPSDEQLKSGTASVVCLLNNFYPRE |
| | | AKVQWKVDNALQSGNSQESVTEQDSKDSTYSLSS |
| | | TLTLSKADYEKHKVYACEVTHQGLSSPVTKSFNRG |
| | | EC |
| 40 | Anti-HER2 Heavy Chain | EVQLVESGGGLVQPGGSLRLSCAASGFNIKDTYIH |
| | | WVRQAPGKGLEWVARIYPTNGYTRYADSVKGRFT |
| | | ISADTSKNTAYLQMNSLRAEDTAVYYCSRWGGDG |
| | | FYAMDYWGQGTLVTVSSASTKGPSVFPLAPSSKST |
| | | SGGTAALGCLVKDYFPEPVTVSWNSGALTSGVHTF |
| | | PAVLQSSGLYSLSSVVTVPSSSLGTQTYICNVNHKP |
| | | SNTKVDKKVEPKSCDKTHTCPPCPAPELLGGPSVFL |
| | | FPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSPGK |
| 41 | Anti-MSLN-1 CDR1 | VNAYG |
| | (Kabat) | |
| 42 | Anti-MSLN-1 CDR2 | IISAGGTTNYADSVKG |
| | (Kabat) | |
| 43 | Anti-MSLN-1 CDR3 | QRRIGMLRDY |
| | (Kabat) | |
| 44 | Anti-MSLN-1 Variable | QVQLVESGGGLVQPGGSLRLSCAASGITFPVNAYG |
| | | WYRQAPGKQRDLVAIISAGGTTNYADSVKGRFTIS |
| | | RDNSKNTLYLQMNSLRAEDTAVYYCYLQRRIGML |
| | | RDYWGQGTQVTVSS |
| 45 | Anti-MSLN-1 | QVQLVESGGGLVQPGGSLRLSCAASGITFPVNAYG |
| | | WYRQAPGKQRDLVAIISAGGTTNYADSVKGRFTIS |
| | | RDNSKNTLYLQMNSLRAEDTAVYYCYLQRRIGML |
| | | RDYWGQGTQVTVSSDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 46 | Anti-MSLN-2 CDR1 | SYVMA |
| | (Kabat) | |
| 47 | Anti-MSLN-2 CDR2 | SINWSSGRLIYADSVKG |
| | (Kabat) | |
| | Anti-MSLN-2 CDR3 | GRY |
| | (Kabat) | |
| 48 | Anti-MSLN-2 Variable | QVQLVESGGGLVQPGGSLRLSCAASGRTLESYVM |
| | | AWFRQAPGKEREAVASINWSSGRLIYADSVKGRFT |
| | | ISRDNSKNTLYLQMNSLRAEDTAVYYCAAGRYWG |
| | | QGTQVTVSS |
| 49 | Anti-MSLN-2 | QVQLVESGGGLVQPGGSLRLSCAASGRTLESYVM |
| | | AWFRQAPGKEREAVASINWSSGRLIYADSVKGRET |
| | | ISRDNSKNTLYLQMNSLRAEDTAVYYCAAGRYWG |
| | | QGTQVTVSSDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYT |
| | | LPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |
| 50 | Anti-TROP2 CDRL1 | KASQDVSIAVA |
| | (Kabat) | |
| 51 | Anti-TROP2 CDRL2 | SASYRYT |
| | (Kabat) | |
| 52 | Anti-TROP2 CDRL3 | QQHYITPLT |
| | (Kabat) | |
| 53 | Anti-TROP2 CDRH1 | NYGMN |
| | (Kabat) | |
| 54 | Anti-TROP2 CDRH2 | WINTYTGEPTYTDDFKG |
| | (Kabat) | |
| 55 | Anti-TROP2 CDRH3 | GGFGSSYWYFDV |
| | (Kabat) | |
| 56 | Anti-TROP2 Light Chain | DIQLTQSPSSLSASVGDRVSITCKASQDVSIAVAWY |
| | Variable Region | QQKPGKAPKLLIYSASYRYTGVPDRFSGSGSGTDFT |
| | | LTISSLQPEDFAVYYCQQHYITPLTFGAGTKVEIK |
| 57 | Anti-TROP2 Heavy Chain | QVQLQQSGSELKKPGASVKVSCKASGYTFTNYGM |
| | Variable Region | NWVKQAPGQGLKWMGWINTYTGEPTYTDDFKGR |
| | | FAFSLDTSVSTAYLQISSLKADDTAVYFCARGGFGS |
| | | SYWYFDVWGQGSLVTVSS |
| 58 | Anti-TROP2 Light Chain | DIQLTQSPSSLSASVGDRVSITCKASQDVSIAVAWY |
| | | QQKPGKAPKLLIYSASYRYTGVPDRFSGSGSGTDFT |
| | | LTISSLQPEDFAVYYCQQHYITPLTFGAGTKVEIKR |
| | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| 59 | Anti-TROP2 Heavy Chain | QVQLQQSGSELKKPGASVKVSCKASGYTFTNYGM |
| | | NWVKQAPGQGLKWMGWINTYTGEPTYTDDFKGR |
| | | FAFSLDTSVSTAYLQISSLKADDTAVYFCARGGFGS |
| | | SYWYFDVWGQGSLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALPAPIEKTISKAKGQPRE |
| | | PQVYTLPPSREEMTKNQVSLTCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 60 | Anti-CD79b-1 CDRL1 | RSSQSLRDSDGKTYLN |
| | (Kabat) | |
| 61 | Anti-CD79b-1 CDRL2 | LVSKLDS |
| | (Kabat) | |
| 62 | Anti-CD79b-1 CDRL3 | WQGTHRPPT |
| | (Kabat) | |
| 63 | Anti-CD79b-1 CDRH1 | DYYMN |
| | (Kabat) | |
| 64 | Anti-CD79b-1 CDRH2 | DIDPGDGDTYYSQKFQG |
| | (Kabat) | |
| 65 | Anti-CD79b-1 CDRH3 | NLYI |
| | (Kabat) | |
| 66 | Anti-CD79b-1 Light Chain | DVVMTQSPLSLPVTLGQPASISCRSSQSLRDSDGKT |
| | Variable Region | YLNWLQQRPGQSPRRLIYLVSKLDSGVPDRFSGSG |
| | | SGTDFTLKISRVEAEDVGVYYCWQGTHRPPTFGQG |
| | | TKLEIK |
| 67 | Anti-CD79b-1 Heavy Chain | QVQLVQSGAEVKKPGASVKLSCKASGYTFTDYYM |
| | Variable Region | NWVRQAPGQRLEWMGDIDPGDGDTYYSQKFQGR |
| | | VTITRDTSASTAYMELSSLRSEDTAVYYCARNLYIY |
| | | GQGTLVTVSS |
| 68 | Anti-CD79b-1 Light Chain | DVVMTQSPLSLPVTLGQPASISCRSSQSLRDSDGKT |
| | | YLNWLQQRPGQSPRRLIYLVSKLDSGVPDRFSGSG |
| | | SGTDFTLKISRVEAEDVGVYYCWQGTHRPPTFGQG |
| | | TKLEIKRTVAAPSVFIFPPSDEQLKSGTASVVCLLN |
| | | NFYPREAKVQWKVDNALQSGNSQESVTEQDSKDS |
| | | TYSLSSTLTLSKADYEKHKVYACEVTHQGLSSPVT |
| | | KSFNRGEC |
| 69 | Anti-CD79b-1 Heavy Chain | QVQLVQSGAEVKKPGASVKLSCKASGYTFTDYYM |
| | | NWVRQAPGQRLEWMGDIDPGDGDTYYSQKFQGR |
| | | VTITRDTSASTAYMELSSLRSEDTAVYYCARNLYIY |
| | | GQGTLVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDKK |
| | | VEPKSCDKTHTCPPCPAPELLGGPSVFLFPPKPKDT |
| | | LMISRTPEVTCVVVDVSHEDPEVKFNWYVDGVEV |
| | | HNAKTKPREEQYNSTYRVVSVLTVLHQDWLNGKE |
| | | YKCKVSNKALPAPIEKTISKAKGQPREPQVYTLPPS |
| | | REEMTKNQVSLTCLVKGFYPSDIAVEWESNGQPEN |
| | | NYKTTPPVLDSDGSFFLYSKLTVDKSRWQQGNVFS |
| | | CSVMHEALHNHYTQKSLSLSPGK |
| 70 | Anti-CD79b-2 CDRL1 | KASQSVDYEGDSFLN |
| | (Kabat) | |
| 71 | Anti-CD79b-2 CDRL2 | AASNLES |
| | (Kabat) | |
| 72 | Anti-CD79b-2 CDRL3 | QQSNEDPLT |
| | (Kabat) | |
| 73 | Anti-CD79b-2 CDRH1 | SYWIE |
| | (Kabat) | |
| 74 | Anti-CD79b-2 CDRH2 | EILPGGGDTNYNEIFKG |
| | (Kabat) | |
| 75 | Anti-CD79b-2 CDRH3 | RVPIRLDY |
| | (Kabat) | |
| 76 | Anti-CD79b-2 Light Chain | DIQLTQSPSSLSASVGDRVTITCKASQSVDYEGDSF |
| | Variable Region | LNWYQQKPGKAPKLLIYAASNLESGVPSRFSGSGS |
| | | GTDFTLTISSLQPEDFATYYCQQSNEDPLTFGQGTK |
| | | VEIK |
| 77 | Anti-CD79b-2 Heavy Chain | EVQLVESGGGLVQPGGSLRLSCAASGYTFSSYWIE |
| | Variable Region | WVRQAPGKGLEWIGEILPGGGDTNYNEIFKGRATF |
| | | SADTSKNTAYLQMNSLRAEDTAVYYCTRRVPIRLD |
| | | YWGQGTLVTVSS |
| 78 | Anti-CD79b-2 Light Chain | DIQLTQSPSSLSASVGDRVTITCKASQSVDYEGDSF |
| | | LNWYQQKPGKAPKLLIYAASNLESGVPSRFSGSGS |
| | | GTDFTLTISSLQPEDFATYYCQQSNEDPLTFGQGTK |
| | | VEIKRTVAAPSVFIFPPSDEQLKSGTASVVCLLNNF |
| | | YPREAKVOWKVDNALQSGNSQESVTEQDSKDSTY |
| | | SLSSTLTLSKADYEKHKVYACEVTHQGLSSPVTKS |
| | | FNRGEC |
| 79 | Anti-CD79b-2 Heavy Chain | EVQLVESGGGLVQPGGSLRLSCAASGYTFSSYWIE |
| | | WVRQAPGKGLEWIGEILPGGGDTNYNEIFKGRATF |
| | | SADTSKNTAYLQMNSLRAEDTAVYYCTRRVPIRLD |
| | | YWGQGTLVTVSSASTKGPSVFPLAPSSKSTSGGTA |
| | | ALGCLVKDYFPEPVTVSWNSGALTSGVHTFPAVLQ |
| | | SSGLYSLSSVVTVPSSSLGTQTYICNVNHKPSNTKV |
| | | DKKVEPKSCDKTHTCPPCPAPELLGGPSVFLFPPKP |
| | | KDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYT |
| | | LPPSREEMTKNQVSLTCLVKGFYPSDIAVEWESNG |
| | | QPENNYKTTPPVLDSDGSFFLYSKLTVDKSRWQQG |
| | | NVFSCSVMHEALHNHYTQKSLSLSPGK |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A compound of Formula (XV): 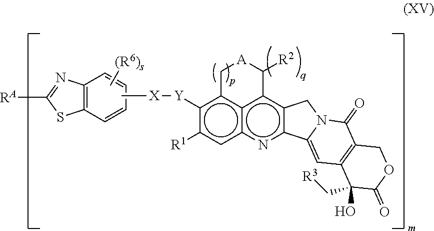
or a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein:
each A is independently O, S, S(O), S(O₂), or N(R⁴);
each X is independently C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
each Y is independently a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene;
each R¹, R², and R³ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a}) NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1d}, —OS(O)₂R^{1d}, —OS(O)NR^{1b}R^{1c}, —OS(O)₂NR^{1b}R^{1c}, —NR^{1b}R^{1c}, —NR^{1d}C(O)R^{1d}, —NR^{1a}C(O)OR^{1d}, —NR^{1a}C(O)NR^{1b}R^{1c}, —NR^{1a}C(O)SR^{1d}, —NR^{1d}C(NR^{1d})NR^{1b}R^{1c}, —NR^{1a}C(S)R^{1d}, —NR^{1a}C(S)OR^{1d}, —NR^{1d}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1d}, —S(O)₂R^{1a}, —S(O)NR^{1b}R^{1c}, or —S(O)₂NR^{1b}R^{1c};
each R⁴ is independently (i) hydrogen; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O) NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
each R⁶ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1d}, —C(O) OR^{1a}, —C(O)NR^{1b}R^{1c}, —C(O)SR^{1d}, —C(NR^{1d})NR^{1b}R^{1c}, —C(S)R^{1d}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR a, —OC(NR^{1a})NR^{1b}R^{1c}, —OC(S)R^{1a}, —OC(S) OR^{1a}, —OC(S)NR^{1b}R^{1c}, —OS(O)R^{1d}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1d}C(S)R^{1d}, —NR^{1a}C(S)OR^{1d}, —NR^{1a}C(S)NR^{1b}R^{1c}, —NR^{1d}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1d}, —S(O)₂R^{1a}, —S(O)NR^{1b}R^{1c}, or —S(O)₂NR^{1b}R^{1c};
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
R^{A} is an antibody or an antigen-binding fragment thereof;
m is an integer of 1, 2, 3, 4, 5, 6, 7, 8, 9, 10, 11, 12, 13, 14, 15, or 16;
each p and q is independently an integer of 0, 1, or 2; with the proviso that the total of p and q is greater than 0; and
each s is independently an integer of 0, 1, 2, or 3;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q, wherein each Q is independently selected from:
(a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a}) NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O) OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, having the structure of Formula (XVI): 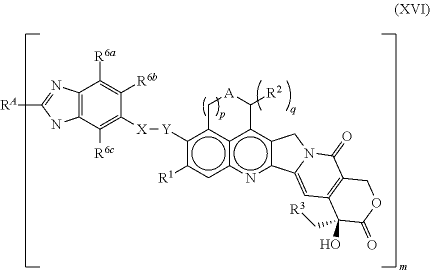
or a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein each R^{6a}, R^{6b}, and R^{6c} is independently hydrogen or R⁶.

3. The compound of claim 1 or 2, wherein each p is an integer of 0.

4. The compound of claim 1 or 2, wherein each p is an integer of 1.

5. The compound of claim 1 or 2, wherein each p is an integer of 2.

6. The compound of any one of claims 1 to 5, wherein each q is an integer of 0.

7. The compound of any one of claims 1 to 5, wherein each q is an integer of 1.

8. The compound of any one of claims 1 to 5, wherein each q is an integer of 2.

9. The compound of claim 2, having the structure of Formula (XVI): 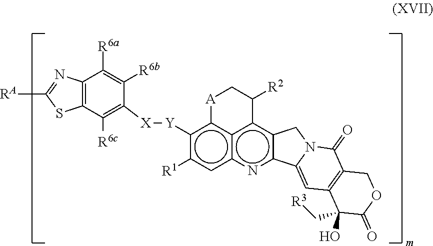
or a diastereomer, a mixture of two or more diastereomers, a tautomer, a mixture of two or more tautomers, or an isotopic variant thereof; or a pharmaceutically acceptable salt, solvate, hydrate, or prodrug thereof.

10. The compound of claim 2, having the structure of Formula (XVIII): 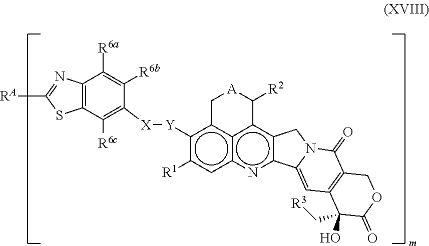
or a diastereomer, a mixture of two or more diastereomers, a tautomer, a mixture of two or more tautomers, or an isotopic variant thereof; or a pharmaceutically acceptable salt, solvate, hydrate, or prodrug thereof.

11. The compound of any one of claims 2 to 10, wherein R⁶, is hydrogen.

12. The compound of any one of claims 2 to 11, wherein R^{6c} is hydrogen.

13. The compound of any one of claims 1 to 12, wherein each X is independently C₁₋₄₀ heteroalkylene, wherein one or more methylene groups are each independently and optionally replaced by a divalent group and each divalent group is independently C₆₋₁₄ arylene, heteroarylene, or heterocyclylene; and wherein each heteroalkylene, arylene, heteroarylene, and heterocyclylene is optionally substituted with one or more substituents Q.

14. The compound of any one of claims 1 to 13, wherein each X is independently: 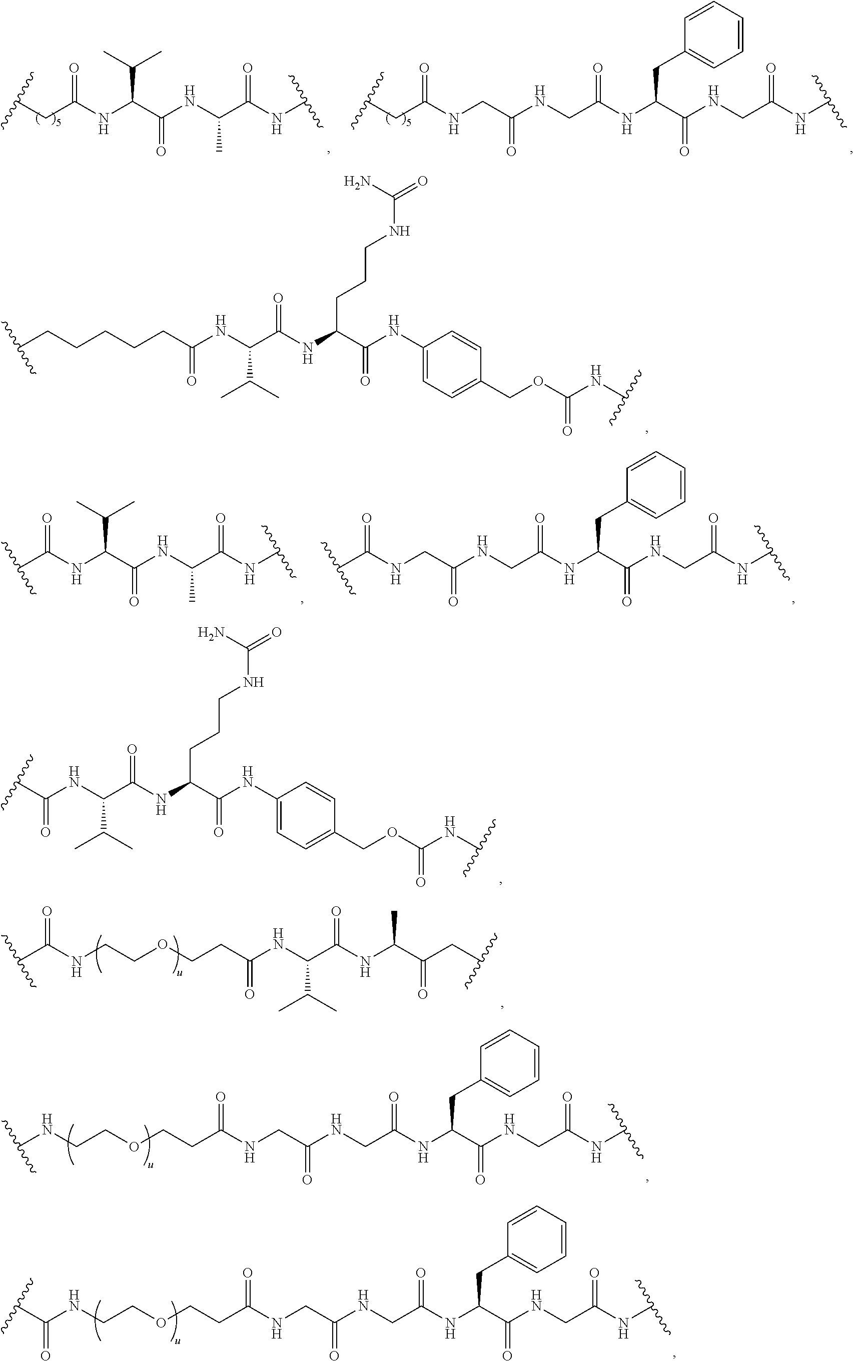 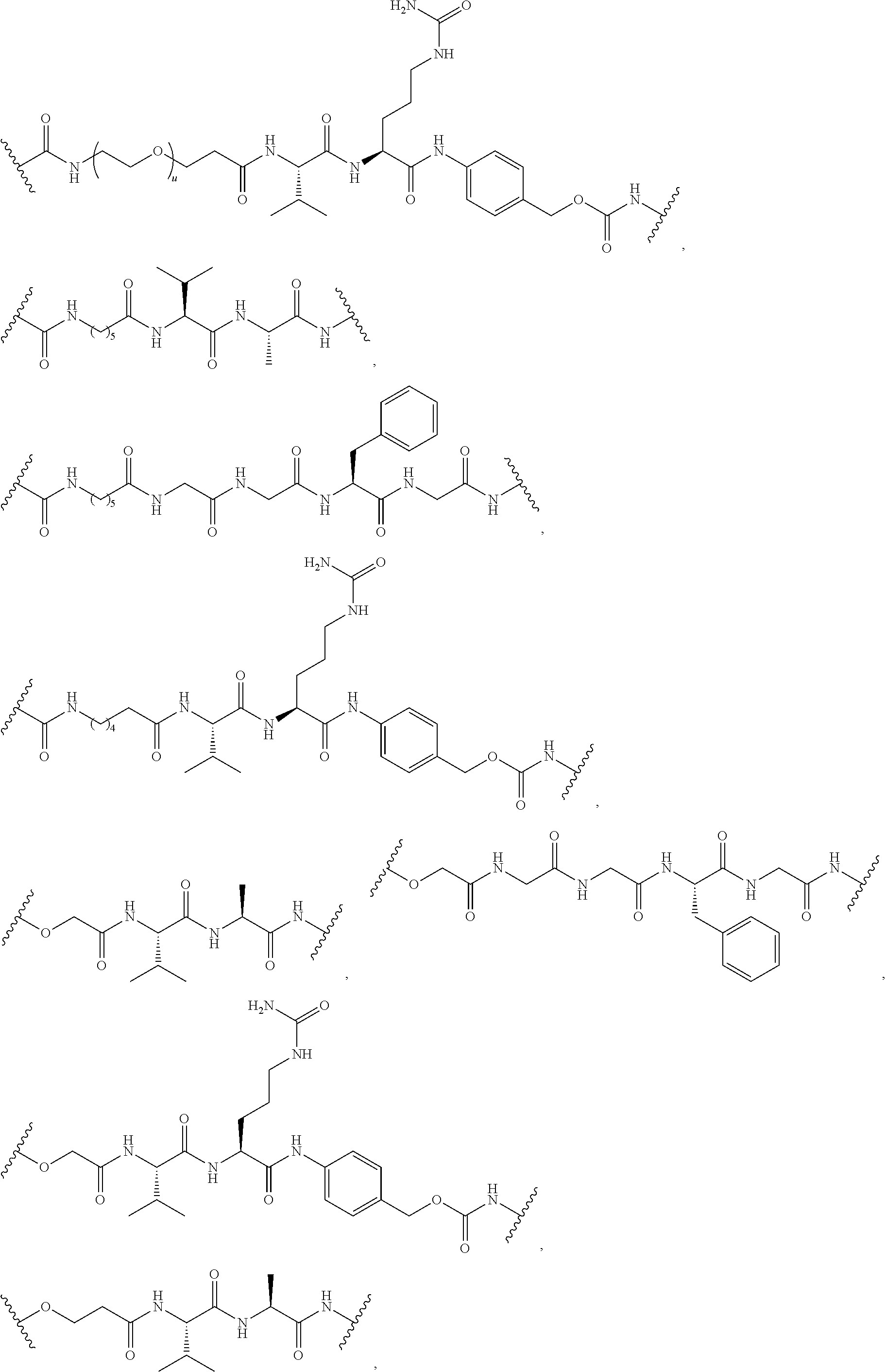 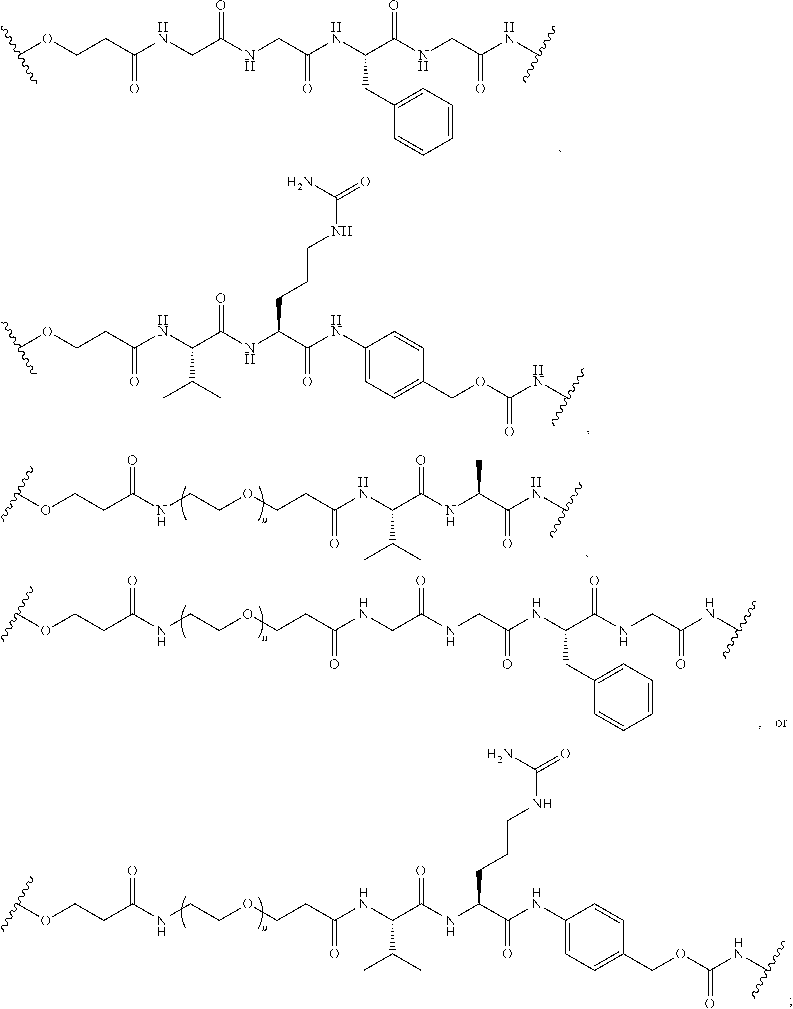
wherein each u is independently an integer of 1, 2, 3, 4, 5, 6, 7, or 8.

15. The compound of any one of claims 1 to 14, wherein each Y is a bond.

16. The compound of any one of claims 1 to 15, wherein each R² is independently (i) hydrogen or deuterium; (ii) heterocyclyl, optionally substituted with one or more substituents Q; or (iii) —NR^{1b}R^{1c} or —NR^{1a} C(O)R^{1a}.

17. The compound of any one of claims 1 to 16, wherein each R² is independently hydrogen, amino, methylamino, acetamido, or hydroxyacetamido.

18. The compound of any one of claims 1 to 17, wherein each R² is hydrogen.

19. The compound of any one of claims 1 to 18, wherein each R³ is independently C₁₋₆ alkyl, C₁₋₆ heteroalkyl, or C₂₋₆ alkenyl, each optionally substituted with one or more substituents Q.

20. The compound of any one of claims 1 to 19, wherein each R³ is independently methyl, fluoromethyl, methoxymethyl, or ethenyl.

21. The compound of any one of claims 1 to 20, wherein each R³ is methyl.

22. The compound of claim 2, wherein the compound is: 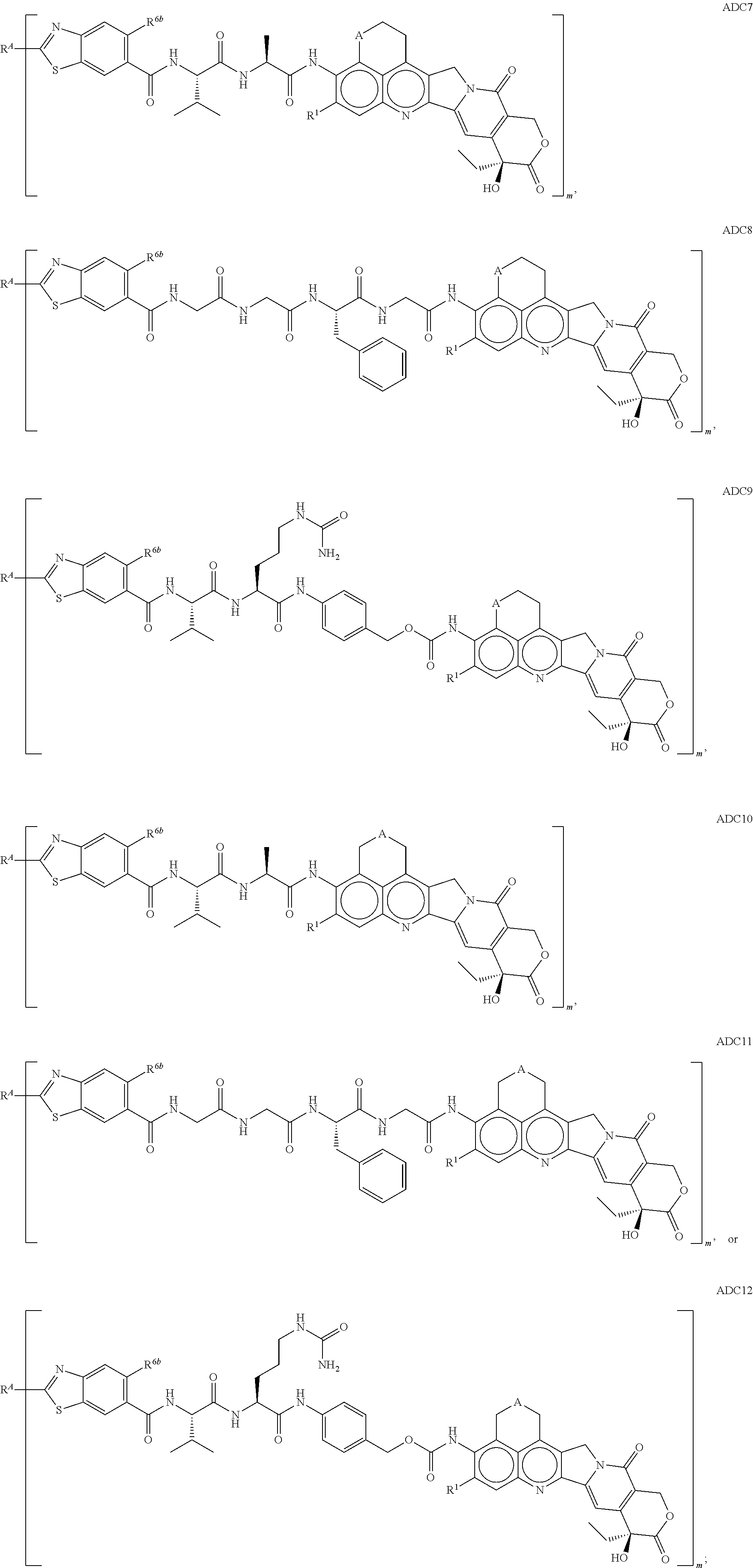
or a diastereomer, a mixture of two or more diastereomers, a tautomer, a mixture of two or more tautomers, or an isotopic variant thereof; or a pharmaceutically acceptable salt, solvate, hydrate, or prodrug thereof.

23. The compound of claim 2, wherein the compound is: 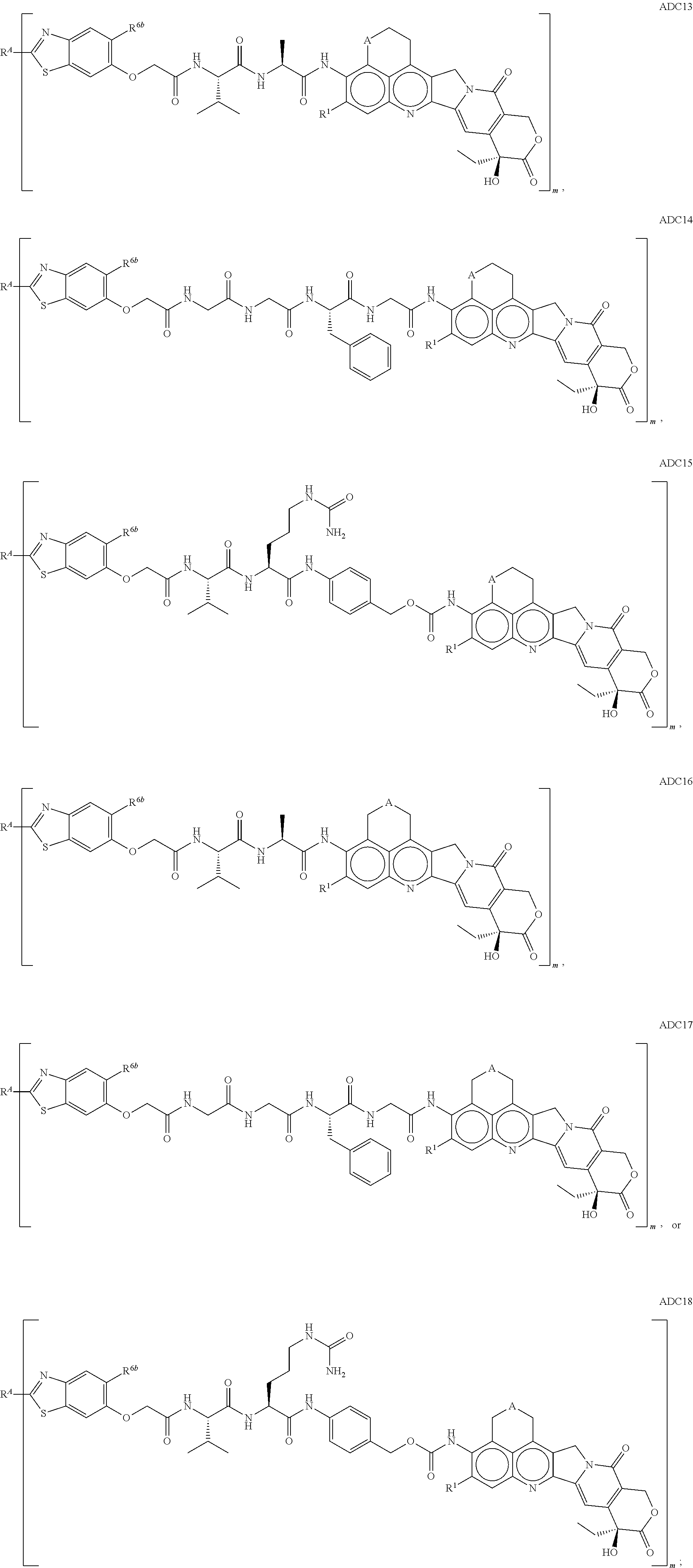
or a diastereomer, a mixture of two or more diastereomers, a tautomer, a mixture of two or more tautomers, or an isotopic variant thereof; or a pharmaceutically acceptable salt, solvate, hydrate, or prodrug thereof.

24. The compound of claim 2, wherein the compound is 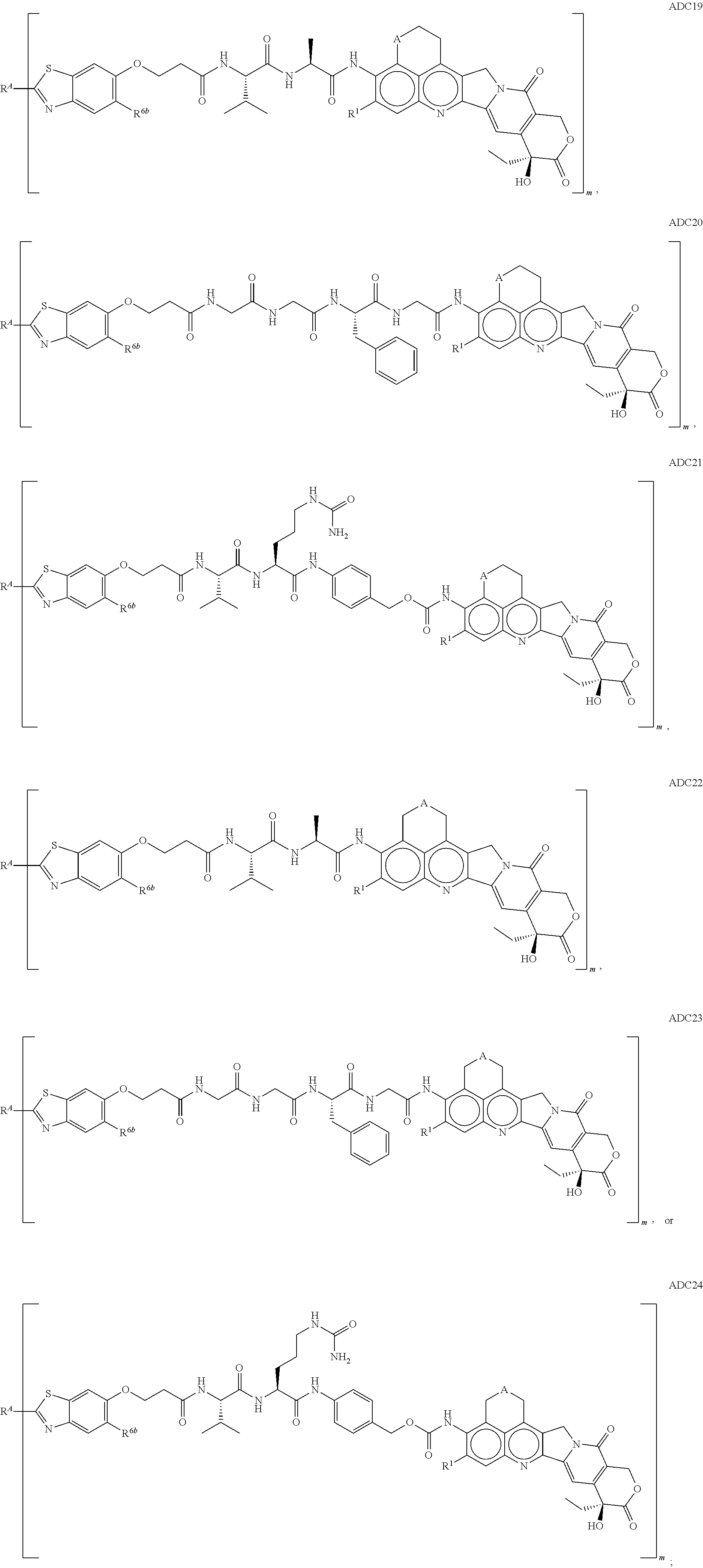
or a diastereomer, a mixture of two or more diastereomers, a tautomer, a mixture of two or more tautomers, or an isotopic variant thereof; or a pharmaceutically acceptable salt, solvate, hydrate, or prodrug thereof.

25. The compound of claim 2, wherein the compound is: 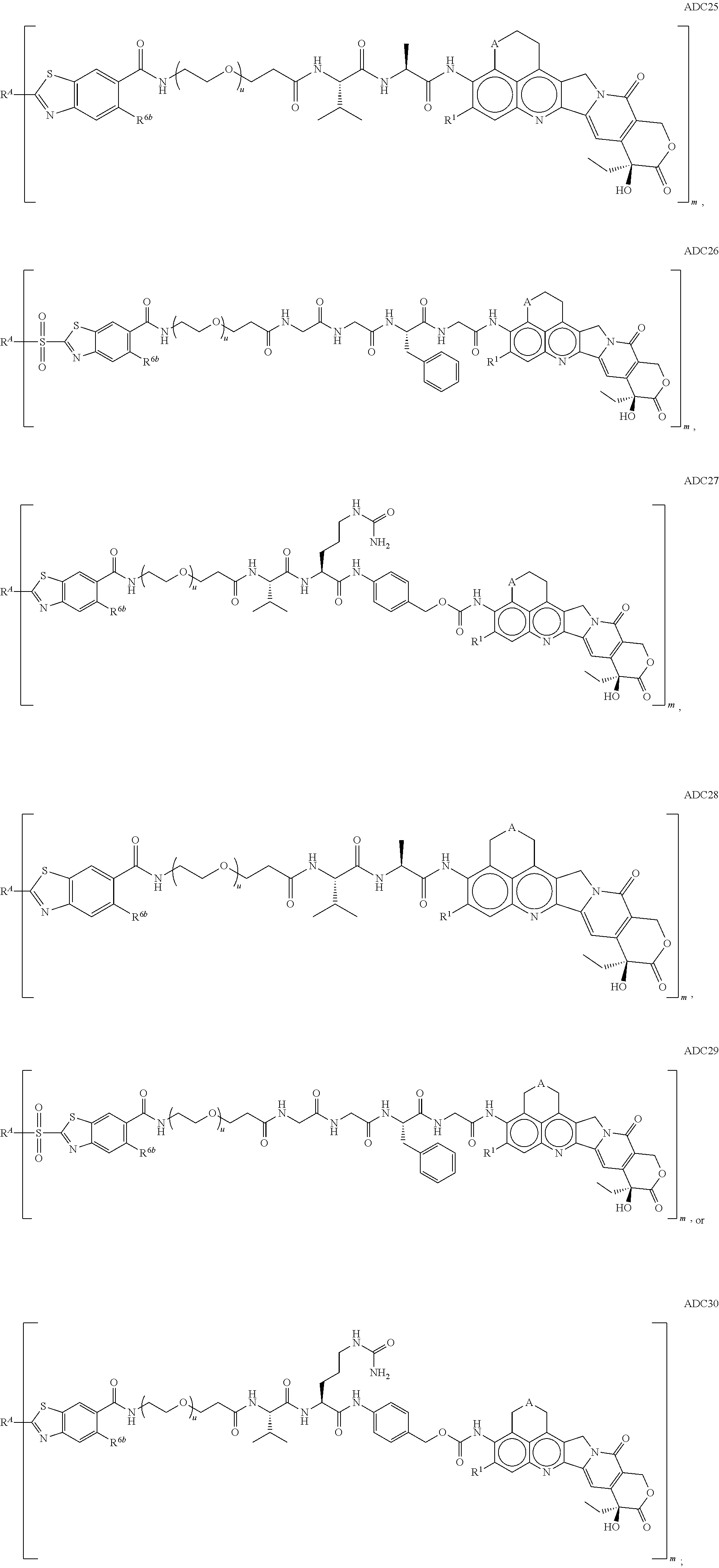
or a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein each u is independently an integer of an integer of 1, 2, 3, 4, 5, 6,7, or 8.

26. The compound of claim 2, wherein the compound is: 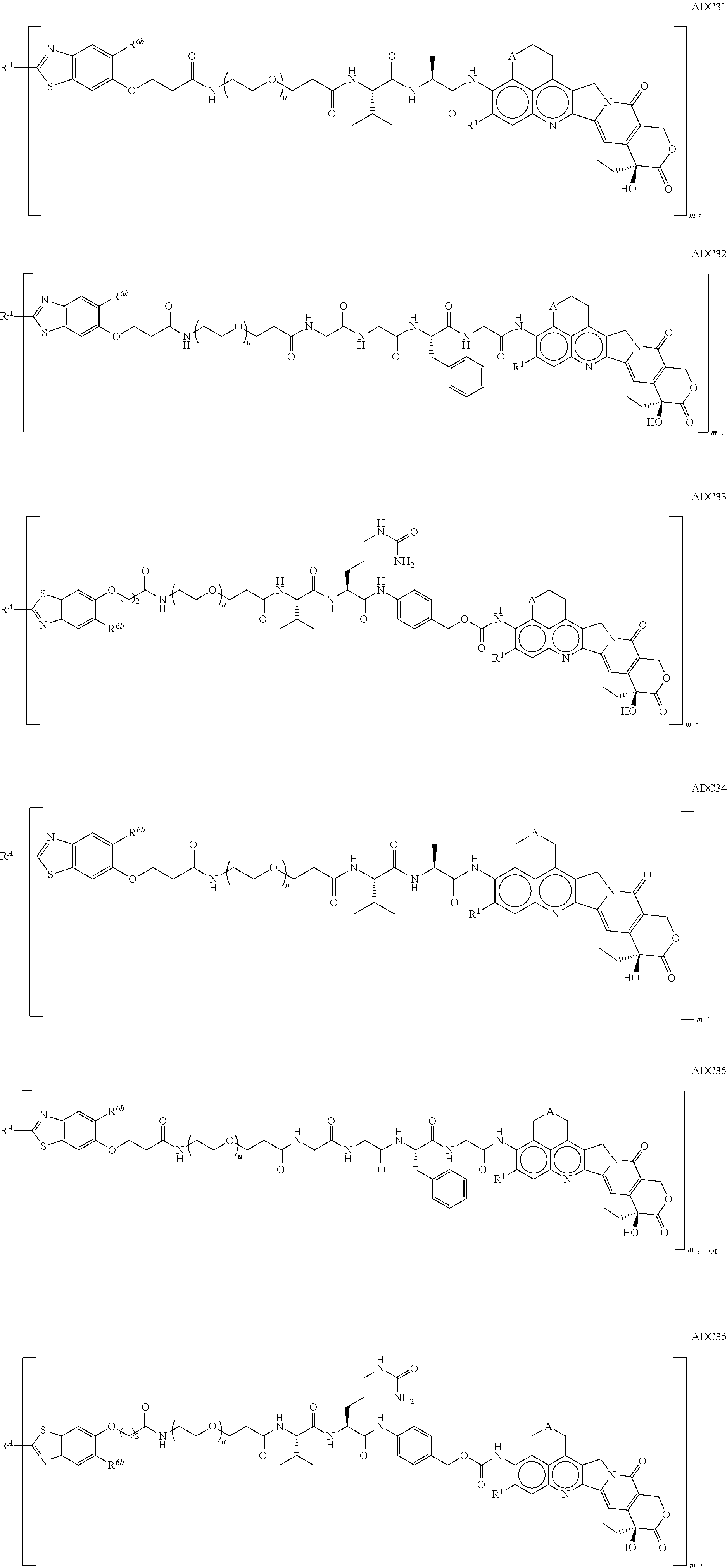
or a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein each a is independently an integer of an integer of 1, 2, 3, 4, 5, 6, 7, or 8.

27. The compound of claim 2, wherein the compound is: 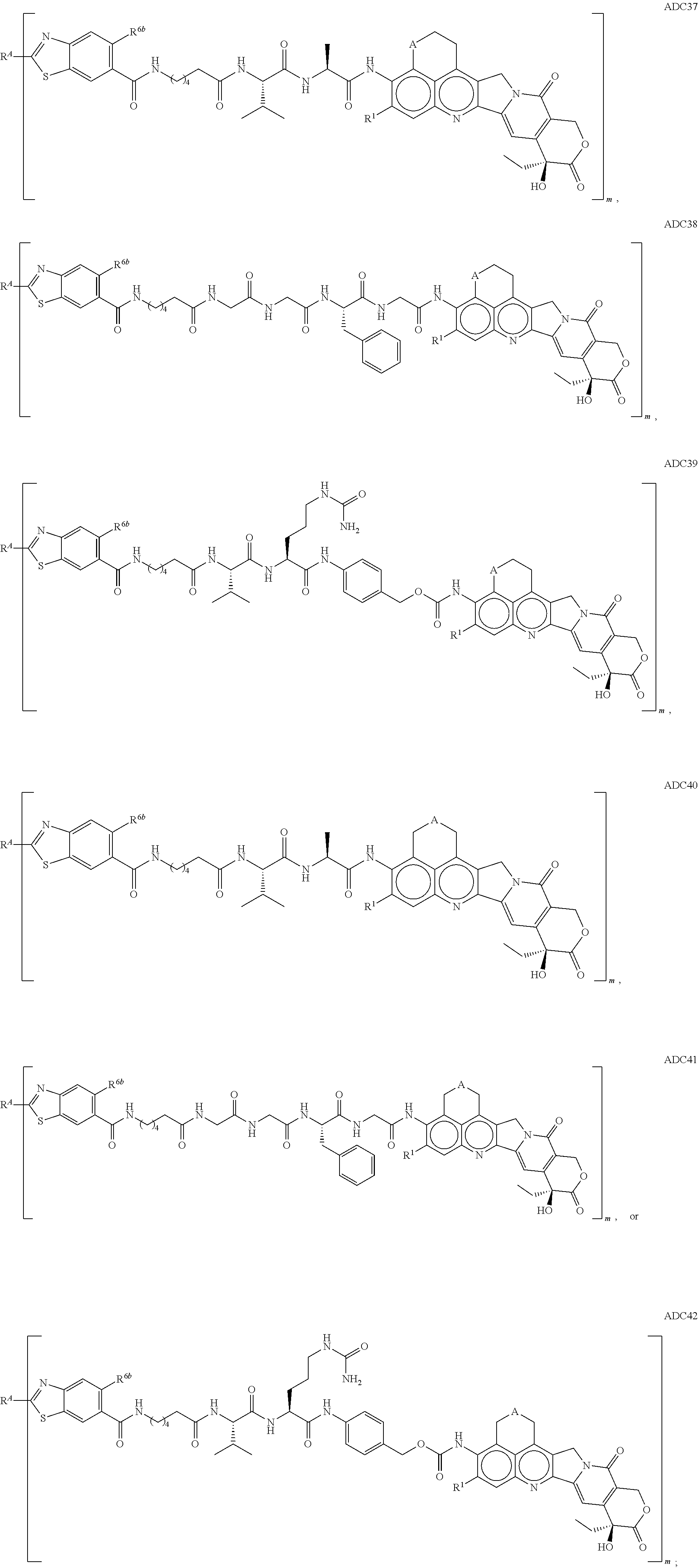
or a diastereomer, a mixture of two or more diastereomers, a tautomer, a mixture of two or more tautomers, or an isotopic variant thereof; or a pharmaceutically acceptable salt, solvate, hydrate, or prodrug thereof.

28. The compound of any one of claims 1 to 27, wherein each R¹ is independently hydrogen or halo.

29. The compound of any one of claims 1 to 28, wherein each R¹ is hydrogen.

30. The compound of any one of claims 1 to 28, wherein each R¹ is independently halo.

31. The compound of any one of claims 1 to 28 and 30, wherein each R¹ is fluoro.

32. The compound of any one of claims 1 to 31, wherein each A is O.

33. The compound of any one of claims 1 to 31, wherein each A is S.

34. The compound of any one of claims 1 to 33, wherein each R^{6b} is hydrogen.

35. The compound of any one of claims 1 to 33, wherein each R^{6b} is independently halo.

36. The compound of any one of claims 1 to 33 and 35, wherein each R^{6b} is fluoro.

37. The compound of any one of claims 1 to 36, wherein R^{A} is a monoclonal antibody or an antigen-binding fragment thereof.

38. The compound of any one of claims 1 to 37, wherein R^{A} is a human, humanized, or chimeric antibody, or an antigen-binding fragment thereof.

39. The compound of any one of claims 1 to 38, wherein R^{A} is an IgG1, IgG2, IgG3, or IgG4 antibody, or an antigen-binding fragment thereof.

40. The compound of any one of claims 1 to 39, wherein R^{A} is anti-B7H3 antibody, anti-CD20 antibody, anti-FOLR1 antibody, anti-HER2 antibody, anti-MSLN antibody, anti-TROP2 antibody, or an antigen-binding fragment thereof.

41. The compound of any one of claims 1 to 40, wherein R^{A} is an anti-B7H3 single domain antibody or an antigen-binding fragment thereof.

42. The compound of claim 41, wherein the anti-B7H3 single domain antibody comprises: (i) a CDR1 of SEQ ID NO: 1, a CDR2 of SEQ ID NO: 2, and a CDR3 of SEQ ID NO: 3; or (ii) a CDR1 of SEQ ID NO: 6, a CDR2 of SEQ ID NO: 7, and a CDR3 of SEQ ID NO: 8.

43. The compound of any one of claims 1 to 40, wherein R^{A} is an anti-CD20 antibody or an antigen-binding fragment thereof.

44. The compound of claim 43, wherein the anti-CD20 antibody comprises a CDRL1 of SEQ ID NO: 11; a CDRL2 of SEQ ID NO: 12; a CDRL3) of SEQ ID NO: 13; a CDRH1 of SEQ ID NO: 14; a CDRH2 of SEQ ID NO: 15; and a CDRH3 of SEQ ID NO: 16.

45. The compound of any one of claims 1 to 40, wherein R^{A} is an anti-FOLR1 antibody or an antigen-binding fragment thereof.

46. The compound of claim 45, wherein the anti-FOLR1 antibody comprises a CDRL1 of SEQ ID NO: 21; a CDRL2 of SEQ ID NO: 22; a CDRL3 of SEQ ID NO: 23; a CDRH1 of SEQ ID NO: 24; a CDRH2 of SEQ ID NO: 25; and a CDRH3 of SEQ ID NO: 26.

47. The compound of any one of claims 1 to 40, wherein R^{A} is an anti-HER2 antibody or an antigen-binding fragment thereof.

48. The compound of claim 47, wherein the anti-HER2 antibody comprises a CDRL1 of SEQ ID NO: 31; a CDRL2 of SEQ ID NO: 32; a CDRL3 of SEQ ID NO: 33; a CDRH1 of SEQ ID NO: 34; a CDRH2 of SEQ ID NO: 35; and a CDRH3 of SEQ ID NO: 36.

49. The compound of claim 47 or 48, wherein the anti-HER2 antibody comprises a light chain variable region of SEQ ID NO: 37 and a heavy chain variable region of SEQ ID NO: 38.

50. The compound of any one of claims 47 to 49, wherein the anti-HER2 antibody comprises a light chain of SEQ ID NO: 39 and a heavy chain of SEQ ID NO: 40.

51. The compound of any one of claims 1 to 40, wherein R^{A} is an anti-MSLN single domain antibody or an antigen-binding fragment thereof.

52. The compound of claim 51, wherein the anti-MSLN single domain antibody comprises: (i) a CDR1 of SEQ ID NO: 41, a CDR2 of SEQ ID NO: 42, and a CDR3 of SEQ ID NO: 43; or (ii) a CDR1 of SEQ ID NO: 46, a CDR2 of SEQ ID NO: 47, and a CDR3 of GRY.

53. The compound of any one of claims 1 to 40, wherein R^{A} is an anti-TROP2 antibody or an antigen-binding fragment thereof.

54. The compound of claim 53, wherein the anti-TROP2 antibody comprises a CDRL1 of SEQ ID NO: 50; a CDRL2 of SEQ ID NO: 51; a CDRL3 of SEQ ID NO: 52; a CDRH1 of SEQ ID NO: 53; a CDRH2 of SEQ ID NO: 54; and a CDRH3 of SEQ ID NO: 55.

55. The compound of any one of claims 1 to 40, wherein R^{A} is an anti-CD79b antibody or an antigen-binding fragment thereof.

56. The compound of claim 55, wherein the anti-CD79b antibody comprises a CDRL1 of SEQ ID NO: 60; a CDRL2 of SEQ ID NO: 61; a CDRL3 of SEQ ID NO: 62; a CDRH1 of SEQ ID NO: 63; a CDRH2 of SEQ ID NO: 64; and a CDRH3 of SEQ ID NO: 65.

57. The compound of claim 55, wherein the anti-CD79b antibody comprises a CDRL1 of SEQ ID NO: 70; a CDRL2 of SEQ ID NO: 71; a CDRL3 of SEQ ID NO: 72; a CDRH1 of SEQ ID NO: 73; a CDRH2 of SEQ ID NO: 74; and a CDRH3 of SEQ ID NO: 75.

58. The compound of any one of claims 1 to 57, wherein m is an integer of 1, 2, 3, 4, 5, 6, 7, 8, 9, 10, 11, or 12.

59. The compound of any one of claims 1 to 58, wherein m is an integer of 4, 5, 6, 7, or 8.

60. The compound of any one of claims 1 to 59, wherein m is an integer of 8.

61. The compound of any one of claims 14 to 21, 25, 26, and 28 to 60, wherein each u is independently an integer of 4 or 8.

62. The compound of any one of claims 14 to 21, 25, 26, and 28 to 61, wherein each u is independently an integer of 4.

63. The compound of claim 1, wherein the compound is an ADC of trastuzumab, selected from ADC-C1 to ADC-C53; and wherein trastuzumab is an anti-HER2 antibody comprising a CDRL1 of SEQ ID NO: 31, a CDRL2 of SEQ ID NO: 32, a CDRL3 of SEQ ID NO: 33, a CDRH1 of SEQ ID NO: 34, a CDRH2 of SEQ ID NO: 35, and a CDRH3 of SEQ ID NO: 36.

64. A pharmaceutical composition comprising the compound of any one of claims 1 to 63, or a diastereomer, a mixture of two or more diastereomers, a tautomer, a mixture of two or more tautomers, or an isotopic variant thereof; or a pharmaceutically acceptable salt, solvate, or hydrate thereof; and a pharmaceutically acceptable excipient.

65. The pharmaceutical composition of claim 64, wherein the composition is in single dosage form.

66. The pharmaceutical composition of claim 64 or 65, wherein the composition is in a parenteral or intravenous dosage form.

67. The pharmaceutical composition of claim 66, wherein the composition is formulated in an intravenous dosage form.

68. A method of treating, preventing, or ameliorating one or more symptoms of a proliferative disease in a subject, comprising administering to the subject in need thereof a therapeutically effective amount of a compound of any one of claims 1 to 63 or a pharmaceutical composition of any one of claims 64 to 67.

69. The method of claim 68, wherein the proliferative disease is cancer.

70. The method of claim 68 or 69, wherein the cancer is relapsed or refractory.

71. The method of any one of claims 68 to 70, wherein the cancer is metastatic.

72. The method of any one of claims 68 to 71, wherein the cancer is drug-resistant.

73. The method of any one of claims 68 to 72, wherein the subject is a human.

74. A method of inhibiting the growth of a cell, comprising contacting the cell with an effective amount of a compound of any one of claims 1 to 63 or a pharmaceutical composition of any one of claims 64 to 67.

75. The method of claim 74, wherein the cell is a cancerous cell.

76. A compound of Formula (IA) 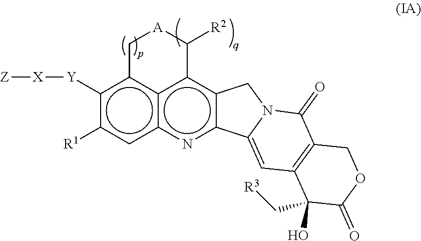
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein:
A is O, S, S(O), S(O₂), or N(R⁴);
X is C₁₋₄₀ alkylene, C₁₋₄₀ heteroalkylene, C₂₋₄₀ alkenylene, C₂₋₄₀ heteroalkenylene, C₂₋₄₀ alkynylene, or C₂₋₄₀ heteroalkynylene, wherein one or more methylene groups are each independently and optionally replaced by a divalent group and each divalent group is independently C₃₋₁₀ cycloalkylene, C₆₋₁₄ arylene, heteroarylene, or heterocyclylene;
Y is a bond, C₁₋₆ alkylene, C₁₋₆ heteroalkylene, C₂₋₆ alkenylene, C₂₋₆ alkynylene, C₃₋₁₀ cycloalkylene, C₆₋₁₄ arylene, C₇₋₁₅ aralkylene, heteroarylene, or heterocyclylene;
Z is 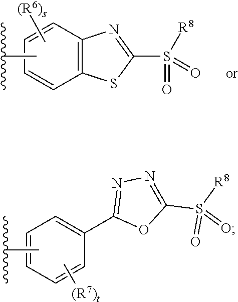
each R¹, R², and R³ is independently (i) hydrogen, deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a}) NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1d}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁴ is (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O) NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —S(O)R^{1a}, —S(O)₂R^{1a}, —S(O)NR^{1b}R^{1c}, or —S(O)₂NR^{1b}R^{1c};
each R⁶ and R⁷ is independently (i) deuterium, cyano, halo, or nitro; (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S) R^{1d}, —C(S)OR^{1a}, —C(S)NR^{1b}R^{1c}, —OR^{1a}, —OC(O)R^{1d}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1d}, —OS(O)₂R^{1a}, —OS(O) NR^{1b}R^{1c}, —OS(O)₂NR^{1b}R^{1c}, —NR^{1b}R^{1c}, —NR^{1d}C(O)R^{1d}, —NR^{1a}C(O) OR^{1d}, —NR^{1a}C(O)NR^{1b}R^{1c}, —NR^{1a}C(O)SR^{1d}, —NR^{1d}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1d}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R⁸ is independently (i) hydrogen or deuterium; or (ii) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, deuterium, C₁₋₆ alkyl, C₁₋₆ heteroalkyl, C₂₋₆ alkenyl, C₂₋₆ alkynyl, C₃₋₁₀ cycloalkyl, C₆ 14 aryl, C₇₋₁₅ aralkyl, heteroaryl, or heterocyclyl;
p and q are each independently an integer of 0, 1, or 2; with the proviso that the total of p and q is greater than 0;
s is an integer of 0, 1, 2, or 3; and
t is an integer of 0, 1, 2, 3, or 4;
wherein each alkyl, alkylene, heteroalkyl, heteroalkylene, alkenyl, alkenylene, heteroalkenylene, alkynyl, alkynylene, heteroalkynylene, cycloalkyl, cycloalkylene, aryl, arylene, aralkyl, aralkylene, heteroaryl, heteroarylene, heterocyclyl, and heterocyclylene is optionally substituted with one or more, in one embodiment, one, two, three, or four, substituents Q, wherein each Q is independently selected from:
(a) deuterium, cyano, halo, imino, nitro, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a}) NR^{b}R^{c}, —OC(S)R^{a}, —OC(S)OR^{a}, —OC(S)NR^{b}R^{c}, —OP(O)(OR^{a})OR^{d}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₁₋₆ heteroalkyl, C₂₋₆ alkenyl, C₂₋₆ alkynyl, C₃₋₁₀ cycloalkyl, C₆ 14 aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₁₋₆ heteroalkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{e}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O) OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

77. The compound of claim 76, having the structure of Formula (IXA): 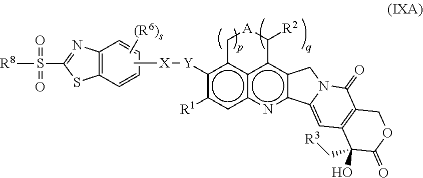
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

78. The compound of claim 76 or 77, having the structure of Formula (XA): 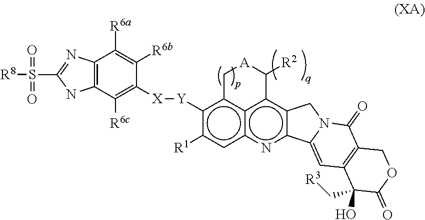
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; wherein each R^{6a}, R^{6b}, and R^{6c} is independently hydrogen or R⁶.

79. The compound of any one of claims 76 to 78, wherein each p is an integer of 0.

80. The compound of any one of claims 76 to 78, wherein each p is an integer of 1.

81. The compound of any one of claims 76 to 78, wherein each p is an integer of 2.

82. The compound of any one of claims 76 to 81, wherein each q is an integer of 0.

83. The compound of any one of claims 83 to 81, wherein each q is an integer of 1.

84. The compound of any one of claims 83 to 81, wherein each q is an integer of 2.

85. The compound of claim 78, having the structure of Formula (XIA) 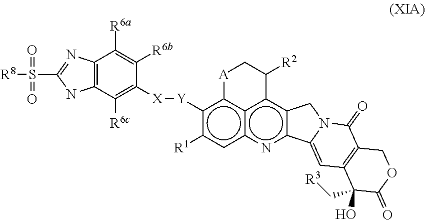
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

86. The compound of claim 78, having the structure of Formula (XIIA) 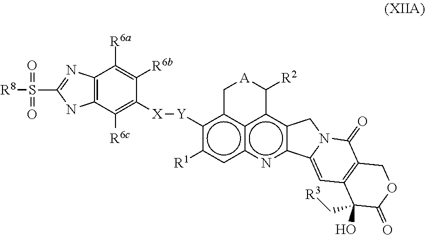
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

87. The compound of any one of claims 78 to 86, wherein R⁶, is hydrogen.

88. The compound of any one of claims 78 to 87, wherein R^{6b} is hydrogen.

89. The compound of any one of claims 78 to 87, wherein R^{6b} is halo.

90. The compound of any one of claims 78 to 87 and 89, wherein R^{6b} is fluoro.

91. The compound of any one of claims 78 to 90, wherein R^{6c} is hydrogen.

92. The compound of any one of claims 76 to 91, wherein A is O.

93. The compound of any one of claims 76 to 91, wherein A is S.

94. The compound of any one of claims 76 to 93, wherein R is hydrogen or halo.

95. The compound of any one of claims 76 to 94, wherein R¹ is hydrogen.

96. The compound of any one of claims 76 to 94, wherein R¹ is halo.

97. The compound of any one of claims 76 to 94 and 96, wherein R¹ is fluoro.

98. The compound of any one of claims 76 to 97, wherein R² is (i) hydrogen or deuterium; (ii) heterocyclyl, optionally substituted with one or more substituents Q; or (iii) —NR^{1b}R^{1c} or —NR^{1a}C(O)R^{1d}.

99. The compound of any one of claims 76 to 98, wherein R² is hydrogen, amino, methylamino, acetamido, or hydroxyacetamido.

100. The compound of any one of claims 76 to 99, wherein R² is hydrogen.

101. The compound of any one of claims 76 to 100, wherein R³ is C₁₋₆ alkyl, C₁₋₆ heteroalkyl, or C₂₋₆ alkenyl, each optionally substituted with one or more substituents Q.

102. The compound of any one of claims 76 to 101, wherein R³ is methyl, fluoromethyl, methoxymethyl, or ethenyl.

103. The compound of any one of claims 76 to 102, wherein R³ is methyl.

104. The compound of any one of claims 76 to 103, wherein X is C₁₋₄₀ heteroalkylene, wherein one or more methylene groups are each independently and optionally replaced by a divalent group and each divalent group is independently C₆₋₁₄ arylene, heteroarylene, or heterocyclylene; and wherein each heteroalkylene, arylene, heteroarylene, and heterocyclylene is optionally substituted with one or more substituents Q.

105. The compound of any one of claims 76 to 104, wherein X is: 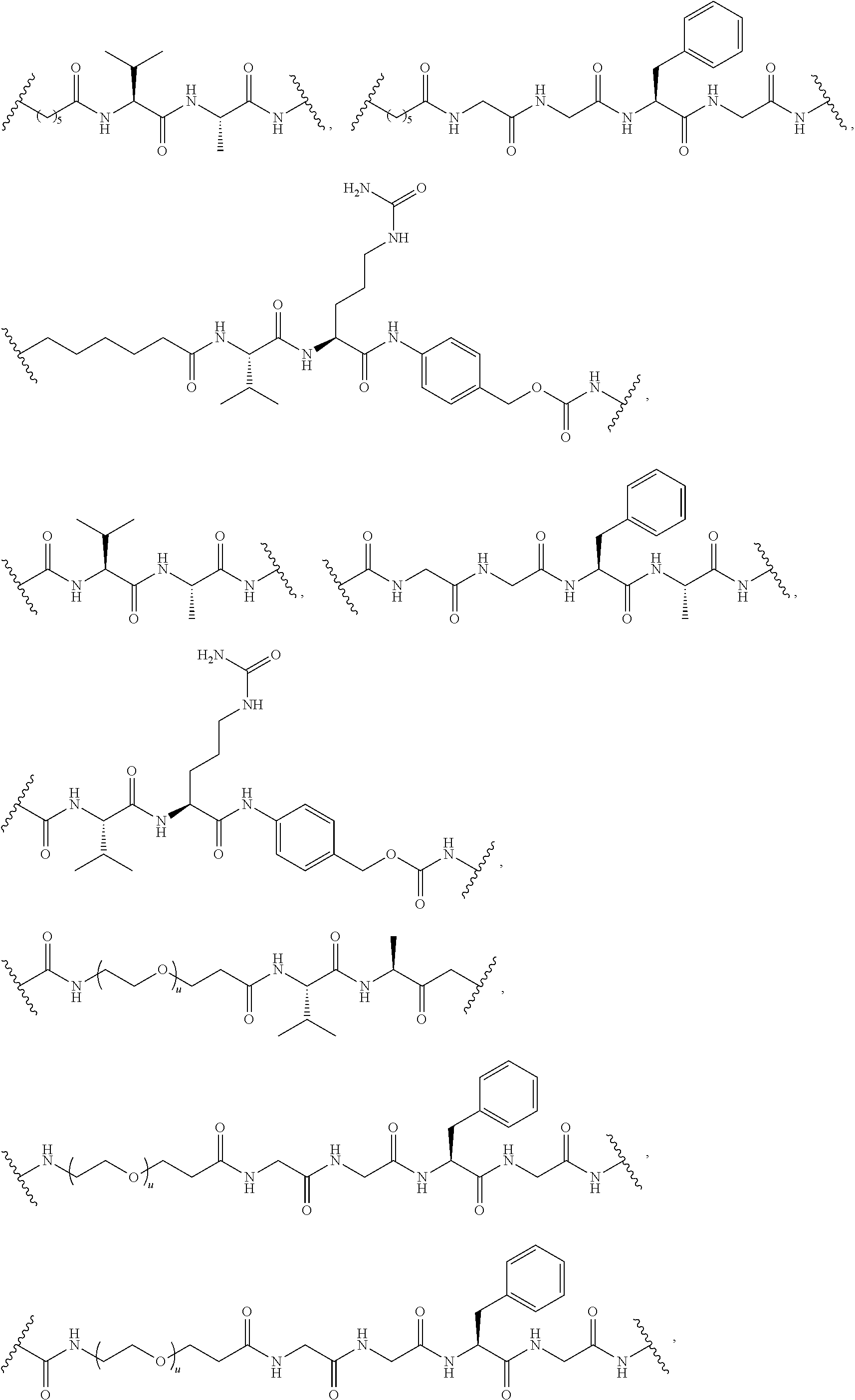 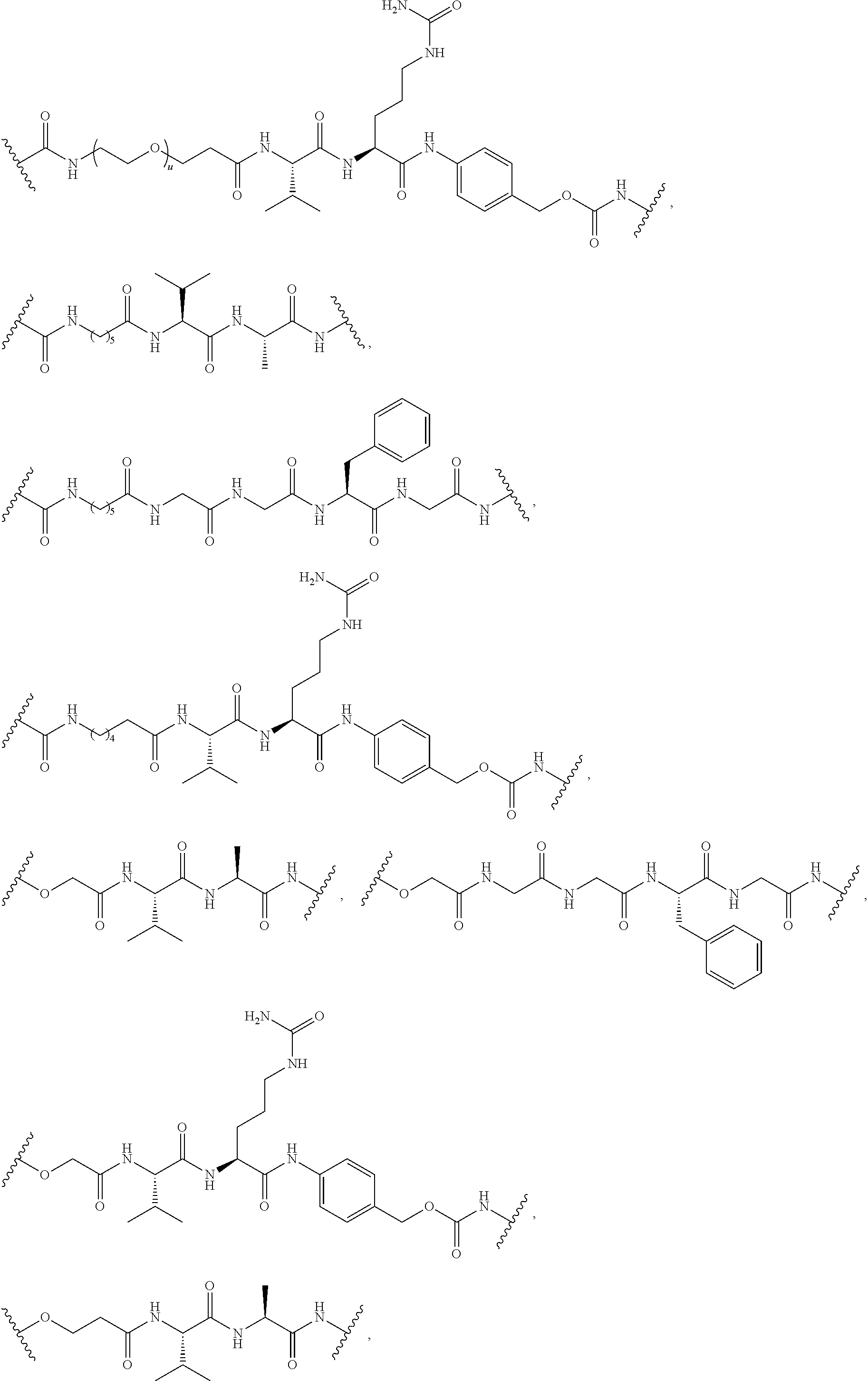 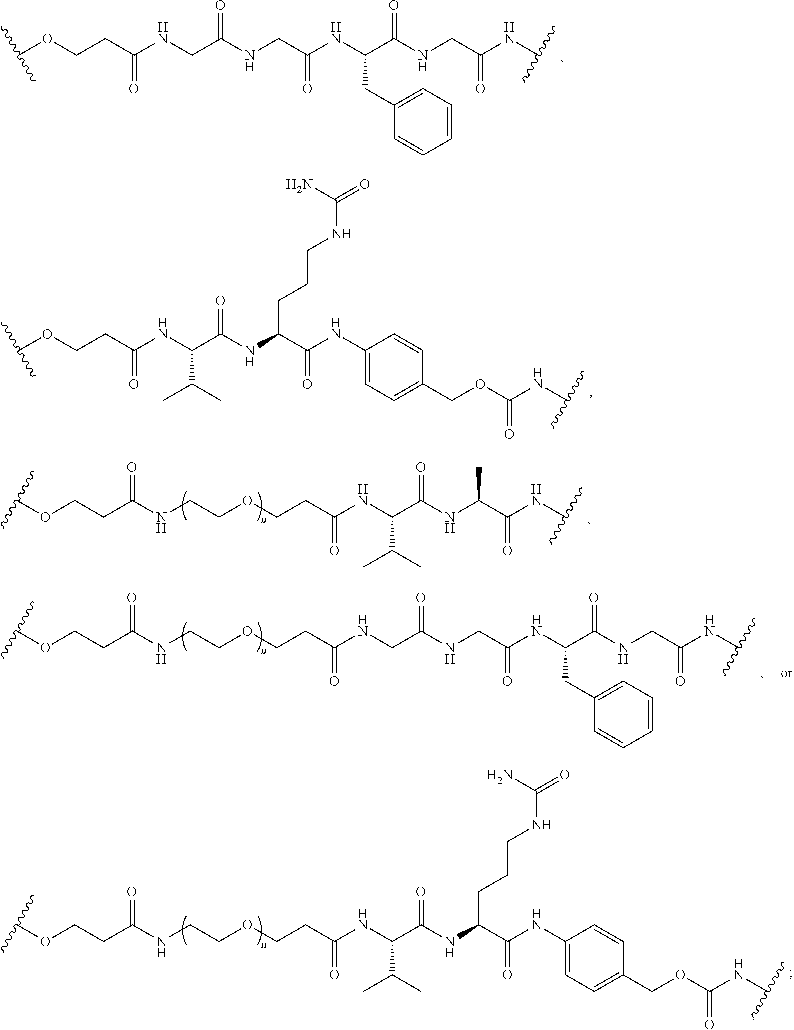
wherein each a is independently an integer of 1, 2, 3, 4, 5, 6, 7, or 8.

106. The compound of any one of claims 76 to 105, wherein Y is a bond.

107. The compound of claim 105 or 106, wherein each a is independently an integer of 4 or 8.

108. The compound of claim 76, wherein the compound is:
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-2-(methylsulfonyl) benzo [d]thiazole-6-carboxamide C1;
N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-2-(methylsulfonyl) benzo [d]-thiazole-6-carboxamide C2;
4-((S)-2-((S)-3-methyl-2-(2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano[4, 3, 2-de]pyrano [3′, 4′: 6, 7]indolizino [1,2-b]quinolin-4-yl) carbamate C3;
2-(cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl)-amino)-1-oxopropan-2-yl) amino)-3-methyl-1-oxobutan-2-yl) benzo [d]thiazole-6-carboxamide C4;
2-(cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxo-ethyl) benzo [d]thiazole-6-carboxamide C5;
4-((S)-2-((S)-2-(2-(cyclopropylsulfonyl) benzo [d]thiazole-6-carboxamido)-3-methyl-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C6;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-2-(methylsulfonyl) benzo [d]thiazole-6-carboxamide C7;
N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-2-(methyl-sulfonyl) benzo [d]thiazole-6-carboxamide C8;
4-((S)-2-((S)-3-methyl-2-(2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C9;
2-(cyclopropylsulfonyl)-N—((S)-1-(((S)-1-(((S)-9-ethyl-5-fluoro-9-hydroxy-10, 13-dioxo-1, 2, 9, 10, 13, 15-hexahydro-12H-pyrano[4, 3, 2-de]pyrano [3′, 4′: 6, 7]indolizino [1,2-b]quinolin-4-yl)-amino)-1-oxopropan-2-yl) amino)-3-methyl-1-oxobutan-2-yl) benzo [d]thiazole-6-carboxamide C10;
2-(cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl) benzo [d]thiazole-6-carboxamide C11;
4-((S)-2-((S)-2-(2-(cyclopropylsulfonyl) benzo [d]thiazole-6-carboxamido)-3-methyl-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C12;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-5-fluoro-2-(methylsulfonyl) benzo [d]-thiazole-6-carboxamide C13;
N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxoethyl) amino)-1-oxo-3-phenylpropan-2-yl) amino)-2-oxoethyl) amino)-2-oxoethyl)-5-fluoro-2-(methylsulfonyl)-benzo [d]thiazole-6-carboxamide C14;
4-((S)-2-((S)-3-methyl-2-(5-fluoro-2-(methylsulfonyl) benzo [d]thiazole-6-carboxamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C15;
2-(cyclopropylsulfonyl)-N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-5-fluorobenzo [d]thiazole-6-carboxamide C16;
2-(cyclopropylsulfonyl)-N-(2-((2-(((S)-1-((2-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl)-5-fluorobenzo [d]thiazole-6-carboxamide C17;
4-((S)-2-((S)-2-(2-(cyclopropylsulfonyl)-5-fluorobenzo [d]thiazole-6-carboxamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1, 2-b]-quinolin-4-yl) carbamate C18;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl)-5-fluoro-2-(methylsulfonyl) benzo [d]-thiazole-6-carboxamide C19;
N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-2-oxo-ethyl) amino)-1-oxo-3-phenylpropan-2-yl) amino)-2-oxoethyl) amino)-2-oxoethyl)-5-fluoro-2-(methylsulfonyl) benzo [d]thiazole-6-carboxamide C20;
4-((S)-2-((S)-3-methyl-2-(5-fluoro-2-(methylsulfonyl) benzo [d]thiazole-6-carboxamido)-butanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C21;
2-(cyclopropylsulfonyl)-N—((S)-1-(((S)-1-(((S)-9-ethyl-5-fluoro-9-hydroxy-10, 13-dioxo-1, 2, 9, 10, 13, 15-hexahydro-12H-pyrano[4, 3, 2-de]pyrano [3′, 4′: 6, 7]indolizino [1,2-b]-quinolin-4-yl) amino)-1-oxopropan-2-yl) amino)-3-methyl-1-oxobutan-2-yl)-5-fluorobenzo [d]-thiazole-6-carboxamide C22;
2-(cyclopropylsulfonyl)-N-(2-((2-(((S)-1-((2-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-2-oxoethyl) amino)-1-oxo-3-phenylpropan-2-yl) amino)-2-oxoethyl) amino)-2-oxoethyl)-5-fluorobenzo [d]thiazole-6-carboxamide C23;
4-((S)-2-((S)-2-(2-(cyclopropylsulfonyl)-5-fluorobenzo [d]thiazole-6-carboxamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C24;
(S)—N—((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(2-((2-(methylsulfonyl) benzo [d]thiazol-6-yl) oxy) acetamido) butanamide C25;
(S)—N-(2-(((S)-9-ethyl-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(2-((2-(methylsulfonyl) benzo [d]thiazol-6-yl) oxy) acetamido) acetamido) acetamido)-3-phenyl-propanamide C26;
4-((S)-2-((S)-3-methyl-2-(2-((2-(methylsulfonyl) benzo [d]thiazol-6-yl) oxy)-acetamido)-butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C27;
(S)—N—((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-3-methyl-2-(3-((2-(methylsulfonyl) benzo [d]thiazol-6-yl) oxy) propanamido) butanamide C28;
(S)—N-(2-(((S)-9-ethyl-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenyl-propanamide C29;
4-((S)-2-((S)-3-methyl-2-(3-((2-(methylsulfonyl) benzo [d]thiazol-6-yl) oxy)-propanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1, 2-b]quinolin-4-yl) carbamate C30;
N-((17S,20S)-21-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluoro-2-(methylsulfonyl)-benzo [d]thiazole-6-carboxamide C31;
N-((17S,20S)-21-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluoro-2-(methylsulfonyl) benzo [d]thiazole-6-carboxamide C32;
2-(cyclopropylsulfonyl)-N-((17S,20S)-21-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C33;
2-(cyclopropylsulfonyl)-N-((17S,20S)-21-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-17-isopropyl-20-methyl-15, 18, 21-trioxo-3, 6, 9, 12-tetraoxa-16, 19-diazahenicosyl)-5-fluorobenzo [d]thiazole-6-carboxamide C34;
N-((29S,32S)-33-(((S)-9-ethyl-9-hydroxy-10, 13-dioxo-1, 2, 9, 10, 13, 15-hexahydro-12H-pyrano [4, 3, 2-de]pyrano [3′, 4′: 6,7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C35;
N-((29S,32S)-33-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluoro-2-(methylsulfonyl) benzo [d]thiazole-6-carboxamide C36;
2-(cyclopropylsulfonyl)-N-((29S,32S)-33-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-29-isopropyl-32-methyl-27, 30, 33-trioxo-3, 6, 9, 12, 15, 18, 21, 24-octaoxa-28, 31-diaza-tritriacontyl)-5-fluorobenzo [d]thiazole-6-carboxamide C37;
2-(cyclopropylsulfonyl)-N-((29S,32S)-33-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-29-isopropyl-32-methyl-27, 30, 33-trioxo-3, 6, 9, 12, 15, 18, 21, 24-octaoxa-28, 31-diazatritriacontyl)-5-fluorobenzo [d]thiazole-6-carboxamide C38;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl)-1-(3-((2-(methylsulfonyl) benzo [d]thiazol-6-yl) oxy)-propanamido)-3, 6, 9, 12-tetraoxapentadecan-15-amide C39;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxo-propan-2-yl) amino)-3-methyl-1-oxobutan-2-yl)-1-(3-((2-(methylsulfonyl) benzo [d]thiazol-6-yl) oxy) propanamido)-3, 6, 9, 12-tetraoxapentadecan-15-amide C40;
N—((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl)-amino)-3-methyl-1-oxobutan-2-yl)-1-(3-((2-(methylsulfonyl) benzo [d]thiazol-6-yl) oxy)-propanamido)-3, 6, 9, 12, 15, 18, 21, 24-octaoxaheptacosan-27-amide C41;
N—((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxo-propan-2-yl) amino)-3-methyl-1-oxobutan-2-yl)-1-(3-((2-(methylsulfonyl) benzo [d]thiazol-6-yl)-oxy) propanamido)-3, 6, 9, 12, 15, 18, 21, 24-octaoxaheptacosan-27-amide C42;
N-(6-(((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1-oxopropan-2-yl) amino)-3-methyl-1-oxobutan-2-yl) amino)-6-oxohexyl)-2-(methylsulfonyl)-benzo [d]thiazole-6-carboxamide C43;
N—((S)-5-benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(methylsulfonyl) benzo [d]thiazole-6-carboxamide C44;
4-((S)-2-((S)-3-methyl-2-(6-(2-(methylsulfonyl) benzo [d]thiazole-6-carboxamido)-hexanamido) butanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) carbamate C45;
2-(cyclopropylsulfonyl)-N-(6-(((S)-1-(((S)-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) amino)-6-oxohexyl)-benzo [d]thiazole-6-carboxamide C46;
N—((S)-5-benzyl-1-(((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl) benzo [d]-thiazole-6-carboxamide C47;
4-((S)-2-((S)-2-(6-(2-(cyclopropylsulfonyl) benzo [d]thiazole-6-carboxamido)-hexanamido)-3-methylbutanamido)-5-ureidopentanamido) benzyl ((S)-9-ethyl-5-fluoro-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]quinolin-4-yl) carbamate C48;
2-(cyclopropylsulfonyl)-N-(6-(((S)-1-(((S)-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]-quinolin-4-yl) amino)-1-oxopropan-2-yl) amino)-3-methyl-1-oxobutan-2-yl) amino)-6-oxohexyl)-5-fluoro-benzo [d]thiazole-6-carboxamide C49;
N—((S)-5-benzyl-1-(((S)-9-ethyl-9-hydroxy-10, 13-dioxo-1, 2, 9, 10, 13, 15-hexa-hydro-12H-pyrano [4, 3, 2-de]pyrano [3′, 4′: 6, 7]indolizino [1,2-b]quinolin-4-yl) amino)-1, 4, 7, 10, 13-pentaoxo-3, 6, 9, 12-tetraazaoctadecan-18-yl)-2-(cyclopropylsulfonyl)-5-fluorobenzo [d]thiazole-6-carboxamide C50;
4-((S)-2-((S)-2-(6-(2-(cyclopropylsulfonyl)-5-fluorobenzo [d]thiazole-6-carboxamido)-hexanamido)-3-methylbutanamido)-5-ureidopentanamido) benzyl ((S)-9-ethyl-9-hydroxy-10, 13-dioxo-1, 2, 9, 10, 13, 15-hexahydro-12H-pyrano [4, 3, 2-de]pyrano [3′, 4′: 6, 7]-indolizino [1,2-b]-quinolin-4-yl) carbamate C51;
(S)—N-(2-(((S)-9-ethyl-5-fluoro-9-hydroxy-10, 13-dioxo-1, 2, 9, 10, 13, 15-hexahydro-12H-pyrano-[4, 3, 2-de]pyrano [3′, 4′: 6, 7]indolizino [1,2-b]quinolin-4-yl) amino)-2-oxoethyl)-2-(2-(2-(3-((2-(methylsulfonyl) benzo [d]thiazol-6-yl) oxy) propanamido) acetamido) acetamido)-3-phenyl-propanamide C52; or
N—((S)-5-benzyl-1-(((S)-9-ethyl-9-hydroxy-10, 13-dioxo-1, 2, 9, 10, 13, 15-hexahydro-12H-pyrano [4, 3, 2-de]pyrano [3′, 4′: 6, 7]indolizino [1,2-b]quinolin-4-yl) amino)-1, 4, 7, 10, 13-pentaoxo-16, 19, 22, 25-tetraoxa-3, 6, 9, 12-tetraazaheptacosan-27-yl)-2-(methylsulfonyl) benzo [d]thiazole-6-carboxamide C53;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.
